(19) United States
(12) Patent Application Publication
(10) Pub. No.: US 2025/0177450 A1
(43) Pub. Date: Jun. 5, 2025

(54) METHODS FOR ADMINISTERING FREEZE-DRIED PLATELET DERIVATIVE COMPOSITIONS TO SUBJECTS TREATED WITH A FACTOR XI INHIBITOR

(71) Applicant: Cellphire, Inc., Rockville, MD (US)

(72) Inventors: Keith Andrew MOSKOWITZ, Westfield, IN (US); Braden Carl ISHLER, Montgomery Village, MD (US); William Matthew DICKERSON, Washington, DC (US); Amber Nicole LEE, Montgomery Village, MD (US); Shan XU, Rockville, MD (US); Stephen Edward AMOS, Buckeystown, MD (US); Rafael JORDA, Merignac (FR); Michael Alexander MATHEWS, Arlington, VA (US)

(73) Assignee: Cellphire, Inc., Rockville, MD (US)

(21) Appl. No.: 19/053,371

(22) Filed: Feb. 13, 2025

Related U.S. Application Data
(63) Continuation-in-part of application No. 17/674,831, filed on Feb. 17, 2022.
(60) Provisional application No. 63/264,227, filed on Nov. 17, 2021.
(60) Provisional application No. 63/276,420, filed on Nov. 5, 2021.
(60) Provisional application No. 63/275,937, filed on Nov. 4, 2021.
(60) Provisional application No. 63/150,334, filed on Feb. 17, 2021.

(51) Int. Cl. A61K 35/19; A61K 9/00; A61K 31/4365; A61K 31/519; A61K 31/616; A61K 31/7076; A61P 7/04

(52) U.S. Cl. CPC A61K 35/19; A61K 9/0019; A61K 31/4365; A61K 31/519; A61K 31/616; A61K 31/7076; A61P 7/04

(57) ABSTRACT

Provided herein are compositions comprising platelet derivatives, and methods for administering the same to a subject in need thereof. Also provided herein are methods and compositions for reducing bleeding in a subject. Such methods can include administering to the subject in need thereof, for example because they have been or are being treated with an anticoagulant agent, for example, a factor XI inhibitor, and/or an antiplatelet, a dose of rehydrated platelet derivative composition comprising platelet derivatives. Various properties of exemplary embodiments of such methods and platelet derivatives used therein, as well as numerous additional aspects and embodiments are provided herein.

### CROSS-REFERENCE TO RELATED APPLICATIONS

This application is a Continuation-In-Part of U.S. patent application Ser. No. 17/674,831, filed on Feb. 17, 2022. U.S. patent application Ser. No. 17/674,831 claims priority to U.S. Provisional Application Ser. No. 63/150,334, filed on Feb. 17, 2021, U.S. Provisional Application Ser. No. 63/275,937, filed on Nov. 4, 2021, U.S. Provisional Application Ser. No. 63/276,420, filed on Nov. 5, 2021, and U.S. Provisional Application Ser. No. 63/264,227, filed on Nov. 17, 2021. The content of each of the applications listed above is incorporated herein by reference in its entirety.

### FIELD OF THE INVENTION

This disclosure relates to the use of platelet derivatives as a treatment for anti-thrombotic agent-induced coagulopathy. The use of anticoagulant agents, and/or antiplatelet agents can result in increased, or uncontrolled bleeding in the subject.

### BACKGROUND

Anticoagulant drugs (also herein called anticoagulant agents) are common in the U.S. adult population and employ multiple mechanisms of inhibiting clotting of blood. Anticoagulant drugs are used to treat and/or prevent a number of cerebrovascular and cardiovascular diseases. Antiplatelet drugs (also herein called antiplatelet agents) are common in the U.S. adult population and employ multiple mechanisms of inhibiting platelet action. Antiplatelet drugs are used to treat and/or prevent a number of cerebrovascular and cardiovascular diseases.

Anticoagulant drugs/agents, and antiplatelet drugs/agents, however, are responsible for many adverse drug-related events (ADEs). Overdose and adverse events related to these drugs carry the risk of serious bleeding and related complications in the patient population. In addition, subjects treated with anticoagulant drugs, and/or antiplatelet drugs face additional complications for surgery, as a subject may need to be tapered off the drugs before surgery, though cessation of therapy could put the subject at an increased risk for heart attack, stroke, or death.

There is therefore a need in the art for the treatment of coagulopathy, such as anticoagulant agent-induced coagulopathy, and/or antiplatelet agent-induced coagulopathy as well as a need for a solution for preparing subjects taking an anticoagulant drug, and/or antiplatelet drug for surgery.

### SUMMARY OF THE INVENTION

Accordingly, the use of anti-thrombotic agents (i.e. antiplatelet agents and/or anti-coagulants) can result in increased bleeding potential of a subject. Here we demonstrate that platelet derivatives can circumvent or overcome this inhibition to restore hemostasis. Accordingly, provided herein are platelet derivatives, in illustrative embodiment freeze-dried platelet derivative (FDPD) and compositions comprising the same, that can reduce this increased bleeding potential of a subject, and in certain illustrative embodiments, circumvent or overcome this inhibition of clotting by such anti-thrombotic agents, to restore hemostasis.

Provided herein in some aspects or embodiments are methods of treating a coagulopathy in a subject, the method including administering to the subject in need thereof an effective amount of a composition including platelets, or in illustrative embodiments platelet derivatives, and in further illustrative embodiments FDPDs. The subject can be in need thereof because, for example, they were administered an anti-coagulant agent, and/or an antiplatelet agent. Various properties of exemplary embodiments of such FDPDs are provided herein.

Provided herein in one aspect is a method for administering platelet derivatives to a subject, the method comprising:
rehydrating a composition in the form of a powder, said composition comprising the platelet derivatives and an incubating agent comprising one or more saccharides, one or more salts, and a buffer to form a rehydrated platelet derivative composition, and
administering a dose of the rehydrated platelet derivative composition to the subject, wherein the dose comprises a first dose of the rehydrated platelet derivatives,
   wherein the subject has been treated or is being treated with aspirin, at least one anticoagulant agent, and at least one other antiplatelet agent, and
   wherein the at least one anticoagulant agent comprises a Factor XI inhibitor.

Further details regarding aspects and embodiments of the present disclosure are provided throughout this patent application. The preceding paragraphs in this Summary section is not an exhaustive list of aspects and embodiments disclosed herein. Sections and section headers are for ease of reading and are not intended to limit combinations of disclosure, such as methods, compositions, and kits or functional elements therein across sections.

### BRIEF DESCRIPTION OF THE DRAWINGS

FIG. 1 shows peak thrombin generation obtained by adding 400×10³/μL FDPDs to warfarin plasma at various INR levels.

FIG. 2 shows endogenous thrombin potential (ETP) values obtained by adding 400×10³/μL FDPDs to plasma at various INR levels.

FIG. 3 shows peak thrombin generation by FDPDs and by fresh platelets in INR 2 warfarin plasma.

FIG. 4 shows the effect on r-time of warfarin plasma samples in a TEG assay as a result of the addition of 300×10³/μL FDPDs.

FIG. 5 shows that FDPDs provide a dose-dependent increase in peak thrombin generation. These data were collected in the background of whole blood with an endogenous platelet count of 150×10³/μL.

FIG. 6 shows a plot of the concentration of platelets or FDPDs versus peak thrombin generation.

FIG. 7A shows a plot of the concentration of platelets, FDPDs, or a combination thereof versus peak thrombin generation in INR-2 plasma.

FIG. 7B shows thrombin generation in INR-1 plasma, INR-2 plasma (treated with warfarin), and INR-2 plasma (treated with warfarin) plus FDPDs (150×10³/μL), for four different batches of FDPDs.

FIG. 8 shows the generation of thrombus by FDPDs in warfarin plasma in a shear-dependent collagen adhesion assay under flow (T-TAS®)

FIG. 9 shows a plot of the time to generation of thrombus increasing with increasing concentrations of rivaroxaban in whole blood (WB).

FIG. 10A shows a plot of the time to generation of thrombus in the presence of 3 μM rivaroxaban decreasing with the addition of FDPDs.

FIG. 10B shows a plot of the time to generation of thrombus in control plasma, in plasma treated with 3 μM rivaroxaban, and in plasma treated with 3 μM rivaroxaban and 300×10³/μL FDPDs.

FIG. 10C shows a plot of the time to generation of occlusion of T-TAS® AR chip from FIG. 10B.

FIG. 11A shows the effect of FDPDs in warfarin plasma (INR=1.6) compared to standard plasma (INR=1.0), measured in terms of R-time (start of clot formation).

FIG. 11B shows the effect of FDPDs in warfarin plasma (INR=1.6) compared to standard plasma (INR=1.0), measured in terms of R-time, plotted on a log-scale x-axis.

FIG. 12A shows the effect of FDPDs in warfarin plasma (INR=1.6) in terms of alpha angle (also called angle).

FIG. 12B shows the effect of FDPDs in warfarin plasma (INR=1.6) in terms of alpha angle (also called angle), plotted on a log-scale x-axis.

FIG. 13 shows the effect of FDPDs in warfarin plasma (INR=1.6) in terms of maximum amplitude (MA).

FIG. 14 shows the effect of FDPDs in warfarin plasma (INR=1.6) in terms of maximum amplitude (MA), plotted on a log-scale x-axis.

FIG. 15 shows a plot of the decrease in lag time for samples with different INR values supplemented FDPDs.

FIG. 16 is an exemplary thromboelastography (TEG) waveform with parameters labeled.

FIG. 17 is a plot of R-time for various INR values of warfarin plasma, with or without supplementation with various concentrations of FDPDs.

FIG. 18 is a plot of activated clotting time in plasma levels of various INR levels, with and without supplemented FDPDs.

FIG. 19 shows the effect of FDPDs on whole blood (normal; INR=2; INR=3; and INR=6.2)

FIG. 20A shows the effect on peak thrombin generation of FDPDs in plasma with INRs of 1 and 2.

FIG. 20B shows the effect on peak thrombin generation of FDPDs in plasma with an INR of 3.

FIG. 20C shows the effect on peak thrombin generation of FDPDs in plasma with INRs of 1 and 6.

FIG. 21A shows the effect on endogenous thrombin potential of FDPDs in plasma with INRs of 1 and 2.

FIG. 21B shows the effect on endogenous thrombin potential of FDPDs in plasma with an INR of 3.

FIG. 21C shows the effect on endogenous thrombin potential of FDPDs in plasma with INRs of 1 and 6.

FIG. 22A shows the effect on peak thrombin generation of FDPDs in plasma with INRs of 1, 2, 3, and 6 (left) and a zoomed-in image of the same data from 0 to 30 nM (right) for a replicate of FDPDs batch 1.

FIG. 22B shows the effect on peak thrombin generation of FDPDs in plasma with INRs of 1, 2, 3, and 6 for a replicate of FDPDs batch 1.

FIG. 22C shows the effect on peak thrombin generation of FDPDs in plasma with INRs of 1, 2, 3, and 6 for a replicate of FDPDs batch 1.

FIG. 22D shows the effect on peak thrombin generation of FDPDs in plasma with INRs of 1, 2, 3, and 6 (left) and a zoomed-in image of the same data from 0 to 2.5 nM (right) for FDPDs batch 2.

FIG. 22E shows the effect on peak thrombin generation of FDPDs in plasma with INRs of 1, 2, and 3 for FDPDs batch 3.

FIG. 23A shows aPTT values for plasma and plasma treated with heparin.

FIG. 23B shows thrombin generation for plasma treated with heparin, with the addition of fresh platelets or FDPDs initiated with PPP low reagent.

FIG. 23C shows thrombin generation for plasma treated with heparin, with the addition of fresh platelets or FDPDs initiated with PRP reagent.

FIG. 24A shows aPTT values for plasma, plasma treated with heparin, and plasma treated with heparin and protamine sulfate.

FIG. 24B shows thrombin generation for plasma treated with heparin and FDPDs, without (relatively flat lines) or with (curves) addition of protamine sulfate, initiated with PPP low reagent.

FIG. 24C shows thrombin generation for plasma treated with heparin and FDPDs, without (relatively flat lines) or with (curves) addition of protamine sulfate, initiated with PRP reagent.

FIG. 25A shows thrombin generation for control plasma, plasma treated with dabigatran, or plasma treated with dabigatran and FDPDs initiated with PRP reagent.

FIG. 25B shows the time to peak (TTP) in a thrombin generation assay for control plasma, plasma treated with dabigatran, or plasma treated with dabigatran and FDPDs initiated with PRP reagent.

FIG. 26A shows the effect of cangrelor and ticagrelor on the occlusion time of Platelet Rich Plasma (PRP) with and without FDPD

FIG. 26B shows the effect of cangrelor and ticagrelor on the occlusion time of Platelet Rich Plasma (PRP) with and without FDPD

FIG. 27 shows the tail snip test for depicting the ability of FDPDs to restore bleeding time in NOD-SCID mice treated with supra-pharmacologic clopidogrel.

FIG. 28 shows the ability of FDPDs to restore bleeding time in New Zealand White Rabbits treated with supra-pharmacologic clopidogrel. Each line represents a different rabbit dosed under identical conditions and with the same dose.

FIG. 29A shows that FDPDs are capable of catalyzing thrombin generation in the presence of 25 ng/ml rivaroxaban.

FIG. 29B shows that FDPDs are capable of partially recovering the endogenous thrombin potential in the presence of 25 ng/ml rivaroxaban.

FIG. 30 shows the occlusion time was partially restored with the addition of FDPDs into rivaroxaban treated whole blood.

FIG. 31A shows the activator thrombin potential by FDPDs in rivaroxaban treated Octaplas.

FIG. 31B shows the maximum thrombin concentration by FDPDs in rivaroxaban treated Octaplas.

FIG. 31C shows the ATG lag time by FDPDs in rivaroxaban treated Octaplas.

FIG. 32A shows the activator thrombin potential by FDPDs in rivaroxaban treated fresh Platelet Rich Plasma (PRP).

FIG. 32B shows the maximum thrombin concentration by FDPDs in rivaroxaban treated fresh Platelet Rich Plasma (PRP).

FIG. 32 C shows the ATG lag time by FDPDs in rivaroxaban treated Platelet Rich Plasma (PRP).

FIG. 33 shows the effect of FDPDs on time to clot in the presence of anticoagulants using the ACT test.

FIG. 34A shows the effect of 0.1 U heparin on thrombin generation, in pooled normal plasma, comparing apheresis units (APU) with FDPDs at 5K and 50K platelets per μL.

FIG. 34B shows the impact of 0.8 U/mL heparin reversed by 4 μg/ml protamine (½ of the recommended reversal doses) with FDPDs at 10K and 50K platelets per μL.

FIG. 34C shows peak height of thrombin generation of samples which were treated with heparin and protamine as described on the x-axis.

FIG. 35 shows the occlusion time with aspirin treatment in the presence of FDPDs versus PRP alone.

FIG. 36 shows the occlusion time with both ticagrelor and aspirin in the presence of FDPDs versus PRP alone.

FIG. 37A shows the aggregation response of FDPDs in the presence of agonists, but in the absence of fresh platelets.

FIG. 37 B shows the aggregation response of platelet-rich plasma (PRP) in the presence of agonists, but in the absence of fresh platelets.

FIG. 37 C shows the comparison of aggregation of FDPDs and PRP in the presence of 20 μM ADP.

FIG. 37D shows the comparison of aggregation of FDPDs and PRP in the presence of 10 μg/ml collagen.

FIG. 37E shows the comparison of aggregation of FDPDs and PRP in the presence of 300 μM epinephrine.

FIG. 37F shows the comparison of aggregation of FDPDs and PRP in the presence of 1 mg/ml ristocetin.

FIG. 37G shows the comparison of aggregation of FDPDs and PRP in the presence of 10 μM TRAP-6.

FIG. 37H shows the comparison of aggregation of FDPDs and PRP in the presence of 5 mg/ml arachidonic acid.

FIG. 38A shows that TRAP-6 peptide is capable of promoting platelet activation by observing expression of CD62P on the apheresis platelets.

FIG. 38B shows that TRAP-6 peptide is not able to increase the expression of CD62P on FDPDs.

FIG. 39 shows the measurement of thrombospondin (TSP-1) by flow cytometry in terms of mean fluorescent intensity (MFI) in resting fresh platelets, activated fresh platelets, and different lots of FDPDs.

FIG. 40 shows the measurement of von Willebrand factor (vWF) by flow cytometry in terms of mean fluorescent intensity (MFI) in resting fresh platelets, activated fresh platelets, and different lots of FDPDs.

FIG. 41 shows the forward scatter (FSC) measured by flow cytometry of apheresis platelets, and FDPDs.

FIG. 42 shows exemplary flow cytometry data of FDPDs unstained (dark data points) or stained (light data points) with an anti-CD-41 antibody.

FIG. 43 shows an exemplary histogram of FDPDs incubated with annexin V with (light data points) and without (dark data points) calcium.

FIG. 44 shows an exemplary histogram of FDPDs incubated with an anti-CD62 antibody (light data points) or with an isotype control (dark data points).

FIG. 45 shows a plot of thrombin peak height for FDPDs in the presence of PRP Reagent containing tissue factor and phospholipids (solid line and long dashes) and control cephalin (dots).

FIG. 46A is a plot of the percent occupancy of particles of different radii in human in-date stored platelets (Batch J) and platelet derivatives (pre-lyophilization) derived therefrom as determined by dynamic light scattering (DLS).

FIG. 46B is a plot of the percent occupancy of particles of different radii in human in-date stored platelets (Batch K) and platelet derivatives (pre-lyophilization) derived therefrom as determined by DLS.

FIG. 46C is a plot of the percent occupancy of particles of different radii in human in-date stored platelets (Batch L) and platelet derivatives (pre-lyophilization) derived therefrom as determined by DLS.

FIG. 47A is a plot of the percent occupancy of particles of different radii in human in-date stored platelets (Batch D) and platelet derivatives (pre-lyophilization) derived therefrom as determined by DLS.

FIG. 47B is a plot of the percent occupancy of particles of different radii in human in-date stored platelets (Batch E) and platelet derivatives (pre-lyophilization) derived therefrom as determined by DLS.

FIG. 47C is a plot of the percent occupancy of particles of different radii in human in-date stored platelets (Batch F) and platelet derivatives (pre-lyophilization) derived therefrom as determined by DLS.

FIG. 48 shows an exemplary histogram comparison of low-plasma FDPDs unstained (black) or stained with an isotype control antibody (dark gray) or a FITC-labeled 9F9 antibody (light gray), and a table showing the mean fluorescence intensity for two replicates.

FIG. 49 shows an exemplary histogram comparison of low-plasma FDPDs unstained (black) or stained with an anti-PAC-1 antibody (light gray), and a table showing the mean fluorescence intensity for two replicates.

FIG. 50A shows an exemplary schematic of a pathogen reduction system.

FIG. 50B shows a plot of the weight of a reaction vessel over time.

FIG. 50C shows a plot of pressure in a reaction vessel over time.

FIG. 51A shows a plot of the percent occupancy of particles of different radii in rehydrated thrombosomes that were (Batch N) or were not (Batch M) treated to remove pathogens, as determined by DLS.

FIG. 51B shows a plot of the percent occupancy of particles of different radii in rehydrated thrombosomes that were (Batch K) or were not (Batch J) treated to remove pathogens, as determined by DLS.

FIG. 52A shows a plot of the percent occupancy of particles of different radii in hIDSPs that were (Batch N) or were not (Batch M) treated to remove pathogens, as determined by DLS.

FIG. 52B shows a plot of the percent occupancy of particles of different radii in hIDSPs and thrombosomes derived therefrom (Batch M) that were not treated to remove pathogens, as determined by DLS.

FIG. 52C shows a plot of the percent occupancy of particles of different radii in hIDSPs and thrombosomes derived therefrom (Batch N) that were treated to remove pathogens, as determined by DLS.

FIG. 53A shows a plot of the percent occupancy of particles of different radii in hIDSPs that were (Batch K) or were not (Batch J) treated to remove pathogens, as determined by DLS.

FIG. 53B shows a plot of the percent occupancy of particles of different radii in hIDSPs and thrombosomes derived therefrom (Batch J) that were not treated to remove pathogens, as determined by DLS.

FIG. 53C shows a plot of the percent occupancy of particles of different radii in hIDSPs and thrombosomes derived therefrom (Batch K) that were treated to remove pathogens, as determined by DLS.

FIG. 54A shows a plot of thrombosome adhesion under shear in whole blood.

FIG. 54B shows a plot of thrombosome adhesion under shear in plasma.

FIG. 54C shows formation of fibrin in a microcapillary channel in the absence of GPRP.

FIG. 54D shows a lack of formation of fibrin in a microcapillary channel in the presence of GPRP.

FIG. 54E shows a plot of the effect of GPRP on thrombosome adhesion under shear in plasma.

FIG. 55 shows the activated partial thromboplastin time (aPTT) activity of anti-FXI antibody and IgG control.

FIG. 56 shows the peak thrombin activity of FDPDs (FPH) in the presence of FXI inhibition (anti-FXI antibody), and IgG control.

FIG. 57 shows the thromboelastography (TEG) activity of FDPDs (FPH) in the presence of FXI inhibition (anti-FXI antibody), and IgG control.

FIG. 58 shows the peak thrombin activity of FDPDs (FPH) in the presence of samples treated with a combination of dual anti-platelet therapy (DAPT) and anti-Factor XI antibody.

FIG. 59 shows the lag time, peak thrombin and time to peak formation of plasma samples with antibody to factor VIII, and with or without the addition of FPH.

FIG. 60 shows the lag time, peak thrombin and time to peak formation of plasma samples with antibody to factor IX, and with or without the addition of FPH.

FIG. 61 shows the lag time, peak thrombin and time to peak formation of normal human plasma (n=3), factor VIII depleted human plasma, and factor VIII depleted human plasma with the addition of 40,000 particles/μL of FPH.

FIG. 62 shows the lag time, peak thrombin and time to peak formation of normal human plasma (n=3), factor IX depleted human plasma, and factor IX depleted human plasma with the addition of 40,000 particles/μL of FPH.

FIG. 63 shows the R-time of increasing amounts of anti-factor VIII antibody with and without 50,000 particles/μL of FPH.

FIG. 64 shows the R-time of increasing amounts of anti-factor IX antibody with and without 50,000 particles/μL of FPH.

### DETAILED DESCRIPTION

Before embodiments of the present invention are described in detail, it is to be understood that the terminology used herein is for the purpose of describing particular embodiments only, and is not intended to be limiting. Unless defined otherwise, all technical and scientific terms used herein have the same meaning as commonly understood by one of ordinary skill in the art to which the term belongs. Although any methods and materials similar or equivalent to those described herein can be used in the practice of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a saccharide” includes reference to one or more saccharides, and equivalents thereof known to those skilled in the art. Furthermore, the use of terms that can be described using equivalent terms include the use of those equivalent terms. Thus, for example, the use of the term “subject” is to be understood to include the terms “patient”, “person”, “animal”, “human”, and other terms used in the art to indicate one who is subject to a medical treatment. The use of multiple terms to encompass a single concept is not to be construed as limiting the concept to only those terms used.

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used herein, the term “coagulopathy” means any derangement of hemostasis resulting in either excessive bleeding or clotting. Coagulopathies caused by administration of an antiplatelet agent and/or anti-coagulant agent to a subject typically includes an increased bleeding potential, or increased bleeding. Thus, methods herein for treating a coagulopathy in illustrative embodiments, are methods for decreasing the bleeding potential of a subject, or decreasing the bleeding in the subject.

As used herein, the term “platelet” can include whole platelets, fragmented platelets, platelet derivatives, or FDPDs. “Platelets” within the above definition may include, for example, platelets in whole blood, platelets in plasma, platelets in buffer optionally supplemented with select plasma proteins, cold stored platelets, dried platelets, cryopreserved platelets, thawed cryopreserved platelets, rehydrated dried platelets, rehydrated cryopreserved platelets, lyopreserved platelets, thawed lyopreserved platelets, or rehydrated lyopreserved platelets. “Platelets” may be “platelets” of mammals, such as of humans, or such as of non-human mammals. As used herein, “preparation agent” can include any appropriate components. In some embodiments, the preparation agent may comprise a liquid medium. In some embodiments the preparation agent may comprise one or more salts selected from phosphate salts, sodium salts, potassium salts, calcium salts, magnesium salts, and any other salt that can be found in blood or blood products, or that is known to be useful in drying platelets, or any combination of two or more of these. In some embodiments, the preparation agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the preparation agent includes from about 0.5 mM to about 100 mM of the one or more salts. In some embodiments, the preparation agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) of the one or more salts. In some embodiments, the preparation agent includes about 5 mM, about 75 mM, or about 80 mM of the one or more salts. In some embodiments, the preparation agent comprises one or more salts selected from calcium salts, magnesium salts, and a combination of the two, in a concentration of about 0.5 mM to about 2 mM.

As used herein, “thrombosomes” (sometimes also herein called “Tsomes” or “Ts”, particularly in the Examples and Figures) are platelet derivatives that have been treated with an incubating agent (e.g., any of the incubating agents described herein) and lyopreserved (i.e. freeze-dried). Thus, thrombosomes are illustrative or target freeze-dried platelet derivatives (FDPDs). Illustrative or target freeze-dried platelet derivative compositions herein (e.g. thrombosomes) typically have at least 1 hemostatic property, and thus can function as hemostatic agents and can be referred to hemostat(s) or hemostatic product(s). Illustrative or target FDPDs and compositions herein comprising the same that have at least 1 hemostatic property can also be referred to as freeze-dried platelet derived hemostat(s) or freeze-dried platelet hemostat(s) (both of which can be abbreviated FDPDH, FDPH or FPH). In some cases, illustrative or target FDPDs such as thrombosomes can be prepared from pooled platelets. In some cases, FDPDs can be prepared from pooled platelets. FDPDs can have a shelf life of 2-3 years in dry form at ambient temperature and can be rehydrated with sterile water within minutes (e.g. 1, 2, 3, 4, 5, 10 15, 20, 25, or 30 minutes) for immediate infusion. One example of FDPDs are THROMBOSOMES®, which are in clinical trials for the treatment of acute hemorrhage in thrombocytopenia patients and are a product of Cellphire, Inc. In non-limiting illustrative embodiments, FDPD compositions, illustrative freeze-dried platelet-derivative (i.e. “FDPD”) compositions, or FPH herein, such as those prepared according to Example 18 herein, are compositions that include a population of platelet derivatives having a reduced propensity to aggregate such that no more than 10% of the platelet derivatives in the population aggregate under aggregation conditions comprising an agonist but no platelets, and wherein the platelet derivatives have a potency of at least 1.5 thrombin generation potency units (tgpu) per 10⁶ platelet derivatives. In non-limiting illustrative embodiments, FDPD compositions, illustrative FDPD compositions herein, or FPH herein, such as those prepared according to Example 18 herein, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are between 0.5 and 2.5 μm in diameter. Furthermore, such illustrative target platelet derivatives, or FPH typically have the ability to generate thrombin in an in vitro thrombin generation assay and/or have the ability to occlude a collagen-coated microchannel in vitro.

As used herein, an “anticoagulant” is an antithrombotic that does not include antiplatelet agents. Typically, agents that inhibit Factor IIa, VIIa, IX, Xa, XI, XIa Tissue Factor, or vitamin K-dependent synthesis of clotting factors (e.g., Factor II, VII, IX, or X) or that activate antithrombin (e.g., antithrombin III) are considered to be anticoagulants. Other mechanisms of anticoagulants are known. Non-limiting examples of anticoagulants include dabigatran, argatroban, hirudin, rivaroxaban, apixaban, edoxaban, fondaparinux, warfarin, heparin, and low molecular weight heparins (e.g., dalteparin, enoxaparin, tinzaparin, ardeparin, nadroparin, reveparin, danaparoid). Additional non-limiting examples of anticoagulants include tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, and fluindione. In some embodiments, the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, and fluindione. In some cases, an anticoagulant agent can be an agent that inhibits factor VIII in a subject. In some cases, an anticoagulant agent can be an agent that inhibits factor IX in a subject. In some cases, an anticoagulant agent can be an agent that inhibits factor XI, or XIa in a subject.

As used herein, an “antiplatelet agent” is an antithrombotic and does not include anticoagulants. Typically, agents that inhibit P2Y receptors (e.g., P2Y12), glycoprotein IIb/IIIa (I.e. CD41), or that antagonize thromboxane synthase or thromboxane receptors, are considered to be antiplatelet agents. Other mechanisms of antiplatelet agents are known. As used herein, aspirin is considered to be an antiplatelet agent but not an anticoagulant. Examples of antiplatelet agents include aspirin (also called acetylsalicylic acid or ASA), cangrelor (e.g., KENGREAL®), ticagrelor (e.g., BRILINTA®), clopidogrel (e.g., PLAVIX®), prasugrel (e.g., EFFIENT®), eptifibatide (e.g., INTEGRILIN®), tirofiban (e.g., AGGRASTAT®), and abciximab (e.g., REOPRO®). For the purpose of this disclosure, antiplatelet agents include agents that inhibit P2Y receptors (e.g., P2Y₁₂), glycoprotein IIb/IIIa, or that antagonize thromboxane synthase or thromboxane receptors. Non-limiting examples of thromboxane A₂ antagonists are aspirin, terutroban, and picotamide. Non-limiting examples of P2Y receptor antagonists include cangrelor, ticagrelor, elinogrel, clopidogrel, prasugrel, and ticlopidine. Non-limiting examples of glycoprotein IIb/IIIa include abciximab, eptifibatide, and tirofiban. NSAIDS (e.g., ibuprofen) are also considered to be antiplatelet agents for the purposes of this disclosure. Other mechanisms of antiplatelet agents are known. Antiplatelet agents also include PAR1 antagonists, PAR4 antagonists GPVI antagonists and alpha2beta1 collagen receptor antagonists. Non-limiting examples of PAR-1 antagonists include vorapaxar and atopaxar. As used herein, aspirin is considered to be an antiplatelet agent but not an anticoagulant. Additional non-limiting examples of antiplatelet agents include cilostazol, prostaglandin E1, epoprostenol, dipyridamole, treprostinil sodium, and sarpogrelate.

Overcoming the effect of an anticoagulant varies according to the anticoagulant drug pharmacological action. In the case of advanced notice, as in a pre-planned surgery, the anti-coagulant dose can sometimes be tailored back before the surgery, however, there may be cases where such a reduction in dose is not advisable. In the case where anti-coagulant need reversing or the hemostatsis needs to be restored quickly (e.g., for emergency surgery), reversal agents or restoration agents are typically slow acting, expensive, or carry significant risk to the patient. Below are some non-limiting examples of reversal agents for marketed anti-coagulants.

Warfarin (e.g., COUMADIN®)—Warfarin works to prevent the activity of vitamin K in the liver which is a necessary co-factor to produce multiple coagulation factors. Warfarin reversal can sometimes be done be by dosing vitamin K or prothrombin complex concentrate (PCC). Vitamin K is low-cost and slow acting (more than 24 hrs PO) but can pose significant risk of inducing thrombosis in the patient, while PCC is expensive at roughly $5000/dose.

Dabigatran (e.g., PRADAXA®)—Dabigatran is a direct inhibitor of thrombin. The monoclonal antibody therapy idarucizumab (e.g., PRAXBIND®, Boehringer-Ingelheim, Germany) at dose of 5 grams (at two dose intervals each 2.5 grams) can typically reverse the effects of dabigatran within a few minutes. One wholesale price is $3482.50 for such a treatment.

Rivaroxaban (e.g., XARELTO®)—Rivaroxaban is a direct Factor Xa inhibitor. Rivaroxaban is reversed by Andexanet Alfa (e.g., ANDEXXA®), a recombinant Factor Xa decoy. This treatment can cost roughly $50,000 for a high-dose treatment.

Apixaban (e.g., ELIQUIS®)—Apixaban is a direct Factor Xa inhibitor. Apixaban is reversed by Andexanet Alfa, a recombinant Factor Xa decoy. This treatment costs roughly can cost $50,000 for a high-dose treatment.

Edoxaban (e.g., SAVAYSA®, LIXIANA®)—Edoxaban is a direct Factor Xa inhibitor. Exoxaban does not have an approved reversal agent. Ciraparantag (aripazine) and Andexanet Alfa have not been clinically proven to be appropriate.

Heparin and low molecular weight heparins are activators of antithrombin III (AT). AT inactivates proteases such as thrombin and Factor Xa. Protamine sulfate is a highly positively-charged polypeptide that binds to the negatively charged heparin and prevents its action on AT. Protamine sulfate is typically dosed at about 1.0 to about 1.5 mg/100 IU of active heparin.

Platelet-derived products are not currently used as a treatment method to counteract the activity of an anticoagulant drug, when such effects can have detrimental consequences to a subject or pose an unacceptable risk to a subject, for example during a surgical procedure or as the results of a traumatic event. There are no currently approved reversal agents or restoration agents for anticoagulant agents or agents that otherwise reduce the bleeding potential of a subject, or restore hemostasis after treatment with an anti-coagulant agent. Treatments for anticoagulant drugs are not necessarily targeted antidotes. Some novel anticoagulant treatments, such as Andexanet Alfa (e.g., ANDEXXA®), have seen some success, yet can be expensive. As such, emergency treatments (pre-op, trauma, and the like) are typically require blanket precautions to avoid or mitigate hemorrhage. Non-limiting examples include infusion of plasma, red blood cells, and anti-fibrinolytics. Platelet derivatives, in illustrative embodiments freeze-dried platelet derivatives (FDPDs) provided herein, overcome this long-standing need, and are an effective alternative or supplement to these general treatments or risk-mitigation strategies.

The results provided in numerous Examples in the Examples section herein demonstrate the impact of a composition comprising FDPDs product in an in vitro model of a subject taking anticoagulant drugs. FDPD compositions and other lyophilized platelet products are designed for infusion into a subject's bloodstream following diagnosis of trauma or hemostatic failure. Regardless of the mechanism of the anticoagulant drugs, FDPDs provided herein are able to decrease the bleeding potential of a subject taking such anticoagulant agents, and in some embodiments, restore normal hemostasis to the subject.

Without being bound by any particular theory, it is believed that certain platelet derivatives, in illustrative embodiments FDPDs provided herein, can work at least in part by providing a procoagulant negatively charged surface to augment thrombin generation above and beyond that suppressed by the anti-coagulants.

Products and methods are described herein for controlling bleeding and improving healing. The compositions, products and methods described herein can also be used to counteract the activity of any of the anticoagulant agents disclosed herein (e.g., as non-limiting examples, warfarin (e.g., COUMADIN®), heparin, LMWH, dabigatran (e.g., PRADAXA®), argatroban, hirudin, rivaroxaban (e.g., XARELTO®), apixaban (e.g., ELIQUIS®), edoxaban (e.g., SAVAYSA®), fondaparinux (e.g., ARIXTRA®). The products and methods disclosed herein in certain embodiments, are directed toward embodiments that can aid in the closure and healing of wounds.

In some embodiments, an antiplatelet agent can be selected from the group consisting of aspirin, cangrelor, ticagrelor, clopidogrel, prasugrel, eptifibatide, tirofiban, abciximab, and combinations thereof. In some embodiments, an antiplatelet agent can be selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, and combinations thereof. In some embodiments, an antiplatelet agent can be selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, sarpogrelate and combinations thereof. In some embodiments, the antiplatelet agent can include multiple antiplatelet agents, such as 2 (or more) of any of the antiplatelet agents described herein. In some embodiments, the antiplatelet agent can be aspirin and clopidogrel.

Cangrelor like clopidogrel, ticagrelor, and prasugrel, blocks the P2Y 12 (ADP) receptor on platelets. Cangrelor can in some cases be used as a representative of this class of drug. Cangrelor, unlike clopidogrel and prasugrel, does not need hepatic metabolism to become biologically active.

Eptifibatide is a peptide therapeutic that blocks the fibrin binding role of GPIIb-IIIa receptor on platelets. The drug is typically administered via IV as a 180 μg/kg bolus followed by 2 μg/kg/min continuous infusion. The blood concentration of eptifibatide is typically about 1-2 μM. Bleeding times generally return to normal within about 1 hour of drug stoppage.

Aspirin is an irreversible cyclooxygenase (COX) inhibitor. The COX enzyme in platelets is responsible for synthesis of thromboxane A2, prostaglandin E2 and prostacyclin (PGI2). Aspirin permanently inactivates the COX enzyme within platelets, and since platelets do not have the nuclear material to synthesize new enzyme, new platelets must be produced to overcome the aspirin effect. Without thromboxane A2, prostaglandin E2, and prostacyclin (PGI2) platelets are limited in their pro-aggregation activity. Many people are maintained on a low dose of aspirin to prevent unwanted clotting events. Aspirin bioavailability largely varies with administration route, with a single 500 mg dose IV at peaks of 500 μM and the same dose orally at 44 μM.

The antiplatelet class of drugs is widely used to prevent unwanted clotting episodes that lead to heart failure, stroke, and the like. In many cases, an antiplatelet drug may need to be reversed or stopped, or bleeding potential needs to be reduced in some other manner in a subject who has an antiplatelet drug in their blood stream, such that a bleeding potential of the subject is increased. In the case of advanced notice, as in a pre-planned surgery situation, the antiplatelet drug dose can sometimes be stopped before the surgery, preventing unwanted bleeding during surgery. In the case where an antiplatelet agent needs reversing quickly, reversal agents are typically not readily available, are expensive, or carry significant risk to the patient. In the case of need for rapid antiplatelet reversal, a platelet transfusion is typically administered, though the response to this is often only partial reversal. The caveat of this course of reversal is that the newly-infused platelets themselves are susceptible to circulating drug antiplatelet activity whereas, in some embodiments, compositions as described herein (e.g., including FDPDs) are not. In some embodiments, compositions as described herein (e.g., including FDPDs) are an active reversal agent. In some embodiments, the hemostatic activity of compositions as described herein (e.g., including FDPDs) does not succumb to antiplatelet drugs.

Some exemplary antiplatelet agents and potential methods of reversal are described below.

Acetylsalicylic acid (ASA; aspirin)—aspirin acts as a COX-1 blocker in platelets, which renders the platelet inactive by irreversibly inhibiting platelet-derived thromboxane formation. Clinically, aspirin is sometimes reversed by a platelet transfusion in emergency situations or by stopping treatment where surgery is scheduled in the future.

Clopidogrel (e.g., PLAVIX®)—clopidogrel acts as to prevent ADP from binding to its receptor on platelets. ADP binding leads to platelet shape change and aggregation. Clopidogrel is non-reversible. Clinically, clopidogrel is sometimes reversed by a platelet transfusion in emergency situations or by stopping treatment where surgery is scheduled in the future.

Cangrelor (e.g., KENGREAL®)—cangrelor acts to prevent ADP from binding to its receptor on platelets. ADP binding leads to platelet shape change and aggregation. Clopidogrel is reversible and platelet function is returned approximately 1 hour after stopping infusion. Clinically it is generally preferred when reversal is needed after a procedure.

Ticagrelor (e.g., BRILINTA®)—ticagrelor acts to prevent ADP from binding to its receptor and acts as an inverse agonist. Ticagrelor is reversible and platelet function can return after approximately 72 hours of the last dosage. Reversal of action of ticagrelor can be affected by the time after the last dose. If the last dose was longer than 24 hours previous, then platelet transfusion can sometimes be therapeutic to reverse the results.

Effient (e.g., PRASUGREL®)—Effient acts to prevent ADP from binding to its receptor and acts as a non-reversable antagonist. It being a non-reversible antagonist, new platelets must be formed to overcoming its effect. Clinically Effient is reversed by a platelet transfusion in emergency situations or by stopping treatment where surgery is scheduled in the future.

Eptifibatide (Integrilin)—Eptifibatide acts to block the GpIIb/IIIa and acts as a reversible antagonist. Clinically, Integrilin is reversed by a platelet transfusion in emergency situations or by stopping treatment where surgery is scheduled in the future.

In certain aspects, provided herein, a composition comprising platelets, or in illustrative embodiments a composition comprising platelet derivatives, which in further illustrative embodiments are FDPDs, may be delivered to a wound on the surface of or in the interior of a patient. In various embodiments, a composition comprising platelets, or in illustrative embodiments a composition comprising platelet derivatives, which in further illustrative embodiments are FDPDs, or in illustrative embodiments a composition comprising platelet derivatives, which in further illustrative embodiments are FDPDs can be applied in selected forms including, but not limited to, adhesive bandages, compression bandages, liquid solutions, aerosols, matrix compositions, and coated sutures or other medical closures. In embodiments, a composition comprising platelets, or in illustrative embodiments a composition comprising platelet derivatives, which in further illustrative embodiments are FDPDs may be administered to all or only a portion of an affected area on the surface of a patient. In other embodiments, a composition comprising platelets, or in illustrative embodiments a composition comprising platelet derivatives, which in further illustrative embodiments are FDPDs may be administered systemically, for example via the blood stream. In embodiments, an application of the platelet derivative can produce hemostatic effects for 2 or 3 days, preferably 5 to 10 days, or most preferably for up to 14 days.

Some aspects provide a method of treating a coagulopathy in a subject, the method comprising administering to the subject in need thereof an effective amount of a composition comprising platelets, or in illustrative embodiments a composition comprising platelet derivatives, which in further illustrative embodiments are FDPDs. In some embodiments, the composition comprising FDPDs further comprises additional components, such as components that were present when such FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

Some aspects provide a method of treating a coagulopathy in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments of any of the methods described herein, the coagulopathy is the result of the presence of an anticoagulant agent, and/or an antiplatelet agent in the blood of a subject. In some embodiments, the coagulopathy is the result of the presence of an anticoagulant agent that comprises a Factor XI inhibitor. In illustrative embodiments, the Factor XI inhibitor can be an antibody against Factor XI. In some embodiments, the Factor XI inhibitor can be a biologic that causes inhibition or loss of function of Factor XI. In some embodiments, the coagulopathy is the result of the presence of an anticoagulant agent, and an antiplatelet agent. In some embodiments, the subject has a reduced level of factor XI in the blood. In some embodiments, the subject has a loss of function of factor XI activity in the blood. In some embodiments, the subject has a reduced activity or function of factor XI. In some embodiments, the subject has been or is being treated with a dual antiplatelet therapy, and at least one anticoagulant agent, in illustrative embodiments, the anticoagulant agent comprises a factor XI inhibitor. Dual antiplatelet therapy can be a treatment to help stop clots from forming, for example, such dual antiplatelet therapy can include a combination of any antiplatelet agents as disclosed herein. In some cases, dual antiplatelet therapy can include aspirin and one other antiplatelet agent, in some embodiments, the other antiplatelet agent can be a P2Y12 inhibitor, for example, ticagrelor, prasugrel, and clopidogrel. Therefore, in some cases, the subject has been treated or is being treated with an anticoagulant agent, for example, a factor XI inhibitor, and a combination of the antiplatelet agents, for example, aspirin and ticagrelor, aspirin and clopidogrel, or aspirin and prasugrel.

In some embodiments, the subject is bleeding, has persistent bleeding (e.g, for more than 1 hour, 2 hours, 4, hours, 8 hours, 12 hours or 24 hours), has uncontrolled bleeding, or has bleeding that is considered life-threatening. In illustrative embodiments, the bleeding is increased or is more difficult to treat, decrease (e.g., decrease the WHO bleeding score for one or more sites of bleeding), or stop because the subject is being or has been treated with an anticoagulant, and/or an antiplatelet agent. In illustrative embodiments, the subject is being treated or has been treated with an anticoagulant, and an antiplatelet agent. In some embodiments, the subject is being treated or has been treated with an anticoagulant, and aspirin. In some embodiments, the subject is being treated or has been treated with an anticoagulant, aspirin, and one other antiplatelet agent. In some embodiments, the subject is bleeding, has persistent bleeding (e.g, for more than 1 hour, 2 hours, 4, hours, 8 hours, 12 hours or 24 hours), has uncontrolled bleeding, or has life-threatening bleeding because the subject has been or is being treated with an anticoagulant agent, in illustrative embodiments, the anticoagulant agent comprises a factor XI inhibitor. In some cases, the factor XI inhibitor can be an agent, such as a biologic, in illustrative embodiments, an antibody that inhibits, or otherwise decreases, or leads to a loss of function of factor XI in a subject, or otherwise leads to the reduction in levels of Factor XI in a subject. In some embodiments, the subject has hemophilia, in illustrative embodiments, hemophilia B. Typically, hemophilia B is a bleeding disorder caused by an insufficient level of factor IX in the subject. In some cases, the subject has an acquired hemophilia B, typically in acquired hemophilia B the factor IX is attacked by the subject's own immune system, thereby leading to abnormal bleeding. In some cases, the subject has congenital hemophilia B, typically, in a subject with congenital hemophilia B the levels of clotting factor IX is low because of genetic mutation. In some cases, the subject has hemophilia B, and the subject is being treated or has been treated with another agent-anticoagulant agent, or antiplatelet agent. In some embodiments, the subject has hemophilia A. Typically, hemophilia A is a bleeding disorder caused by an insufficient level of factor VIII in the subject. In some cases, the subject has an acquired hemophilia A, typically in acquired hemophilia A the factor VIII is attacked by the subject's own immune system, thereby leading to abnormal bleeding. In some cases, the subject has congenital hemophilia A, typically, in a subject with congenital hemophilia A the levels of clotting factor VIII is low because of genetic mutation. In some cases, the subject has hemophilia A, and the subject is being treated or has been treated with another agent-anticoagulant agent, or antiplatelet agent. In some embodiments, the subject has been or is being treated with a dual antiplatelet therapy, and at least one anticoagulant agent, in illustrative embodiments, the anticoagulant agent comprises a factor XI inhibitor. Dual antiplatelet therapy can be a treatment to help stop clots from forming, for example, such dual antiplatelet therapy can include a combination of any antiplatelet agents as disclosed herein. In some cases, dual antiplatelet therapy can include aspirin and one other antiplatelet agent, in some embodiments, the other antiplatelet agent can be a P2Y12 inhibitor, for example, ticagrelor, prasugrel, and clopidogrel. Therefore, in some cases, the subject has been treated or is being treated with an anticoagulant agent, for example, a factor XI inhibitor, and a combination of the antiplatelet agents, for example, aspirin and ticagrelor, aspirin and clopidogrel, or aspirin and prasugrel.

Some aspects provide a method of treating coagulopathy in a subject, wherein the subject has been treated or is being treated with an anticoagulant agent, the method comprising administering to the subject in need thereof an effective amount of a composition comprising platelets, or in illustrative embodiments a composition comprising platelet derivatives, which in further illustrative embodiments are FDPDs and an incubating agent comprising one or more salts, a buffer, optionally a cryoprotectant, and optionally an organic solvent.

Some aspects provide a method of treating coagulopathy in a subject, wherein the subject has been treated or is being treated with an anticoagulant agent, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some aspects provide a method of restoring normal hemostasis in a subject, the method comprising administering to the subject in need thereof an effective amount of a composition comprising platelets, or in illustrative embodiments a composition comprising platelet derivatives, which in further illustrative embodiments are FDPDs. In some embodiments, the composition comprising FDPDs further comprises additional components, such as components that were present when such FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

Some aspects provide a method of restoring normal hemostasis in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some aspects provide a method of restoring normal hemostasis in a subject, wherein the subject has been treated or is being treated with an anticoagulant agent, the method comprising administering to the subject in need thereof an effective amount of a composition comprising platelets, or in illustrative embodiments a composition comprising platelet derivatives, which in further illustrative embodiments are FDPDs. In some embodiments, the composition comprising FDPDs further comprises additional components, such as components that were present when such FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

Some embodiments provide a method of restoring normal hemostasis in a subject, wherein the subject has been treated or is being treated with an anticoagulant agent, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Compositions as described herein can also be administered to prepare a subject for surgery, in some cases. For some patients taking an anticoagulant agent, it may be difficult or impossible to reduce the dosage of the anticoagulant agent before surgery (e.g., in the case of trauma or other emergency surgery). For some patients taking an anticoagulant agent, it may be inadvisable to reduce the dosage of the anticoagulant agent before surgery (e.g., if the patient would be at risk of a thrombotic event (e.g., deep vein thrombosis, pulmonary embolism, or stroke) if the dosage of the anticoagulant agent were reduced over time.

Accordingly, some embodiments provide a method of preparing a subject for surgery, the method comprising administering to the subject in need thereof an effective amount of a composition comprising platelets, or in illustrative embodiments a composition comprising platelet derivatives, which in further illustrative embodiments are FDPDs. In some embodiments, the composition comprising FDPDs further comprises additional components, such as components that were present when such FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

Some embodiments provide a method of preparing a subject for surgery, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of preparing a subject for surgery, wherein the subject has been treated or is being treated with an anticoagulant agent, the method comprising administering to the subject in need thereof an effective amount of a composition comprising platelets, or in illustrative embodiments a composition comprising platelet derivatives, which in further illustrative embodiments are FDPDs. In some embodiments, the composition comprising FDPDs further comprises additional components, such as components that were present when such FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

Some embodiments provide a method of preparing a subject for surgery, wherein the subject has been treated or is being treated with an anticoagulant agent, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, a surgery can be an emergency surgery (e.g., in the case of trauma) or a scheduled surgery.

In some embodiments of any of the methods described herein, treatment with an anticoagulant can be stopped (e.g., in preparation for surgery). In some embodiments, treatment with an anticoagulant can continue.

In some embodiments of any of the aspects and embodiments herein, the method herein is a method for reducing bleeding in a subject, wherein the method comprises administering a dose, a first dose, or an effective amount of platelet derivatives in a rehydrated platelet derivative composition to a subject, wherein the subject has been or is being treated or administered with an anticoagulant agent, and an antiplatelet agent.

In one aspect, provided herein is a composition comprising platelets or platelet derivatives and an aqueous medium, wherein the aqueous medium has a protein concentration less than or equal to 50% of the protein concentration of donor apheresis plasma.

In one aspect, provided herein is a platelet derivative composition in the form of a powder, comprising a population of platelet derivatives having a reduced propensity to aggregate, such that no more than 25%, and in non-limiting illustrative embodiments, no more than 10%, of the platelet derivatives in the population aggregate under aggregation conditions comprising an agonist but no platelets, and wherein the platelet derivatives are capable of generating thrombin, and in certain embodiments have a potency of at least 0.5, 1.0, and in non-limiting illustrative embodiments 1.5 thrombin generation potency units (TGPU) per 10⁶ platelet derivatives.

In one aspect, provided herein is a platelet derivative composition in the form of a powder, comprising a population of platelet derivatives having a reduced propensity to aggregate, wherein no more than 25%, and in non-limiting illustrative embodiments, no more than 10%, of the platelet derivatives in the population aggregate under aggregation conditions comprising an agonist but no platelets; and having one or more, two or more, or all of the following characteristics of a super-activated platelet selected from: a. the presence of thrombospondin (TSP) on their surface at a level that is greater than on the surface of resting platelets; b. the presence of von Willebrand factor (vWF) on their surface at a level that is greater than on the surface of resting platelets; c. an inability to increase expression of a platelet activation marker in the presence of an agonist as compared to the expression of the platelet activation marker in the absence of an agonist.

In one aspect, provided herein is a platelet derivative composition in the form of a powder, comprising platelet derivatives, wherein less than 15%, and in certain non-limiting illustrative embodiments less than 5% of the CD 41-positive platelet derivatives are microparticles, in non-limiting illustrative embodiments having a diameter of less than 1 μm, and in certain non-limiting illustrative embodiments less than 0.5 μm, and wherein the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, comprising a population of platelet derivatives comprising CD 41-positive platelet derivatives, wherein the population comprises platelet derivatives having a reduced propensity to aggregate such that no more than 25%, and in non-limiting illustrative embodiments, no more than 10%, of the platelet derivatives in the population aggregate under aggregation conditions comprising an agonist but no platelets, wherein the platelet derivatives have an inability to increase expression of a platelet activation marker in the presence of an agonist as compared to the expression of the platelet activation marker in the absence of the agonist, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives; and wherein less than 15%, and in certain non-limiting illustrative embodiments less than 5% of the CD 41-positive platelet derivatives are microparticles having a diameter of less than 1 μm, and in certain non-limiting illustrative embodiments less than 0.5 μm.

In one aspect, provided herein is a platelet derivative composition in the form of a powder, comprising a population of platelet derivatives having a reduced propensity to aggregate, such that no more than 25%, and in non-limiting illustrative embodiments, no more than 10%, of the platelet derivatives in the population aggregate under aggregation conditions comprising an agonist but no platelets, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a platelet derivative composition in the form of a powder, comprising a population of platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm, and comprising platelet derivatives having one or more of, two or more of, three or more of, and in illustrative embodiments all of the following: a reduced propensity to aggregate, in certain embodiments such that no more than 25%, and in illustrative embodiments no more than 10% of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, comprising trehalose in the range of 20-35% by weight, polysucrose in the range of 45-60% by weight, and platelet derivatives in the range of 0.5-20% by weight, wherein the platelet derivatives to microparticles have a numerical ratio of at least 95:1 in the platelet derivative composition, and wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, comprising trehalose in the range of 20-35% by weight, polysucrose in the range of 45-60% by weight, and platelet derivatives in the range of 0.5-20% by weight, wherein the platelet derivative composition comprises a population of platelet derivatives having a reduced propensity to aggregate such that no more than 25%, and in non-limiting illustrative embodiments, no more than 10%, of the platelet derivatives in the population aggregate under aggregation conditions comprising an agonist but no platelets, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a plurality of containers each containing a platelet derivative composition in the form of a powder, wherein the platelet derivative composition in each container comprises a population of platelet derivatives having a reduced propensity to aggregate such that no more than 25%, and in non-limiting illustrative embodiments, no more than 10% of the platelet derivatives in the population aggregate under aggregation conditions comprising an agonist but no platelets, wherein the platelet derivative compositions in each container are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, wherein the platelet derivatives have an inability to increase expression of a platelet activation marker in the presence of an agonist as compared to the expression of the platelet activation marker in the absence of the agonist, wherein the plurality of containers comprise the platelet derivative composition from at least 2 different lots in separate containers, and wherein one or more of: the amount of plasma protein in the powder of any two containers chosen from different lots, differs by less than 10%, 5%, 2%, or 1%, and the amount of microparticles that are less than 1 μm, and in certain non-limiting illustrative embodiments less than 0.5 μm in the powder of any two containers chosen from different lots, differs by less than 10%, 5%, 2%, or 1%.

In one aspect, provided herein is a plurality of containers each filled with a platelet derivative composition in the form of a powder, wherein the platelet derivative composition comprises trehalose in the range of 20-35% by weight; polysucrose in the range of 45-60% by weight; and platelet derivatives in the range of 0.5-20% by weight, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, and a population of platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm.

To reiterate, any embodiments herein in this section and in this specification and associated claims, can be combined and/or used in any of the aspects herein and in combination with any of the other embodiments herein. Furthermore, a “powder” recited in any aspect or embodiment can alternatively be a solid, or a composition comprising less than 1% water content in such aspect or embodiment.

In certain illustrative embodiments of a composition, or in some compositions used in or formed by a process, the platelet derivatives in a composition, as a non-limited example a powder, and/or formed by a process disclosed herein, are surrounded by a compromised plasma membrane, are positive for CD 41, and/or are 0.5 to 2.5 μm in diameter. In some embodiments, the composition comprises platelet derivatives such that at least 95% platelet derivatives positive for CD 41 have a diameter in the range of 0.5 to 2.5 μm. Such diameter can be measured, for example by flow cytometry technique as known to a skilled artisan in the art.

In some aspects, a platelet derivative composition, in illustrative embodiment a freeze-dried platelet derivative composition, FDPDs or HLA-characterized FDPDs, FPH or HLA-characterized FPH, including, but not limited to, those of any of the aspects or embodiments herein, or the platelet derivative composition prepared by any of the processes disclosed herein, can be administered, or delivered to a subject to control bleeding. Thus, such administering can be performed by administering an effective dose of the platelet derivative composition. Such effective dose in illustrative embodiments, includes multiple individual doses, or a continuous dose. In illustrative embodiments, the subject has an indication and thus is afflicted with a disorder or disease that could benefit from delivery of such platelet derivative compositions, during treatment for such disorder or disease, during a surgical procedure, during a transplantation procedure, or during the treating of the subject with an antiplatelet agent, and/or an anticoagulant agent. In some embodiments, platelet derivatives as disclosed herein have short circulation half-life, for example, less than 20 minutes, 15 minutes, 10 minutes, 8 minutes, 6 minutes, 5 minutes, 4 minutes, or 3 minutes, when administered to a subject, such as a mammal, in illustrative embodiments, a human. Therefore, in order to have a desirable effect of the platelet derivatives in the subject, methods herein can comprise administering more than 1, 2, 3, 4, 5, or more doses or therapeutically effective doses of the platelet derivatives herein to the subject in a given span of time. It will be understood that in order to maintain a certain population of platelet derivatives in the subject for observing the beneficial effect of the platelet derivatives in spite of short half-life circulation, methods herein can include administering multiple doses of the platelet derivatives to the subject in a span of time or at a frequency of time until bleeding is reduced, stopped, or a beneficial effect is observed. In some embodiments, a method herein can include administering the platelet derivatives to the subject as a continuous infusion over a span of time until bleeding is reduced, stopped, or a beneficial effect is observed. Such a span of time for continuous infusion can include, in some embodiments, a span of time equal to any time frame provided herein for multiple doses. In some embodiments, continuous infusion is performed for 5, 10, 15, 30, or 45 minutes, or for 1, 2, 3, 4, 6, 8, 12, or 18 hours, or for 1, 2, 3, 4, 5, 6, or 7 days. In some cases, administering can be performed by administering the platelet derivatives as a continuous infusion and also administering more than one dose the platelet derivatives in an intermittent regime until the bleeding or the bleeding potential of the subject is reduced, stopped, hemostasis is restored in the subject, or otherwise any beneficial effects of administering are observed.

A person of skill in the art can contemplate treating a subject, such as, controlling bleeding, reducing bleeding or bleeding potential of the subject, or addressing a disorder or a condition occurred by any of the indications disclosed herein using platelet derivatives as described herein as a medicament in several doses in a span of time for treating the subject. In illustrative embodiments, the subject is being treated or has been treated with an anticoagulant agent, and/or an antiplatelet agent, in some embodiments, the antiplatelet agent can include aspirin. Alternatively, or in combination, in some embodiments, administering of platelet derivatives as described herein can be performed as a continuous infusion procedure. In some embodiments, administering of platelet derivatives, FDPDs, or FPH, as disclosed herein, is performed for at least, or at a maximum of 2, 3, 4, 5, 6, 7, 8, 9, or 10 doses in a 4, 8, 12, 24, 48 or 72-hour period of treatment. For example, a specific dose of platelet derivatives can be decided as per the requirement of a subject, for example based on the weight of the subject, and the specific dose can be provided to the subject as a continuous infusion procedure with or without an interval between continuous infusion doses. In some embodiments, administering can be performed as a continuous infusion procedure until the bleeding and/or bleeding potential of the subject is reduced as compared to the bleeding or bleeding potential before the administering. In some embodiments, administering can be performed as a continuous infusion until the bleeding in the subject is stopped. In some embodiments, administering of platelet derivatives as described herein can be performed at regular intervals. For example, a single, double, or more doses of platelet derivatives as described herein and as per the requirement of a subject, can be administered to a subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours until a desired outcome, for example reduced or termination of bleeding, is achieved. For example, between 2 in the lower end and 7, 8, 10, 12, 15, 20 doses in the higher end, or between 3 in the lower end and 7, 8, 10, 12, 15, 20 doses in the higher end of platelet derivatives, can be administered to the recipient subject within 1 hour, 30 minutes, 20 minutes, 15 minutes, 10 minutes or 5 minutes, and such administration can be guided by the effect of the dosing on bleeding of the subject, for example at one or more specific sites of bleeding. For example, the dosing can be performed until the bleeding at one or more sites decreases and/or stops. Such bleeding determination can be made, for example, by visual inspection. The decrease can be for example, a decrease that is observable by visual inspection, a decrease that is detected by a measurement, a decrease that is no longer considered life threatening, and/or a decrease such that the bleeding is considered minor. In some embodiments, total number of doses administered to the subject can be in the range of 2-100, 2-80, 2-60, 2-50, 2-40, 2-30, 2-20, 2-15, 2-10, 2-8, 3-100, 3-80, 3-60, 3-50, 3-40, 3-30, 3-20, 3-15, 3-10, 3-8, 4-100, 4-80, 4-60, 4-50, 4-40, 4-30, 4-20, 4-15, 4-10, 4-8, 5-20, 5-15, 5-10, 5-8, 10-100, 20-100, 30-100, 40-100, 50-100, 60-100, 70-100, 80-100, 10-90, 10-80, 10-70, 10-60, 10-50, 10-40, 10-30, 10-20, or 10-15, in illustrative embodiments, within a given span of time as disclosed herein, for example, in a span of time from administering a first dose of platelet derivatives until the bleeding is controlled, stopped, bleeding potential is reduced, or any other beneficial parameter according to a therapeutic indication of the subject. In some embodiments, each of the doses administered according to any of the embodiments or aspects herein can have the same dosage of platelet derivatives/kg of the subject, or can have various dosage of platelet derivatives/kg of the subject, such that one particular dose (platelet derivatives/kg) can be different than the other doses succeeding or preceding number the particular dose. In some embodiments, the dosages in any number of doses being administered to a subject herein can include any therapeutic effective dosage (platelet derivatives/kg) as disclosed herein. In some embodiments, the specific dose for any dose of a multi-dose regimen can be influenced, guided, and/or changed, based on the outcome that is detected or observed, in illustrative embodiments, a bleeding related outcome. Thus, for example if a first dose is administered to a patient and no change in bleeding is observed, one or more additional doses can be administered within any of the timeframes provided herein, that is higher than the first dose. Furthermore, in some embodiments the desired outcome is to maintain the reduced or cessation of bleeding, when prior doses of platelet derivatives (e.g., FDPDs) successfully reduced or stopped the bleeding.

In some embodiments, the doses can be administered at a regular interval for 36 hours, 48 hours, or 72 hours from the start of the first dose. In some embodiments, administering of platelet derivatives as described herein can be performed as a mixed procedure in which the continuous infusion can be interrupted with a specific dose of platelet derivatives followed by a specific interval as per the requirement. In some embodiments, administering of platelet derivatives as described herein can be performed in a maximum of 2, 3, 4, 5, 6, 7, 8, 9, or 10 doses in a 24-hour period. In some embodiments, administering of platelet derivatives as described herein is performed in a maximum of 2, 3, 4, 5, 6, 7, 8, 9, or 10 doses in a 72-hour period of treatment. In some embodiments, methods herein comprise administering 2-50, 2-45, 2-40, 2-35, 2-30, 2-25, 2-20, 2-15, 2-10, 2-8, 2-6, 5-50, 10-50, 15-50, 20-50, 25-50, 3-15, 3-12, 3-10, 3-9, 3-8, 4-25, 4-20, 4-18, 4-15, 5-25, 5-20, 5-15, or 5-10 doses of platelet derivatives to a subject within 1, 2, 3, 4, or 8 hours, for example, after administering a first dose of platelet derivatives. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-25, 4-20, 4-18, 4-15, 4-12, 4-10, 4-8, 5-25, 5-20, 5-15, 5-10, or 5-8 doses of platelet derivatives to a subject within 2 hours. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-15, 4-12, 4-10, 4-8, 5-25, 5-20, 5-15, or 5-10 doses of platelet derivatives to a subject within 5 hours. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-25, 4-20, 4-18, 4-15, 4-12, 4-10, 4-8, 5-25, 5-20, 5-15, 5-10, or 5-8 doses of platelet derivatives to a subject within 12 hours. In some embodiments, methods herein comprise administering 2-80, 2-60, 2-50, 2-45, 2-40, 2-35, 2-30, 2-25, 2-20, 2-15, 2-10, 2-8, 3-50, 3-45, 3-30, 3-20, 3-15, 3-12, 3-9, 4-20, 4-15, 4-12, 4-10, 4-8, 5-50, 5-45, 5-40, 5-35, 5-30, 5-25, 5-20, 5-15, 5-10, 15-80, 15-75, 15-70, 15-60, 15-45, 15-40, 15-35, 15-30, 15-25, or 15-20 doses of platelet derivatives to a subject within 24 hours.

In some situations that benefit from controlling of bleeding in a subject in a short span of time, for example, during a surgery or a transplantation procedure, or in situations where the antiplatelet agent including aspirin, and/or the anticoagulant agent cannot be stopped, or the dosage cannot be reduced, methods herein can include administering more than 2, 4, or 10 doses of platelet derivatives, for example 2-20, 2-15, 2-12, 2-10, 2-8, 3-20, 3-18, 3-15, 3-12, 3-10, 4-20, 4-15, 4-10, 5-20, 5-15, or 5-10 doses every 2, 5, 10, 15, 20, 25, 30, 45 minutes, or more frequently, at the same or at variable frequencies within a timeframe until bleeding is controlled or reduced, bleeding potential is reduced, beneficial effect is observed in a subject, or until the surgery is completed. Thus, it will be understood that a frequency of a certain number of doses within a time period or more frequency, can be dosing at the same frequency or at a variable frequency within the given timeframe. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 5 minutes, or more frequently, in illustrative embodiments, every 5 minutes until bleeding is controlled or reduced, bleeding potential is reduced, beneficial effect is observed in a subject, or until the surgery is completed. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 10 minutes, or more frequently, in illustrative embodiments, every 10 minutes until bleeding is controlled or reduced, bleeding potential is reduced, beneficial effect is observed in a subject, or until the surgery is completed. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 15-45 minutes until bleeding is controlled or reduced, bleeding potential is reduced, beneficial effect is observed in a subject, or until the surgery is completed. For example, administering can include 2-20 doses every 15-20 minutes, 15-30 minutes, or 15-45 minutes from the time of a first dose. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 15 minutes to 1 hour until bleeding is controlled or reduced, bleeding potential is reduced, beneficial effect is observed in a subject, or until the surgery is completed. For example, administering can include 2-20 doses every 15-20 minutes, 15-30 minutes, 15-45 minutes, 20 minutes-1 hour, 30 minutes-1 hour, or 45 minutes-1 hour from the time of a first dose.

In some embodiments, methods herein include administering, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 2 minutes, for example, from the time of a first dose, until bleeding is controlled or reduced, bleeding potential is reduced, beneficial effect is observed in a subject, or until the surgery is completed. In some embodiments, methods herein include administering multiple times 2-20, 2-18, 2-15, 2-12, 2-10, 2-8, 2-6, 2-4, 3-20, 3-18, 3-15, 3-12, 3-9, 4-20, 4-15, 4-12, 4-10, 4-8, 5-20, 5-15, or 5-10 doses of platelet derivatives to a subject within 5 minutes, for example, from the time of a first dose until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 10 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 15 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 30 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 45 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 1 hour until bleeding is controlled or reduced, bleeding potential is reduced, beneficial effect is observed in a subject, or until the surgery is completed. In some embodiments, methods herein include administering, in illustrative embodiments, multiple times 2-50, 2-45, 2-40, 2-35, 2-30, 2-25, 2-20, 2-15, 2-10, 2-8, 2-6, 5-50, 5-45, 5-40, 5-35, 5-30, 5-25, 5-20, 5-15, or 5-10 doses of platelet derivatives to a subject within 20 minutes to 45 minutes until bleeding is controlled or reduced, bleeding potential is reduced, beneficial effect is observed in a subject, or until the surgery is completed. A skilled artisan can understand that administering multiple times includes administering more than one time, for example, 2 times, 3 times, 4 times, 5 times, 6 times or more doses as disclosed herein. For example, administering multiple times includes 2-10 times, 2-9 times, 2-8 times, 2-6 times, or 2-4 times as disclosed herein. In some embodiments, administering of platelet derivatives can be performed at a frequency of at least one dose every 15 minutes or more frequently. For example, administering can be performed at a frequency of at least one dose every 15 minutes or more frequently starting from the first dose until the bleeding or the bleeding potential of the subject is reduced as compared to the bleeding or the bleeding potential before the administering. In some embodiments, the administering of any number of doses as disclosed herein can be performed until the bleeding stops. In some embodiments, the administering of any number of doses as disclosed herein can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently. It will be understood that the starting point for measuring dosing intervals between two doses is the time point at the moment after the entire first dose of the two doses is administered to the subject. Since it may take some time to administer each dose to a subject, a time-frame can be calculated after the end of infusion or other administration route, of a first dose or a previous dose. In some embodiments, administering each dose of platelet derivatives, FDPDs, or FPH can take at least 30 seconds, 1 minute, 1.5 minutes, 2 minutes, or 3 minutes. In some embodiments, administering each dose can take 10 seconds to 10 minutes, 10 seconds to 5 minutes, 10 seconds to 3 minutes, 20 seconds to 3 minutes, 30 seconds to 3 minutes, or 1 minute to 3 minutes. Further, in some embodiments, the subject involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion defined as two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

In some embodiments of any of the aspects or embodiments herein that include a composition for use in controlling bleeding, or a method for controlling bleeding, a dose (which can be referred to herein as a single dose or an individual dose) can be administered to a subject from one vial or by combining the contents of more than one vial each containing platelet derivatives, FDPDs or FPH. In some embodiments, a single dose can be administered to a subject by combining the contents of 2 to 6, 2 to 5, or 2 to 4 vials, each vial containing platelet derivatives, FDPDs or FPH. In some embodiments, a single dose, in illustrative embodiments of at least 1×10⁷/kg, 1×10⁸/kg, 1×10⁹/kg, 1.2×10⁹/kg, 1.4×10⁹/kg, or 1.6×10⁹/kg, can be administered to a subject by combining the contents of 2 vials, 3 vials, or 4 vials, each containing platelet derivatives, FDPDs, or FPH. In some embodiments, each vial containing platelet derivatives, FDPDs or FPH can have a volume in the range of 5-100 ml, 10-90 ml, 25-75 ml, or 5-40 ml, in illustrative embodiments, have a volume of 20 ml, 25 ml, 30 ml, 35 ml, or 40 ml.

A person of skill in the art can contemplate the effective dose of platelet derivatives that can be effective to treat a subject in need thereof. The need may differ based on the condition of the subject. The effective dosage can be categorized into a) low dosage; b) medium dosage; and c) high dosage. In some embodiments, a medicament or a method of treating a subject can have the effective dose as low, medium, or high dosage of platelet derivatives that can broadly range from 1.0×10⁷ on the low end of the range to 1.0×10¹⁰/kg, 1.0×10¹¹/kg or 1.0×10¹²/kg of the subject on the high end of the range. In some embodiments, a dose, an effective dose, or a therapeutically effective dose of platelet derivatives, FDPDs, FPH, as disclosed herein can be administered to a subject or a recipient as a part of a surgical procedure. In other words, platelet derivatives, FDPDs, or FPH can be provided to a subject or a recipient during a surgery, before a surgery, or as a follow-up after the surgery. For example, any dose of platelet derivatives, FDPDs, or FPH as disclosed herein can be administered during a surgery, for example, due to an increased risk of bleeding, or if bleeding, or unusually heavy bleeding is observed.

In some embodiments of any of the aspects or embodiments herein that include a composition for use for controlling bleeding of a subject, a method for controlling bleeding in a subject, or administering platelet derivatives to a subject herein, a dose or single dose of platelet derivatives, in illustrative embodiments for multiple administration can be in the range of 1.0×10⁷ to 1.0×10¹²/kg, 1.0×10⁸ to 1.0×10¹²/kg, 1.0×10⁹ to 1.0×10¹²/kg, 1.0×10⁷ to 1.0×10¹¹/kg, 1.0×10⁷ to 1.0×10¹⁰/kg, 1.0×10⁸ to 1.0×10¹¹/kg, or 1.0×10⁹ to 1.0×10¹¹/kg of the subject. In illustrative embodiments, a dose or a single dose of platelet derivatives can be in the range of 1.0×10⁹ to 1.0×10¹¹/kg, 1.2×10⁹ to 1.0×10¹¹/kg, 1.4×10⁹ to 1.0×10¹¹/kg, 1.0×10⁹ to 8.0×10¹⁰/kg, 1.0×10⁹ to 6.0×10¹⁰/kg, 1.0×10⁹ to 5.0×10¹⁰/kg, 1.0×10⁹ to 4.0×10¹⁰/kg, 1.0×10⁹ to 3.0×10¹⁰/kg, or 1.0×10⁹ to 2.0×10¹⁰/kg. In some embodiments, an effective dose can depend on the number of times a dose herein is to be administered to the subject until the bleeding is reduced, stopped, or otherwise a beneficial outcome is reached. For example, a dose or a single dose can be in the range of 1.0×10⁸ to 1.0×10¹²/kg. However, such single dose may not be effective at reducing or stopping bleeding. Thus, a second dose in such non-limiting example, can be administered and if bleeding is reduced or stopped, depending on the desired or recited outcome, then the combined two doses provide the effective dose. As such, the effective dose in this non-limiting example, is in the range of 2.0×10⁸ to 2.0×10¹²/kg. And such two doses provide a total dose in the range of 2.0×10⁸ to 2.0×10¹²/kg. In another non-limiting example, if 10 doses are administered to reduce bleeding, and that was the desired or recited outcome, and a dose or a single dose administered is in the range of 1.0×10⁹ to 1.0×10¹²/kg, then the effective dose is in the range of 1.0×10⁹ to 1.0×10¹³/kg of the subject. And the total dose, regardless of whether it was therapeutically effective, is in the range of 1.0×10⁹ to 1.0×10¹³/kg of the subject. Accordingly, in some embodiments, a total dose and/or an effective dose administered to a subject is in the range of 2.0×10⁷ to 1.0×10¹³/kg, 1.0×10⁸ to 1.0×10¹³/kg, 1.0×10⁹ to 1.0×10¹³/kg, 2.0×10⁷ to 1.0×10¹¹/kg, 2.0×10⁷ to 1.0×10¹⁰/kg, 1.0×10⁸ to 1.0×10¹¹/kg, or 1.0×10⁹ to 1.0×10¹¹/kg of the subject.

In some embodiments, 2-20, 3-20, 4-20, 5-20 doses of platelet derivatives can be administered to a subject within 15 minutes, 30 minutes, 45 minutes, 1 hour, 2 hours, 3 hours, 4 hours, 5 hours, or 6 hours, or more frequently wherein each dose can be of at least 1×10⁸/kg, 5×10⁸/kg, 1×10⁹/kg, 1.2×10⁹/kg, 1.4×10⁹/kg, or 1.6×10⁹/kg of the subject. In some embodiments, administering can be done by administering at least a single dose of at least 1×10⁸/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least a single dose of at least 5×10⁸/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least a single dose of at least 1×10⁹/kg, in illustrative embodiments, at least 1.5×10⁹/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can include administering 3, 4, 5, 6 or more doses, for example, each dose of at least 1×10⁸/kg of the subject within 15 minutes, 30 minutes, 45 minutes, 1 hours, 2, 3, 4, 5, or 6 hours, or more frequently. In some embodiments, administering can be done by administering at least 2 doses of 1×10⁸/kg to 1×10¹⁰/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least 2 doses of 1×10⁸/kg to 1×10¹⁰/kg of the subject multiple times at a frequency of every 5 minutes, 15 minutes, 30 minutes, 45 minutes, 1 hour, 2, 3, 4, 5, 6 hours, or more frequently starting from a first dose, in illustrative embodiments, a total number of doses administered can be in the range of 2-30, 2-25, 2-20, 2-15, 2-10, 2-8, 3-30, 3-25, 3-20, 2-18, 3-15, 3-12, 3-9, 4-30, 4-25, 4-20, 4-15, 4-12, 4-8, 5-30, 5-25, 5-20, 5-15, or 5-10 within a period of 24 hours from a first dose. It will be understood a total dose and/or effective dose administered in aspects herein, can be any range that equals a range of doses and amounts per dose, provided herein.

In some embodiments of any of embodiments or aspects herein, that include administering platelet derivatives, FDPDs, or FPH to a subject, administering comprises topical administration, parenteral administration, or a combination of a topical and parenteral administration. Administration via parenteral route can comprise intravenous (IV), intraperitoneal (IP), subcutaneous (SC), intramuscular (IM), or intradermal (ID). In some embodiments, parenteral administration comprises intravenous administration. In some embodiments, parenteral administration comprises subcutaneous, intraperitoneal, intramuscular, or intradermal. Topical administration route can comprise a topical application of platelet derivatives, FDPD, or FPH directly at the site of bleeding. In some embodiments, platelet derivatives, FDPD, or FPH can be prepared in various forms including, but not limited to, particulate, powder, solution, gel, and matrix. In some embodiments, administering herein can comprise a combination of parenteral administration, in illustrative embodiments, intravenous administration, and topical administration, in illustrative embodiments, administering topically at a site of bleeding of the subject. In some cases, the site of the parenteral administration can vary depending upon the type and extent of bleeding of the subject. For example, parenteral administration can be done near the site of bleeding. In some cases, during the surgery, or after surgery, parenteral administration can be done near the site of incision to control the bleeding. In some embodiments, administering comprises a combination of topical and parenteral administration, in illustrative embodiments, intravenous administration at the site of injury, incision, or otherwise bleeding. For example, in some cases, administering can be done in a manner such that topical administration and the parenteral administration, in illustrative embodiments, intravenous administration is alternately done. The dose ranges of platelet derivatives, FDPD, or FPH for topical administration can be any of the ranges as disclosed herein. In some embodiments, the doses of parenteral administration can be the same or different as compared to the doses of topical administration at the site of injury, incision, or otherwise bleeding.

In some embodiments, a platelet derivative composition as described herein can be administered or delivered to a subject, such as a subject afflicted with any one or combination of indications as described herein, and the dose of a platelet derivative composition can be in the range of 1.0×10⁷ to 1.0×10¹² particles/kg of the subject. For example, in some embodiments, a dose, or a single dose of a composition comprising platelets, platelet derivatives (e.g., FDPDs), or FPH can include between about or exactly 1.0×10⁷ on the low end of the range to 1.0×10¹² particles (e.g. FDPDs)/kg of a subject on the high end of the range, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹¹ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, 5.0×10⁷ to 1.0×10¹¹ particles (e.g. FDPDs)/kg of a subject, 5.0×10⁹ to 1.0×10¹⁰ particles (e.g. FDPDs)/kg of a subject), or 1.0×10¹⁰ to 1.0×10¹¹ or 1.0×10¹² particles (e.g. FDPDs)/kg of a subject on the high end of the range. In some embodiments, the dose can be in the range of 250 and 5000 TGPU per kg of the subject.

In some embodiments, a platelet derivative composition, such as that provided in any aspect or embodiment herein, can be administered or delivered to a subject afflicted with any one or combination of indications/diseases as disclosed herein, and the dose, or a single dose of a platelet derivative composition comprising FDPDs, or FPH can be in the range of 1.0×10⁷ on the low end of the range to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, or 5.0×10⁹ to 1.0×10¹⁰ particles (e.g. FDPDs)/kg of a subject). In some embodiments, a dose of a composition comprising platelets or platelet derivatives (e.g., FDPDs) can be a range of between about or exactly 1.0×10⁸, 5.0×10⁸, 1.0×10⁹, 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 5.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, or 5.0×10⁹ on the low end of the range to 1.0×10¹⁰ particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, or 1.2×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end of the range.

In some embodiments of any aspect or embodiment herein a therapeutically effective dose or effective dose or amount of the platelet derivatives in a platelet derivative composition is in the range of 1.5×10⁷ to 5.0×10¹⁰/kg, 2.0×10⁷ to 1.0×10¹⁰/kg, 2.5×10⁷ to 5.0×10⁹/kg, 2.75×10⁷ to 3.0×10⁹/kg, 2.8×10⁷ to 4.0×10⁹/kg, 3.0×10⁷ to 4.0×10⁹/kg, 5×10⁷ to 4.0×10⁹/kg, 6×10⁷ to 3.0×10⁹/kg, 9×10⁷ to 3.0×10⁹/kg, 1.0×10⁸ to 2.0×10⁹/kg, or 1.3×10⁸ to 1.8×10⁹/kg of the subject. In some embodiments, a therapeutically effective dose or effective dose or amount of the platelet derivatives in a platelet derivative composition is in the range of 5.0×10⁷ to 1.0×10⁹/kg, 1.0×10⁸ to 5.0×10⁸/kg, 1.2×10⁸ to 2.5×10⁸/kg, 1.6×10⁸ to 2.2×10⁸/kg, or 1.7×10⁸ to 2.0×10⁸/kg of the subject. In some embodiments, the platelet derivatives in a platelet derivative composition is 1.1×10⁸/kg, 1.2×10⁸/kg, 1.3×10⁸/kg, 1.4×10⁸/kg, 1.5×10⁸/kg, 1.6×10⁸/kg, 1.7×10⁸/kg, 1.8×10⁸/kg, 1.9×10⁸/kg, 2.0×10⁸/kg, 2.1×10⁸/kg, 2.2×10⁸/kg. 2.3×10⁸/kg, 2.4×10⁸/kg, or 2.5×10⁸/kg of the subject.

In some embodiments of any aspect or embodiment herein a therapeutically effective dose or effective dose or amount of the platelet derivatives in a platelet derivative composition is in the range of 5.1×10⁸ to 9.9×10⁸/kg, 5.5×10⁸ to 9.5×10⁸/kg, 5.8×10⁸ to 9.3×10⁸/kg, 6.1×10⁸ to 9.0×10⁸/kg, 6.5×10⁸ to 8.8×10⁸/kg, 6.8×10⁸ to 8.5×10⁸/kg, 7.0×10⁸ to 8.4×10⁸/kg, 7.5×10⁸ to 8.3×10⁸/kg, 7.8×10⁸ to 8.2×10⁸/kg, or 7.9×10⁸ to 8.1×10⁸/kg of the subject. In some embodiments, a therapeutically effective dose or effective dose or amount of the platelet derivatives in a platelet derivative composition is 7.5×10⁸/kg, 7.6×10⁸/kg, 7.7×10⁸/kg, 7.8×10⁸/kg, 7.9×10⁸/kg, 8.0×10⁸/kg, 8.1×10⁸/kg, 8.2×10⁸/kg, 8.3×10⁸/kg, 8.4×10⁸/kg, or 8.5×10⁸/kg of the subject.

In some embodiments of any aspect or embodiment herein a dose, a therapeutically effective dose, or effective dose or amount of the platelet derivatives in a platelet derivative composition is in the range of 1.0×10⁹ to 1.0×10¹⁰/kg, 1.1×10⁹ to 8.0×10⁹/kg, 1.2×10⁹ to 7.0×10⁹/kg, 1.2×10⁹ to 6.0×10⁹/kg, 1.2×10⁹ to 5.0×10⁹/kg, 1.3×10⁹ to 4.0×10⁹/kg, 1.3×10⁹ to 3.0×10⁹/kg, 1.3×10⁹ to 2.5×10⁹/kg, 1.4×10⁹ to 1.9×10⁹/kg, 1.50×10⁹ to 1.75×10⁹/kg, or 1.55×10⁹ to 1.70×10⁹/kg of the subject. In some embodiments, a dose, a therapeutically effective dose or effective dose or amount of the platelet derivatives in a platelet derivative composition is 1.1×10⁹/kg, 1.2×10⁹/kg, 1.3×10⁹/kg, 1.4×10⁹/kg 1.5×10⁹/kg, 1.55×10⁹/kg, 1.56×10⁹/kg, 1.57×10⁹/kg, 1.58×10⁹/kg, 1.59×10⁹/kg, 1.6×10⁹/kg, 1.61×10⁹/kg, 1.62×10⁹/kg, 1.63×10⁹/kg, 1.64×10⁹/kg, 1.65×10⁹/kg, 1.66×10⁹/kg, 1.7×10⁹/kg, 1.8×10⁹/kg, 1.9×10⁹/kg, or 2.0×10⁹/kg of the subject.

In some embodiments of any aspect, or embodiment herein a therapeutically effective dose or effective dose or amount of the platelet derivatives in a platelet derivative composition is in the range of 1.0×10⁷ to 1.0×10¹⁴ particles/kg of the subject, 1.6×10⁷ to 1.0×10¹⁴ particles (e.g. FDPDs/kg of subject, 1.6×10⁷ to 8×10¹³ particles (e.g. FDPDs/kg of a subject), 1.6×10⁷ to 5.1×10¹³ particles (e.g. FDPDs/kg of a subject), 1.6×10⁷ to 3.0×10¹³ particles (e.g. FDPDs)/kg of a subject, 1.6×10⁷ to 1.0×10¹³ particles (e.g. FDPDs)/kg of a subject, 1.6×10⁷ to 8.0×10¹² particles (e.g. FDPDs)/kg of a subject, 1.6×10⁷ to 5.0×10¹² particles (e.g. FDPDs)/kg of a subject, 1.6×10⁷ to 3.0×10¹² particles (e.g. FDPDs)/kg of a subject, 1.6×10⁷ to 1.0×10¹² particles (e.g. FDPDs)/kg of a subject, 1.6×10⁷ to 8.0×10¹¹ particles (e.g. FDPDs)/kg of a subject, 1.6×10⁷ to 5.0×10¹¹ particles (e.g. FDPDs)/kg of a subject, 1.6×10⁷ to 3.0×10¹¹ particles (e.g. FDPDs)/kg of a subject, 1.6×10⁷ to 1.0×10¹¹ particles (e.g. FDPDs)/kg of a subject, 1.6×10⁷ to 8.0×10¹⁰ particles (e.g. FDPDs)/kg of a subject, 1.6×10⁷ to 5.0×10¹⁰ particles (e.g. FDPDs)/kg of a subject, 1.6×10⁷ to 3.0×10¹⁰ particles (e.g. FDPDs)/kg of a subject, 1.6×10⁷ to 8.0×10¹⁰ particles (e.g. FDPDs)/kg of a subject, 1.6×10⁷ to 5.0×10¹⁰ particles (e.g. FDPDs)/kg of a subject, 1.6×10⁷ to 3.0×10¹⁰ particles (e.g. FDPDs)/kg of a subject, 1.6×10⁷ to 8.0×10¹⁹ particles (e.g. FDPDs)/kg of a subject, 5.0×10⁷ to 1.0×10¹⁴ particles (e.g. FDPDs)/kg of a subject, 8.0×10⁷ to 1.0×10¹⁴ particles (e.g. FDPDs)/kg of a subject, 1.0×10⁸ to 1.0×10¹⁴ particles (e.g. FDPDs)/kg of a subject, 3.0×10⁸ to 1.0×10¹⁴ particles (e.g. FDPDs)/kg of a subject, 5.0×10⁸ to 1.0×10¹⁴ particles (e.g. FDPDs)/kg of a subject), 8.0×10⁸ to 1.0×10¹⁴ particles (e.g. FDPDs)/kg of a subject), 1.0×10⁹ to 1.0×10¹⁴ particles (e.g. FDPDs)/kg of a subject), 3.0×10⁹ to 1.0×10¹⁴ particles (e.g. FDPDs)/kg of a subject), 5.0×10⁹ to 1.0×10¹⁴ particles (e.g. FDPDs)/kg of a subject), 8.0×10⁹ to 1.0×10¹⁴ particles (e.g. FDPDs)/kg of a subject), 1.0×10¹⁰ to 1.0×10¹⁴ particles (e.g. FDPDs)/kg of a subject), 3.0×10¹⁰ to 1.0×10¹⁴ particles (e.g. FDPDs)/kg of a subject), 5.0×10¹⁰ to 1.0×10¹⁴ particles (e.g. FDPDs)/kg of a subject), 8.0×10¹⁰ to 1.0×10¹⁴ particles (e.g. FDPDs)/kg of a subject), 1.0×10¹¹ to 1.0×10¹⁴ particles (e.g. FDPDs)/kg of a subject), 5.0×10¹¹ to 1.0×10¹⁴ particles (e.g. FDPDs)/kg of a subject), 8.0×10¹¹ to 1.0×10¹⁴ particles (e.g. FDPDs)/kg of a subject), 1.0×10¹² to 1.0×10¹⁴ particles (e.g. FDPDs)/kg of a subject), 3.0×10¹² to 1.0×10¹⁴ particles (e.g. FDPDs)/kg of a subject), 5.0×10¹² to 1.0×10¹⁴ particles (e.g. FDPDs)/kg of a subject), or 8.0×10¹² to 1.0×10¹⁴ particles (e.g. FDPDs)/kg of a subject).

In some embodiments, a medicament or a method of treating a subject can have a low, medium, or high dosage of platelet derivatives that has a potency in the range of 250 to 5000 TGPU per kg of the subject.

In some embodiments of any aspect or embodiment herein a therapeutically effective dose or an effective dose or amount of the platelet derivatives is an amount that has a potency in the range of 250 to 5000 TGPU per kg, 270 to 4500 TGPU per kg, 280 to 4300 TGPU per kg, 290 to 4100 TGPU per kg, 300 to 3800 TGPU per kg, 310 to 3500 TGPU per kg, or 320 to 3000 TGPU per kg of the subject. In some embodiments, a therapeutically effective dose or effective dose or amount of the platelet derivatives is an amount that has a potency in the range of 275 to 500 TGPU per kg, 280 to 450 TGPU per kg, 290 to 400 TGPU per kg, 300 to 375 TGPU per kg, 310 to 350 TGPU per kg, or 320 to 340 TGPU per kg of the subject. In some embodiments of any aspect or embodiment herein a therapeutically effective dose or effective dose or amount of the platelet derivatives is an amount that has a potency of 270 TGPU per kg, 280 TGPU per kg, 290 TGPU per kg, 300 TGPU per kg, 310 TGPU per kg, 320 TGPU per kg, 330 TGPU per kg, 340 TGPU per kg, or 350 TGPU per kg of the subject.

In some embodiments of any aspect or embodiment herein a therapeutically effective dose or effective dose or amount of the platelet derivatives is an amount that has a potency in the range of 1001 to 2000 TGPU per kg, 1200 to 2000 TGPU per kg, 1300 to 1950 TGPU per kg, 1400 to 1900 TGPU per kg, 1500 to 1900 TGPU per kg, 1600 to 1900 TGPU per kg, 1700 to 1900 TGPU per kg, 1750 to 1875 TGPU per kg, or 1800 to 1850 TGPU per kg of the subject. In some embodiments, a therapeutically effective dose or effective dose or amount of the platelet derivatives is an amount that has a potency of 1780 TGPU per kg, 1790 TGPU per kg, 1800 TGPU per kg, 1810 TGPU per kg, 1820 TGPU per kg, 1830 TGPU per kg, 1840 TGPU per kg, or 1850 TGPU per kg of the subject.

In some embodiments of any aspect or embodiment herein a therapeutically effective dose or effective dose or amount of the platelet derivatives is an amount that has a potency in the range of 2001 to 3500 TGPU per kg, 2300 to 3300 TGPU per kg, 2500 to 3100 TGPU per kg, 2600 to 3100 TGPU per kg, 2700 to 3100 TGPU per kg, 2800 to 3100 TGPU per kg, 2850 to 3050 TGPU per kg, 2900 to 3000 TGPU per kg, or 2940 to 2990 TGPU per kg of the subject. In some embodiments, a therapeutically effective dose or effective dose or amount of the platelet derivatives is an amount that has a potency of 2910 TGPU per kg, 2920 TGPU per kg, 2930 TGPU per kg, 2940 TGPU per kg, 2950 TGPU per kg, 2960 TGPU per kg, 2970 TGPU per kg, 2980 TGPU per kg, 2990 TGPU per kg, 3000 TGPU per kg, or 3100 TGPU per kg of the subject.

In certain embodiments, any of the dose ranges provided herein, and in illustrative embodiments those that include less than 1×10¹¹ particles/kg, or any of the ranges provided herein, for example those provided in the paragraph immediately above or any aspect or embodiment that includes an “administering” step, can be administered more than 1 time to a subject. For example, a dose range of between 1.0×10⁷ particles to about 1.0×10¹⁰ particles, can be administered between 2 and 10 times, or between 2 and 8 times, or between 2 and 6 times, or between 3 and 8 times, or between 3 and 6 times, or between 4 and 6 times in a timeframe between within 1, 2, 3, 4, 5, or 7 days from the first dose. And in some embodiments a single (e.g., 1×10¹⁰), double (e.g., 2×10¹⁰), triple (e.g., 3×10¹⁰), or higher dose can be administered.

In a method of treating a subject with platelet derivatives as described herein, there can be several endpoints that determine if the subject is treated. A method of treating can have one or more primary endpoints. A method can additionally have one or more secondary endpoints. In some embodiments, in a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In illustrative embodiments, a method or a medicament as described herein leads to cessation or decrease in bleeding at bleeding sites other than primary bleeding site at 24 hours after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition. In some embodiments, the bleeding in a subject is a non-compressible bleeding or a non-compressible hemorrhage. A non-compressible hemorrhage is a type of hemorrhage that is inaccessible to a tourniquet or pressure dressing. In some embodiments, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject. In some illustrative embodiments, the increase can be at least 5000 platelets/μl.

In some embodiments of any of the aspects and embodiments herein that include platelet derivatives in a hydrated or rehydrated form, the protein concentration, or plasma protein concentration, is in the range of 0.01%-50%, 5%-50%, 5%-30%, 5-15%, 8%-10%, 7%-10%, or 3-7% of the protein concentration of donor apheresis plasma. In some embodiments of a composition or in some compositions used in or formed by a process herein, the protein concentration, or plasma protein concentration is less than or equal to 30%, 25%, 20%, 15%, 10%, 5%, 4%, 3%, 2%, or 1% of the protein concentration of donor apheresis plasma. In some embodiments of a composition or a process herein, the protein concentration, or plasma protein concentration is less than or equal to 15%, 14%, 13%, 12%, 11%, 10%, 9%, 8%, 7%, 6%, 5%, 4%, 3%, 2%, 1%, 0.1%, or 0.01%. In some exemplary embodiments, the protein concentration, or plasma protein concentration is less than 3% or 4%. In some embodiments, the protein concentration, or plasma protein concentration is between 0.01% and 20%, 0.01% and 15%, 0.01% and 10%, 0.01% and 5%, 0.1% and 20%, 0.1% and 15%, 0.1% and 10%, 0.1% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments of a composition or a process herein, the protein concentration is in the range of 0.01-15%, 0.1-15%, 1-15%, 1-10%, 0.01-10%, 3-12%, or 5-10%. In some embodiments, the absorbance at 280 nm is less than or equal to 2.0 AU, or 1.90 AU, or 1.80 AU, or 1.7 AU, or 1.66 AU, or 1.6 AU when measured using a path length of 0.5 cm.

In some embodiments of any of the aspects and embodiments herein that include platelet derivatives in a powdered form, the protein concentration is in the range of 0.01-15%, 0.1-15%, 1-15%, 1-10%, 0.01-10%, 3-12%, or 5-10%. In some embodiments, the protein concentration is less than or equal to 25%, 20%, 15%, 10%, 7.5%, 5%, 2.5%, 1%, or 0.1%.

In some embodiments of any of the aspects and embodiments herein that include a composition, or in some compositions used in or formed by a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments of the composition, a percentage of beads positive for HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments, the composition is negative for the antibodies selected from the group consisting of HLA Class I antibodies, HLA Class II antibodies, and HNA antibodies based on a regulatory agency approved test for the respective antibodies. In some embodiments, the composition is negative for HLA Class I antibodies, HLA Class II antibodies, and HNA antibodies based on a regulatory agency approved test for the respective antibodies.

In some embodiments of any of the aspects and embodiments herein that include a composition, or in some compositions used in or formed by a process that includes a population of platelet derivatives in a hydrated or rehydrated form, comprises trehalose in the range of 0.4-35%, or 1-35%, or 2-30%, or 1-10%, or 1-5%, or 0.5-5%. In an exemplary embodiment, the composition comprises 3.5% trehalose.

In some embodiments of any of the aspects and embodiments herein that include a composition, or in some compositions used in or formed by a process that includes a platelet composition in a powdered form, comprises trehalose having a weight percentage in the range of 10-60%, 15-55%, 20-60%, 20-50%, 25-60%, 25-50%, 10-50%, 20-40%, or 20-35%. In some embodiments, the weight percentage of trehalose can vary on the weight percentage of other components in the composition like, polysucrose, platelet derivatives, plasma protein, and buffering agents.

In some embodiments of any of the aspects and embodiments herein that include a composition, or in some compositions used in or formed by a process that includes a population of platelet derivatives in a hydrated or rehydrated form, comprises polysucrose in the range of 2-8%, 2.25-7.75%, 2.5-7.5%, or 2.5-6.5%. In an exemplary embodiment, the composition comprises 3% polysucrose. In another exemplary embodiment, the composition comprises 6% polysucrose. In some embodiments, polysucrose is polysucrose 70 kDa. In some embodiments, polysucrose is a polysucrose 400 kDa.

In some embodiments of any of the aspects and embodiments herein that include a composition, or in some compositions used in or formed by a process that includes a platelet composition in a powdered form, comprises polysucrose having a weight percentage in the range of 20-80%, 25-75%, 30-70%, 35-65%, 30-80%, or 45-60%. In some embodiments, the weight percentage of trehalose can vary on the weight percentage of other components in the composition like, trehalose, platelet derivatives, plasma protein, and buffering agents.

In some embodiments of any of the aspects and embodiments herein that include a composition, or in some compositions used in or formed by a process that includes a platelet composition in a powdered form, comprises trehalose and polysucrose having a combined weight percentage in the range of 30-95%, 35-95%, 40-90%, 40-90%, 45-90%, or 60-95%.

In some embodiments of any of the aspects and embodiments herein that include a composition or in some compositions used in or formed by a process herein, comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 MW to 400,000 MW, 100,000 MW to 400,000 MW, 200,000 MW to 400,000 MW, 80,000 MW to 350,000 MW, 100,000 MW to 300,000 MW, 100,000 MW to 200,000 MW, 120,000 MW to 200,000 MW. In some exemplary embodiments, polysucrose has a molecular weight of 150,000 MW, 160,000 MW, 170,000 MW, 180,000 MW, 190,000 MW, or 200,000 MW.

In some embodiments of any of the aspects and embodiments herein that include a composition or in some compositions used in or formed by a process herein, comprises platelet derivatives that are positive for at least one platelet activation marker selected from the group consisting of phosphatidylserine (PS), and CD 62. Typically, phosphatidylserine (PS) can be detected by using Annexin V. Accordingly, Annexin V positivity, or the platelet derivatives positive for Annexin V can refer to the binding of Annexin V to the platelet derivatives. In some embodiments, the platelet derivatives are positive for at least one platelet marker selected from the group consisting of CD 41, CD 42, and CD 61. In some embodiments, the platelet derivatives are positive for CD 47. In some embodiments, the platelet derivatives are positive for Annexin V. In some embodiments, the platelet derivatives are positive for Annexin V. In some embodiments, at least 25%, 50%, or 75% of the platelet derivatives in the platelet derivative composition are Annexin V positive. In some embodiments, the platelet derivatives are positive for CD 41. In some embodiments, at least 30%, 35%, 40%, 45%, 50%, 55%, 60%, or 65% of the platelet derivatives in the platelet derivative composition are CD41 positive. In some embodiments, at least 90%, 91%, 92%, 93%, 94%, 95% 96%, 97%, 98%, or 99% platelet derivatives that are positive for CD 41 have a size in the range of 0.5-2.5 μm. In some exemplary embodiments, at least 95% platelet derivatives that are positive for CD 41 have a size in the range of 0.5-2.5 μm. In some embodiments, the platelet derivatives are positive for CD 42. In some embodiments, at least 65%, 80%, or 90% of the platelet derivatives in the platelet derivative composition are CD42 positive. In some embodiments, the platelet derivatives are positive for CD 47. In some embodiments, at least 8%, 10%, 15%, or 20% of the platelet derivatives in the platelet derivative composition are CD47 positive. In some embodiments, the platelet derivatives are positive for CD 62. In some embodiments, at least 10%, 50%, 80%, or 90% of the platelet derivatives in the platelet derivative composition are CD62 positive. In some embodiments, the platelet derivatives in the platelet derivative composition are positive for CD41, CD62, and Annexin V. In some embodiments, the platelet derivatives in the platelet derivative composition are at least 50% platelet derivatives are positive for CD41, at least 70% platelet derivatives are positive for CD62, and at least 70% platelet derivatives are positive for Annexin V.

In some embodiments of any of the aspects and embodiments herein that include a composition or in some compositions used in or formed by a process herein, the platelet derivatives have fibrinogen associated with their cell membrane. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets.

In some embodiments of any of the aspects and embodiments herein that include a composition or in some compositions used in or formed by a process herein that includes a population of platelet derivatives in a hydrated or rehydrated form, the platelet derivatives in the platelet derivative composition retain at least 10%, or 15%, or 20% of the lactate dehydrogenase activity of donor apheresis platelets. In some embodiments, the aqueous medium has a lactate concentration of less than 2.0 mmol/L, or 1.5 mmol/L. In some embodiments, the lactate concentration is in the range of 0.4 to 1.3 mmol/L, or 0.5 to 1.0 mmol/L.

In some embodiments of any of the aspects and embodiments herein that include a composition, or in some compositions used in or formed by a process that includes a population of platelet derivatives a powdered form, the platelet derivative composition comprises no more than 0.1%, 0.2%, 0.3%, 0.4%, 0.5%, 0.6%, 0.7%, 0.8%, 0.9%, 1%, 1.5%, 2%, 2.5%, 3%, 3.5%, 4.0%, 4.5%, or 4.9% residual moisture. In some embodiments, wherein the platelet derivative composition is in a powdered form, the platelet derivative composition comprises residual moisture in the range of 0.1-2%, 0.2-1.5%, 0.5-1.5%, 0.75-1.25%, 2-3%, 2.5-4.9%, 3-4.5%, 1.5-3%, or 1-2% residual moisture. In some illustrative embodiments, the platelet derivative composition comprises no more than 0.5% residual moisture.

In some embodiments of any of the aspects and embodiments herein that include a composition, or in some compositions used in or formed by a process that includes a plurality of containers each filled with a platelet derivative composition in the form of a powder, the platelet derivative composition in at least one of the plurality of containers comprises or is associated with a first protein from a first gene that has a different amino acid sequence than found in all the versions of the first protein from the first gene in the platelet derivative composition in one or more other containers of the plurality.

In some embodiments of any of the aspects and embodiments herein that include a composition, or in some compositions used in or formed by a process that includes a plurality of containers each filled with a platelet derivative composition in the form of a powder, the at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one protein of a collection of protein gene products from a corresponding collection of encoding genes. In some embodiments, the different amino acid sequences differ at one or more residues corresponding to amino acid residues encoded by a non-synonymous single nucleotide polymorphism (SNP). In some embodiments, the platelet derivative composition is in a container, and wherein the container is filled with at least one inert gas.

In some embodiments of any of the aspects and embodiments herein that include a composition, or in some compositions used in or formed by a process that includes a plurality of containers each filled with a platelet derivative composition in the form of a powder, the amount of plasma protein in the powder of any two containers chosen from different lots, differs by less than 50%, 40%, 30%, 20%, 10%, 5%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the amount of microparticles that are less than 0.5 μm in the powder of any two containers chosen from different lots, differs by less than 50%, 40%, 30%, 20%, 10%, 5%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one, two, three, four, or five protein of a collection of protein gene products from a corresponding collection of encoding genes. In some embodiments, the different amino acid sequences differ at one or more residues corresponding to amino acid residues encoded by a non-synonymous single nucleotide polymorphism (SNP).

In some embodiments of any of the aspects and embodiments herein that includes a plurality of containers each filled with a platelet derivative composition in the form of a powder, the containers can vary in size from 5-100 ml, 10-90 ml, 25-75 ml, or 5-40 ml. In some illustrative embodiments, the size of vials is 30 ml. In some other illustrative embodiments, the size of vials is 10 ml. In some embodiments, enough of the starting material comprising platelet composition is processed for platelet derivative composition as described herein in order to pack around 200 vials of 10 ml each. The number of vials in which the end product of platelet derivative composition can vary with the manufacturing requirements and the amount of starting material.

In some embodiments of any of the aspects and embodiments herein that include a composition, or in some compositions used in or formed by a process that includes a plurality of containers each filled with a platelet derivative composition in the form of a powder.

In some embodiments of any of the aspects and embodiments herein that include a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopeni purpura, trauma, or a combination thereof. In some embodiments, the composition is passed through a filter of 18 μm before administering to the subject.

In some embodiments, the platelet derivative composition of any of the aspects or embodiments herein is provided for use in the treatment of a disorder selected from the group consisting of alopecia areata, Von Willebrand Disease, hemophilia, thrombasthenia, thrombocytopenia, thrombocytopeni purpura, trauma, or a combination thereof.

In some embodiments, the platelet derivatives as described herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject. In some embodiments, the platelet derivative composition of any of the aspects or embodiments herein is provided for use in wound healing in a subject.

The platelet derivative composition as described herein can be contained in containers/vials, which further can be packed into a plurality of containers for shipping to a customer, which can be part of a commercialization process to fulfill an order for such platelet derivative composition. The containers, in certain embodiments, are 5 ml vials, 10 ml vials, 20 ml vials, 25 ml vs, 30 ml vials, 40 ml vials, 50 ml vials, 60 ml vials, 75 ml vials, 100 ml vials, 125 ml vials, 150 ml vials, 200 ml vials, or 250 ml vials. The vial(s) can be a cryovial, or a cryotube especially in illustrative embodiments where the TFF-treated composition that includes platelets is lyophilized to obtain the platelet derivative composition in the form of a powder, which further can be baked or not baked after it is lyophilized. In some embodiments, the volume of the containers in a plurality of containers (e.g. vials or tubes), which for example can be all from one lot, or from more than one lot (e.g. 2, 3, 4, 5, 6, 7, 8, 9 or 10 lots), can vary from one or more than one size between 10-100 ml. Typically, the volume of the vial/container in embodiments where the platelet derivative is a freeze-dried solid/powder, is 1× the volume of, or 1.10, 1.25, 1.5, 2, 2.5, 3, 4 or 5 times the volume of a composition that was filled in the vial before lyophilization, and/or the volume in which the powder in the vials will be rehydrated, which is an illustrative embodiment. Thus, the maximum volume of such vials can be the same or more than the volume of the composition that was filled inside prior to lyophilization or the volume in which the platelet derivative composition in the form of a powder can be rehydrated. For example, in one non-limiting embodiment, a vial with a maximum capacity of 100 ml, can be used to fill 10 ml of a TFF-treated composition that includes platelets for lyophilization. In certain embodiments, the capacity of a vial in which a TFF-treated composition that includes platelets is lyophilized, is 1-2.5 times and in other embodiments, 1-2 times, 1-3 times, 1-4 times, 1-5 times, and in certain illustrative embodiments, 1.1 to 2 times or 1.25 to 2 times the volume of a TFF-treated composition that is lyophilized therein.

The TFF-treated platelet composition before lyophilization, or in some embodiments, the platelet derivative composition obtained after the lyophilization step, with or without post-lyophilization heat treatment (baking), can be filled into a plurality of vessels or other powder and liquid-holding containers, such as vials, in a sterile manner. In some embodiments, the containers can vary in volume from 5-100 ml, 10-90 ml, 25-75 ml, or 5-40 ml. In some embodiments, the volume of containers can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers can be above 100 ml, for example, 125 ml, 150 ml, 175 ml, or 200 ml. The platelet derivative composition as described herein can be filled in vials of different volumes as per the commercialization requirements. A plurality (or collection) of containers having the platelet derivative composition as per any of the embodiments herein, obtained by lyophilizing the composition that includes platelets during one process (e.g. TFF or other process) for preparing a platelet derivative, can be referred to as a “batch” or a “lot”. In some embodiments, a batch/lot can have 10-500 vials, 25-450 vials, 50-350 vials, 100-300 vials, or 150-250 vials. In some embodiments, a batch/lot can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the number of vials per batch/lot can be increased to more than 500 as per the requirements, for example, 600, 700, 800, 900, or 1000 vials. In some embodiments, the number of vials can be 10-1000, 50-1000, 100-900, 200-800, 100-500, 100-400, 150-700, or 150-500 vials. The containers in a batch/lot can have a volume in the range of 5-100 ml, for example, such that a lot has several containers with the same volume or containers with different volumes. For example, 200 vials/containers in a batch/lot can have a volume of 10 ml each, 100 vials/containers in the same or a separate batch/lot can have a volume of 20 ml each, 100 vials/containers in the same or another batch/lot can have a volume of 30 ml each, or 300 vials/containers in the same or a different batch/lot can have a volume of 10 ml each. The number of containers (e.g. vials) in which a platelet derivative composition as per one of the embodiments or aspects described herein can be packed in a batch/lot can vary with manufacturing requirements, the requirements of downstream processes, for example clinical processes, and the amount of starting material comprising platelet composition.

The quantity of platelet derivatives that is present in a batch/lot can vary based on the units of starting material comprising platelets that is used to produce the platelet derivatives. Certain methods provided herein, such as the TFF methods provided herein, allow more platelet units to be used to make platelet derivatives with the characteristics provided herein than prior methods. This is the result, for example, of the ability to reduce the level of certain components in a platelet composition starting material, such as HLA antibodies, HNA antibodies, and/or microparticles, to very low levels, as provided herein. Accordingly, the starting material comprising platelets, the corresponding composition (e.g. TFF-treated composition) that is lyophilized in illustrative embodiments, and the resulting platelet derivative composition powder, can vary, such that for example, in some embodiments, the starting material, the TFF-treated composition, and/or the resulting platelet derivative composition powder can include 10-500 units of platelets or platelet derivatives (e.g. 0.5 to 2.5 μm in diameter), with one unit being 3×10¹¹ platelets or platelet derivatives. In some embodiments, the starting platelet material, the composition to be lyophilized, and/or the platelet derivative (e.g. 0.5 to 2.5 μm in diameter) composition can include, for example, 20-500 units, 30-400 units, 40-350 units, or 50-200 units of platelets or platelet derivatives, respectively. In some embodiments, the platelet units in the starting platelet composition can be a pooled platelet product from multiple donors as described herein, or multiple batches of processed platelet compositions, such as TFF-treated compositions comprising platelets, can be pooled before lyophilization. In some embodiments, there can be 1×10⁹ to 1×10¹⁶ platelets in a starting platelet composition for processing, in a platelet composition that is lyophilized, and/or of platelet derivatives in a platelet derivative composition that is produced after lyophilization, per batch/lot. In some embodiments, the platelet-containing starting composition, the platelet composition that is lyophilized, and/or the platelet derivatives that are produced, typically after lyophilization per batch/lot can vary from 1×10¹⁰ to 1×10¹⁵, 1×10¹¹ to 1×10¹⁵, 1×10¹² to 1×10¹⁶, 1×10¹³ to 1×10¹⁵ or 1×10¹³ to 1×10¹⁴.

In certain illustrative embodiments, platelet derivative compositions that are present in a liquid, or in illustrative embodiments, a solid form such as a dried powder in the plurality of containers (e.g. vials), in illustrative embodiments of a 1 or more lots, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are 0.5 to 2.5 μm in diameter. Such platelet derivatives and platelet derivative compositions comprising the same, can have additional characteristics disclosed herein for such derivatives and compositions.

Processes provided herein for producing platelet derivative compositions, provide better lot to lot consistency than prior processes. For example, TFF methods provided herein provide improved lot to lot variability with respect to the components of compositions that include platelet derivatives prepared therein, in illustrative embodiments, compositions that include freeze-dried platelet derivates. Such freeze-dried platelet derivatives can be one of, or the main active ingredient(s). In some embodiments, a plurality of containers provided herein comprise the platelet derivative composition from at least 2 different lots in separate containers. In some embodiments, the amount of plasma protein in the powder of any two containers chosen from different lots, differs by less than 50%, 40%, 30%, 25%, or 20%, and in illustrative embodiments less than 10%, 5%, 2%, 1%, or 0.5%. The TFF process is highly controllable and can be stopped at a certain A280 for example, from 2.0 AU to 0.01 AU, or when it reaches 15% to 0.01% protein concentration in the composition that is to be lyophilized (e.g. TFF-treated composition), therefore, the plasma protein content can be very consistent not only within the containers/vials of a lot, but even between lots as well. Since different lots of platelet derivative compositions provided herein are typically prepared from platelets from different subjects or different combinations of subjects (e.g. pooled platelets from 2, 3, 4, 5, 6, 7, 8, 9, 10, 15, 20, 25, 30, 40, 50, 60, 70, 75, 80, or 100 subjects), different lots in illustrative embodiments differ in amino acid sequence of at least one (e.g., 1, 2, 3, 4, 5, 6, 7, 8, 9, 10, 1-100, or 1-10) of the proteins in, on, and/or associated with platelet derivatives of the compositions therein between the lots. In illustrative embodiments, these one or more amino acid differences occur at one or more (e.g., 1, 2, 3, 4, 5, 6, 7, 8, 9, 10, 1-100, or 1-10) of the site(s) of non-synonymous SNPs. In certain embodiments, such non-synonymous SNPs have a minor allele frequency of less than or equal to 5%. In some embodiments, such pooled platelets are provided by processes provided herein, for example because HLA or HNA antibody levels can be reduced to very low or non-existent levels. Thus, not only can platelets be pooled from more subjects, before processing to form platelet derivatives, but those subjects can be males or females. As a result, in certain embodiments, within a lot, greater than 10%, 20%, 25%, 30%, or 40%, and in illustrative embodiments greater than 50%, 60%, 70%, 75%, 80%, 90%, or 95% of non-synonymous SNPs in one or more proteins that are bound to or otherwise associated with or part of a platelet derivative, are present for SNPs with a minor allele frequency of greater than 5%, in certain embodiments including SNPs on a mammalian X and Y chromosome.

In some embodiments, the amount of microparticles that are less than 0.5 μm in the powder of any two containers chosen from different lots, differs in amount by less than 10%, 5%, 2%, or 1%. Since, for example, a TFF process disclosed herein is very controllable, the concentration of microparticles to be obtained in the platelet derivative composition can be optimized, for example, by performing scattering intensity studies at different time points. Once the desired level is achieved, the TFF-treated composition can be lyophilized and packed in the vials with or without the baking step.

In some embodiments, the percentage by weight of platelet derivative in the powder of any two containers chosen from different lots, differs by less than 10%, 5%, 2%, or 1%. The TFF process can be optimized to achieve a pre-determined level of platelet derivatives in the TFF-treated composition. Such a TFF-treated composition when lyophilized gives a platelet composition in the form of a powder having a certain weight percentage of platelet derivatives. Since, the TFF process is controllable, in some embodiments, there can be a minimum or a negligible variation in the weight percentages of the platelet derivatives in any two containers chosen from different lots.

In some embodiments, at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

As per one of the embodiments, a platelet derivative composition as described herein can be prepared from multiple donors of a single species, for example, mammals, such as for example canine, equine, porcine and in illustrative embodiments humans that are genetically different, in order to obtain a platelet derivative composition to prepare allogenic platelet derivatives, an allogenic platelet derivative product, and/or a composition comprising allogenic platelet derivatives. Such a platelet derivative composition can be filled in vials and a plurality of such vials can be packaged in containers, for example boxes for commercialization as described herein, to obtain a commercial product that is a composition comprising allogeneic platelet derivatives. The allogenic platelet derivatives as described herein, in some embodiments, can be a U.S. FDA-approved product comprising an allogenic platelet derivative composition. In some embodiments, a platelet derivative composition as described herein can be a European EMA-approved product comprising an allogenic platelet derivative composition. In some other embodiments, a platelet derivative composition as described herein can be a China FDA-approved product comprising an allogenic platelet derivative composition.

In some embodiments, platelets are pooled from a plurality of donors before they are used as starting material for a process for producing a platelet derivative as provided herein. Such platelets pooled from a plurality of donors can be also referred herein to as pooled platelets. In some embodiments, the donors are more than 5, such as more than 10, such as more than 20, such as more than 50, such as up to about 100 donors. In some embodiments, the donors are from 5 to 100, such as from 10 to 50, such as from 20 to 40, such as from 25 to 35. Pooled platelets can be used to make any of the platelet derivative compositions as described herein. The platelets can be pooled wherein the platelets are donated by mammalian (e.g. bovine, feline, porcine, canine, and in illustrative embodiments, human) subjects. In some embodiments, the gender of the subjects can be male or female. In some embodiments, the donor can vary from any number of male to any number of female subjects, for example, from a total of 100 donors, any number can be female donors, ranging from 0-100, 5-95, 10-90, 20-80, 30-70, or 40-60, and the rest can be male donors. In some other embodiments, the donor can be a non-human animal. In some embodiments, the donor can be a canine, equine, porcine, bovine, or feline subject.

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

Platelet derivatives in certain illustrative aspects and embodiments herein are surrounded by a compromised plasma membrane. In these illustrative aspects and embodiments, the platelet derivatives lack an integrated membrane around them. Instead, the membrane has pores on them that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The compromised membrane of the platelet derivatives can also be determined by flow cytometry studies.

Platelet or platelet derivatives (e.g., thrombosomes) as described herein can retain some metabolic activity, for example, as evidenced by lactate dehydrogenase (LDH) activity. In some cases, platelets or platelet derivatives (e.g., thrombosomes) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., thrombosomes) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

Platelet derivatives as described herein can have several applications in terms of treating a subject suffering with a disorder selected from the group consisting of alopecia areata, Von Willebrand Disease, hemophilia, thrombasthenia, thrombocytopenia, thrombocytopenia purpura, trauma, or a combination thereof. In some embodiments, the platelet derivatives can be used to treat clotting-related disorders. The platelet derivatives as described herein can be used both as a topical application and systemic administration. In some embodiments, there is provided a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenia purpura, trauma, or a combination thereof. In some embodiments, a platelet derivative composition is passed through a filter of 18 μm before administering to the subject. A skilled artisan would be able to appreciate that the platelet derivative composition in the form of a powder which would be commercialized in vials would be rehydrated with an appropriate amount of a solution before administering to a subject. In some embodiments, such a rehydrated platelet derivative composition is passed through a filter of 18 μm before administering to the subject.

In some embodiments, the platelet derivatives as described herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject and/or a wound of the subject.

In some embodiments, the administering can include administering topically. Administering can include administering parenterally. Administering can include administering intravenously. Administering can include administering intramuscularly. Administering can include administering intrathecally. Administering can include administering subcutaneously. Administering can include administering intraperitoneally.

In some embodiments of any of the methods described herein, the subject may or may not be also treated with an anticoagulant reversal agent (e.g., idarucizumab, Andexanet Alfa, Ciraparantag (aripazine), protamine sulfate, vitamin K). In some embodiments, the subject is not also treated with an anticoagulant reversal agent. In some embodiments, the subject is also treated with an anticoagulant reversal agent. It will be understood that an anticoagulant reversal agent can be chosen based on the anticoagulant administered to the subject.

Some embodiments provide a method of ameliorating the effects of an anticoagulant in a subject, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of ameliorating the effects of an anticoagulant in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, treatment with an anticoagulant can be stopped (e.g., in preparation for surgery) in illustrative embodiments before the composition comprising platelet derivatives is administered to the subject. In some embodiments, treatment with an anticoagulant can continue in illustrative embodiments for a time period after the composition comprising platelet derivatives is administered to the subject. Such a time period can include 1, 2, 3, 4, 5, 6, or 7 days, or 1, 2, 3, or 4 weeks, or 1, 2, or 3 months or longer.

Some embodiments provide a method of ameliorating the effects of an anticoagulant agent in a subject, the method comprising administering to the subject in need thereof an effective amount of a composition comprising platelets, or in illustrative embodiments a composition comprising platelet derivatives, which in further illustrative embodiments are FDPDs and an incubating agent comprising one or more salts, a buffer, optionally a cryoprotectant, and optionally an organic solvent.

Some aspects provide a method of ameliorating the effects of an anticoagulant agent in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, the effects of an anticoagulant agent may need to be ameliorated due to an incorrect dosage of an anticoagulant agent. For example, in some embodiments, the effects of an anticoagulant agent can be ameliorated following an overdose of the anticoagulant agent. In some embodiments, the effects of an anticoagulant agent may need to be ameliorated due to a potential for interaction with another drug (e.g., a second anticoagulant agent). For example, in some embodiments, the effects of an anticoagulant agent can be ameliorated following an erroneous dosing of two or more drugs, at least one of which is an anticoagulant agent.

In some embodiments of any of the methods described herein, the composition can further comprise an active agent, such as an anti-fibrinolytic agent. Non-limiting examples of anti-fibrinolytic agents include ε-aminocaproic acid (EACA), tranexamic acid, aprotinin, aminomethylbenzoic acid, and fibrinogen. In some embodiments, platelets or platelet derivatives can be loaded with an active agent, such as an anti-fibrinolytic agent.

Compositions comprising FDPDs herein, in certain embodiments have the surprising property that they can reduce bleeding or the bleeding potential and in illustrative embodiments, restore hemostasis in a subject whose blood has an elevated bleeding potential, independent of whether a laboratory test for bleeding potential of the subject is negative or positive after administration of the FDPDs. Such elevated bleeding potential in illustrative embodiments is typically because an effective amount of anticoagulant agent was delivered to the subject and is in the blood of the subject. Accordingly, in any of the aspects herein, in some embodiments, the composition comprising FDPDs has the property that it is capable of reducing the bleeding potential of the subject, independent of whether a post-administering evaluation of bleeding potential, if performed, yields a normal or abnormal result. In some embodiments such post-administering evaluation comprises an in vitro laboratory test performed on a sample taken or drawn at a time period, for example, between 1 and 4, or 1 and 3, or 1 and 2 hours after administering the composition comprising FDPDs to the subject. In other embodiments of any of the aspects herein, wherein the composition comprising FDPDs has the property that it is capable of reducing the bleeding potential of a subject such that normal hemostasis is restored in a subject having an increased bleeding potential, independent of whether a post-administering evaluation of bleeding potential yields a normal or abnormal result. In some embodiments, such post-administering evaluation if performed, comprises an in vitro laboratory test performed on a sample taken or drawn at a time period, for example, between 1 and 4, or 1 and 3, or 1 and 2 hours after administering the composition comprising FDPDs to the subject. The time period, can be for example, within 0 minutes and 72 hours, or between 10 minutes and 72 hours, or between 10 minutes and 48 hours, or between 10 minutes 24 hours, or between 10 minutes and 4 hours, or between 10 minutes and 1 hour, or between 10 minutes and 30 minutes, or between 30 minutes and 24 hours, or between 30 minutes and 4 hours, or between 30 minutes and 1 hour after administering the composition comprising the platelet derivatives (e.g. FDPDs) to the subject. The lab test in certain embodiments, is one or more, or two or more, or three or more of the bleeding parameters disclosed herein.

In any of the aspects herein, in some embodiments the composition comprising platelet derivatives (e.g. FDPDs) has the property that it is capable of reducing the bleeding or the bleeding potential of a subject having an elevated bleeding potential, or increased bleeding that can be life-threatening. Furthermore, the composition comprising FDPDs typically has the additional and surprising property, that after being administered to the subject in an effective amount, for example for reducing the bleeding or the bleeding potential of the subject, the subject may have an abnormal value for one or more in vitro lab tests, for example of one or more clotting parameters in a post-administering evaluation performed using an, or the in vitro laboratory test performed on a blood sample taken between 15 minutes and 4 hours, 30 minutes and 4 hours, 1 hour and 4 hours, or taken between 15 minutes and 2 hours, 30 minutes and 2 hours, or 1 hour and 2 hours, or taken between 15 minutes and 1 hour or 30 minutes and 1 hour, after administering the composition comprising FDPDs. In some embodiments of this embodiment, the composition comprising FDPDs has the property that it is capable of reducing the bleeding or the bleeding potential of a subject to about or at a normal hemostasis or about or at the hemostasis level of the subject when not taking the anticoagulant agent. Yet, in these embodiments, the composition comprising FDPDs retains the additional and surprising property, that after being administered to the subject in the effective amount, such a property is independent of a post-administering lab test for bleeding potential. Thus, in some embodiments, the subject would have an abnormal value for the one or more clotting parameters in a post-administering evaluation performed using an, or the in vitro laboratory test performed on a blood sample taken between 1 and 4 hours, or any of the time ranges recited immediately above, after administering the composition comprising FDPDs. It will be understood that in methods that include compositions comprising FDPDs with such properties, or any properties that include an evaluation or test, no testing actually needs to be performed to practice such methods unless such testing step is actually recited as a step of the method.

In certain embodiments the composition comprising FDPDs comprises a population of FDPDs having a reduced propensity to aggregate such that no more than 2%, 3%, 4%, 5%, 7.5%, 10%, 12.5%, 15%, 17.5%, 20%, 22.5%, or 25% of the FDPDs in the population aggregate under aggregation conditions comprising an agonist but no platelets. In certain embodiments the FDPDs have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles.

In certain embodiments the FDPDs have one or more characteristics of super-activated platelets. Such characteristics can include one or more of the following:
A) the presence of thrombospondin (TSP) on their surface at a level that is greater than on the surface of resting platelets;
B) the presence of von Willebrand factor (vWF) on their surface at a level that is greater than on the surface of resting platelets; and
C) an inability to increase expression of a platelet activation marker in the presence of an agonist as compared to the expression of the platelet activation marker in the absence of an agonist.

In some embodiments less than 5% of a population of FDPDs, and in illustrative embodiments CD 41-positive FDPDs are microparticles having a diameter of less than 0.5 μm. Platelet derivatives herein have been observed to have numerous surprising properties, as disclosed in further detail herein. It will be understood, as illustrated in the Examples provided herein, that although platelet derivatives in some aspects and embodiments are in a solid, such as a powder form, the properties of such platelet derivatives can be identified, confirmed, and/or measured when a composition comprising such platelet derivatives is in liquid form.

In some embodiments, the platelets or platelet derivatives (e.g., FDPDs) have a particle size (e.g., diameter, max dimension) of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets or platelet derivatives (e.g., FDPDs), have a particle size in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets or platelet derivatives (e.g., FDPDs), are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets or platelet derivatives (e.g., FDPDs) are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

Platelets or platelet derivatives (e.g., FDPDs) as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ FDPDs/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, populations of platelet derivatives (e.g., FDPDs), such as those prepared by methods described herein and included in compositions herein, can have an average CD41 percent positivity of at least 55% (e.g., at least 60%, at least 65%, at least 67%, at least 70%, at least 75%, at least 80%, at least 85%, at least 90%, or at least 95%). In some embodiments, at least 90%, 91%, 92%, 93%, 94%, 95%, 96%, 97%, 98%, 99% platelet derivatives that are positive for CD 41 have a size in the range of 0.5-2.5 μm. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., FDPDs), such as those described herein, can have an average CD42 percent positivity of at least 65% (e.g., at least 67%, at least 70%, at least 75%, at least 80%, at least 85%, at least 90%, or at least 95%). In some embodiments, at least 90%, 91%, 92%, 93%, 94%, 95%, 96%, 97%, 98%, or 99% platelet derivatives that are positive for CD 42 have a size in the range of 0.5-2.5 μm. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm.

As another example, platelets or platelet derivatives (e.g., FDPDs), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., FDPDs), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm. In some embodiments, at least 90%, 91%, 92%, 93%, 94%, 95%, 96%, 97%, 98%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm. Typically, Annexin V positivity can be measured by measuring the binding of Annexin V to the platelet derivatives, FPDPs, or FPH herein. In some embodiments, Annexin V can bind to a cell surface marker on the platelet derivatives, FDPDs, or FPH, in illustrative embodiments, Annexin V binds to phosphatidylserine (PS) expressed or present on the surface of the platelet derivatives, FDPDs, or FPH herein. In illustrative embodiments, PS can be a platelet activation marker, and in some embodiments, at least 50%, 55%, 60%, 65%, 70%, 75%, 80%, 85%, 90%, 95%, or 99% of the platelet derivatives, FDPDs, or FPH herein express PS, or has PS on the surface, or positive for PS. An increased amount of the platelet activation markers, such as phosphatidylserine (PS) on the platelets, or the platelet derivatives indicate the state of activeness of the platelets, or the platelet derivatives. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist. In some embodiments, the platelet derivatives, FDPDs, or FPH herein show an inability to increase expression of a platelet activation marker in the presence of an agonist as compared to the expression of the platelet activation marker in the absence of an agonist, in illustrative embodiments, the platelet activation marker comprises phosphatidylserine (PS). In further illustrative embodiments, the agonist comprises thrombin receptor activator peptide, such as thrombin receptor activator peptide 6 (TRAP-6). In some embodiments, the platelet activation marker or the cell surface marker-phosphatidylserine (PS) can be detected by the binding of Annexin V to the platelets, the platelet derivatives, or the FDPDs. Not to be limited by theory, it is understood that Annexin V binds specifically to phosphatidylserine (PS) present or expressed on the surface of the platelets, platelet derivatives, the FDPDs, or the FPH as disclosed herein.

As another example, platelets or platelet derivatives (e.g., FDPDs), such as those prepared by methods described herein, can have an average CD47 percent positivity of at least about 8% (e.g., at least about 10%, 15%, 20%, 25%, 30%, 35%, 40%, 45%, 50%, or 55%).

Platelets or platelet derivatives (e.g., FDPDs) as described herein can be capable of generating thrombin, for example, when in the presence of a reagent containing tissue factor and phospholipids. For example, in some cases, platelets or platelet derivatives (e.g., FDPDs) (e.g., at a concentration of about 4.8×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 pM, 0.5 pM, 1 pM, 2 pM, 5 pM or 10 pM) and optionally phospholipids. For example, in some cases, platelets or platelet derivatives (e.g., FDPDs) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of a reagent containing tissue factor and (e.g., at 0.25 pM, 0.5 pM, 1 pM, 2 pM, 5 pM or 10 pM) and optionally phospholipids. In some cases, platelets or platelet derivatives (e.g., FDPDs) (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., FDPDs). In some cases, platelets or platelet derivatives (e.g., FDPDs) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., FDPDs).

Platelets or Platelet derivatives (e.g., FDPDs) as described herein can be capable of generating thrombin, for example, when in the presence of a reagent containing tissue factor and phospholipids. For example, in some cases, platelets or platelet derivatives (e.g., FDPDs) can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., FDPDs) can have a potency of between 1.2 and 2.5 TPGU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TPGU per 10⁶ particles). TPGU can be calculated as follows: TGPU/million particles=[TPH in nM]*[Potency Coefficient in IU/(nM)]/[0.576 million particles in the well]. Similarly, the Potency Coefficient for a sample of thrombin can be calculated as follows: Potency Coefficient=Calculated Calibrator Activity (IU)/Effective Calibrator Activity (nM). In some cases, the calibrator activity can be based on a WHO international thrombin standard.

Platelets or platelet derivatives (e.g., FDPDs) as described herein can be capable of clotting, as determined, for example, by using a total thrombus-formation analysis system (T-TAS®). In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 80) of less than 14 minutes (e.g., less than 13.5, 13, 12.5, 12, 11.5, or 11 minutes), for example, in platelet-reduced citrated whole blood. In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood.

Platelets or platelet derivatives (e.g., FDPDs) as described herein can be capable of thrombin-induced trapping in the presence of thrombin. In some cases, platelets or platelet derivatives (e.g., FDPDs) as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., FDPDs) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin. Thrombin-induced trapping can be determined by any appropriate method, for example, light transmission aggregometry. Without being bound by any particular theory, it is believed that the thrombin-induced trapping is a result of the interaction of fibrinogen present on the surface of the platelet derivatives with thrombin.

Platelets For platelet derivatives (e.g., FDPDs) as described herein can be capable of co-aggregating, for example, in the presence of an aggregation agonist, and fresh platelets. Non-limiting examples of aggregation agonists include, collagen, epinephrine, ristocetin, arachidonic acid, adenosine di-phosphate, and thrombin receptor associated protein (TRAP). In some cases, platelets or platelet derivatives (e.g., FDPDs) as described herein can have a percent co-aggregation of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of an aggregation agonist, and fresh platelets. In some cases, platelets or platelet derivatives (e.g., FDPDs) as described herein can have a percent co-aggregation of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of an aggregation agonist. Percent co-aggregation can be determined by any appropriate method, for example, light transmission aggregometry.

Platelet derivative compositions, which in certain illustrative embodiments herein are FDPD compositions, comprise a population of platelet derivatives (e.g. FDPDs) having a reduced propensity to aggregate under aggregation conditions comprising an agonist but no fresh platelets, compared to the propensity of fresh platelets and/or activated to aggregate under these conditions. Platelet derivatives (e.g., FDPDs) as described herein in illustrative embodiments, display a reduced propensity to aggregate under aggregation conditions comprising an agonist but no fresh platelets, compared to the propensity of fresh platelets and/or activated platelets to aggregate under these conditions. Surprisingly, such FDPDs have the ability to increase clotting and aggregation of platelets in in vitro and in vivo assays, in the presence of anti-thrombotic agents such as anti-coagulants and antiplatelet agents, under conditions where such anti-thrombotic agents reduce clotting and/or aggregation, including in the presence of two of such agents. It is noteworthy that aggregation of platelet derivatives is different from co-aggregation in that aggregation conditions typically do not include fresh platelets, whereas co-aggregation conditions include fresh platelets. Exemplary aggregation and co-aggregation conditions are provided in the Examples herein. Thus, in some embodiments, the platelet derivatives as described herein have a higher propensity to co-aggregate in the presence of fresh platelets and an agonist, while having a reduced propensity to aggregate in the absence of fresh platelets and an agonist, compared to the propensity of fresh platelets to aggregate under these conditions. In some embodiments, a platelet derivative composition comprises a population of platelet derivatives having a reduced propensity to aggregate, wherein no more than 2%, 3%, 4%, 5%, 7.5%, 10%, 12.5%, 15%, 17.5%, 20%, 22.5%, or 25% of the platelet derivatives in the population aggregate under aggregation conditions comprising an agonist but no platelets, in illustrative embodiments no fresh platelets. In some embodiments, the population of platelet derivatives aggregate in the range of 2-30%, 5-25%, 10-30%, 10-25%, or 12.5-25% of the platelet derivatives under aggregation conditions comprising an agonist but no platelets, in illustrative embodiments no fresh platelets.

As provided in Examples herein, exemplary aggregation conditions and related methods include treating FDPD sample preparations at room temperature with an agonist at a final agonist concentration of 20 μM ADP, 0.5 mg/mL arachidonic acid, 10 μg/mL collagen, 200 μM epinephrine, 1 mg/mL ristocetin, and 10 μM TRAP-6 and measured by LTA, for example, 5 minutes after agonist addition to the FDPD sample, which can be compared to LTA measurements of the sample prior to agonist addition.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP), in illustrative embodiments, the agonist is thrombin receptor associated protein (TRAP), or TRAP-6. In some embodiments, the platelet activation marker is selected from the group consisting of phosphatidylserine (PS), and CD 62. In illustrative embodiments, the platelet activation marker comprises phosphatidylserine (PS), and the agonist comprises thrombin receptor activator peptide, such as thrombin receptor activator peptide 6 (TRAP-6). In some embodiments, the platelet derivatives as described herein show an inability to increase expression of phosphatidylserine (PS) in the presence of TRAP. Typically, phosphatidylserine (PS) can be detected by the binding of Annexin V to the platelet derivatives. Not to be limited by theory, Annexin V specifically binds to the phosphatidylserine (PS) on the platelet derivatives. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

Thrombospondin P is a glycoprotein secreted from the α-granules of platelets upon activation. In the presence of divalent cations, the secreted protein binds to the surface of the activated platelets and is responsible for the endogenous lectin-like activity associated with activated platelets. In some embodiments, the platelet derivatives have the presence of thrombospondin (TSP-1) on their surface at a level that is greater than that presence on the surface of resting platelets, activated platelets, or lyophilized fixed platelets. In some embodiments, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

Von Willebrand factor (vWF) P is a multimeric glycoprotein that plays a major role in blood coagulation. vWF serves as a bridging molecule that promotes platelet binding to sub-endothelium and other platelets, thereby promoting platelet adherence and aggregation. vWF also binds to collagens to facilitate clot formation at sites of injury. In some embodiments, the platelet derivatives as described herein have the presence of von Willebrand factor (vWF) on their surface at a level that is greater than that on the surface of resting platelets, activated platelets, or lyophilized fixed platelets. In some embodiments, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

Platelet derivatives, in illustrative embodiments FDPDs, in further illustrative aspects and embodiments herein are surrounded by a compromised plasma membrane. In these further illustrative aspects and embodiments, the platelet derivatives lack an integrated membrane around them. Instead, the membrane surrounding such platelet derivatives (e.g. FDPDs) comprises pores that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. Furthermore, such platelet derivatives (e.g. FDPDs) are not believed to be capable of mitochondrial activation or glycolysis.

A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The increased permeability can be identified by targeting IgG antibodies against a stable intracellular antigen. One non-limiting type of stable intracellular antigen is β tubulin. The compromised membrane of the platelet derivatives can also be determined by flow cytometry studies.

Platelet or platelet derivatives (e.g., FDPDs) as described herein can retain some metabolic activity, for example, as evidenced by lactate dehydrogenase (LDH) activity. In some cases, platelets or platelet derivatives (e.g., FDPDs) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., FDPDs) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

Clotting parameters of blood (e.g., the subject's blood) can be assessed at any appropriate time during the methods described herein. For example, one or more clotting parameters of blood can be assessed before administration of a composition comprising platelets, or in illustrative embodiments a composition comprising platelet derivatives, which in further illustrative embodiments are FDPDs as described herein, e.g., in order to determine the need for administration of a composition comprising platelets or platelet derivatives as described herein. For example, such clotting parameters can be assessed using a pre-administration evaluation or test, such as an in vitro lab test. Such test can be performed on a liquid sample, for example a blood sample, taken within 7, 5, 3, 2, or 1 day, or within 12, 8, 6, 4, 2, or 1 hour before administering a composition comprising platelet derivatives to the subject. As another example, one or more clotting parameters of blood can be assessed after administration of a composition comprising platelets or platelet derivatives as described herein, e.g., in order to determine the effectiveness of the administered composition, to determine whether additional administration of the composition is warranted, or to determine whether it is safe to perform a surgical procedure. Such post-administering evaluation or test can be performed on a liquid sample, for example a blood sample, taken within 7, 5, 3, 2, or 1 day, or within 12, 8, 6, 4, 2, or 1 hour after administering a composition comprising platelet derivatives to the subject.

Accordingly, any of the methods described herein can include steps of assessing one or more clotting parameters of blood before administration of a composition comprising platelets or platelet derivatives as described herein, assessing one or more clotting parameters of blood after administration of a composition comprising platelets or platelet derivatives as described herein, or both.

Any appropriate method can be used to assess (or evaluate) clotting parameters of blood. Non-limiting examples of methods include the World Health Organization (WHO) bleeding scale, prothrombin time (PT) assay, international normalized ratio (INR), thrombin generation (TGA; which can be used to generate parameters such as, e.g., peak thrombin, endogenous thrombin potential (ETP), and lag time), thromboelastography (TEG), multiple electrode aggregometry, light transmission aggregometry (LTA), activated clotting time (ACT), and partial thromboplastin time (PTT or aPTT).

The WHO bleeding scale was developed to help clinicians and researchers assess bleeding, particularly in the context of toxicity reporting in cancer treatment, but it is also used in other contexts.

Prothrombin time (PT) is a measure of how long it takes blood to clot, typically in the presence of Tissue Factor. In some cases, PT can be affected by laboratory reagents, so a normalized ratio (INR) is more frequently used.

The activated partial thromboplastin time (aPTT) is a measure of how long it takes blood to clot, typically in the presence of an activator such as silica, celite, kaolin, or ellagic acid. In some cases, aPTT can be affected by laboratory reagents, so INR is sometimes used instead of or in addition to aPTT.

The thrombin generation assay measured the production of thrombin after sample activation via a pro-coagulation agent resulting of thrombin enzymatic cleavage of a fluorescent peptide and release of fluorescent molecule. The peak thrombin is a measure of the maximum thrombin produced, lag time, the time to start of thrombin production, and ETP as the total thrombin potentially produced. INR is a standard method of determining dosing, see equation below, where “PT(x)” is the result of the prothrombin time assay, while the ISI constant is dependent on the manufacturer of the Tissue Factor used in the prothrombin time assay.

$$INR = {( \frac{PT ( patient )}{PT ( normal )} )}^{ISI constant}$$

Warfarin inhibits the synthesis of four major plasma proteins that are integral to healthy clot formation. A therapeutic maintenance dose of warfarin is typically targeted to an INR of about 2.0 to about 3.0. Thrombosomes present a unique treatment to restore hemostasis in the presence of warfarin-type drugs. Warfarin dose can be expressed by INR, a ratio that increases with the amount of warfarin (1 is a normal value).

In some embodiments, a subject has an INR of more than 2.0 (e.g., at least 2.2, at least 2.4, at least 2.5, at least 2.6, at least 2.8, at least 3.0, at least 3.2, at least 3.4, at least 3.5, at least 3.6, at least 3.8, at least 4.0, at least 4.2, at least 4.4, at least 4.5, at least 4.6, at least 4.8, or at least 5.0) before administration of a composition comprising platelets such as lyophilized platelets or platelet derivatives as described herein. In some embodiments, a subject (e.g., a subject being treated with an anticoagulant, such as warfarin) has an INR of from 2.0 to 3.0, such as from 2.2 to 2.8, such as from 2.4 to 2.6, such as 2.5. In some embodiments, the subject has an INR of more than 2.0 (e.g., at least 2.2, at least 2.4, at least 2.5, at least 2.6, at least 2.8, at least 3.0, at least 3.2, at least 3.4, at least 3.5, at least 3.6, at least 3.8, at least 4.0, at least 4.2, at least 4.4, at least 4.5, at least 4.6, at least 4.8, or at least 5.0), within about 1 week, about 5 days, about 3 days, about 36 hours, about 24 hours, about 18 hours, about 12 hours, about 8 hours, about 6 hours, about 4 hours, about 2 hours, or about 1 hour before the administering.

In some embodiments, a subject has a lower INR (or a normal INR) after administration of a composition comprising platelets such as lyophilized platelets or platelet derivatives as described herein. For example, a subject can have an INR of 3.0 or less (e.g., less than 2.8, less than 2.6, less than 2.5, less than 2.4, less than 2.2, less than 2.0, less than 1.8, less than 1.6, less than 1.5, less than 1.4, less than 1.2, or less than 1.0) after administration of a composition comprising platelets or platelet derivatives ad described herein. In some embodiments, the subject has an INR of 3.0 or less (e.g., less than 2.8, less than 2.6, less than 2.5, less than 2.4, less than 2.2, less than 2.0, less than 1.8, less than 1.6, less than 1.5, less than 1.4, less than 1.2, or less than 1.0), within about 1 week, about 5 days, about 3 days, about 36 hours, about 24 hours, about 18 hours, about 12 hours, about 8 hours, about 6 hours, about 4 hours, about 2 hours, or about 1 hour after the administering.

In some embodiments, a patient can have a peak thrombin of about 60 nM to about 170 nM, such as about 65 nM to about 170 nM, such as about 65 nM to about 120 nM, such as about 80 nM, before administration of a composition comprising platelets or platelet derivatives as described herein.

Thrombin clotting time (TCT) is a measure of how long it takes blood to clot, after an excess of thrombin has been added.

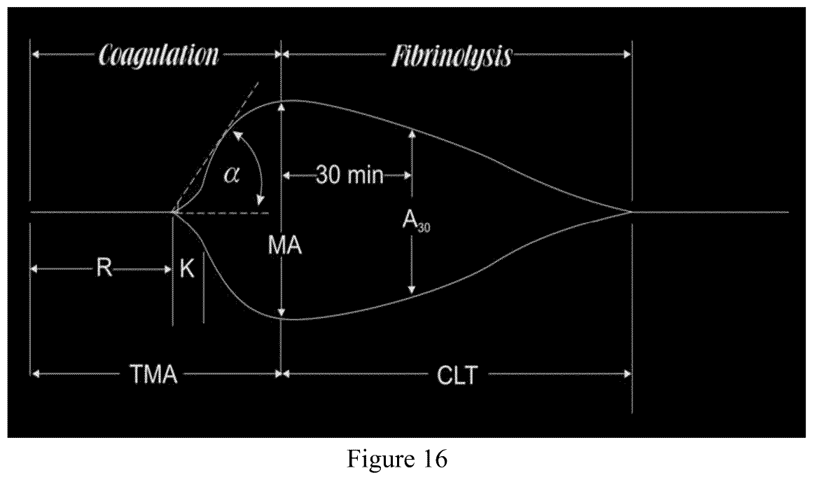

TEG assesses intrinsic hemostasis via plots of clot strength over time. Calcium chloride (CaCl₂) is typically used as the initiating reagent. A TEG waveform (see, e.g., FIG. 16) has multiple parameters that can provide information about clotting.

$$R - time = reaction time ( s ) - time of latency from start of test to initial$$

$$fibrin formation .$$

$$K = kinetics ( s ) - speed of initial fibrin formation , time taken to$$

$$achieve a certain level of clot strength ( e . g . , an amplitude of 20 mm )$$

$$alpha angle = slope of line between R and K - measures the rate of$$

$$clot formation .$$

$$MA = maximum amplitude ( mm ) - represents the ultimate strength of$$

$$the fibrin clot .$$

$${A}_{30} = amplitude 30 minutes after maximum amplitude is reached -$$

$$represents rate of lysis phase .$$

In hypocoagulable blood states, R-time increases and MA decreases. R-time typically provides a broader response range than MA.

Multiple electrode aggregometery (MEA) can also be used to evaluate clotting parameters of blood. MEA measures changes in electrical impedance when platelets aggregate on metal electrodes. Typically, aggregation agonists such as ADP, epinephrine, collagen, or ristocetin are used to initiate aggregation.

Light transmission aggregometry (LTA) is sometimes used to evaluate clotting parameters of blood; unaggregated blood allows little light to pass through, but aggregation (typically initiated by an agonist) results in an increase in aggregation.

In the Total Thrombus-formation Analysis System (T-TAS®, FUJIMORI KOGYO CO., LTD), the sample is forced through collagen-coated microchannels using mineral oil. Changes in pressure are used to assess thrombus formation. The Occlusion Start Time is time it takes to reach 10 kPa, and the Occlusion Time=time it takes to each 480 kPa using an AR chip (e.g., Zacros Item No, TC0101). According to the manufacturer, an AR chip can be used for analyzing the formation of a mixed white thrombus consisting chiefly of fibrin and activated platelets. It has a flow path (300 μm wide by 50 μm high) coated with collagen and tissue factors and can be used to analyze the clotting function and platelet function. In comparison, a PL chip can be used for analyzing the formation of a platelet thrombus consisting chiefly of activated platelets. A PL chip has a flow path coated with collagen only and can be used to analyze the platelet function.

The ACT assay is the most basic, but possibly most reliable, way to measure clotting time (t_{ACT}), determined by a magnet's resistance to gravity as a clot forms around it. Typical donor blood has a t_{ACT}˜200-300s using only CaCl₂.

Exemplary normal ranges for some of these clotting parameters are shown below in Table E1. Typically, a value outside of the ranges shown below is considered to be abnormal.

TABLE E1

| Evaluation | Exemplary Normal Range |
|---|---|
| Bleeding (WHO scale) | 0⁽⁴⁾ |
| LTA (percent aggregation) | |
| 5 μmol/L ADP | 70 ± 10⁽¹⁾ |
| 2 μg/mL collagen | 80 ± 13⁽¹⁾ |
| 1 mmol/L arachidonic acid | 77 ± 10⁽¹⁾ |
| 2 mmol/L arachidonic acid | 80 ± 11⁽¹⁾ |
| 5 mmol/L arachidonic acid | 78 ± 5⁽¹⁾ |
| TEG | |
| 1 mmol/L arachidonic acid (% aggregation) | 95 ± 9⁽¹⁾ |
| MA (mm) | 50-60⁽⁶⁾ |
| R-time (minutes) | 7.5-1⁽⁶⁾ |
| K (minutes) | 3-6⁽⁶⁾ |
| Alpha angle (degrees) | 45-45⁽⁶⁾ |
| INR | 0.8-1.2⁽²⁾ |
| PT (seconds) | 10-14⁽⁵⁾ |
| aPTT (seconds) | 22-35⁽²⁾ |
| TCT (seconds) | 20-30⁽²⁾ |
| t_{ACT} (seconds) | 200-300 |
| MEA (Units) | |
| ADP-induced | 53-122⁽³⁾ |
| Arachidonic acid-induced | 76-136⁽³⁾ |

⁽¹⁾DiChiara, et al. “The effect of aspirin dosing on platelet function in diabetic and nondiabetic patients: an analysis from the aspirin-induced platelet effect (ASPECT) study.” Diabetes 56.12 (2007): 3014-3019.
⁽²⁾Thrombosis Canada. Use And Interpretation Of Laboratory Coagulation Tests In Patients Who Are Receiving A New Oral Anticoagulant (Dabigatran, Rivaroxaban, Apixaban). 2013
⁽³⁾Beynon, et al. “Multiple electrode aggregometry in antiplatelet-related intracerebral haemorrhage.” Journal of Clinical Neuroscience 20.12 (2013): 1805-1806.
⁽⁴⁾Rodeghiero, Francesco, et al. “Standardization of bleeding assessment in immune thrombocytopenia: report from the International Working Group.” Blood 121.14 (2013): 2596-2606.
⁽⁵⁾Cleveland Clinic “Prothrombin Time (PT) test” https://my.clevelandclinic.org/health/diagnostics/17691-prothrombin-time-pt-test#results-and-follow-up. Accessed 15 Feb. 2021.
⁽⁶⁾Bose and Hravnak. “Thromboelastography: a practice summary for nurse practitioners treating hemorrhage.” The Journal for Nurse Practitioners 11.7 (2015): 702-709.

Some embodiments provide a method of increasing thrombin generation in a subject, the method comprising administering to the subject in need thereof an effective amount of a composition comprising platelets, or in illustrative embodiments a composition comprising platelet derivatives, which in further illustrative embodiments are FDPDs and an incubating agent comprising one or more salts, a buffer, optionally a cryoprotectant, and optionally an organic solvent.

Some embodiments, provide a method of increasing thrombin generation in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of increasing peak thrombin in a subject, the method comprising administering to the subject in need thereof an effective amount of a composition comprising platelets, or in illustrative embodiments a composition comprising platelet derivatives, which in further illustrative embodiments are FDPDs and an incubating agent comprising one or more salts, a buffer, optionally a cryoprotectant, and optionally an organic solvent.

Some embodiments provide a method of increasing peak thrombin in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, prior to the administering, the peak thrombin of the subject was below 66 nM (e.g., below 64 nM, 62 nM, 60 nM, 55 nM, 50 nM, 45 nM, 40 nM, 35 nM, 30 nM, 25 nM, 20 nM, 15 nM, 10 nM, or 5 nM). In some embodiments, after the administering, the peak thrombin of the subject is above 66 nM (e.g., above 68 nM, 70 nM, 75 nM, 80 nM, 85 nM, 90 nM, 95 nM, 100 nM, 110 nM, 120 nM, 130 nM, 140 nM, or 150 nM). In some embodiments, after the administering, the peak thrombin of the subject is between 66 and 166 nM. Peak thrombin can be measured by any appropriate method.

In some embodiments a composition as provided herein, or a composition produced by a method described herein can be administered to a subject because of an abnormal result in an evaluation of one or more clotting parameters, e.g., indicating that the subject is in a hypocoagulable state.

Some embodiments include a method of treating a coagulopathy, or reducing bleeding in a subject that is being administered or has been administered an anticoagulant agent, and/or at least one antiplatelet agent, the method including: (a) determining that the subject has an abnormal result for evaluation of one or more clotting parameters; and (b) after (a), administering to the subject in need thereof an effective amount of a composition including platelets or platelet derivatives and an incubating agent including one or more salts, a buffer, optionally a cryoprotectant, and optionally an organic solvent.

Some embodiments include a method of treating a coagulopathy, or reducing bleeding in a subject that is being administered or has been administered an anticoagulant agent, and/or at least one antiplatelet agent, the method including: (a) determining that the subject an abnormal result for evaluation of one or more clotting parameters; and (b) after (a), administering to the subject in need thereof an effective amount of a composition prepared by a process including incubating platelets with an incubating agent including one or more salts, a buffer, optionally a cryoprotectant, and optionally an organic solvent, to form the composition.

Some embodiments include a method of treating a coagulopathy, or reducing bleeding in a subject that is being administered or has been administered an anticoagulant, and/or at least one antiplatelet agent, the method including: administering to the subject in need thereof an effective amount of a composition including platelets or platelet derivatives and an incubating agent including one or more salts, a buffer, optionally a cryoprotectant, and optionally an organic solvent, wherein before the administering, the subject has been determined to have an abnormal result for evaluation of one or more clotting parameters.

Some embodiments include a method of treating a coagulopathy, or reducing bleeding in a subject that is being administered or has been administered an anticoagulant agent, and/or at least one antiplatelet agent, the method including: administering to the subject in need thereof an effective amount of a composition including platelets or platelet derivatives and an incubating agent including one or more salts, a buffer, optionally a cryoprotectant, and optionally an organic solvent, wherein before the administering, the subject has been determined to have an abnormal result for evaluation of one or more clotting parameters.

Some embodiments include a method of treating a coagulopathy, or reducing bleeding in a subject that is being administered or has been administered an anticoagulant agent, and/or at least one antiplatelet agent, the method including: administering to the subject in need thereof an effective amount of a composition prepared by a process including incubating platelets with an incubating agent including one or more salts, a buffer, optionally a cryoprotectant, and optionally an organic solvent, to form the composition, wherein before the administering, the subject has been determined to have an abnormal result for evaluation of one or more clotting parameters.

In some embodiments of any of the methods herein, a subject has been administered an anticoagulant agent or is being administered an anticoagulant agent in any appropriate time frame. For example, in some cases, a subject has been administered an anticoagulant agent and/or a composition comprising platelet derivatives, in illustrative embodiments FDPDs, before the effect of a prior dose of the anticoagulant agent wears off. For example, in some cases, a subject is being administered an anticoagulant agent and the effect of the anticoagulant agent has not worn off. As another example, in some cases, a subject has been administered an anticoagulant agent (e.g., the most recent dose) within about 1 week, about 5 days, about 3 days, about 36 hours, about 24 hours, about 18 hours, about 12 hours, about 8 hours, about 6 hours, about 4 hours, about 2 hours, or about 1 hour. As another example, in some cases, a subject is being administered an anticoagulant agent and the last dose (e.g., the most recent dose as prescribed by a medical professional or self-administered by the subject) was within about 1 week, about 5 days, about 3 days, about 36 hours, about 24 hours, about 18 hours, about 12 hours, about 8 hours, about 6 hours, about 4 hours, about 2 hours, or about 1 hour. In some embodiments, the subject in addition to the anticoagulant agent, has been administered, or is being administered an antiplatelet agent in any appropriate time frame. In some embodiments, a subject is being administered an antiplatelet agent, and the effect of the anticoagulant agent that was previously administered has not worn off. In some embodiments, a subject has been administered an antiplatelet agent (e.g., the most recent dose) within about 1 week, about 5 days, about 3 days, about 36 hours, about 24 hours, about 18 hours, about 12 hours, about 8 hours, about 6 hours, about 4 hours, about 2 hours, or about 1 hour. As another example, in some cases, a subject is being administered an antiplatelet agent and the last dose (e.g., the most recent dose as prescribed by a medical professional or self-administered by the subject) was within about 1 week, about 5 days, about 3 days, about 36 hours, about 24 hours, about 18 hours, about 12 hours, about 8 hours, about 6 hours, about 4 hours, about 2 hours, or about 1 hour.

In some embodiments of any of the methods herein, determination of an abnormal result for the evaluation of one or more clotting parameters can be at any appropriate time. For example, determination of an abnormal result for the evaluation of one or more clotting parameters can be before the abnormal result returns to a normal result. As another example, determination of an abnormal result for the evaluation of one or more clotting parameters can be within about 1 week, about 5 days, about 3 days, about 36 hours, about 24 hours, about 18 hours, about 12 hours, about 8 hours, about 6 hours, about 4 hours, about 2 hours, or about 1 hour of the administering.

In some embodiments, the method can further include determining the result of the evaluation one or more clotting parameters following the administering. For example, in some embodiments, the evaluation of the one or more clotting parameters following the administering shows a normal result for at least one of the one or more clotting parameters. In some embodiments, the result of the evaluation of the one or more clotting parameters following the administering is improved from the result of the evaluation of the one or more parameters prior to the administering.

In some cases, a subject might be administered an anticoagulant agent, but they were not supposed to be, for example, if a subject is confused, or if a medical error occurs. In some such cases, a subject can be administered any of the compositions provided herein, or a composition produced by any of the methods described herein.

Some embodiments include a method of treating a coagulopathy in a subject, the method including: (a) determining that the subject, contrary to medical instruction, was administered an anticoagulant agent; and (b) administering to the subject in need thereof an effective amount of a composition including platelets or platelet derivatives and an incubating agent including one or more salts, a buffer, optionally a cryoprotectant, and optionally an organic solvent.

Some embodiments include a method of treating a coagulopathy in a subject, the method including: (a) determining that the subject, contrary to medical instruction, was administered an anticoagulant agent; and (b) administering to the subject in need thereof an effective amount of a composition prepared by a process including incubating platelets with an incubating agent including one or more salts, a buffer, optionally a cryoprotectant, and optionally an organic solvent, to form the composition.

Some embodiments include a method of treating a coagulopathy in a subject, the method including: administering to the subject in need thereof an effective amount of a composition including platelets or platelet derivatives and an incubating agent including one or more salts, a buffer, optionally a cryoprotectant, and optionally an organic solvent, wherein the subject is determined to have been administered an anticoagulant agent contrary to medical instruction.

Some embodiments include a method of treating a coagulopathy in a subject, the method including: administering to the subject in need thereof an effective amount of a composition prepared by a process including incubating platelets with an incubating agent including one or more salts, a buffer, optionally a cryoprotectant, and optionally an organic solvent, to form the composition, wherein the subject is determined to have been administered an anticoagulant agent contrary to medical instruction.

Some embodiments include a method of restoring normal hemostasis in a subject, the method including: (a) determining that the subject, contrary to medical instruction, was administered an anticoagulant agent; and (b) administering to the subject in need thereof an effective amount of a composition including platelets or platelet derivatives and an incubating agent including one or more salts, a buffer, optionally a cryoprotectant, and optionally an organic solvent.

Some embodiments include a method of restoring normal hemostasis in a subject, the method including: (a) determining that the subject, contrary to medical instruction, was administered an anticoagulant agent; and (b) administering to the subject in need thereof an effective amount of a composition prepared by a process including incubating platelets with an incubating agent including one or more salts, a buffer, optionally a cryoprotectant, and optionally an organic solvent, to form the composition.

Some embodiments include a method of restoring normal hemostasis in a subject, the method including: administering to the subject in need thereof an effective amount of a composition including platelets or platelet derivatives and an incubating agent including one or more salts, a buffer, optionally a cryoprotectant, and optionally an organic solvent, wherein the subject is determined to have been administered an anticoagulant agent contrary to medical instruction.

Some embodiments include a method of restoring normal hemostasis in a subject, the method including: administering to the subject in need thereof an effective amount of a composition prepared by a process including incubating platelets with an incubating agent including one or more salts, a buffer, optionally a cryoprotectant, and optionally an organic solvent, to form the composition, wherein the subject is determined to have been administered an anticoagulant agent contrary to medical instruction.

In some embodiments of any of the methods herein, determining that the subject has been administered an anticoagulant agent contrary to medical instruction can be at any appropriate time. For example, determining that the subject has been administered an anticoagulant agent contrary to medical instruction can be before the anticoagulant agent wears off. As another example, determining that the subject has been administered an anticoagulant agent contrary to medical instruction can be within about 1 week, about 5 days, about 3 days, about 36 hours, about 24 hours, about 18 hours, about 12 hours, about 8 hours, about 6 hours, about 4 hours, about 2 hours, or about 1 hour of the administering a composition provided herein or a composition produced by a method described herein.

Administration of the anticoagulant agent can include any appropriate method, including self-administering by the subject or administering by a medical professional.

Medical instruction can be any appropriate method, including verbal instruction, written instruction, or both verbal and written instruction.

In some cases, a subject may have been administered or is being administered a second agent that affects (e.g., decreases) platelet function. For example, such an administration can put the subject into a hypocoagulable state.

Some embodiments include a method of treating a coagulopathy in a subject, the method including: (a) determining that the subject was administered an anticoagulant agent and a second agent that decreases platelet function; and (b) administering to the subject in need thereof an effective amount of a composition including platelets or platelet derivatives and an incubating agent including one or more salts, a buffer, optionally a cryoprotectant, and optionally an organic solvent.

Some embodiments include a method of treating a coagulopathy in a subject, the method including: (a) determining that the subject was administered an anticoagulant agent and a second agent that decreases platelet function; and (b) administering to the subject in need thereof an effective amount of a composition prepared by a process including incubating platelets with an incubating agent including one or more salts, a buffer, optionally a cryoprotectant, and optionally an organic solvent, to form the composition.

Some embodiments include a method of treating a coagulopathy in a subject, the method including: administering to the subject in need thereof an effective amount of a composition including platelets or platelet derivatives and an incubating agent including one or more salts, a buffer, optionally a cryoprotectant, and optionally an organic solvent, wherein the subject is determined to have been administered an anticoagulant agent and a second agent that decreases platelet function.

Some embodiments include a method of treating a coagulopathy in a subject, the method including: administering to the subject in need thereof an effective amount of a composition prepared by a process including incubating platelets with an incubating agent including one or more salts, a buffer, optionally a cryoprotectant, and optionally an organic solvent, to form the composition, wherein the subject is determined to have been administered an anticoagulant agent and a second agent that decreases platelet function.

Some embodiments include a method of restoring normal hemostasis in a subject, the method including: (a) determining that the subject was administered an anticoagulant agent and a second agent that decreases platelet function; and (b) administering to the subject in need thereof an effective amount of a composition including platelets or platelet derivatives and an incubating agent including one or more salts, a buffer, optionally a cryoprotectant, and optionally an organic solvent.

Some embodiments include a method of restoring normal hemostasis in a subject, the method including: (a) determining that the subject was administered an anticoagulant agent and a second agent that decreases platelet function; and (b) administering to the subject in need thereof an effective amount of a composition prepared by a process including incubating platelets with an incubating agent including one or more salts, a buffer, optionally a cryoprotectant, and optionally an organic solvent, to form the composition.

Some embodiments include a method of restoring normal hemostasis in a subject, the method including: administering to the subject in need thereof an effective amount of a composition including platelets or platelet derivatives and an incubating agent including one or more salts, a buffer, optionally a cryoprotectant, and optionally an organic solvent, wherein the subject is identified as having been administered an anticoagulant agent and a second agent that decreases platelet function.

Some embodiments include a method of restoring normal hemostasis in a subject, the method including: administering to the subject in need thereof an effective amount of a composition prepared by a process including incubating platelets with an incubating agent including one or more salts, a buffer, optionally a cryoprotectant, and optionally an organic solvent, to form the composition, wherein the subject is identified as having been administered an anticoagulant agent and a second agent that decreases platelet function.

In some embodiments, administration of the second agent is stopped (for example, if the benefits of stopping the second agent outweigh the costs of stopping the second agent). In some embodiments, administration of the second agent is continued (for example, if removal of the second agent would be detrimental to the subject, as can be the case with certain medications, such as antidepressants).

The second agent can be any appropriate second agent that affects (e.g., decreases) platelet function. For example, a second agent can include (or be selected from the group consisting of) an antihypertensive, a proton pump inhibitor, or a combination thereof. As another example, a second agent can include (or be selected from the group consisting of) a chemotherapeutic agent, an antibiotic, a cardiovascular agent, a H2 antagonist, a neuropsychiatric agent, or a combination thereof. In some embodiments, the second agent can include (or be) an antidepressant (e.g., a selective serotonin reuptake inhibitor (SSRI), a serotonin antagonist and reuptake inhibitor (SARI), a serotonin and norepinephrine reuptake inhibitor (SNRI), or a combination thereof). In some embodiments, the second agent is not an anticoagulant.

In some embodiments of any of the methods provided herein, administration of the anticoagulant agent is stopped. In some embodiments of any of the methods provided herein, administration of the anticoagulant agent is continued.

In cases, such as certain emergency situations, it can be impossible to timely determine whether a subject is being administered an anticoagulant agent. In some such cases, a composition provided herein or a composition produced by a method provided herein can be administered to a subject to prevent a coagulopathy. In some embodiments, a composition provided herein or a composition produced by a method provided herein can be administered to a subject to mitigate the potential for a coagulopathy in the subject.

Some embodiments include a method of preventing or mitigating the potential for a coagulopathy in a subject, the method including: (a) determining that information regarding whether the subject was administered an anticoagulant agent is unavailable; and (b) administering to the subject an effective amount of a composition including platelets or platelet derivatives and an incubating agent including one or more salts, a buffer, optionally a cryoprotectant, and optionally an organic solvent.

Some embodiments include a method of preventing or mitigating the potential for a coagulopathy in a subject, the method including: (a) determining that information regarding whether the subject was administered an anticoagulant agent is unavailable; and (b) administering to the subject an effective amount of a composition prepared by a process including incubating platelets with an incubating agent including one or more salts, a buffer, optionally a cryoprotectant, and optionally an organic solvent, to form the composition.

Some embodiments include method of preventing or mitigating the potential for a coagulopathy in a subject, the method including: administering to the subject in need thereof an effective amount of a composition including platelets or platelet derivatives and an incubating agent including one or more salts, a buffer, optionally a cryoprotectant, and optionally an organic solvent, wherein the subject has been determined to be a subject for which information regarding whether the subject was administered an anticoagulant agent is unavailable.

Some embodiments include a method of preventing or mitigating the potential for a coagulopathy in a subject, the method including: administering to the subject in need thereof an effective amount of a composition prepared by a process including incubating platelets with an incubating agent including one or more salts, a buffer, optionally a cryoprotectant, and optionally an organic solvent, to form the composition, wherein the subject has been determined to be a subject for which information regarding whether the subject was administered an anticoagulant agent is unavailable.

In some embodiments of any of the methods herein, determining that information regarding whether the subject was administered an anticoagulant agent is unavailable can be at any appropriate time. For example, determining that information regarding whether the subject was administered an anticoagulant agent is unavailable can be within about 1 week, about 5 days, about 3 days, about 36 hours, about 24 hours, about 18 hours, about 12 hours, about 8 hours, about 6 hours, about 4 hours, about 2 hours, or about 1 hour of the administering a composition provided herein or a composition produced by a method described herein.

There are several reasons that information regarding whether the subjected was administered an anticoagulant agent is unavailable. For example, a reason can include that the subject cannot be identified, that the medical history of the subject is unavailable, that the subject is in need of emergency treatment, that the subject is in need of emergency surgery, that the subject has an emergency surgery, or a combination thereof.

In some embodiments of any of the methods provided herein, the method can further include determining that the subject has an abnormal result for one or more evaluations of clotting parameters. In some embodiments of any of the methods provided herein the subject has been determined to have an abnormal result for one or more evaluations of clotting parameters.

In some cases, before an abnormal result was determined, the subject was previously identified as having a normal result for at least one of the one or more clotting parameters.

In some embodiments of any of the methods provided herein, the method can further include determining the result of the evaluation one or more clotting parameters following the administering of a composition provided herein or a composition produced by a method provided herein. In some such cases, the evaluation of the one or more clotting parameters following the administering shows a normal result, such as defined in Table E1 for at least one of the one or more clotting parameters. In some embodiments, the result of the evaluation of the one or more clotting parameters following the administering is improved from the result of the evaluation of the one or more parameters prior to the administering.

In some embodiments, the subject is identified as having an abnormal result for one or more evaluations of clotting parameters during surgery (e.g., emergency surgery or scheduled surgery)

An evaluation of one or more clotting parameters can be any appropriate evaluation of clotting parameters, such as any of the evaluations of clotting parameters provided herein. In some embodiments, an evaluation of clotting parameters can be selected from the group consisting of the World Health Organization (WHO) bleeding scale, prothrombin time (PT) assay, international normalized ratio (INR), thrombin generation (TGA), thromboelastography (TEG), multiple electrode aggregometry (MEA), light transmission aggregometry (LTA), activated clotting time (ACT), and partial thromboplastin time (e.g., PTT or aPTT), subparameters thereof, and a combination of any thereof.

In some embodiments, the one or more clotting parameters includes an evaluation of bleeding (e.g., performed based on the World Health Organization (WHO) bleeding scale). In some embodiments, before the administering, the subject has bleeding of grade 2, 3, or 4 based on the WHO bleeding scale. In some embodiments, after the administering, the subject has bleeding of grade 0 or 1 based on the WHO bleeding scale. In some embodiments, after the administering, the subject has bleeding of one grade less, based on the WHO bleeding scale, than before the administering. In some embodiments, after the administering, the subject has bleeding of two grades less, based on the WHO bleeding scale, than before the administering. In some embodiments, after the administering, the subject has bleeding of three grades less, based on the WHO bleeding scale, than before the administering.

In some embodiments, the one or more clotting parameters includes an evaluation of prothrombin time (PT). In some embodiments the abnormal results for PT comprises a PT of greater than about 14 seconds (e.g., greater than about 15 seconds, 18 seconds, 20 seconds, 25 seconds, or more). In some embodiments, after the administering, the subject has a decrease in PT of at least 1 second (e.g., at least 2, 3, 4, 5, 6, 7, 8, 9, 10, or more, seconds). In some embodiments, after the administering, the subject has a normal PT, such as defined in Table E1.

In some embodiments, the one or more clotting parameters includes an evaluation of activated partial thromboplastin time (aPTT). In some embodiments, the abnormal result for aPTT comprises an aPTT of greater than about 40 seconds (e.g., greater than about 43 seconds, 45 seconds, 50 seconds, 55 seconds, 60 seconds, 65 seconds, 70 seconds, or more). In some embodiments, after the administering, the subject has a decrease in aPTT of at least 5 seconds (e.g., at least 10, 15, 20, 25, 30, or more, seconds). In some embodiments, after the administering, the subject has a normal aPTT, such as defined in Table E1.

In some embodiments, the one or more clotting parameters includes an evaluation of thrombin clot time (TCT). In some embodiments, the abnormal result for TCT comprises a TCT of greater than about 35 seconds (e.g., greater than about 38 seconds, 40 seconds, 45 seconds, 50 seconds, 55 seconds, 60 seconds, or more). In some embodiments, after the administering, the subject has a decrease in TCT of at least 5 seconds (e.g., at least 10, 15, 20, 25, 30, or more, seconds. In some embodiments, after the administering, the subject has a normal TCT, such as defined in Table E1.

In some embodiments, the evaluation of the one or more clotting parameters includes thromboelastography (TEG). In some embodiments, the abnormal result for TEG comprises a maximum amplitude (MA) of less than about 50 mm (e.g., less than about 48 mm, 45 mm, 40 mm, 35 mm, or less). In some embodiments, after the administering, the subject has an increase in MA of at least 5 mm (e.g., at least 10, 15, 20, 25, 30, or more, mm). In some embodiments, after the administering, the subject has a normal MA, such as defined in Table E1. In some embodiments, the abnormal result for TEG comprises a percent aggregation (in the presence of 1 mmol/L arachidonic acid) of less than about 85% (e.g., less than about 83%, 80%, 75%, 70%, or less). In some embodiments, after the administering, the subject has an increase in percent aggregation (in the presence of 1 mmol/L arachidonic acid) of at least 2 percentage points (e.g., at least, 3, 5, 8, 10, 12, 15, 18, 20, or more, percentage points). In some embodiments, after the administering, the subject has a normal percent aggregation (in the presence of 1 mmol/L arachidonic acid), such as defined in Table E1. In some embodiments, the TEG is used to evaluate adenosine diphosphate-induced platelet-fibrin clot strength. In some embodiments, the TEG is used to evaluate arachidonic acid-induced platelet-fibrin clot strength.

In some embodiments, the evaluation of one or more clotting parameters includes multiple electrode aggregometry (MEA). In some embodiments the abnormal result for MEA comprises an abnormal result for ADP-induced platelet activity. In some embodiments the abnormal result for MEA comprises a result of less than about 50 units (U) (e.g., less than about 48, 45, 40, 35, or less, U) for ADP-induced platelet activity. In some embodiments, after the administering, the subject has an increase in ADP-induced platelet activity by at least 5 U (e.g., at least 8, 10, 15, 20, or more U). In some embodiments, after the administering, the subject has a normal value for ADP-induced platelet activity, such as defined in Table E1. In some embodiments, the abnormal result for MEA comprises an abnormal result for arachidonic acid-induced platelet activity. In some embodiments, the abnormal result for MEA comprises a result of less than about 70 units (U) (e.g., less than about 68, 65, 60, 55, 50, 45, or less, U) for arachidonic acid-induced platelet activity. In some embodiments, after the administering, the subject has an increase in arachidonic acid-induced platelet activity by at least 5 (e.g., at least 8, 10, 15, 20, or more, units). In some embodiments, after the administering, the subject has a normal value for arachidonic acid-induced platelet activity, such as defined in Table E1.

In some embodiments, the evaluation of one or more clotting parameters includes light transmission aggregometry (LTA). In some embodiments, the abnormal result for LTA includes, in the presence of 5 μmol/L adenosine diphosphate, a percent aggregation of less than about 60% (e.g., less than about 58%, 55%, 50%, 45%, or less). In some embodiments, the abnormal result for LTA includes, in the presence of 2 μg/mL collagen, a percent aggregation of less than about 65% (e.g., less than about 63%, 60%, 55%, 50%, 45%, or less).). In some embodiments, the abnormal result for LTA includes, in the presence of 1 mmol/L arachidonic acid, a percent aggregation of less than about 65% (e.g., less than about 63%, 60%, 55%, 50%, 45%, or less).). In some embodiments, the abnormal result for LTA includes, in the presence of 2 mmol/L arachidonic acid, a percent aggregation of less than about 69% (e.g., less than about 67%, 65%, 60%, 55%, 50%, 45%, or less).). In some embodiments, the abnormal result for LTA includes, in the presence of 5 mmol/L arachidonic acid, a percent aggregation of less than about 73% (e.g., less than about 70%, 65%, 60%, 55%, 50%, or less). In some embodiments, after the administering, the subject has an increase in percent aggregation (in the presence of 5 μmol/L adenosine diphosphate) of at least 2 percentage points (e.g., at least 3, 5, 8, 10, 12, or more, percentage points). In some embodiments, after the administering, the subject has a normal percent aggregation (in the presence of 5 μmol/L adenosine diphosphate), such as defined in Table E1. In some embodiments, after the administering, the subject has an increase in percent aggregation (in the presence of 2 μg/mL collagen) of at least 2 percentage points (e.g., at least 3, 5, 8, 10, 12, or more, percentage points). In some embodiments, after the administering, the subject has a normal percent aggregation (in the presence of 2 μg/mL collagen), such as defined in Table E1. In some embodiments, after the administering, the subject has an increase in percent aggregation (in the presence of 1 mmol/L arachidonic acid) of at least 2 percentage points (e.g., at least 3, 5, 8, 10, 12, or more, percentage points). In some embodiments after the administering, the subject has a normal percent aggregation (in the presence of 1 mmol/L arachidonic acid), such as defined in Table E1. In some embodiments, after the administering, the subject has an increase in percent aggregation (in the presence of 2 mmol/L arachidonic acid) of at least 2 percentage points (e.g., at least 3, 5, 8, 10, 12, or more, percentage points). In some embodiments, after the administering, the subject has a normal percent aggregation (in the presence of 2 mmol/L arachidonic acid), such as defined in Table E1. In some embodiments, after the administering, the subject has an increase in percent aggregation (in the presence of 5 mmol/L arachidonic acid) of at least 2 percentage points (e.g., at least, 3, 5, 8, 10, 12, or more, percentage points). In some embodiments, after the administering, the subject has a normal percent aggregation (in the presence of 5 mmol/L arachidonic acid), such as defined in Table E1.

In some embodiments, an additional anticoagulant agent reversal agent can be administered to a subject in addition to a composition provided herein or a composition produced by a method described herein. The additional anticoagulant agent reversal agent can be administered in any order with the composition provided herein or the composition produced by a method provided herein. In some embodiments, the administering of the composition occurs concurrently with administering of the additional anticoagulant agent reversal agent. In some embodiments, the administering of the composition occurs after administering of the additional anticoagulant agent reversal agent. In some embodiments, the administering of the composition occurs before administering of the additional anticoagulant agent reversal agent. In some embodiments, the additional anticoagulant reversal agent comprises protamine, protamine sulfate, or a combination thereof. In some embodiments, the additional anticoagulant reversal agent is selected from the group consisting of protamine, protamine sulfate, vitamin K, prothrombin complex concentrate (PCC), idarucizumab, Andexanet Alfa, and combinations thereof.

In one aspect of any of the embodiments herein, the subject does not have cancer.

An “effective amount” as used herein is an amount of the composition that comprises an amount of platelets, typically platelet derivatives, which in illustrative embodiments are FDPDs, effective in treating the subject. Such treating, for example with respect to methods for treating a coagulopathy or methods for counteracting the effect of an anti-thrombotic agent (i.e. an antiplatelet agent and/or an anti-coagulant) of a subject herein reduces the bleeding or the bleeding potential of the subject. In some embodiments, the bleeding or the bleeding potential can be reduced to such an extent that normal hemostasis is restored for the subject, such as to a level for that subject without any anticoagulant agent in their body, at least for a period of time. Thus, in some embodiments, an effective amount of a composition comprising platelet derivatives, for example FDPDs, is an amount that results in reduced bleeding or the bleeding potential of a subject, which in some embodiments results in normal hemostasis, for any period of time. In some embodiments, the bleeding or the bleeding potential is reduced for at least 10, 20, 30, 40, 50 or 60 minutes after being administered a dose of an effective amount of the platelet derivatives, for example the FDPDs, or a second, third, fourth. Fifth, or sixth dose of composition comprising platelet derivatives that each, or two or more, or all, cumulatively add up to an effective dose.

Such an amount of platelets or typically platelet derivatives (e.g., FDPDs) includes any appropriate dosage of a composition comprising the platelet derivatives as described herein that can be administered to the subject, in illustrative embodiments that results in reduced bleeding or reduced bleeding potential of a subject. For example, in some embodiments, a dose of a composition comprising platelets or platelet derivatives (e.g., FDPDs) can include between about or exactly 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, or 5.0×10⁹ to 1.0×10¹⁰ particles (e.g. FDPDs)/kg of a subject). In some embodiments an effective amount of a composition comprising FDPDs is an activity-based amount that has a potency between 250 and 5000 TGPU per kg of a subject. Such activity-based amount can be combined with any of the particle number ranges/kg in this paragraph.

In certain embodiments, any of the dose ranges provided above, and in illustrative embodiments those that include less than 1×10¹¹ particles/kg, can be administered more than 1 time to a subject. For example, a dose range of between 1.0×10⁷ particles to about 1.0×10¹⁰ particles, can be administered between 2 and 10 times, or between 2 and 8 times, or between 2 and 6 times, or between 3 and 8 times, or between 3 and 6 times, or between 4 and 6 times in a timeframe between within 1, 2, 3, 4, 5, or 7 days from the first dose.

In some embodiments of the methods herein, the composition is administered topically. In some embodiments, topical administration can include administration via a solution, cream, gel, suspension, putty, particulates, or powder. In some embodiments, topical administration can include administration via a bandage (e.g. an adhesive bandage or a compression bandage) or medical closure (e.g., sutures, staples)); for example the platelet derivatives (e.g., lyopreserved platelets (e.g., FDPDs)) can be embedded therein or coated thereupon), as described in PCT Publication No. WO2017/040238 (e.g., paragraphs [013]-[069]), corresponding to U.S. patent application Ser. No. 15/776,255, the entirety of which is herein incorporated by reference.

In some embodiments of the methods herein, the composition is administered parenterally. In some illustrative embodiments of the methods herein, the composition is administered intravenously. In some embodiments of the methods herein, the composition is administered intramuscularly. In some embodiments of the methods herein, the composition is administered intrathecally. In some embodiments of the methods herein, the composition is administered subcutaneously. In some embodiments of the methods herein, the composition is administered intraperitoneally.

In some embodiments of the methods herein, the composition is dried prior to the administration step. In illustrative embodiments of the methods herein, the composition is freeze-dried prior to the administration step. Such FDPD composition in illustrative embodiments, is prepared according to methods provided in the Examples herein. In illustrative embodiments of the methods herein, the composition is rehydrated following the drying or freeze-drying step, for example within 24, 12, 8, 6, 4, 3, 2, or 1 hour, or within 30, 20. 15, 10, or 5 minutes before being administered to a subject.

In some embodiments, the anticoagulant is selected from the group consisting of an anti-factor IIa agent such as dabigatran (e.g., PRADAXA®), argatroban, or hirudin; an anti-factor Xa agent such as rivaroxaban (e.g., XARELTO®), apixaban (e.g., ELIQUIS®), edoxaban (e.g., SAVAYSA®), or fondaparinux (e.g., ARIXTRA®); a traditional anticoagulant such as warfarin (e.g., COUMADIN®) and heparin/LMWH (low molecular weight heparins); supplements such as herbal supplements, and a combination thereof. Examples of supplements include ginger, ginseng, ginkgo, green tea, kava, saw palmetto, boldo (Peumus boldus), Danshen (Salvia miltiorrhiza), Dong quai (Angelica sinensis) papaya (Carica papaya), fish oil, vitamin E garlic, coenzyme CoQ10, glucosamine, and glucosamine-condroitin sulfate.

The prescribing information for each of the FDA-approved anticoagulants provided herein is incorporated by reference in its entirety. Such prescribing information includes, for example:

HIGHLIGHTS OF PRESCRIBING INFORMATION for COUMADIN® (warfarin sodium), Revised December 2019.

HIGHLIGHTS OF PRESCRIBING INFORMATION for PRADAXA® (dabigatran etexilate mesylate), Revised July 2020.

HIGHLIGHTS OF PRESCRIBING INFORMATION for XARELTO® (rivaroxaban), Revised January 2021.

HIGHLIGHTS OF PRESCRIBING INFORMATION for SAVAYSA® (edoxaban), Revised January 2015.

HIGHLIGHTS OF PRESCRIBING INFORMATION for ARIXTRA® (fondaparinux sodium), Revised August 2017.

HIGHLIGHTS OF PRESCRIBING INFORMATION for HEPARIN SODIUM IN 0.45% SODIUM CHLORIDE INJECTION, Revised February 2020.

HIGHLIGHTS OF PRESCRIBING INFORMATION for BEVYXXA® (betrixaban), Revised June 2017.

PRESCRIBING INFORMATION for REFLUDAN® (lepirudin), as of October 2004.

HIGHLIGHTS OF PRESCRIBING INFORMATION for ANGIOMAX® (bivalirudin), Revised July 2019.

PRESCRIBING INFORMATION for SINTROM® (acenocoumarol), May 2009.

In some embodiments, the anticoagulant is dabigatran (e.g., PRADAXA®).

In some embodiments, the anticoagulant is argatroban.

In some embodiments, the anticoagulant is hirudin.

In some embodiments, the anticoagulant is rivaroxaban (e.g., XARELTO®).

In some embodiments, the anticoagulant is apixaban (e.g., ELIQUIS®).

In some embodiments, the anticoagulant is edoxaban (e.g., SAVAYSA®).

In some embodiments, the anticoagulant is fondaparinux (e.g., ARIXTRA®).

In some embodiments, the anticoagulant is heparin or a low molecular weight heparin (LMWH).

In some embodiments, the anticoagulant is warfarin (e.g., COUMADIN®).

In some embodiments, the anticoagulant is tifacogin.

In some embodiments, the anticoagulant is Factor VIIai.

In some embodiments, the anticoagulant is SB249417.

In some embodiments, the anticoagulant is pegnivacogin (with or without anivamersen).

In some embodiments, the anticoagulant is TTP889.

In some embodiments, the anticoagulant is idraparinux.

In some embodiments, the anticoagulant is idrabiotaparinux.

In some embodiments, the anticoagulant is SR23781A.

In some embodiments, the anticoagulant is apixaban.

In some embodiments, the anticoagulant is betrixaban.

In some embodiments, the anticoagulant is lepirudin.

In some embodiments, the anticoagulant is bivalirudin.

In some embodiments, the anticoagulant is ximelagatran.

In some embodiments, the anticoagulant is phenprocoumon.

In some embodiments, the anticoagulant is acenocoumarol.

In some embodiments, the anticoagulant an indandione.

In some embodiments, the anticoagulant is fluindione.

In some embodiments, the anticoagulant is a supplement.

In some embodiments, the anticoagulant is an herbal supplement.

In some embodiments, the anticoagulant includes dabigatran at a dosage of about 70 mg to about 230 mg (e.g., about 75 mg, about 110 mg, or about 150 mg) once or twice daily.

In some embodiments, the anticoagulant includes argatroban at a dosage of about 100 to about 150 mg (e.g., about 125 mg) by injection or infusion.

In some embodiments, the anticoagulant includes a hirudin at dosage of about 0.3 to about 0.5 mg/kg body weight of the subject (e.g., about 0.4 mg/kg body weight of the subject) as an initial bolus dose, or about 0.1 to about 0.2 mg/kg body weight of the subject (e.g., about 0.15 mg/kg body weight of the subject) once a day intravenously following the initial bolus dose.

In some embodiments, the anticoagulant includes rivaroxaban at a dosage of about 2 mg to about 25 mg (e.g., about 2.5 mg, 10 mg, 15 mg, 20 mg, or 25 mg) once or twice daily.

In some embodiments, the anticoagulant includes apixaban at a dosage of about 2 mg to about 10 mg (e.g., about 2.5 mg, 5 mg, 7.5 mg, or 10 mg) once or twice daily.

In some embodiments, the anticoagulant includes edoxaban at a dosage of about 15 mg to about 75 mg (about 15 mg, 30 mg, 45 mg, 60 mg, or 75 mg) once daily.

In some embodiments, the anticoagulant includes fondaparinux at a dosage of about 2 mg to about 12 mg (e.g., about 2.5 mg, 5 mg, 7.5 mg, or 10 mg) once daily subcutaneously.

In some embodiments, the anticoagulant includes warfarin at a dosage of about 0.5 mg to about 10 mg (e.g., about 1 mg, 2 mg, 2.5 mg, 3 mg, 4 mg, 5 mg, 6 mg, 7.5, or 10 mg) once daily. In some cases, individualized dosing of warfarin is performed to target an INR of about 2.0 to about 3.5 (e.g., about 2.0 to about 3.0, about 2.5, or about 3.0).

In some embodiments, the anticoagulant includes heparin at a dosage of about 5,000 units to about 50,000 units (e.g., about 10,000 to about 40,000 units, or about 20,000 to about 40,000 units) over a period of 24 hours.

In some embodiments, the anticoagulant includes a low molecular weight heparin at a dosage of about 20 to about 100 mg (e.g., about 30 mg, 40 mg, 50 mg, or 60 mg) once daily.

In some embodiments, the anticoagulant includes apixaban at a dosage of about 2 mg to about 7 mg (e.g., about 2.5 mg, about 3 mg, or about 5 mg) twice daily.

In some embodiments, the anticoagulant includes betrixaban at an initial dosage of about 150 mg to about 170 mg (e.g., about 160 mg), or a dosage of about 70 mg to about 90 mg (e.g., about 80 mg) once daily.

In some embodiments, the anticoagulant includes lepirudin at dosage of about 0.3 to about 0.5 mg/kg body weight of the subject (e.g., about 0.4 mg/kg body weight of the subject) as an initial bolus dose, or about 0.1 to about 0.2 mg/kg body weight of the subject (e.g., about 0.15 mg/kg body weight of the subject) once a day intravenously following the initial bolus dose.

In some embodiments, the anticoagulant includes bivalirudin at a dosage of about 0.7 to about 0.8 mg/kg body weight of the subject (e.g., about 0.75 mg/kg body weight of the subject) as an initial bolus dose, or about 1.7 to about 1.8 mg/kg/hr (e.g., about 1.75 mg/kg/hr) following the initial bolus dose.

In some embodiments, the anticoagulant includes phenprocoumon at an initial dosage of about 10 to about 20 mg (e.g., about 12 mg or about 15 mg), a second dosage of about 5 mg to about 10 mg (e.g., about 6 mg, or about 9 mg), or a following dosage of about 1 to about 7 mg (e.g., about 3 mg or about 6 mg) per day.

In some embodiments, the anticoagulant includes acenocoumarol at an initial dosage of about 6 to about 14 mg (e.g., about 8 to about 12 mg) or a following dosage of about 3 to about 10 mg (e.g., about 4 to about 8 mg) once a day.

In some embodiments, rehydrating the composition comprising platelet derivatives comprises adding to the platelet derivatives (e.g. FDPDs) an aqueous liquid. In some embodiments, the aqueous liquid is water. In some embodiments, the aqueous liquid is an aqueous solution (e.g., a buffer). In some embodiments, the aqueous liquid is a saline solution. In some embodiments, the aqueous liquid is a suspension.

In some embodiments, the rehydrated platelet derivatives (e.g., FDPDs) have coagulation factor levels showing all individual factors (e.g., Factors VII, VIII and IX) associated with blood clotting at 40 international units (IU) or greater.

In some embodiments, the platelet derivatives (e.g., FDPDs) have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelet derivatives (e.g., FDPDs), have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the platelets, typically platelet derivatives, and in illustrative embodiments FDPDs, have a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets, or in illustrative embodiments platelet derivatives (e.g., FDPDs), have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets, or in illustrative embodiments platelet derivatives (e.g., FDPDs), are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets, or in illustrative embodiments platelet derivatives (e.g., FDPDs) are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some illustrative embodiments, a microparticle can be a particle having a particle size (e.g., diameter, max dimension) of less than about 0.5 μm (less than about 0.45 μm or 0.4 μm) In some cases, a microparticle can be a particle having a particle size of about 0.01 μm to about 0.5 μm (e.g., about 0.02 μm to about 0.5 μm).

Compositions comprising platelets or platelet derivatives (e.g., FDPDs), such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, or 0.5%) of the total scattering intensity of all particles from about 1 nm to about 60,000 nm in radius in the composition. In some embodiments, the platelet derivative composition comprises a population of platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives have a diameter in the range of 0.5-2.5 μm, and the microparticles have a diameter less than 0.5 μm.

In some embodiments, platelets are isolated, for example in a liquid medium, for example prior to processing to form platelet derivatives, or prior to directly administering to a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, platelets are prepared prior to treating a subject as described herein. In some embodiments, the platelets are lyophilized. In some embodiments, the platelets are cryopreserved.

In some embodiments, the platelets or pooled platelets may be acidified to a pH of about 6.0 to about 7.4 prior to the incubation with the incubating agent. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose (ACD).

In some embodiments, tangential flow filtration (TFF) is used to process platelets for making platelet derivatives, in illustrative embodiments FDPDs, for use in aspects here. For example, TFF can be used for concentration and/or buffer or other solution exchange, such that platelets are suspended at an appropriate concentration range in an appropriate medium, for example an incubating agent and/or a lyophilizing agent, or an incubating agent which is or comprises a lyophilizing agent, for example before the composition is dried to form platelet derivatives, or in illustrative embodiments, before the platelet composition is freeze-dried to form FDPDs.

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×10³ to about 4000×10³ platelets/μL (e.g., about 1000×10³ to about 2000×10³, about 2000×10³ to about 3000×10³, or about 4000×10³ platelets/μL). In some embodiments, a concentration step can be carried out using the TFF apparatus.

The concentration of platelets or platelet derivatives (e.g., FDPDs) can be determined by any appropriate method. For example, a counter can be used to quantitate concentration of blood cells in suspension using impedance (e.g., a Beckman Coulter AcT 10 or an ACT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain the platelet derivatives and allow the microparticles to pass through the membrane. The pore size in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×10³ to 20,000×10³. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). As another example, an endpoint can be a platelet concentration of about 1000×10³ to about 2500 platelets/μL (e.g., about 1000×10³ to about 2000×10³, about 1500×10³ to about 2300×10³, or about 1700×10³ to about 2300×10³ platelets/μL). In some embodiments, an endpoint can be a concentration of platelets in the TFF-treated composition are at least 100×10³ platelets/μL, 200×10³ platelets/μL, 400×10³ platelets/μL, 1000×10³ platelets/μL, 1250×10³ platelets/μL, 1500×10³ platelets/μL, 1750×10³ platelets/μL, 2000×10³ platelets/μL, 2250×10³ platelets/μL, 2500×10³ platelets/μL, 2750×10³ platelets/μL, 3000×10³ platelets/μL, 3250×10³ platelets/μL, 3500×10³ platelets/μL, 3750×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, 20,000×10³ platelets/μL. In some embodiments, the platelets or platelet derivatives in the TFF-treated composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, FDPDs. However, in some cases, a TFF-treated composition can be considered to be a final blood product.

In some embodiments, a blood product can be prepared using centrifugation of a blood product (e.g., an unprocessed blood product (e.g., donor apheresis material (e.g., pooled donor apheresis material)), or a partially processed blood product (e.g., a blood product that has undergone TFF)). In some embodiments, a blood product can be prepared without centrifugation of a blood product (e.g., an unprocessed blood product (e.g., donor apheresis material), or a partially processed blood product (e.g., a blood product that has undergone TFF)). Centrifugation can include any appropriate steps. In some embodiments, centrifugation can include a slow acceleration, a slow deceleration, or a combination thereof. In some embodiments, centrifugation can include centrifugation at about 1400×g to about 1550×g (e.g., about 1400 to about 1450×g, about 1450 to about 1500×g, or 1500 to about 1550×g, about 1400×g, about 1410×g, about 1430×g, about 1450×g, about 1470×g, about 1490×g, about 1500×g, about 1510×g, about 1530×g, or about 1550×g). In some embodiments, the duration of centrifugation can be about 10 min to about 30 min (e.g., about 10 to about 20 min, about 20 to about 30 min, about 10 min, about 20 min, or about 30 min).

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, FDPDs can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, FDPDs can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

In some embodiments, the platelets are isolated prior to the incubation with the incubating agent and/or lyophilizing agent. In some embodiments, the incubating agent is or comprises a lyophilizing agent as disclosed in more detail herein. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

An incubating agent can include any appropriate components. In some embodiments, the incubating agent may comprise a liquid medium. In some embodiments the incubating agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the incubating agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the incubating agent includes from about 0.5 mM to about 100 mM of the one or more salts. In some embodiments, the incubating agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) about of the one or more salts. In some embodiments, the incubating agent includes about 5 mM, about 60 mM, about 65 mM, about 70 mM, about 75 mM, or about 80 mM of the one or more salts. In some embodiments, the incubating agent comprises one or more salts selected from calcium salts, magnesium slats, and a combination of the two, in a concentration of about 0.5 mM to about 2 mM.

Preferably, these salts are present in the composition comprising platelets or platelet derivatives, such as freeze-dried platelets, at an amount that is about the same as is found in whole blood.

In some embodiments, the incubating agent further comprises a carrier protein. In some embodiments, the carrier protein comprises human serum albumin, bovine serum albumin, or a combination thereof. In some embodiments, the carrier protein is present in an amount of about 0.05% to about 1.0% (w/v).

The incubating agent may be any buffer that is non-toxic to the platelets and provides adequate buffering capacity to the solution at the temperatures at which the solution will be exposed during the process provided herein. Thus, the buffer may comprise any of the known biologically compatible buffers available commercially for example phosphate buffers such as phosphate buffered saline (PBS), bicarbonate/carbonic acid buffers such as sodium-bicarbonate buffer, N-2-hydroxyethylpiperazine-N′-2-ethanesulfonic acid (HEPES), and tris-based buffers such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the incubating agent includes one or more buffers, e.g., N-2-hydroxyethylpiperazine-N′-2-ethanesulfonic acid (HEPES), or sodium-bicarbonate (NaHCO₃). In some embodiments, the incubating agent includes from about 5 to about 100 mM of the one or more buffers. In some embodiments, the incubating agent includes from about 5 to about 50 mM (e.g., from about 5 mM to about 40 mM, from about 8 mM to about 30 mM, about 10 mM to about 25 mM) about of the one or more buffers. In some embodiments, the incubating agent includes about 10 mM, about 20 mM, about 25 mM, or about 30 mM of the one or more buffers.

In some embodiments, the incubating agent includes one or more saccharides, such as monosaccharides and disaccharides, including sucrose, maltose, trehalose, glucose, mannose, dextrose, and xylose. In some embodiments, the saccharide is a monosaccharide. In some embodiments, the saccharide is a disaccharide. In some embodiments, the saccharide comprises a monosaccharide, a disaccharide, or a combination thereof. In some embodiments, the saccharide is a non-reducing disaccharide. In some embodiments, the saccharide comprises sucrose, maltose, trehalose, glucose (e.g., dextrose), mannose, or xylose. In some embodiments, the saccharide comprises trehalose. In some embodiments, the incubating agent comprises a starch. In some embodiments, the incubating agent includes polysucrose, a polymer of sucrose and epichlorohydrin. In some embodiments, the incubating agent includes from about 10 mM to about 1,000 mM of the one or more saccharides. In some embodiments, the incubating agent includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM. In some embodiments, the one or more saccharides is the lyophilizing agent; for example, in some embodiments, the lyophilizing agent comprises trehalose, polysucrose, or a combination thereof. In some embodiments, the incubating agent comprises polysucrose in a concentration in the range of 0.01 to 5 mM, 0.01 to 4 mM, 0.01 to 3 mM, 0.01 to 2 mM, 0.01 to 1 mM, 0.02 to 1 mM, 0.05 to 1 mM, 0.075 to 1 mM, 0.1 to 1 mM, 0.1 to 0.9 mM, 0.02 to 5 mM, 0.02 to 4 mM, 0.02 to 3 mM, or 0.02 to 2 mM. In some embodiments, the polysucrose is a polysucrose 70 kDa. In illustrative embodiments, the polysucrose is a polysucrose 400 kDa.

In some embodiments the composition comprising platelets or platelet derivatives, (e.g., FDPDs), may comprise one or more of water or a saline solution. In some embodiments the composition comprising platelets or platelet derivatives, such as freeze-dried platelets, may comprise DMSO.

In some embodiments, the incubating agent comprises an organic solvent, such as an alcohol (e.g., ethanol). In such an incubating agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. In some embodiments, the organic solvent comprises ethanol, DMSO, or a combination thereof. The presence of organic solvents, such as ethanol, can be beneficial in the processing of platelets, platelet derivatives, or FDPDs (e.g., freeze-dried platelet derivatives).

In some embodiments the incubating agent is incubated into the platelets in the presence of an aqueous medium. In some embodiments the incubating agent is incubated in the presence of a medium comprising DMSO.

In some embodiments, one or more other components may be incubated in the platelets. Exemplary components may include Prostaglandin E1 or Prostacyclin and or EDTA/EGTA to prevent platelet aggregation and activation during the incubating process.

Non-limiting examples of incubating agent compositions that may be used are shown in Tables 1-5.

TABLE 1
Buffer

| | Concentration |
|---|---|
| | (mM unless |
| | otherwise |
| Component | specified) |
| NaCl | 75.0 |
| KCl | 4.8 |
| HEPES | 9.5 |
| NaHCO₃ | 12.0 |
| Dextrose | 3 |
| Trehalose | 100 |
| Ethanol (optional) | 1% (v/v) |

TABLE 2
Buffer A

| | Concentration |
|---|---|
| | (mM unless |
| | specified |
| Component | otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

TABLE 3
Buffer B can used when incubating
platelets, e.g., for flow cytometry.
Such an incubation can be done
at room temperature in the dark.
Albumin is an optional
component of Buffer B.
Buffer B

| | Concentration |
|---|---|
| | (mM unless |
| | otherwise |
| Component | specified) |
| Buffer and Salts | Table 4 (below) |
| BSA | 0.35% |
| Dextrose | 5 |
| pH | 7.4 |

TABLE 4
Concentration of HEPES
and of Salts in Buffer B

| | Concentration |
|---|---|
| | (mM unless |
| | otherwise |
| Component | specified) |
| HEPES | 25 |
| NaCl | 119 |
| KCl | 5 |
| CaCl₂ | 2 |
| MgCl₂ | 2 |
| glucose | 6 g/l |

Table 4 is another exemplary incubating agent. The pH can be adjusted to 7.4 with NaOH. Albumin is an optional component of Buffer B.

TABLE 5
Tyrode's HEPES Buffer (plus PGE1)

| | Concentration |
|---|---|
| Component | (mM) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostaglandin E1 (PGE1) | 1 μg/ml |

Table 5 is another exemplary incubating agent.

In some embodiments, platelets (e.g., apheresis platelets, platelets isolated from whole blood, pooled platelets, or a combination thereof) are incubated with the incubating agent for different durations at or at different temperatures from 15-45° C., or about 37° C.

In some embodiments, platelets (e.g., apheresis platelets, platelets isolated from whole blood, pooled platelets, or a combination thereof) form a suspension in an incubating agent and/or a lyophilizing agent, or an incubating agent that is or comprises a lyophilizing agent, comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

The platelets (e.g., apheresis platelets, platelets isolated from whole blood, pooled platelets, or a combination thereof) may be incubated with the incubating agent, for example that is or comprises a lyophilizing agent, for different durations, such as, for example, for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the platelets may be incubated with the incubating agent for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the platelets may be incubated with the incubating agent for from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs. In some embodiments, the platelets, the platelet derivatives, or the FDPDs are incubated with the incubating agent for a period of time of 5 minutes to 48 hours, such as 10 minutes to 24 hours, such as 20 minutes to 12 hours, such as 30 minutes to 6 hours, such as 1 hour minutes to 3 hours, such as about 2 hours.

In some embodiments, the platelets (e.g., apheresis platelets, platelets isolated from whole blood, pooled platelets, or a combination thereof) are incubated with the incubating agents at different temperatures. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours. In some embodiments, the platelets are incubated with the incubating agent for different durations as disclosed herein, and at temperatures from 15-45° C., or about 37° C.

In some embodiments, platelets (e.g., apheresis platelets, platelets isolated from whole blood, pooled platelets, or a combination thereof) are loaded with one or more active agents. In some embodiments, the platelets can be loaded with an anti-fibrinolytic agent. Non-limiting examples of anti-fibrinolytic agents include &-aminocaproic acid (EACA), tranexamic acid, aprotinin, aminomethylbenzoic acid, and fibrinogen.

Loading platelets (e.g., apheresis platelets, platelets isolated from whole blood, pooled platelets, or a combination thereof) with an active agent (e.g., an anti-fibrinolytic agent) can be performed by any appropriate method. See, for example, PCT Publication Nos. WO2020113090A1, WO2020113101A1, WO2020113035A1, and WO2020112963A1. Generally, the loading includes contacting the platelets with the anti-fibrinolytic agent. In some embodiments, the loading can be performed by combining the active agent with the incubating agent. In some embodiments, the loading can be performed in a separate step from the incubating step. For example, the loading can be performed in a step prior to the incubation step. In some such embodiments, the active agent can be supplied to the platelets as a solution or suspension in any of the incubation agents described herein, which may or may not be the same as the incubating agent used in the incubating step. In some embodiments, the loading step can be performed during the incubation step. In some such embodiments, the active agent can be added to the incubation agent (e.g., as a solid or in a solution or suspension) during the incubation). In some embodiments, the loading step can be performed in a step following the incubation step. In some such embodiments, be supplied to the platelets as a solution or suspension in any of the incubation agents described herein, which may or may not be the same as the incubating agent used in the incubating step.

An active agent can be applied to the platelets in any appropriate concentration. In some embodiments, an active agent can be applied to the platelets (e.g., as part of the incubating agent or another solution or suspension) in a concentration of about 1 μM to about 100 mM (e.g., about 1 μM to about 10 μm, about 1 μM to about 50 μM, about 1 μM to about 100 μM, about 1 μM to about 500 μM, about 1 μM to about 1 mM, about 1 μM to about 10 mM, about 1 μM to about 25 mM, about 1 μM to about 50 mM, about 1 μM to about 75 mM, about 10 μM to about 100 mM, about 50 μM to about 100 mM, about 100 μM to about 100 mM, about 500 μM to about 100 mM, about 1 mM to about 100 mM, about 10 mM to about 100 mM, about 25 mM to about 100 mM, about 50 mM to about 100 mM, about 75 mM to about 100 mM, about 10 μM to about 100 mM, about 200 μM to about 1 mM, about 800 μM to about 900 μM, about 400 μM to about 800 μM, about 500 μM to about 700 μM, about 600 μM, about 5 mM to about 85 mM, about 20 mM to about 90 mM, about 25 mM to about 75 mM, about 30 mM to about 90 mM, about 35 mM to about 65 mM, about 40 mM to about 60 mM, about 50 mM to about 60 mM, about 40 mM to about 70 mM, about 45 mM to about 55 mM, or about 50 mM).

In some embodiments, the method further comprises drying the platelets. In some embodiments, the drying step comprises lyophilizing the platelets. In some embodiments, the drying step comprises freeze-drying the platelets. In some embodiments, the method further comprises rehydrating the platelets obtained from the drying step.

In some embodiments, the platelets are cold stored, cryopreserved, or lyophilized (e.g., to produce FDPDs) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a incubating agent according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | | | | | | | Pressure |
|---|---|---|---|---|---|---|---|
| | Step | Temp. Set | | Type | Duration | | Set |
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the platelets that are obtained as disclosed herein, to produce platelet derivatives for use in any of the aspects or embodiments herein, such as the step of freeze-drying the platelets that are obtained as disclosed herein, to produce FDPDs for use in any of the aspects or embodiments herein comprises incubating the platelets with a lyophilizing agent (e.g., a non-reducing disaccharide). Accordingly, in some embodiments, the methods for preparing platelets further comprise incubating the platelets with a lyophilizing agent. In some embodiments the lyophilizing agent is a saccharide. In some embodiments the saccharide is a disaccharide, such as a non-reducing disaccharide.

In some embodiments, the platelets are incubated with a lyophilizing agent for a sufficient amount of time and at a suitable temperature to incubate the platelets with the lyophilizing agent. In some embodiments, the incubating agent is the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

An exemplary saccharide for use in the compositions disclosed herein is trehalose. Regardless of the identity of the saccharide, it can be present in the composition that is dried to form platelet derivatives, or freeze-dried to form, FDPDs, and in rehydrated compositions of such dried platelet derivatives and FDPDs, in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

Within the process provided herein for making the compositions provided herein, addition of the lyophilizing agent can be the last step prior to drying. However, in some embodiments, the lyophilizing agent is added at the same time or before other components of the composition, such as a salt, a buffer, optionally a cryoprotectant, or other components. In some embodiments, the lyophilizing agent is added to the incubating agent, thoroughly mixed to form a drying solution, dispensed into a drying vessel (e.g., a glass or plastic serum vial, a lyophilization bag), and subjected to conditions that allow for drying of the solution to form a dried composition.

The step of incubating the platelets with a cryoprotectant can include incubating the platelets for a time suitable for loading, as long as the time, taken in conjunction with the temperature, is sufficient for the cryoprotectant to come into contact with the platelets and, preferably, be incorporated, at least to some extent, into the platelets. In embodiments, incubation is carried out for about 1 minute to about 180 minutes or longer.

The step of incubating the platelets with a cryoprotectant can include incubating the platelets and the cryoprotectant at a temperature that, when selected in conjunction with the amount of time allotted, is suitable for incubating. In general, the composition is incubated at a temperature above freezing for at least a sufficient time for the cryoprotectant to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets or platelet derivatives (e.g., FDPDs) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., FDPDs) for at least 30 minutes at a temperature higher than 30° C.; heating the dried platelets or platelet derivatives (e.g., FDPDs) for at least 10 hours at a temperature higher than 50° C.; heating the dried platelets or platelet derivatives (e.g., FDPDs) for at least 18 hours at a temperature higher than 75° C.; and heating the dried platelets or platelet derivatives (e.g., FDPDs) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., FDPDs).

In some embodiments, the platelets or platelet derivatives (e.g., FDPDs) prepared as disclosed herein by a process comprising incubation with an incubating agent have a storage stability that is at least about equal to that of the platelets prior to the incubation.

In some embodiments, the method further comprises cryopreserving the platelets or platelet derivatives prior to administering the platelets or platelet derivatives (e.g., with an incubating agent, e.g., an incubating agent described herein).

In some embodiments, the method further comprises drying a composition comprising platelets or platelet derivatives, (e.g., with an incubating agent e.g., an incubating agent described herein) prior to administering the platelets or platelet derivatives (e.g., FDPDs). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with an incubating agent e.g., an incubating agent described herein) prior to administering the platelets or platelet derivatives (e.g., FDPDs) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the FDPDs prior to administering the platelets, platelet derivatives, or FDPDs (e.g., with an incubating agent, e.g., an incubating agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., FDPDs) (e.g., with an incubating agent e.g., an incubating agent described herein) prior to administering the platelets or platelet derivatives (e.g., FDPDs). For example, in some embodiments, the method further comprises drying (e.g., freeze-drying) a composition comprising platelets or platelet derivatives (e.g., with an incubating agent e.g., an incubating agent described herein) (e.g., to form FDPDs) prior to administering the platelets or platelet derivatives (e.g., FDPDs). In some embodiments, the method may further comprise rehydrating the composition obtained from the drying step.

In some embodiments, provided herein is a composition comprising platelets or platelet derivatives (e.g., FDPDs), polysucrose and trehalose made by the process of obtaining fresh platelets, optionally incubating the platelets in DMSO, isolating the platelets by centrifugation, resuspending the platelets in an incubating agent which comprises trehalose and ethanol thereby forming a first mixture, incubating the first mixture, mixing polysucrose with the first mixture, thereby forming a second mixture, and lyophilizing the second mixture to form a freeze dried composition comprising platelets or platelet derivatives (e.g., FDPDs), polysucrose and trehalose.

In some embodiments, provided herein is a method of making a freeze-dried platelet composition comprising platelets or platelet derivatives (e.g., FDPDs), polysucrose and trehalose comprising obtaining fresh platelets, optionally incubating the platelets in DMSO, isolating the platelets by centrifugation, resuspending the platelets in a incubating agent which comprises trehalose and ethanol thereby forming a first mixture, incubating the first mixture, mixing polysucrose with the first mixture, thereby forming a second mixture, and lyophilizing the second mixture to form a freeze-dried composition comprising platelets or platelet derivatives (e.g., FDPDs), polysucrose and trehalose.

In some embodiments, provided herein is a process for making freeze-dried platelets, the process comprising incubating isolated platelets in the presence of at least one saccharide under the following conditions: a temperature of from 20° C. to 42° C. for about 10 minutes to about 180 minutes, adding to the platelets at least one cryoprotectant, and lyophilizing the platelets, wherein the process optionally does not include isolating the platelets between the incubating and adding steps, and optionally wherein the process does not include exposing the platelets to a platelet activation inhibitor. The cryoprotectant can be a polysugar (e.g., polysucrose). The process can further include heating the lyophilized platelets at a temperature of 70° C. to 80° C. for 8 to 24 hours. The step of adding to the platelets at least one cryoprotectant can further include exposing the platelets to ethanol. The step of incubating isolated platelets in the presence of at least one saccharide can include incubating in the presence of at least one saccharide. The step of incubating isolated platelets in the presence of at least one saccharide can include incubating in the presence of at least one saccharide. The conditions for incubating can include incubating for about 100 minutes to about 150 minutes. The conditions for incubating can include incubating for about 110 minutes to about 130 minutes. The conditions for incubating can include incubating for about 120 minutes. The conditions for incubating can include incubating at 35° C. to 40° C. The conditions for incubating can include incubating at 37° C. The conditions for incubating can include incubating at 35° C. to 40° C. for 110 minutes to 130 minutes. The conditions for incubating can include incubating at 37° C. for 120 minutes. The at least one saccharide can be trehalose, sucrose, or both trehalose and sucrose. The at least one saccharide can be trehalose. The at least one saccharide can be sucrose.

In some embodiments, provided herein is a method of preparing freeze-dried platelets, the method including providing platelets, suspending the platelets in a salt buffer that includes about 100 mM trehalose and about 1% (v/v) ethanol to make a first composition, incubating the first composition at about 37° C. for about 2 hours, adding polysucrose (e.g., polysucrose 400) to a final concentration of about 6% (w/v) to make a second composition, lyophilizing the second composition to make freeze-dried platelets, and heating the freeze-dried platelets at 80° C. for 24 hours.

Specific embodiments disclosed herein may be further limited in the claims using “consisting of” or “consisting essentially of” language.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

Provided herein in one aspect is a method for treating a coagulopathy, wherein the subject has a coagulopathy and/or has an increased risk of coagulopathy, as a result of being administered or having been administered a Factor XI inhibitor, the method comprises:
optionally determining in a pre-administering evaluation, that the subject has an abnormal value for one or more clotting parameters; and
administering to the subject having an increased potential for bleeding, an effective amount of a composition comprising freeze-dried platelet derivatives (FDPDs) such that a bleeding potential of the subject is reduced, thereby treating the coagulopathy. In illustrative embodiments, the composition comprising FDPDs comprises a population of FDPDs having a property of a reduced propensity to aggregate as compared to platelets under in vitro aggregation conditions consisting of 1 mg/ml ristocetin, a buffer, and citrated plasma, as measured by light transmission aggregometry 5 minutes after ristocetin addition, such that no more than 10% of the FDPDs in the population of FDPDs aggregate under the in vitro aggregation conditions. In illustrative embodiments, the FDPDs when at a concentration of at least 70×10³ particles/μl, results in an occlusion time of less than 14 minutes in a total thrombus-formation analysis system (T-TAS). In illustrative embodiments, at least 50% of the FDPDs are CD 41-positive-FDPDs, wherein less than 5% of the CD 41-positive FDPDs are microparticles having a diameter of less than 0.5 μm. In illustrative embodiments, the coagulopathy is bleeding or an increased potential for bleeding, wherein the subject is bleeding and/or has an increased potential for bleeding before the administering, and such bleeding and/or risk of bleeding is reduced, for example as discussed herein in more detail, or stopped after the administering. In some embodiments, the subject is bleeding as a result of being administered or having been administered an antiplatelet agent in addition to the anticoagulant. In some embodiments, the antiplatelet agent comprises aspirin and at least one other antiplatelet agent.

Provided herein in one aspect is a method of administering platelet derivatives to a subject, wherein the subject has been treated or is being treated with at least one anticoagulant, and at least one antiplatelet agent. In some embodiments, the subject has been treated or is being treated with at least one anticoagulant, aspirin, and at least one antiplatelet agent. In some embodiments, the anticoagulant is selected from agents that inhibit Factor IIa, VIIa, IX, Xa, XI, Tissue Factor, or vitamin K-dependent synthesis of clotting factors (e.g., Factor II, VII, IX, or X) or that activate antithrombin (e.g., antithrombin III). In illustrative embodiments, the anticoagulant comprises Factor XI inhibitor. In illustrative embodiments, the subject has been treated or is being treated with one anticoagulant, one antiplatelet agent, and aspirin, in illustrative embodiments, the anticoagulant agent comprises a factor VIII inhibitor, a factor IX inhibitor, or a factor XI inhibitor. In illustrative embodiments, the anticoagulant comprises a Factor XI inhibitor. In some embodiments, the factor XI inhibitor can be a biologic or an antibody that inhibits or creates a loss of or reduced function of factor XI in a subject. In some embodiments, the factor XI inhibitor can be any agent that is approved by a regulatory agency, or is undergoing a clinical trial. In illustrative embodiments, the factor XI inhibitor can be one or more inhibitor selected from IONIS-FXI, FXI-ASO, osocimab, abelacimab, milvexian, xisomab 3G3, fesomersen, asundexian, and MK-2060.

Provided herein in one aspect is a method for administering platelet derivatives to a subject, the method comprising:
rehydrating a composition in the form of a powder, said composition comprising the platelet derivatives and an incubating agent comprising one or more saccharides, one or more salts, and a buffer to form a rehydrated platelet derivative composition, and
administering a dose of the rehydrated platelet derivative composition to the subject, wherein the dose comprises a first dose of the rehydrated platelet derivatives, and
wherein the subject has been treated or is being treated with at least one anticoagulant agent, and/or at least one antiplatelet agent. In illustrative embodiments, the first dose comprises an amount of rehydrated platelet derivatives between 1.0×10⁷ particles/kg and 1.0×10¹¹ particles/kg of the subject. In some embodiments, the administering comprises administering parenterally, and in illustrative embodiments, comprises administering intravenously. In some embodiments, the subject has been treated or is being treated with 1, 2, 3, 4, or more anticoagulant agents. In some embodiments, the subject has been treated or is being treated with 1, 2, 3, 4, or more antiplatelet agents. In some embodiments, the subject has been treated or is being treated with one anticoagulant agent, and 1, 2, or 3 antiplatelet agents.

Provided herein in one aspect is a method for treating a subject, the method comprising:
rehydrating a composition in the form of a powder, said composition comprising the platelet derivatives and an incubating agent comprising one or more saccharides, one or more salts, and a buffer to form a rehydrated platelet derivative composition, and
administering intravenously an effective amount of the rehydrated platelet derivative composition to the subject, wherein the subject has been treated or is being treated with at least one anticoagulant agent, and at least one antiplatelet agent.

In some embodiments of any of the aspects herein, the at least one anticoagulant agent is selected from the group consisting of Factor XI inhibitor, Factor IIa inhibitor, Factor VIIa inhibitor, Factor IX inhibitor, and Factor Xa inhibitor. In some embodiments, the at least one anticoagulant agent is a Factor XI inhibitor.

Provided herein in one aspect is a method for administering platelet derivatives to a subject, the method comprising:
rehydrating a composition in the form of a powder, said composition comprising the platelet derivatives and an incubating agent comprising one or more saccharides, one or more salts, and a buffer to form a rehydrated platelet derivative composition, and
administering a dose of the rehydrated platelet derivative composition to the subject, wherein the dose comprises a first dose of the rehydrated platelet derivatives,
   wherein the subject has been treated or is being treated with aspirin, at least one anticoagulant agent, and at least one other antiplatelet agent, and
   wherein the at least one anticoagulant agent comprises a Factor XI inhibitor. In illustrative embodiments, the administering is done intravenously. In illustrative embodiments, the first dose comprises an amount of rehydrated platelet derivatives between 1.0×10⁷ particles/kg and 1.0×10¹¹ particles/kg of the subject.

Provided herein in one aspect is a method for administering platelet derivatives to a subject, the method comprising:
rehydrating a composition in the form of a powder, said composition comprising the platelet derivatives and an incubating agent comprising one or more saccharides, one or more salts, and a buffer to form a rehydrated platelet derivative composition, and
administering a dose of the rehydrated platelet derivative composition to the subject, wherein the dose comprises a first dose of the rehydrated platelet derivatives,
   wherein the subject has been treated or is being treated with at least one anticoagulant agent, and
   wherein the at least one anticoagulant agent comprises a Factor XI inhibitor. In illustrative embodiments, the subject has been or is being treated with an antiplatelet agent, in addition to the Factor XI inhibitor. In illustrative embodiments, the method is a method for reducing, stopping, and/or controlling bleeding in the subject. In illustrative embodiments, the administering is done intravenously. In illustrative embodiments, the first dose comprises an amount of rehydrated platelet derivatives between 1.0×10⁷ particles/kg and 1.0×10¹¹ particles/kg of the subject. In some embodiments, the at least one antiplatelet agent is aspirin. In some embodiments, the antiplatelet agent is an antiplatelet agent other than aspirin. In some embodiments, the antiplatelet agent is 1, 2, or 3 of any of the antiplatelet agents as disclosed herein. In illustrative embodiments, the subject is being treated or has been treated with a factor XI inhibitor, aspirin, and ticagrelor. In some embodiments, the subject is being treated or has been treated with a factor XI inhibitor, aspirin, and any other antiplatelet agent that can be a part of dual antiplatelet therapy along with aspirin. In some embodiments, the subject is being treated or has been treated with a factor XI inhibitor, and two antiplatelet agents.

In some embodiments of any of the aspects herein, the administering the dose comprises administering between 2 and 10 doses of the rehydrated platelet derivative composition, wherein each dose that is administered between 2 and 10 times, 2 and 9 times, 2 and 8 times, 2 and 6 times, 2 and 5 times, 3 and 10 times, 5 and 10 times, or 4 and 8 times is an amount of rehydrated platelet derivatives between 1.0×10⁷ particles/kg and 1.0×10¹¹ particles/kg of the subject. In some embodiments, the dose comprises an amount of the rehydrated platelet derivatives between 1.0×10⁹ particles/kg and 1.0×10¹⁰ particles/kg of the subject.

In some embodiments of any of the aspects herein, the at least one other antiplatelet agent is selected from the group consisting of cangrelor, ticagrelor, clopidogrel, prasugrel, eptifibatide, tirofiban, abciximab, terutroban, picotamide, ibuprofen, vorapaxar, atopaxar, cilostazol, prostaglandin E1, epoprostenol, dipyridamole, treprostinil sodium, and sarpogrelate. In some embodiments, the at least one other antiplatelet agent is selected from the group consisting of cangrelor, ticagrelor, clopidogrel, and prasugrel. In some embodiments, the at least one antiplatelet agent is selected from the group consisting of aspirin, cangrelor, ticagrelor, clopidogrel, prasugrel, eptifibatide, tirofiban, abciximab, terutroban, picotamide, ibuprofen, vorapaxar, atopaxar, cilostazol, prostaglandin E1, epoprostenol, dipyridamole, treprostinil sodium, and sarpogrelate. In some embodiments, the at least one antiplatelet agent comprises aspirin. In some embodiments, the subject is being treated with one anticoagulant agent that is a Factor XI inhibitor, aspirin, and one other antiplatelet agent. In some embodiments, the subject is being treated with one anticoagulant agent that is a Factor XI inhibitor, and one antiplatelet agent. In some embodiments, the subject is being treated with one anticoagulant agent that is a Factor XI inhibitor, and two antiplatelet agents.

In some embodiments of any of the aspects herein, the subject has a bleeding of grade 2, 3, or 4 based on the WHO bleeding scale before the administering of the rehydrated platelet derivative composition. In some embodiments, after the administering of the rehydrated platelet derivative composition, the subject has a reduced bleeding such that the subject has one grade, two grades, or three grades less bleeding based on the WHO bleeding scale as compared to the bleeding before the administering of the rehydrated platelet derivative composition. In some embodiments, the administering the rehydrated platelet derivative composition restores hemostasis in the subject. In illustrative embodiments, the administering leads to reduced bleeding as compared to before the administering. In illustrative embodiments, the administering leads to stopping of the bleeding.

In some embodiments of any of the aspects herein, the subject is bleeding before the administering of the rehydrated platelet derivative composition, and the administering the rehydrated platelet derivative composition restores hemostasis in the subject.

In some embodiments of any of the aspects herein, the anticoagulant agent, aspirin, or the other antiplatelet agent is present in the blood of the subject in an amount sufficient to cause a delay in the time which it takes to stop the bleeding in the subject. In some embodiments, the anticoagulant agent, aspirin, or the antiplatelet agent is present at a highest concentration (Cmax) in the blood of the subject within 8, 7, 6, 5, 4, 3, 2 hours, 1 hour, 45, 40, 30, 20, 15, or 10 minutes of administering the dose of the rehydrated platelet derivatives.

In some embodiments of any of the aspects herein, the subject is being treated with the anticoagulant agent, and the antiplatelet agent such that a recent dose of the anticoagulant agent, or the antiplatelet agent is administered to the subject within 1 week, 6 days, 5 days, 4, days, 3 days, 2 days, 1 day, 20 hours, 18 hours, 16 hours, 14 hours, 12 hours, 10 hours, 8 hours, 6 hours, 4 hours, 3 hours, 2 hours, 1 hour, 45 minutes, 30 minutes, or 15 minutes of administering the dose of the rehydrated platelet derivatives.

In some embodiments of any of the aspects herein, the one or more saccharides comprise trehalose in an amount in the range of 10 mM to 500 mM, and polysucrose in an amount in the range of 3% to 10%, w/v.

In some embodiments of any of the aspects herein, the platelet derivative composition in the form of the powder is prepared by a process, comprising: performing tangential flow filtration (TFF) of a platelet composition comprising platelets in a preparation agent comprising a buffering agent, trehalose in an amount in the range of 10 mM to 500 mM, and polysucrose in an amount in the range of 3% to 10%, thereby preparing a TFF-treated composition comprising at least 1000×10³ platelets/μl in an aqueous medium having less than or equal to 7.5% plasma protein and having less than 5.0% microparticles by scattering intensity; freeze drying the TFF-treated composition comprising platelets in the aqueous medium to form a freeze-dried platelet derivative composition comprising platelet derivatives; and heating the freeze-dried platelet derivative composition at a temperature in the range of 60° C. to 90° C. for at least 1 hour to not more than 36 hours to thermally treat the platelet derivatives in the freeze-dried platelet derivative composition to form the platelet derivative composition in the form of the powder.

Provided herein in one aspect is a method of treating a subject, the method comprising administering to the subject in need thereof, an effective amount of any of the platelet derivative compositions disclosed herein, or the platelet derivative composition prepared by any of the methods disclosed herein, wherein the subject has been treated or is being treated with an antiplatelet agent and/or an anti-coagulant, and wherein the method for treating is a) a method for restoring normal hemostasis in the subject; b) for treating a coagulopathy in the subject; or c) for preparing the subject for surgery. In some embodiments, the method of treating is the method of restoring normal hemostasis in the subject. In some embodiments, the method of treating is the method for treating the coagulopathy in the subject. In some embodiments, the method of treating is the method for preparing the subject for surgery. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, a supplement, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, sarpogrelate, and a combination thereof. In some embodiments of any of the methods for treating aspects herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof. In some embodiments of any of the methods for treating aspects herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof. In some embodiments of any of the methods for treating aspects herein, the administering comprises administering topically, parenterally, intravenously, intramuscularly, intrathecally, subcutaneously or intraperitoneally. In some embodiments of any of the methods for treating aspects herein, where the composition is in the form of a powder, the method further includes, before the administering, rehydrating the composition. In some embodiments provided herein is use of any of the platelet derivative compositions herein, in the manufacture of a kit for performing any of the methods of treating provided herein. In illustrative embodiments, the anticoagulant comprises a Factor XI inhibitor. In some embodiments of any of the methods herein, the subject has been or is being treated with more than one antiplatelet agents, for example, 2, 3, or 4 antiplatelet agents. In some embodiments, the subject has been or is being treated with aspirin, and one or more than one antiplatelet agents other than aspirin. In some embodiments, the subject has been or is being treated with aspirin, one other antiplatelet agent in addition to one or more anticoagulant agent, in illustrative embodiments, the anticoagulant agent comprises a Factor XI inhibitor.

In any of the aspects and embodiments herein, the subject is treated with aspirin, and at least one anticoagulant agent. In some embodiments, the subject is treated with at least one other antiplatelet agent, and at least one other anticoagulant agent in addition to aspirin, and the at least one anticoagulant agent. In some embodiments, the subject is treated with aspirin, and at least one anticoagulant agent, wherein the anticoagulant agent comprises a factor XI inhibitor. In some embodiments, the subject is treated with aspirin, and a factor XI inhibitor. In some embodiments, the subject is treated with aspirin, a factor XI inhibitor, and at least one other antiplatelet agent, and/or at least one other anticoagulant agent.

Provided herein in one aspect is a method of treating a coagulopathy in a subject, the method including administering to the subject in need thereof an effective amount of a composition including platelets, or in illustrative embodiments platelet derivatives, and in further illustrative embodiments FDPDs, thereby treating the coagulopathy. In some embodiments, the coagulopathy is the result of the subject being administered an anti-coagulant, which in illustrative embodiments is a Factor XI inhibitor. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, the method including administering to the subject in need thereof an effective amount of a composition. In illustrative embodiments, the subject has a coagulopathy as the result of being administered a Factor XI inhibitor. In illustrative embodiments, the subject is bleeding, has uncontrolled bleeding, or has persistent bleeding before the administering, and such bleeding is reduced after the administering, for example by WHO grade 1, grade 2, or grade 3, and in illustrative embodiments where the bleeding is reduced to a WHO grade 1 or less, virtually stopped or stopped. In some embodiments, the composition is prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in illustrative embodiments FDPDs, thereby treating the coagulopathy. In illustrative embodiments, the composition comprising the platelet derivatives is administered such that the bleeding and/or the bleeding potential of the subject is reduced, and in illustrative embodiments such that normal hemostasis is restored in the subject. In some embodiments, the bleeding in the subject is stopped after the administering of the platelet derivatives.

In one aspect, provided herein is a method of restoring normal hemostasis in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject. In some embodiments, the bleeding is reduced to a level such that it is no longer life-threatening. In one aspect, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition comprises platelet derivatives, and in further illustrative embodiments FDPDs, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject. In some embodiments, the bleeding in the subject is stopped after the administering of the platelet derivatives.

In one aspect, provided herein is a method of preparing a subject for surgery, the method including administering to the subject in need thereof an effective amount of a composition including platelets, or in illustrative embodiments platelet derivatives, and in further illustrative embodiments FDPDs. Various properties of exemplary embodiments of such FDPDs are provided herein, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject. Implementations can include one or more of the following features. The surgery can be an emergency surgery. The surgery can be a scheduled surgery.

In one aspect, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in further illustrative embodiments FDPDs, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject. Various properties of exemplary embodiments of such FDPDs are provided herein. Implementations can include one or more of the following features. The surgery can be an emergency surgery. The surgery can be a scheduled surgery.

In one aspect, provided herein is a method of ameliorating the effects of an anticoagulant agent in a subject, the method including administering to the subject in need thereof an effective amount of a composition platelets, or in illustrative embodiments platelet derivatives, and in further illustrative embodiments FDPDs, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject.

In one aspect, provided herein is a method of ameliorating the effects of an anticoagulant agent, and/or an antiplatelet agent in a subject, the method including administering to the subject in need thereof an effective amount of a composition prepared by a process including incubating platelets with an incubating agent including one or more salts, a buffer, optionally a cryoprotectant, and optionally an organic solvent, to form the composition, wherein the composition includes platelet derivatives, and in further illustrative embodiments FDPDs, thereby treating the coagulopathy. In illustrative embodiments, the composition comprising the platelet derivatives is administered such that the bleeding or the bleeding potential of the subject is reduced, and in illustrative embodiments such that normal hemostasis is restored in the subject.

In one aspect, provided herein is a method of treating a coagulopathy in a subject, or of restoring hemostasis in a subject, or of reducing the bleeding or the bleeding potential of a subject that is being administered, or has been administered, an anticoagulant agent, and/or antiplatelet agent, the method comprising: administering to the subject in need thereof an effective amount of a composition comprising platelet derivatives, thereby treating the coagulopathy, restoring hemostasis, or reducing bleeding in the subject. In illustrative embodiments, the platelet derivatives are freeze-dried platelet derivatives (FDPDs). In further illustrative embodiments, the composition comprising the platelet derivatives is administered such that the bleeding or the bleeding potential of the subject is reduced, and in illustrative embodiments such that normal hemostasis is restored in the subject.

In another aspect, provided herein is a method of treating a coagulopathy in a subject, or of restoring hemostasis in a subject, or of reducing the bleeding or the bleeding potential of a subject, wherein the subject is being administered, or has been administered, an anticoagulant agent, the method comprising administering to the subject in need thereof an effective amount of the composition comprising FDPDs, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy. In further illustrative embodiments, the composition comprising the FDPDs is administered such that the bleeding or the bleeding potential of the subject is reduced, and in illustrative embodiments such that normal hemostasis is restored in the subject.

In another aspect, provided herein is a method of preventing or mitigating the potential for a coagulopathy in a subject, the method comprises: (a) determining that information regarding whether the subject was administered an anticoagulant agent is unavailable; and (b) administering to the subject an effective amount of a composition comprising freeze-dried platelet derivatives (FDPDs). In some embodiments of such a method, information regarding whether the subject was administered an anticoagulant agent is unavailable for a reason comprising that the subject cannot be identified. In some embodiments of the method, information regarding whether the subject was administered an anticoagulant agent is unavailable for a reason comprising that the medical history of the subject is unavailable. In further embodiments information regarding whether the subject was administered an anticoagulant agent is unavailable for a reason comprising that the subject is in need of emergency treatment.

In another aspect, provided herein is a method of treating a coagulopathy in a subject or of reducing the bleeding or the bleeding potential of a subject, or of restoring hemostasis in a subject, wherein the method comprises: administering to the subject in need thereof an effective amount of a composition comprising platelet derivatives, in illustrative embodiments, FDPDs, wherein the subject before the administering the composition comprising platelet derivatives, was administered an anticoagulant agent and a second agent that decreases platelet function, thereby treating the coagulopathy. In further illustrative embodiments, the composition comprising the platelet derivatives is administered such that the bleeding or the bleeding potential of the subject is reduced, and in illustrative embodiments such that normal hemostasis is restored in the subject. In illustrative embodiments, before the administering of the composition comprising FDPDs the subject was in need thereof because of an increased risk of bleeding due to, or as a result of being administered the anti-platelet agent and the second agent.

In another aspect, provided herein is a composition comprising freeze-dried platelet derivatives (FDPDs) for treating a coagulopathy in a subject, wherein the treating comprises:
administering to the subject in need thereof, an effective amount of the composition comprising FDPDs such that the bleeding or the bleeding potential, or risk of bleeding of the subject is reduced,
wherein the subject was administered an anticoagulant agent and a second agent that decreases platelet function, and
wherein the subject is in need thereof because of an increased potential for, or risk of bleeding, or the subject is bleeding due to, or as a result of being administered the anticoagulant agent and the second agent,
thereby treating the coagulopathy. In some embodiments, the second agent comprises an antiplatelet agent, and in illustrative embodiments, the antiplatelet agent comprises aspirin, and one other antiplatelet agent other than aspirin.

In another aspect, provided herein is a composition comprising freeze-dried platelet derivatives (FDPDs) for treating a coagulopathy in a subject having an increased potential for, or risk of bleeding as a result of being administered or having been administered an anticoagulant, wherein the treating comprises:
administering to the subject having the increased potential for, or risk of bleeding, an effective amount of the composition comprising FDPDs such that the bleeding or the bleeding potential or risk of bleeding of the subject is reduced,
wherein the composition comprising FDPDs comprises a population of FDPDs having a reduced propensity to aggregate such that no more than 10% of the FDPDs in the population aggregate under aggregation conditions comprising an agonist but no platelets,
thereby treating the coagulopathy.

In some embodiments of any of the aspects and embodiments herein that includes administering platelet derivatives, FDPDs, or FPH to a subject, comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours. In illustrative embodiments, the subject has been or is being administered an antiplatelet agent. In illustrative embodiments, the subject has been or is being administered an anticoagulant agent. In illustrative embodiments, the subject has been or is being administered an antiplatelet agent and anticoagulant agent. In illustrative embodiments, the subject has been or is being administered aspirin, and an anticoagulant agent. In illustrative embodiments, the administering comprises administering until the bleeding is reduced such that it is no longer considered life threatening, or the bleeding is stopped, or normal hemostasis is restored in the subject. In illustrative embodiments, a single dose includes at least 1×10⁸/kg of the subject. In some embodiments of any of the aspects or embodiments herein that include compositions comprising platelet derivatives for use for controlling bleeding in a subject, or include methods for controlling bleeding in a subject comprise administering at least one dose of platelet derivatives every 1 hour, or more frequently, in illustrative embodiments, the administration comprises administering a total of 2 to 30 doses, 3 to 30 doses, 4 to 30 doses, 5 to 30 doses, 5 to 25 doses, 5 to 20 doses, 5 to 15 doses, 5 to 10 doses in a 24-hour period, until bleeding of the subject is reduced as compared to the bleeding before the administration. In some embodiments of any of the aspects or embodiments herein that include compositions comprising platelet derivatives for use for controlling bleeding in a subject, or include methods for controlling bleeding in a subject, wherein said composition is administered to the subject multiple times by administering 2 to 10 doses of the platelet derivatives, in illustrative embodiments at a frequency of every 1 hour, or more frequently, until bleeding of the subject is reduced as compared to the bleeding before the administering, and wherein the platelet derivatives have the ability to generate thrombin in vitro in the presence of tissue factor and phospholipids. In some embodiments, the administering is performed until bleeding of the subject is stopped. In some embodiments, the administering is performed at a frequency of every 30 minutes, or more frequently. In some embodiments, the administering is performed at a frequency of every 15 minutes, or more frequently. In some embodiments, the administering is performed as the continuous infusion. In some embodiments, the administering is performed at a frequency of every 45 minutes, or more frequently. In some embodiments, the administering is performed by administering 3 to 10 doses at the frequency. In some embodiments, the administering is performed until bleeding of the subject is reduced that is observable by visual inspection. In some embodiments, the administering is performed until bleeding of the subject is reduced such that it is no longer considered life threatening. In some embodiments, the administering is performed until bleeding of the subject is reduced such that the bleeding is considered minor. In some embodiments, the administering is performed until it is determined that bleeding of the subject is stopped by a visual inspection. In some embodiments, the administering is performed until bleeding of the subject is stopped at a primary site. In some embodiments, the administering is performed until bleeding of the subject is stopped at any other site than a primary site of bleeding. In some embodiments, the administering is done by a topical administration of the composition. In some embodiments, the administering is done by a parenteral administration of the composition. In illustrative embodiments, the parenteral administration is done by intravenous administration. In some embodiments, the administering includes administering one or multiple doses of platelet derivatives provided herein, by each of a parenteral and a topical route of administration. In some embodiments, the administering further comprises a topical administration of a second composition, and wherein the second composition comprises platelet derivatives. In some embodiments, the second composition is the same as the composition administered by the parenteral route. In other embodiments, the second composition is different from the composition administered by the parenteral route. In some embodiments, the administering comprises the parenteral administration and the topical administration in an alternate manner. In some embodiments, a single dose or an effective dose of the platelet derivatives is in the range of 1.0×10⁷ to 1.1×10¹⁴/kg, 1.0×10⁸ to 1.1×10¹⁴/kg, or 1.5×10⁹ to 1.1×10¹⁴/kg of the subject. In some embodiments, a total of 2 to 20, 2 to 12, 4 to 10, 5 to 8, 5 to 20, or 5 to 15 doses are administered to the subject.

In some embodiments, for example of aspects wherein a subject was administered the anticoagulant agent and the second agent that decreases platelet function, such a method further comprises before the administering the composition comprising FDPDs, determining that the subject was administered the anticoagulant agent and the second agent that decreases platelet function. In some embodiments, the anticoagulant agent is a first anticoagulant agent and the second agent is a second anticoagulant agent. In some embodiments, the first anticoagulant agent and the second anti-platelet agent are each different anticoagulant agents selected from dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, and a combination thereof. In some embodiments, the first anticoagulant agent and the second anti-platelet agent have different mechanisms of action. In some embodiments, the first anticoagulant agent and the second anti-platelet agent are each different anticoagulant agents selected from dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, and a combination thereof.

In some embodiments of any of the aspects herein, before, immediately before, at the moment before, at the moment of, and/or at an initial time of, the administering of the composition comprising platelet derivatives, for example FDPDs, the subject was or is at an increased risk of bleeding due to being administered or having been administered the anti-platelet agent, or the subject is bleeding. Furthermore, the subject can be at an increased risk of bleeding at 7, 6, 5, 4, 3, 2, or 1 day, 12 hours, 8 hours, 4 hours, 2 hours, 1 hour or 45, 30, 15, 10, 5, 4, 3, 2, or 1 minute before the administering of the composition comprising the platelet derivatives. In some optional embodiments, this is confirmed by laboratory testing. However, in some embodiments no laboratory testing of bleeding risk or any clotting parameter is performed 7, 6, 5, 4, 3, 2, or 1 day or sooner before and/or after the administering of the composition comprising the platelet derivatives. Bleeding risk is typically decreased after administration of an effective dose of the composition comprising platelet derivatives, in illustrative embodiments FDPDs. Furthermore, the subject may remain at an increased risk of bleeding even after the administering of the composition comprising platelet derivatives (e.g. FDPDs), for example for 1, 2, 3, 4, 5, 10, 15, 20, 30, or 45 minutes, or 1, 2, 3, 4, 5, or 8 hours, or longer after the administering, depending on how long it takes for the FDPDs to decrease the risk in the subject after they are administered. Furthermore, in some embodiments, the administration of the composition comprising the platelet derivatives (e.g. FDPDs) decreases but does not completely resolve the increased risk of bleeding in the subject.

In some embodiments, for example of aspects wherein a subject was administered the anticoagulant agent and the second agent that decreases platelet function, administration of the second agent is stopped, for example before administrating the composition comprising the platelet derivatives. In other embodiments of such aspects, administration of the second agent is continued, for example after administering the composition comprising the platelet derivatives.

In certain embodiments of any of the aspects provided herein, the method further comprises before administering the composition comprising platelet derivatives, determining in a pre-administering evaluation, that the subject has an abnormal value for one or more clotting parameters. The pre-administration evaluation, in illustrative embodiments, is an in vitro laboratory test.

In certain embodiments of any of the aspects provided herein, the anticoagulant agent is selected from dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, and a combination thereof. In other embodiments, the anticoagulant agent is selected from dabigatran, argatroban, hirudin, rivaroxaban, apixaban, edoxaban, fondaparinux, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, and a combination thereof.

In certain embodiments of any of the aspects provided herein, the FDPDs comprise (detectable amounts of) a biomolecule (e.g. receptor) targeted by the anti-platelet reversal agent that was administered or is being administered to the subject. In some embodiments the receptor is selected from a P2Y receptor (e.g., the P2Y12 receptor), glycoprotein IIb (i.e. CD41), glycoprotein IIIa (CD61), the glycoprotein IIb/IIIa complex, thromboxane synthase or thromboxane receptors, PAR1, PAR4, VPVI, or collagen receptor (e.g. alpha2beta1 collagen receptor). Provided in other sections herein are examples of anti-platelet agents that target these specific biomolecules. In certain embodiments, at least 55%, 60%, 70%, 75%, 80%, 85%, 90%, or 95% of the platelet derivatives, in illustrative embodiments FDPDs, are positive for (i.e. have detectable levels of) a biomolecule targeted by the anti-platelet agent administered to the subject and/or detectable in the blood of the subject. As noteworthy non-limiting examples, the anti-platelet agent inhibits the glycoprotein CDIIb/IIIa complex, and at least 55%, 60%, 70%, 75%, 80%, 85%, 90%, or 95% of the platelet derivatives, in illustrative embodiments FDPDs, are CD41 positive (i.e. comprise detectable CD41) and/or are positive for the CDIIb/IIIa complex.

In certain embodiments of any of the aspects provided herein, the composition comprising FDPDs comprises a population of FDPDs having a reduced propensity to aggregate such that no more than 2%, 3%, 4%, 5%, 7.5%, 10%, 12.5%, 15%, 17.5%, 20%, 22.5%, or 25% of the FDPDs in the population aggregate under aggregation conditions comprising an agonist but no platelets. In certain embodiments of any of the aspects provided herein, including for example, embodiments where the composition comprises FDPDs comprises a population of FDPDs having a reduced propensity to aggregate such that no more than 10% of the FDPDs in the population aggregate under aggregation conditions comprising an agonist but no platelets, the FDPDs have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., FDPDs) can have a potency of between 1.2 and 2.5 TPGU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TPGU per 10⁶ particles).

In certain embodiments of any of the aspects provided herein, including for example, embodiments where the composition comprises FDPDs comprises a population of FDPDs having a reduced propensity to aggregate such that no more than 10% of the FDPDs in the population aggregate under aggregation conditions comprising an agonist but no platelets, the FDPDs have having one or more characteristics of a super-activated platelet selected from
A) the presence of thrombospondin (TSP) on their surface at a level that is greater than on the surface of resting platelets;
B) the presence of von Willebrand factor (vWF) on their surface at a level that is greater than on the surface of resting platelets; and
C) an inability to increase expression of a platelet activation marker in the presence of an agonist as compared to the expression of the platelet activation marker in the absence of an agonist.

In certain embodiments of any of the aspects provided herein wherein the composition comprising FDPDs comprises a population of FDPDs comprising CD 41-positive FDPDs, including non-limiting embodiments where the population comprises FDPDs have a reduced propensity to aggregate such that no more than 10% of the FDPDs in the population aggregate under aggregation conditions comprising an agonist but no platelets, the FDPDs have an inability to increase expression of a platelet activation marker in the presence of an agonist as compared to the expression of the platelet activation marker in the absence of the agonist. In some embodiments of such methods, the FDPDs have a potency of at least 1.5 thrombin generation potency units (TGPU) per 10⁶ platelet derivatives. In some embodiments of such methods, less than 5% of the CD 41-positive FDPDs are microparticles having a diameter of less than 0.5 μm.

In certain embodiments of any of the aspects provided herein, including non-limiting embodiments where the population comprises FDPDs have a reduced propensity to aggregate such that no more than 10% of the FDPDs in the population aggregate under aggregation conditions comprising an agonist but no platelets, the FDPDs further have one or both of: the presence of thrombospondin (TSP) on their surface at a level that is greater than on the surface of resting platelets; and the presence of von Willebrand factor (vWF) on their surface at a level that is greater than on the surface of resting platelets.

In certain embodiments of any of the aspects provided herein, the composition comprising FDPDs comprises a population of FDPDs comprising
a population of platelet derivatives comprising CD 41-FDPDs, wherein less than 5% of the CD 41-positive FDPDs are microparticles having a diameter of less than 0.5 μm, and comprising platelet derivatives having one or more, or in illustrative embodiments all of the following characteristics:
a reduced propensity to aggregate such that no more than 10% of the platelet derivatives in the population aggregate under aggregation conditions comprising an agonist but no platelets;
an inability to increase expression of a platelet activation marker in the presence of an agonist as compared to the expression of the platelet activation marker in the absence of the agonist;
the presence of thrombospondin (TSP) on their surface at a level that is greater than on the surface of resting platelets;
the presence of von Willebrand factor (vWF) on their surface at a level that is greater than on the surface of resting platelets; and
a potency of at least 1.5 thrombin generation potency units (TGPU) per 10⁶ platelet derivatives.

In some embodiments of any aspects herein, the platelet derivatives, and in illustrative embodiments FDPDs, are surrounded by a compromised plasma membrane. In such embodiments, the platelet derivatives lack an integrated membrane around them. In such embodiments, the platelet derivatives are not surrounded by an integrated membrane. Instead, the membrane comprises pores that are larger than pores observed on living cells. Thus, such platelet derivatives have a reduced ability to, or are unable to transduce signals from the external environment into a response inside the particle that are typically transduced in living platelets. Furthermore, such platelet derivatives (e.g. FDPDs) are not believed to be capable of mitochondrial activation or glycolysis.

In some embodiments of any aspects herein, the effective amount of the composition comprising FDPDs is between 1.0×10⁷ to 1.0×10¹¹ particles or FDPDs/kg of the subject. In some embodiments of any of the aspects herein, the effective amount of the composition comprising FDPDs is between 1.6×10⁷ to 5.1×10⁹ particles or FDPDs/kg of the subject. In some embodiments of any of the aspects herein, which can be combined with either of the above embodiments with ranges of particles or FDPDs/kg, the effective amount of the composition comprising FDPDs is an amount that has a potency between 250 and 5000 TGPU per kg of the subject. Further examples of effective amounts are provided in a different section herein.

In one aspect, provided herein is a method of treating a coagulopathy in a subject, or of restoring hemostasis in a subject, reducing bleeding, or of reducing bleeding potential of a subject that is being administered, or has been administered, an anticoagulant agent, and an antiplatelet agent, the method comprising: administering to the subject in need thereof an effective amount of a composition comprising platelet derivatives, thereby treating the coagulopathy, wherein the composition comprising FDPDs has the property that it is capable of reducing the bleeding, reducing the bleeding potential of the subject, independent of whether a post-administering evaluation of bleeding potential, if performed, would yield a normal or abnormal result.

In any of the aspects herein, in some embodiments, the composition comprising FDPDs has the property that it is capable of reducing the bleeding potential of the subject, or bleeding in the subject, independent of whether a post-administering evaluation of bleeding potential, if performed, would yield a normal or abnormal result. In some optional embodiments, which in some embodiments is performed and in some embodiments is not performed, such post-administering evaluation comprises an in vitro laboratory test performed on a sample taken or drawn in a time period after administering the composition comprising FDPDs to the subject. In other embodiments of any of the aspects herein, wherein the composition comprising FDPDs has the property that it is capable of reducing the bleeding or the bleeding potential of a subject having an increased bleeding or bleeding potential, and in some embodiments an abnormal value for one or more clotting parameters in an in vitro laboratory test, such that normal hemostasis is restored in the subject, independent of whether a post-administering evaluation of bleeding potential, if performed would yield a normal or abnormal result. In some embodiments, such post-administering evaluation comprises an in vitro laboratory test performed on a sample taken or drawn in a time period after administering the composition comprising FDPDs to the subject. The time period, can be for example, within 0 minutes and 72 hours, or between 10 minutes and 72 hours, or between 10 minutes and 48 hours, or between 10 minutes 24 hours, or between 10 minutes and 4 hours, or between 10 minutes and 1 hour, or between 10 minutes and 30 minutes, or between 30 minutes and 24 hours, or between 30 minutes and 4 hours, or between 30 minutes and 1 hour after administering the composition comprising the platelet derivatives (e.g. FDPDs) to the subject. For example, the time period in certain embodiments is between 1 and 4 hours after administering the composition comprising the platelet derivatives (e.g. FDPDs). In some embodiments of any of the aspects herein, a pre or post administration of the composition comprising platelet derivatives is not performed, for example during the recited time periods above.

In any of the aspects herein, in some embodiments the composition comprising platelet derivatives (e.g. FDPDs) has the property that it is capable of reducing the bleeding potential of a subject having an increased or elevated bleeding potential, or reducing bleeding in the subject. In some embodiments, such increased or elevated bleeding potential can be determined by abnormal value for one or more clotting parameters in an in vitro laboratory test performed on a sample taken within 0 minutes and 72 hours, or between 10 minutes and 72 hours, or between 10 minutes and 48 hours, or between 10 minutes 24 hours, or between 10 minutes and 4 hours, or between 10 minutes and 1 hour, or between 10 minutes and 30 minutes, or between 30 minutes and 24 hours, or between 30 minutes and 4 hours, or between 30 minutes and 1 hour before administering the composition comprising the platelet derivatives (e.g. FDPDs). Furthermore, the composition comprising FDPDs typically has the additional and surprising property, that after being administered to the subject in an effective amount, for example for reducing the bleeding potential of the subject, the subject has an abnormal value for the one or more in vitro lab tests, for example of one or more clotting parameters in a post-administering evaluation performed using an, or the in vitro laboratory test performed on a blood sample taken between 15 minutes and 4 hours, 30 minutes and 4 hours, 1 hour and 4 hours, or taken between 15 minutes and 2 hours, 30 minutes and 2 hours, or 1 hour and 2 hours, or taken between 15 minutes and 1 hour or 30 minutes and 1 hour, after administering the composition comprising FDPDs. In some subembodiments of this embodiment, the composition comprising FDPDs has the property that it is capable of reducing the bleeding potential of a subject to about or at a normal hemostasis or about or at the hemostasis level of the subject when not taking the anticoagulant agent. Yet, in these embodiments, the composition comprising FDPDs retains the additional and surprising property, that after being administered to the subject in the effective amount, such a property is independent of a post-administering lab test for bleeding potential. Thus, in some embodiments, the subject would have an abnormal value for the one or more clotting parameters in a post-administering evaluation performed using an, or the in vitro laboratory test performed on a blood sample taken between 1 and 4 hours, or any of the time ranges recited immediately above, after administering the composition comprising FDPDs. It will be understood that in methods that include compositions comprising FDPDs with such properties, or any properties that include an evaluation or test, no testing actually needs to be performed to practice such methods unless such testing step is actually recited as a method step.

In any of the aspects herein, in illustrative embodiments the composition comprising platelet derivatives or FDPDs further comprises additional components, such as components that were present when such platelet derivatives were dried, or FDPDs were freeze-dried. Such additional components can include components of an incubating agent comprising one or more salts, a buffer, and in certain embodiments a cryoprotectant (also called a lyophilizing agent) and/or an organic solvent. For example, such compositions can comprise one or more saccharides, as provided further herein, which in illustrative embodiments include trehalose and in further illustrative embodiments include polysucrose.

In any of the aspects herein, in some embodiments the FDPDs are prepared using centrifugation. In some illustrative embodiments, the FDPDs are prepared using TFF, in further illustrative embodiments without isolating platelets by centrifugation during the process.

In some embodiments of any of the aspects herein, the method further includes determining the value of one or more clotting parameters in a post-administering evaluation, wherein the post-administering evaluation is performed following the administering. In some embodiments the post-administering evaluation of the one or more clotting parameters shows a normal value for at least one of the one or more clotting parameters. In further embodiments the method the result of the post-administering evaluation of the one or more clotting parameters is improved from the result of the evaluation of the one or more parameters prior to the administering.

In further embodiments of the method the administering of the anticoagulant agent contrary to medical instruction is self-administering by the subject, is administered by another, or is administering by a medical professional.

In some embodiments of any of the aspects or embodiments herein that include a second agent, typically a second agent that decreases platelet function, the second agent is selected from the group consisting of an antihypertensive, a proton pump inhibitor, and a combination thereof. In some embodiments the second agent is selected from the group consisting of a chemotherapeutic agent, an antibiotic, a cardiovascular agent, a H2 antagonist, a neuropsychiatric agent and a combination thereof. In some embodiments the second agent comprises an antidepressant. In further embodiments the antidepressant is selected from the group consisting of a selective serotonin reuptake inhibitor (SSRI), a serotonin antagonist and reuptake inhibitor (SARI), a serotonin and norepinephrine reuptake inhibitor (SNRI), and a combination thereof. In some embodiments the second agent is not an anticoagulant and in some embodiments, the second agent is not an antiplatelet agent.

In any of the aspects herein, in some embodiments, administration of the anticoagulant agent is stopped before or when a composition comprising platelet derivatives (e.g. FDPDs) is administer to a subject. In some aspects of the method, administration of the anticoagulant agent is continued after a composition comprising platelet derivatives (e.g. FDPDs) is administer to a subject.

In any of the aspects herein, in some embodiments further comprise determining that the subject has an abnormal value for one or more clotting parameters in a pre-administering evaluation. In some aspects of the method, the method comprises determining the value of one or more clotting parameters in a post-administering evaluation. In some embodiments the post-administering evaluation of the one or more clotting parameters shows a normal result for at least one of the one or more clotting parameters. In some embodiments the result of the post-administering evaluation of the one or more clotting parameters is improved from the result of the evaluation of the one or more parameters prior to the administering.

In any of the aspects herein, in some embodiments, the subject is identified as having an abnormal result for one or more pre-administering evaluations of clotting parameters during surgery. In some embodiments the surgery is an emergency surgery. In some embodiments the surgery is a scheduled surgery.

In any of the aspects herein, in some embodiments, the clotting parameters includes an evaluation of bleeding. In some embodiments the evaluation of bleeding is performed based on the World Health Organization (WHO) bleeding scale. In some embodiments of the method, before administering, the subject has bleeding of grade 2, 3, or 4 based on the WHO bleeding scale; In some embodiments of the method, after administering, the subject has bleeding of grade 0 or 1 based on the WHO bleeding scale. In some embodiments, after the administering, the subject has bleeding of one grade less, based on the WHO bleeding scale, than before the administering. In some embodiments, after the administering, the subject has bleeding of two grades less, based on the WHO bleeding scale, than before the administering. In some embodiments, after the administering, the subject has bleeding of three grades less, based on the WHO bleeding scale, than before the administering.

In any of the aspects herein, in some embodiments, evaluation of the clotting parameters includes an evaluation of prothrombin time (PT). In some embodiments, abnormal results for PT comprises a PT of greater than about 14 seconds. In some embodiments, after the administering, the subject has a decrease in PT of at least 1, 2, 3, 4, 5, or more, seconds. In some embodiments, after the administering, the subject has a normal PT.

In any of the aspects herein, in some embodiments, the one or more clotting parameters includes an evaluation of activated partial thromboplastin time (aPTT). In some embodiments, the abnormal result for aPTT comprises an aPTT of greater than about 40 seconds. In some embodiments, after the administering, the subject has a decrease in aPTT of at least 5, 10, 15, 20, or more, seconds. In some embodiments, after the administering, the subject has a normal aPTT.

In any of the aspects herein, in some embodiments, the one or more clotting parameters includes an evaluation of thrombin clot time (TCT). In some embodiments, the abnormal result for TCT comprises a TCT of greater than about 35 seconds. In some embodiments, after the administering, the subject has a decrease in TCT of at least 5, 10, 15, 20, or more, seconds. In some embodiments, after the administering, the subject has a normal TCT.

In any of the aspects herein, in some embodiments, the evaluation of the one or more clotting parameters is measured using thromboelastography (TEG). In some embodiments, the abnormal result for TEG comprises a maximum amplitude (MA) of less than about 50 mm. In some embodiments, after the administering, the subject has an increase in MA of at least 5, 10, 15, 20, or more, mm. In some embodiments, after the administering, the subject has a normal MA.

In any of the aspects herein, in some embodiments, the abnormal result for TEG comprises a percent aggregation (in the presence of 1 mmol/L arachidonic acid) of less than about 85%. In some embodiments, after the administering, the subject has an increase in percent aggregation (in the presence of 1 mmol/L arachidonic acid) of at least 2, 3, 5, 8, 10, 12, or more, percentage points. In some embodiments, after the administering, the subject has a normal percent aggregation (in the presence of 1 mmol/L arachidonic acid). In some embodiments, the TEG is used to evaluate adenosine diphosphate-induced platelet-fibrin clot strength. In some aspects of the method, the TEG is used to evaluate arachidonic acid-induced platelet-fibrin clot strength.

In any of the aspects herein, in some embodiments, the one or more clotting parameters is measured using multiple electrode aggregometry (MEA). In some embodiments, the abnormal result using MEA comprises an abnormal result for ADP-induced platelet activity. In some embodiments, the abnormal result for MEA comprises a result of less than about 50 units (U) for ADP-induced platelet activity. In some embodiments, after the administering, the subject has an increase in ADP-induced platelet activity by 5, 10, 15, 20, or more units. In some embodiments, after the administering, the subject has a normal value for ADP-induced platelet activity. In some embodiments, the abnormal result for MEA comprises an abnormal result for arachidonic acid-induced platelet activity. In some embodiments, the abnormal result for MEA comprises a result of less than about 70 units (U) for arachidonic acid-induced platelet activity. In some embodiments, after the administering, the subject has an increase in arachidonic acid-induced platelet activity by 5, 10, 15, 20, or more units. In some embodiments, after the administering, the subject has a normal value for arachidonic acid-induced platelet activity.

In any of the aspects herein, in some embodiments, the one or more clotting parameters is measured using light transmission aggregometry (LTA).

In any of the aspects herein, in some embodiments, the abnormal result for LTA comprises one or more of the following: (a) in the presence of 5 μmol/L adenosine diphosphate, a percent aggregation of less than about 60%; (b) in the presence of 2 μg/mL collagen, a percent aggregation of less than about 65%; (c) in the presence of 1 mmol/L arachidonic acid, a percent aggregation of less than about 65%; (d) in the presence of 2 mmol/L arachidonic acid, a percent aggregation of less than about 69%; or (e) in the presence of 5 mmol/L arachidonic acid, a percent aggregation of less than about 73%.

In any of the aspects herein, in some embodiments, after the administering, the subject has an increase in percent aggregation (in the presence of 5 μmol/L adenosine diphosphate) of at least 2, 3, 5, 8, 10, 12, or more, percentage points. In some embodiments, after the administering, the subject has a normal percent aggregation (in the presence of 5 μmol/L adenosine diphosphate).

In any of the aspects herein, in some embodiments, after the administering, the subject has an increase in percent aggregation (in the presence of 2 μg/mL collagen) of at least 2, 3, 5, 8, 10, 12, or more, percentage points. In some embodiments, after the administering, the subject has a normal percent aggregation (in the presence of 2 μg/mL collagen).

In any of the aspects herein, in some embodiments, the administering, the subject has an increase in percent aggregation (in the presence of 1 mmol/L arachidonic acid) of at least 2, 3, 5, 8, 10, 12, or more, percentage points. In some embodiments, after the administering, the subject has a normal percent aggregation (in the presence of 1 mmol/L arachidonic acid). In some embodiments, after the administering, the subject has an increase in percent aggregation (in the presence of 2 mmol/L arachidonic acid) of at least 2, 3, 5, 8, 10, 12, or more, percentage points. In some embodiments, after the administering, the subject has a normal percent aggregation (in the presence of 2 mmol/L arachidonic acid). In some embodiments, after the administering, the subject has an increase in percent aggregation (in the presence of 5 mmol/L arachidonic acid) of at least 2, 3, 5, 8, 10, 12, or more, percentage points. In some embodiments, after the administering, the subject has a normal percent aggregation (in the presence of 5 mmol/L arachidonic acid).

In any of the aspects herein, in some embodiments, the method further comprises administering to the subject an additional anticoagulant agent reversal agent. In some embodiments, the administering of the composition occurs concurrently with administering of the additional anticoagulant agent reversal agent. In some embodiments, the administering of the composition occurs after administering of the additional anticoagulant agent reversal agent. In some embodiments, the administering of the composition occurs before administering of the additional anticoagulant agent reversal agent.

In some embodiments of any aspects herein, the anti-coagulant reversal agent is selected from the group consisting of protamine, protamine sulfate, and a combination thereof. In some embodiments, of any of the aspects herein the anti-coagulant reversal agent is selected from the group consisting of protamine, protamine sulfate, vitamin K, prothrombin complex concentrate (PCC), idarucizumab, Andexanet Alfa, and combinations thereof.

In any of the aspects herein, in some embodiments, the composition further comprises an anti-fibrinolytic agent. In some embodiments, the anti-fibrinolytic agent is selected from the group consisting of ε-aminocaproic acid (EACA), tranexamic acid, aprotinin, aminomethylbenzoic acid, fibrinogen, and a combination thereof. In some embodiments, the platelets or platelet derivatives are loaded with the anti-fibrinolytic agent.

In any of the aspects herein, in some embodiments, administering comprises administering topically, parenterally, intravenously, intramuscularly, intrathecally, subcutaneously, intraperitoneally, or a combination thereof.

In any of the aspects herein, in some embodiments, the composition is dried prior to the administration step. In some embodiments, the composition is rehydrated following the drying step.

In any of the aspects herein, in some embodiments, the composition is freeze-dried prior to the administration step. In some embodiments, the composition is rehydrated following the freeze-drying step.

In any of the aspects herein, in some embodiments, the incubating agent comprises one or more salts selected from phosphate salts, sodium salts, potassium salts, calcium salts, magnesium salts, and a combination of two or more thereof. In some embodiments, the incubating agent comprises a carrier protein. In some embodiments the incubating agent comprises a buffer that comprises HEPES, sodium bicarbonate (NaHCO₃), or a combination thereof.

In any of the aspects herein, in some embodiments, the composition comprises one or more saccharides. In some embodiments, the one or more saccharides comprise trehalose. In some embodiments, the one or more saccharides comprise polysucrose. In some embodiments, the one or more saccharides comprise dextrose.

In some aspects of the method, the composition comprises an organic solvent.

In some embodiments of any of the aspects herein, the anticoagulant agent, or the antiplatelet agent is present in the subject at the time the composition comprising the FDPDs is administered at a level that increases the bleeding potential and increases the risk of bleeding of the subject, increases the bleeding in the subject, or leads to the bleeding in the subject that is at a level considered as life-threatening. In some embodiments, the anticoagulant agent is present at a Cmax within 2, 4, 5, 7, 8, 10, 12, 15, 30 or 45 minutes, or within 1, 2, 3, 4, 6, or 8 hours of the time the composition comprising the FDPDs is administered or the time the first or last dose of the composition comprising the FDPDs is administered.

In any of the aspects herein, in some embodiments, some aspects of the method, the subject does not have cancer.

Any of the method aspects herein, can be uses for a composition comprising platelet derivatives (e.g. FDPDs) provided herein, or uses for a kit comprising such composition, as set out in the following aspects that include such “use” language. It will be understood that where such aspects refer to FDPDs, they could refer to platelet derivatives instead. It will be further understood that such aspects can include any of the elements provided herein for method aspects, and any of the embodiments provided herein. For example, administering of an effective amount of composition comprising platelet derivatives (e.g. FDPDs) can be such that the bleeding or the bleeding potential of the subject is reduced, and in illustrative embodiments normal hemostasis is restored, or the bleeding is reduced such that it is not considered life-threatening, or the bleeding is stopped. Where such aspects refer to a disorder, the disorder in illustrative embodiments, is a bleeding disorder. Such disorder can be identified, for example, because a sample from a subject having such disorder yields an abnormal value for one or more clotting parameters.

Provided herein in some embodiments is a method of treating a coagulopathy in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of treating a coagulopathy in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, provided herein is a method of restoring normal hemostasis in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, provided herein is a method of preparing a subject for surgery, the method including administering to the subject in need thereof an effective amount of a composition including platelets or platelet derivatives and an incubating agent including one or more salts, a buffer, optionally a cryoprotectant, and optionally an organic solvent. Implementations can include one or more of the following features. The surgery can be an emergency surgery. The surgery can be a scheduled surgery.

In some embodiments, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, to form the composition. Implementations can include one or more of the following features. The surgery can be an emergency surgery. The surgery can be a scheduled surgery.

In some embodiments of the above methods, the subject has been treated or is being treated with an anticoagulant agent. In some embodiments, treatment with the anticoagulant agent can be stopped. In some embodiments, treatment with the anticoagulant agent can be continued.

In some embodiments, provided herein is a method of ameliorating the effects of an anticoagulant agent in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of ameliorating the effects of an anticoagulant agent in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, the effects of the anticoagulant agent can be the result of an overdose of the anticoagulant agent.

Some embodiments of any of the methods herein can include one or more of the following features. Administering can include administering topically. Administering can include administering parenterally. Administering can include administering intravenously. Administering can include administering intramuscularly. Administering can include administering intrathecally. Administering can include administering subcutaneously. Administering can include administering intraperitoneally. The composition can be dried prior to the administration step. The composition can be rehydrated following the drying step. The composition can be freeze-dried prior to the administration step. The composition can be rehydrated following the freeze-drying step. The incubating agent can include one or more salts selected from sodium salts, potassium salts, calcium salts, magnesium salts, and a combination of two or more thereof. The incubating agent can include a carrier protein. The buffer can include HEPES, sodium bicarbonate (NaHCO₃), or a combination thereof. The composition can include one or more saccharides. The one or more saccharides can include trehalose. The one or more saccharides can include polysucrose. The one or more saccharides can include dextrose. The composition can include an organic solvent. The platelets or platelet derivatives can include FDPDs. In some embodiments of any aspects herein, before the administering the subject had an INR of at least 3.0 or 4.0. In other embodiments of any of the aspects herein, after the administering, the subject has an INR of 3.0 or less, 2.0 or less, less than 3.0, or less than 2.0.

Further non-limiting example embodiments are provided in numbered form as follows:

Embodiment 1 is a method of treating a coagulopathy in a subject, the method comprising administering to the subject in need thereof an effective amount of a composition comprising platelets or platelet derivatives and an incubating agent comprising one or more salts, a buffer, optionally a cryoprotectant, and optionally an organic solvent. In some embodiments, before such administering step, the method includes an additional step of rehydrating a powder composition to form the composition, typically a liquid composition, that is administered to the subject. In illustrative embodiments, the coagulopathy includes and/or is bleeding of the subject. In some embodiments, the bleeding is because the subject has been treated or is being treated with an anticoagulant agent, and/or an antiplatelet agent. In illustrative embodiments, the anticoagulant agent comprises a Factor XI inhibitor. In some embodiments, the subject has been treated or is being treated with an anticoagulant agent, and an antiplatelet agent, in illustrative embodiments, the antiplatelet agent includes an aspirin, and in further illustrative embodiments, the antiplatelet agent includes aspirin, and one other antiplatelet agent other than aspirin. In some embodiments, the bleeding in the subject is reduced (e.g., WHO score is reduced by at least 1, 2 or 3 grades) or stopped in at least 1, 2, many, most, virtually all, or all sites of bleeding on the subject, after, in illustrative embodiments within 4 hours, 2 hours, 1 hour, 45 minutes, 30 minutes, 15 minutes, 10 minutes or 5 minutes after the administering of the platelet derivatives.

Embodiment 2 is a method of treating a coagulopathy in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 3 is a method of restoring normal hemostasis in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 4 is a method of restoring normal hemostasis in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 5 is a method of preparing a subject for surgery, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 6 is a method of preparing a subject for surgery, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 7 is the method of any one of embodiments 5-6, wherein the surgery is an emergency surgery.

Embodiment 8 is the method of any one of embodiments 5-6, wherein the surgery is a scheduled surgery.

Embodiment 9 is the method of any one of embodiments 1-8, wherein the subject has been treated or is being treated with an anticoagulant.

Embodiment 10 is the method of embodiment 9, wherein treatment with the anticoagulant is stopped.

Embodiment 11 is the method of embodiment 9, wherein treatment with the anticoagulant is continued.

Embodiment 12 is a method of ameliorating the effects of an anticoagulant in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 13 is a method of ameliorating the effects of an anticoagulant in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 14 is the method of embodiment 12 or embodiment 13, wherein the effects of the anticoagulant are the result of an overdose of the anticoagulant.

Embodiment 15 is the method of any one of embodiments 1-14, wherein the composition further comprises an anti-fibrinolytic agent.

Embodiment 16 is the method of embodiment 15, wherein the anti-fibrinolytic agent is selected from the group consisting of 8-aminocaproic acid (EACA), tranexamic acid, aprotinin, aminomethylbenzoic acid, fibrinogen, and a combination thereof.

Embodiment 17 is the method of embodiment 15 or embodiment 16, wherein the platelets or platelet derivatives are loaded with the anti-fibrinolytic agent.

Embodiment 18 is the method of any one of embodiments 9-17, wherein the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof.

Embodiment 19 is the method of any one of embodiments 9-17, wherein the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof.

Embodiment 20 is the method of embodiment 18 or embodiment 19, wherein the anticoagulant is warfarin.

Embodiment 21 is the method of embodiment 18 or embodiment 19, wherein the anticoagulant is heparin.

Embodiment 22 is the method of any one of embodiments 1-21, wherein before the administering, the subject had an INR of at least 4.0.

Embodiment 23 is the method of embodiment 22, wherein after the administering, the subject has an INR of 3.0 or less.

Embodiment 24 is the method of embodiment 22, wherein after the administering, the subject has an INR of 2.0 or less.

Embodiment 25 is the method of any one of embodiments 1-21, wherein before the administering, the subject had an INR of at least 3.0.

Embodiment 26 is the method of embodiment 25, wherein after the administering, the subject has an INR of 2.0 or less.

Embodiment 27 is the method of any one of embodiments 1-26, wherein administering comprises administering topically.

Embodiment 28 is the method of any one of embodiments 1-26, wherein administering comprises administering parenterally.

Embodiment 29 is the method of any one of embodiments 1-26, wherein administering comprises administering intravenously.

Embodiment 30 is the method of any one of embodiments 1-26, wherein administering comprises administering intramuscularly.

Embodiment 31 is the method of any one of embodiments 1-26, wherein administering comprises administering intrathecally.

Embodiment 32 is the method of any one of embodiments 1-26, wherein administering comprises administering subcutaneously.

Embodiment 33 is the method of any one of embodiments 1-26, wherein administering comprises administering intraperitoneally.

Embodiment 34 is the method of any one of embodiments 1-33, wherein the composition is dried prior to the administration step.

Embodiment 35 is the method of embodiment 34, wherein the composition is rehydrated following the drying step.

Embodiment 36 is the method of any one of embodiments 1-34, wherein the composition is freeze-dried prior to the administration step.

Embodiment 37 is the method of embodiment 36, wherein the composition is rehydrated following the freeze-drying step.

Embodiment 38 is the method of any one of embodiments 1-37, wherein the incubating agent comprises one or more salts selected from phosphate salts, sodium salts, potassium salts, calcium salts, magnesium salts, and a combination of two or more thereof.

Embodiment 39 is the method of any one of embodiments 1-38, wherein the incubating agent comprises a carrier protein.

Embodiment 40 is the method of any one of embodiments 1-39, wherein the buffer comprises HEPES, sodium bicarbonate (NaHCO3), or a combination thereof.

Embodiment 41 is the method of any one of embodiments 1-40, wherein the composition comprises one or more saccharides.

Embodiment 42 is the method of embodiment 41, wherein the one or more saccharides comprise trehalose.

Embodiment 43 is the method of embodiment 41 or embodiment 42, wherein the one or more saccharides comprise polysucrose.

Embodiment 44 is the method of any one of embodiments 41-43, wherein the one or more saccharides comprise dextrose.

Embodiment 45 is the method of any one of embodiments 1-44, wherein the composition comprises an organic solvent.

Embodiment 46 is the method of any one of embodiments 1-45, wherein the platelets or platelet derivatives comprise freeze-dried platelet derivatives.

Embodiment 47 is a method of treating a coagulopathy in a subject that is being administered or has been administered an anticoagulant, the method comprising:
(a) determining that the subject has an abnormal result for evaluation of one or more clotting parameters; and
(b) after (a), administering to the subject in need thereof an effective amount of a composition comprising platelets or platelet derivatives and an incubating agent comprising one or more salts, a buffer, optionally a cryoprotectant, and optionally an organic solvent.

Embodiment 48 is a method of treating a coagulopathy in a subject that is being administered or has been administered an anticoagulant, the method comprising:
(a) determining that the subject an abnormal result for evaluation of one or more clotting parameters; and
(b) after (a), administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 49 is a method of treating a coagulopathy in a subject that is being administered or has been administered an anticoagulant, the method comprising:
administering to the subject in need thereof an effective amount of a composition comprising platelets or platelet derivatives and an incubating agent comprising one or more salts, a buffer, optionally a cryoprotectant, and optionally an organic solvent, wherein before the administering, the subject has been determined to have an abnormal result for evaluation of one or more clotting parameters.

Embodiment 50 is a method of treating a coagulopathy in a subject that is being administered or has been administered an anticoagulant, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein before the administering, the subject has been determined to have an abnormal result for evaluation of one or more clotting parameters.

Embodiment 51 is a method of any one of embodiments 47-50, further comprising determining the result of the evaluation one or more clotting parameters following the administering.

Embodiment 52 is the method of embodiment 51, wherein the evaluation of the one or more clotting parameters following the administering shows a normal result for at least one of the one or more clotting parameters.

Embodiment 53 is the method of embodiment 51, wherein the result of the evaluation of the one or more clotting parameters following the administering is improved from the result of the evaluation of the one or more parameters prior to the administering.

Embodiment 54 is a method of treating a coagulopathy in a subject, the method comprising:
(a) determining that the subject, contrary to medical instruction, was administered an anticoagulant; and
(b) administering to the subject in need thereof an effective amount of a composition comprising platelets or platelet derivatives and an incubating agent comprising one or more salts, a buffer, optionally a cryoprotectant, and optionally an organic solvent.

Embodiment 55 is a method of treating a coagulopathy in a subject, the method comprising:
(a) determining that the subject, contrary to medical instruction, was administered an anticoagulant; and
(b) administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 56 is a method of treating a coagulopathy in a subject, the method comprising:
administering to the subject in need thereof an effective amount of a composition comprising platelets or platelet derivatives and an incubating agent comprising one or more salts, a buffer, optionally a cryoprotectant, and optionally an organic solvent, wherein the subject is determined to have been administered an anticoagulant contrary to medical instruction.

Embodiment 57 is a method of treating a coagulopathy in a subject, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein the subject is determined to have been administered an anticoagulant contrary to medical instruction.

Embodiment 58 is a method of restoring normal hemostasis in a subject, the method comprising:
(a) determining that the subject, contrary to medical instruction, was administered an anticoagulant; and
(b) administering to the subject in need thereof an effective amount of a composition comprising platelets or platelet derivatives and an incubating agent comprising one or more salts, a buffer, optionally a cryoprotectant, and optionally an organic solvent.

Embodiment 59 is a method of restoring normal hemostasis in a subject, the method comprising:
(a) determining that the subject, contrary to medical instruction, was administered an anticoagulant; and
(b) administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 60 is a method of restoring normal hemostasis in a subject, the method comprising:
administering to the subject in need thereof an effective amount of a composition comprising platelets or platelet derivatives and an incubating agent comprising one or more salts, a buffer, optionally a cryoprotectant, and optionally an organic solvent, wherein the subject is determined to have been administered an anticoagulant contrary to medical instruction.

Embodiment 61 is a method of restoring normal hemostasis in a subject, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein the subject is determined to have been administered an anticoagulant contrary to medical instruction.

Embodiment 62 is the method of any one of embodiments 54-61, wherein the administering of the anticoagulant contrary to medical instruction is self-administering by the subject.

Embodiment 63 is the method of any one of embodiments 54-61, wherein the administering of the anticoagulant contrary to medical instruction is administering by a medical professional.

Embodiment 64 is the method of any one of embodiments 54-63, wherein the medical instruction is verbal instruction by a medical professional.

Embodiment 65 is the method of any one of embodiments 54-63, wherein the medical instruction is written instruction.

Embodiment 66 is a method of treating a coagulopathy in a subject, the method comprising:
(a) determining that the subject was administered an anticoagulant and a second agent that decreases platelet function; and
(b) administering to the subject in need thereof an effective amount of a composition comprising platelets or platelet derivatives and an incubating agent comprising one or more salts, a buffer, optionally a cryoprotectant, and optionally an organic solvent.

Embodiment 67 is a method of treating a coagulopathy in a subject, the method comprising
(a) determining that the subject was administered an anticoagulant and a second agent that decreases platelet function; and
(b) administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 68 is a method of treating a coagulopathy in a subject, the method comprising:
administering to the subject in need thereof an effective amount of a composition comprising platelets or platelet derivatives and an incubating agent comprising one or more salts, a buffer, optionally a cryoprotectant, and optionally an organic solvent, wherein the subject is determined to have been administered an anticoagulant and a second agent that decreases platelet function.

Embodiment 69 is a method of treating a coagulopathy in a subject, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein the subject is determined to have been administered an anticoagulant and a second agent that decreases platelet function.

Embodiment 70 is a method of restoring normal hemostasis in a subject, the method comprising:
(a) determining that the subject was administered an anticoagulant and a second agent that decreases platelet function; and
(b) administering to the subject in need thereof an effective amount of a composition comprising platelets or platelet derivatives and an incubating agent comprising one or more salts, a buffer, optionally a cryoprotectant, and optionally an organic solvent.

Embodiment 71 is a method of restoring normal hemostasis in a subject, the method comprising:
(a) determining that the subject was administered an anticoagulant and a second agent that decreases platelet function; and
(b) administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 72 is a method of restoring normal hemostasis in a subject, the method comprising:
administering to the subject in need thereof an effective amount of a composition comprising platelets or platelet derivatives and an incubating agent comprising one or more salts, a buffer, optionally a cryoprotectant, and optionally an organic solvent, wherein the subject is identified as having been administered an anticoagulant and a second agent that decreases platelet function.

Embodiment 73 is a method of restoring normal hemostasis in a subject, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein the subject is identified as having been administered an anticoagulant and a second agent that decreases platelet function.

Embodiment 74 is a method of any one of embodiments 66-73, wherein administration of the second agent is stopped.

Embodiment 75 is a method of any one of embodiments 66-73, wherein administration of the second agent is continued.

Embodiment 76 is a method of any one of embodiments 66-75, wherein the second agent is selected from the group consisting of an antihypertensive, a proton pump inhibitor, and a combination thereof.

Embodiment 77 is a method of any one of embodiments 66-75, wherein the second agent is selected from the group consisting of a chemotherapeutic agent, an antibiotic, a cardiovascular agent, a H2 antagonist, a neuropsychiatric agent, and a combination thereof.

Embodiment 78 is a method of any one of embodiments 66-75, wherein the second agent comprises an antidepressant.

Embodiment 79 is a method of embodiment 78, wherein the antidepressant is selected from the group consisting of a selective serotonin reuptake inhibitor (SSRI), a serotonin antagonist and reuptake inhibitor (SARI), a serotonin and norepinephrine reuptake inhibitor (SNRI), and a combination thereof.

Embodiment 80 is a method of any one of embodiments 66-79, wherein the second agent is not an antiplatelet agent.

Embodiment 81 is a method of any one of embodiments 47-80, wherein administration of the anticoagulant is stopped.

Embodiment 82 is a method of any one of embodiments 47-80, wherein administration of the anticoagulant is continued.

Embodiment 83 is a method of preventing or mitigating the potential for a coagulopathy in a subject, the method comprising:
(a) determining that information regarding whether the subject was administered an anticoagulant is unavailable; and
(b) administering to the subject an effective amount of a composition comprising platelets or platelet derivatives and an incubating agent comprising one or more salts, a buffer, optionally a cryoprotectant, and optionally an organic solvent.

Embodiment 84 is a method of preventing or mitigating the potential for a coagulopathy in a subject, the method comprising:
(a) determining that information regarding whether the subject was administered an anticoagulant is unavailable; and
(b) administering to the subject an effective amount of a composition prepared by a process comprising incubating platelets with an incubating agent comprising one or more salts, a buffer, optionally a cryoprotectant, and optionally an organic solvent, to form the composition.

Embodiment 85 is a method of preventing or mitigating the potential for a coagulopathy in a subject, the method comprising:
administering to the subject in need thereof an effective amount of a composition comprising platelets or platelet derivatives and an incubating agent comprising one or more salts, a buffer, optionally a cryoprotectant, and optionally an organic solvent, wherein the subject has been determined to be a subject for which information regarding whether the subject was administered an anticoagulant is unavailable.

Embodiment 86 is a method of preventing or mitigating the potential for a coagulopathy in a subject, the method comprising:
administering to the subject in need thereof an effective amount of a composition prepared by a process comprising incubating platelets with an incubating agent comprising one or more salts, a buffer, optionally a cryoprotectant, and optionally an organic solvent, to form the composition, wherein the subject has been determined to be a subject for which information regarding whether the subject was administered an anticoagulant is unavailable.

Embodiment 87 is the method of any one of embodiments 83-86, wherein information regarding whether the subject was administered an anticoagulant is unavailable for a reason comprising that the subject cannot be identified.

Embodiment 88 is the method of any one of embodiments 83-87, wherein information regarding whether the subject was administered an anticoagulant is unavailable for a reason comprising that the medical history of the subject is unavailable.

Embodiment 89 is the method of any one of embodiments 83-88, wherein information regarding whether the subject was administered an anticoagulant is unavailable for a reason comprising that the subject is in need of emergency treatment.

Embodiment 90 is the method of any one of embodiments 83-89, wherein information regarding whether the subject was administered an anticoagulant is unavailable for a reason comprising that the subject is in need of emergency surgery.

Embodiment 91 is the method of any one of embodiments 83-90, wherein information regarding whether the subject was administered an anticoagulant is unavailable for a reason comprising that the subject has an emergency surgery.

Embodiment 92 is the method of any one of embodiments 54-91, wherein the method further comprises determining that the subject has an abnormal result for one or more evaluations of clotting parameters.

Embodiment 93 is the method of any one of embodiments 54-91, wherein the subject has been determined to have an abnormal result for one or more evaluations of clotting parameters.

Embodiment 94 is the method of any one of embodiments 92-93, wherein the subject was previously identified as having a normal result for at least one of the one or more clotting parameters.

Embodiment 95 is the method of any one of embodiments 92-94, further comprising determining the result of the evaluation one or more clotting parameters following the administering.

Embodiment 96 is the method of embodiment 50, wherein the evaluation of the one or more clotting parameters following the administering shows a normal result for at least one of the one or more clotting parameters.

Embodiment 97 is the method of embodiment 50, wherein the result of the evaluation of the one or more clotting parameters following the administering is improved from the result of the evaluation of the one or more parameters prior to the administering.

Embodiment 98 is the method of any one of embodiments 47-53 or 92-97, wherein the subject is identified as having an abnormal result for one or more evaluations of clotting parameters during surgery.

Embodiment 99 is the method of embodiment 98, wherein the surgery is an emergency surgery.

Embodiment 100 is the method of embodiment 98, wherein the surgery is a scheduled surgery.

Embodiment 101 is the method of any one of embodiments 47-53 or 92-100, wherein evaluation of the one or more clotting parameters includes an evaluation of bleeding.

Embodiment 102 is the method of embodiment 101, wherein the evaluation of bleeding is performed based on the World Health Organization (WHO) bleeding scale.

Embodiment 103 is the method of embodiment 102, wherein before the administering, the subject has bleeding of grade 2, 3, or 4 based on the WHO bleeding scale.

Embodiment 104 is the method of embodiment 103, wherein after the administering, the subject has bleeding of grade 0 or 1 based on the WHO bleeding scale.

Embodiment 105 is the method of embodiment 102, wherein after the administering, the subject has bleeding of one grade less, based on the WHO bleeding scale, than before the administering.

Embodiment 106 is the method of embodiment 102, wherein after the administering, the subject has bleeding of two grades less, based on the WHO bleeding scale, than before the administering.

Embodiment 107 is the method of embodiment 102, wherein after the administering, the subject has bleeding of three grades less, based on the WHO bleeding scale, than before the administering.

Embodiment 108 is the method of any one of embodiments 47-53 or 92-107, wherein the one or more clotting parameters includes an evaluation of prothrombin time (PT).

Embodiment 109 is the method of embodiment 108, wherein the abnormal results for PT comprise a PT of greater than about 14 seconds.

Embodiment 110 is the method of embodiment 102 or embodiment 103, wherein after the administering, the subject has a decrease in PT of at least 1, 2, 3, 4, 5, or more, seconds.

Embodiment 111 is the method of any one of embodiments 108-110, wherein after the administering, the subject has a normal PT.

Embodiment 112 is the method of any one of embodiments 47-53 or 92-111, wherein the one or more clotting parameters includes an evaluation of activated partial thromboplastin time (aPTT).

Embodiment 113 is the method of embodiment 112, wherein the abnormal result for aPTT comprises an aPTT of greater than about 40 seconds.

Embodiment 114 is the method of embodiment 112 or 113, wherein after the administering, the subject has a decrease in aPTT of at least 5, 10, 15, 20, or more, seconds.

Embodiment 115 is the method of embodiment 112-114, wherein after the administering, the subject has a normal aPTT.

Embodiment 116 is the method of any one of embodiments 47-53 or 92-115, wherein the one or more clotting parameters includes an evaluation of thrombin clot time (TCT).

Embodiment 117 is the method of embodiment 116, wherein the abnormal result for TCT comprises a TCT of greater than about 35 seconds.

Embodiment 118 is the method of embodiment 116 or 117, wherein after the administering, the subject has a decrease in TCT of at least 5, 10, 15, 20, or more, seconds.

Embodiment 119 is the method of embodiment 116-118, wherein after the administering, the subject has a normal TCT.

Embodiment 120 is the method of any one of embodiments 47-53 or 92-119, wherein the evaluation of the one or more clotting parameters includes thromboelastography (TEG).

Embodiment 121 is the method of embodiment 120, wherein the abnormal result for TEG comprises a maximum amplitude (MA) of less than about 50 mm.

Embodiment 122 is the method of embodiment 120 or embodiment 121, wherein after the administering, the subject has an increase in MA of at least 5, 10, 15, 20, or more, mm.

Embodiment 123 is the method of any one of embodiments 120-122, wherein after the administering, the subject has a normal MA.

Embodiment 124 is the method of any one of embodiments 120-123, wherein the abnormal result for TEG comprises a percent aggregation (in the presence of 1 mmol/L arachidonic acid) of less than about 85%.

Embodiment 125 is the method of embodiment 124, wherein after the administering, the subject has an increase in percent aggregation (in the presence of 1 mmol/L arachidonic acid) of at least 2, 3, 5, 8, 10, 12, or more, percentage points.

Embodiment 126 is the method of embodiment 124 or embodiment 125, wherein after the administering, the subject has a normal percent aggregation (in the presence of 1 mmol/L arachidonic acid).

Embodiment 127 is the method of any one of embodiments 120-126 wherein the TEG is used to evaluate adenosine diphosphate-induced platelet-fibrin clot strength.

Embodiment 128 is the method of any one of embodiments 120-126, wherein the TEG is used to evaluate arachidonic acid-induced platelet-fibrin clot strength.

Embodiment 129 is the method of any one of embodiments 47-53 or 92-128, wherein the one or more clotting parameters includes multiple electrode aggregometry (MEA).

Embodiment 130 is the method of embodiment 129, wherein the abnormal result for MEA comprises an abnormal result for ADP-induced platelet activity.

Embodiment 131 is the method of embodiment 130, wherein the abnormal result for MEA comprises a result of less than about 50 units (U) for ADP-induced platelet activity.

Embodiment 132 is the method of embodiment 130 or embodiment 131, wherein after the administering, the subject has an increase in ADP-induced platelet activity by 5, 10, 15, 20, or more units.

Embodiment 133 is the method of any one of embodiments 129-132, wherein after the administering, the subject has a normal value for ADP-induced platelet activity.

Embodiment 134 is the method of any one of embodiments 129-133, wherein the abnormal result for MEA comprises an abnormal result for arachidonic acid-induced platelet activity.

Embodiment 135 is the method of embodiment 134, wherein the abnormal result for MEA comprises a result of less than about 70 units (U) for arachidonic acid-induced platelet activity.

Embodiment 136 is the method of embodiment 134 or embodiment 135, wherein after the administering, the subject has an increase in arachidonic acid-induced platelet activity by 5, 10, 15, 20, or more units.

Embodiment 137 is the method of any one of embodiments 134-136, wherein after the administering, the subject has a normal value for arachidonic acid-induced platelet activity.

Embodiment 138 is the method of any one of embodiments 47-53 or 92-137, wherein the one or more clotting parameters includes light transmission aggregometry (LTA).

Embodiment 139 is the method of embodiment 138, wherein the abnormal result for LTA comprises one or more of the following:
(a) in the presence of 5 μmol/L adenosine diphosphate, a percent aggregation of less than about 60%;
(b) in the presence of 2 μg/mL collagen, a percent aggregation of less than about 65%;
(c) in the presence of 1 mmol/L arachidonic acid, a percent aggregation of less than about 65%;
(d) in the presence of 2 mmol/L arachidonic acid, a percent aggregation of less than about 69%; or
(e) in the presence of 5 mmol/L arachidonic acid, a percent aggregation of less than about 73%.

Embodiment 140 is the method of embodiment 139, wherein after the administering, the subject has an increase in percent aggregation (in the presence of 5 μmol/L adenosine diphosphate) of at least 2, 3, 5, 8, 10, 12, or more, percentage points.

Embodiment 141 is the method of embodiment 139 or embodiment 140, wherein after the administering, the subject has a normal percent aggregation (in the presence of 5 μmol/L adenosine diphosphate).

Embodiment 142 is the method of embodiment 139, wherein after the administering, the subject has an increase in percent aggregation (in the presence of 2 μg/mL collagen) of at least 2, 3, 5, 8, 10, 12, or more, percentage points.

Embodiment 143 is the method of embodiment 139 or embodiment 142, wherein after the administering, the subject has a normal percent aggregation (in the presence of 2 μg/mL collagen).

Embodiment 144 is the method of embodiment 139, wherein after the administering, the subject has an increase in percent aggregation (in the presence of 1 mmol/L arachidonic acid) of at least 2, 3, 5, 8, 10, 12, or more, percentage points.

Embodiment 145 is the method of embodiment 139 or embodiment 144, wherein after the administering, the subject has a normal percent aggregation (in the presence of 1 mmol/L arachidonic acid).

Embodiment 146 is the method of embodiment 139, wherein after the administering, the subject has an increase in percent aggregation (in the presence of 2 mmol/L arachidonic acid) of at least 2, 3, 5, 8, 10, 12, or more, percentage points.

Embodiment 147 is the method of embodiment 139 or embodiment 146, wherein after the administering, the subject has a normal percent aggregation (in the presence of 2 mmol/L arachidonic acid).

Embodiment 148 is the method of embodiment 139, wherein after the administering, the subject has an increase in percent aggregation (in the presence of 5 mmol/L arachidonic acid) of at least 2, 3, 5, 8, 10, 12, or more, percentage points.

Embodiment 149 is the method of 139 or embodiment 148, wherein after the administering, the subject has a normal percent aggregation (in the presence of 5 mmol/L arachidonic acid).

Embodiment 150 is the method of any one of embodiments 47-149, wherein the method further comprises administering to the subject an additional anticoagulant reversal agent.

Embodiment 151 is the method of embodiment 150, wherein the administering of the composition occurs concurrently with administering of the additional anticoagulant reversal agent.

Embodiment 152 is the method of embodiment 150, wherein the administering of the composition occurs after administering of the additional anticoagulant reversal agent.

Embodiment 153 is the method of embodiment 150, wherein the administering of the composition occurs before administering of the additional anticoagulant reversal agent.

Embodiment 154 is the method of any one of embodiments 150-153, wherein the additional anticoagulant reversal agent comprises protamine, protamine sulfate, or a combination thereof.

Embodiment 155 is the method of any one of embodiments 150-153, wherein the additional anticoagulant reversal agent is selected from the group consisting of protamine, protamine sulfate, vitamin K, prothrombin complex concentrate (PCC), idarucizumab, Andexanet Alfa, and combinations thereof.

Embodiment 156 is the method of any one of embodiments 47-155, wherein the composition further comprises an anti-fibrinolytic agent.

Embodiment 157 is the method of embodiment 156, wherein the anti-fibrinolytic agent is selected from the group consisting of 8-aminocaproic acid (EACA), tranexamic acid, aprotinin, aminomethylbenzoic acid, fibrinogen, and a combination thereof.

Embodiment 158 is the method of embodiment 156 or embodiment 157, wherein the platelets or platelet derivatives are loaded with the anti-fibrinolytic agent.

Embodiment 159 is the method of any one of embodiments 47-158, wherein the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof.

Embodiment 160 is the method of any one of embodiments 47-158, wherein the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof.

Embodiment 161 is the method of embodiment 159 or embodiment 160, wherein the anticoagulant is warfarin.

Embodiment 162 is the method of embodiment 159 or embodiment 160, wherein the anticoagulant is heparin.

Embodiment 163 is the method of any one of embodiments 47-162, wherein before the administering, the subject had an INR of at least 4.0.

Embodiment 164 is the method of embodiment 163, wherein after the administering, the subject has an INR of 3.0 or less.

Embodiment 165 is the method of embodiment 164, wherein after the administering, the subject has an INR of 2.0 or less.

Embodiment 166 is the method of any one of embodiments 47-162, wherein before the administering, the subject had an INR of at least 3.0.

Embodiment 167 is the method of embodiment 166, wherein after the administering, the subject has an INR of 2.0 or less.

Embodiment 168 is the method of any one of embodiments 47-167, wherein administering comprises administering topically, parenterally, intravenously, intramuscularly, intrathecally, subcutaneously, intraperitoneally, or a combination thereof.

Embodiment 169 is the method of any one of embodiments 47-168, wherein the composition is dried prior to the administration step.

Embodiment 170 is the method of embodiment 169, wherein the composition is rehydrated following the drying step.

Embodiment 171 is the method of any one of embodiments 47-170, wherein the composition is freeze-dried prior to the administration step.

Embodiment 172 is the method of embodiment 171, wherein the composition is rehydrated following the freeze-drying step.

Embodiment 173 is the method of any one of embodiments 47-172, wherein the incubating agent comprises one or more salts selected from phosphate salts, sodium salts, potassium salts, calcium salts, magnesium salts, and a combination of two or more thereof.

Embodiment 174 is the method of any one of embodiments 47-173, wherein the incubating agent comprises a carrier protein.

Embodiment 175 is the method of any one of embodiments 47-174, wherein the buffer comprises HEPES, sodium bicarbonate (NaHCO₃), or a combination thereof.

Embodiment 176 is the method of any one of embodiments 47-175, wherein the composition comprises one or more saccharides.

Embodiment 177 is the method of embodiment 176, wherein the one or more saccharides comprise trehalose.

Embodiment 178 is the method of embodiment 176 or embodiment 177, wherein the one or more saccharides comprise polysucrose.

Embodiment 179 is the method of any one of embodiments 176-178, wherein the one or more saccharides comprise dextrose.

Embodiment 180 is the method of any one of embodiments 47-179, wherein the composition comprises an organic solvent.

Embodiment 181 is the method of any one of embodiments 47-180, wherein the platelets or platelet derivatives comprise thrombosomes.

Embodiment 182 is the method of any one of embodiments 47-181, wherein the anticoagulant comprises dabigatran at a dosage of about 100 mg to about 230 mg once or twice daily.

Embodiment 183 is the method of any one of embodiments 47-182, wherein the anticoagulant comprises argatroban at a dosage of about 100 to about 150 mg by injection or infusion.

Embodiment 184 is the method of any one of embodiments 47-183, wherein the anticoagulant comprises a hirudin at dosage of about 0.3 to about 0.5 mg/kg body weight of the subject as an initial bolus dose, or about 0.1 to about 0.2 mg/kg body weight of the subject once a day intravenously following the initial bolus dose.

Embodiment 185 is the method of any one of embodiments 47-184, wherein the anticoagulant comprises rivaroxaban at a dosage of about 2 mg to about 25 mg once or twice daily.

Embodiment 186 is the method of any one of embodiments 47-185, wherein the anticoagulant comprises apixaban at a dosage of about 2 mg to about 10 mg once or twice daily.

Embodiment 187 is the method of any one of embodiments 47-186, wherein the anticoagulant comprises edoxaban at a dosage of about 25 mg to about 70 mg once daily.

Embodiment 188 is the method of any one of embodiments 47-187, wherein the anticoagulant comprises fondaparinux at a dosage of about 2 mg to about 12 mg once daily subcutaneously.

Embodiment 189 is the method of any one of embodiments 47-188, wherein the anticoagulant comprises warfarin at a dosage of about 0.5 mg to about 10 mg once daily.

Embodiment 190 is the method of any one of embodiments 47-189, wherein the anticoagulant comprises heparin at a dosage of about 5,000 units to about 50,000 units over a period of 24 hours.

Embodiment 191 is the method of any one of embodiments 47-190, wherein the anticoagulant comprises a low molecular weight heparin at a dosage of about 30 to about 100 mg once daily.

Embodiment 192 is the method of any one of embodiments 47-191, wherein the anticoagulant comprises apixaban at a dosage of about 2 mg to about 7 mg twice daily.

Embodiment 193 is the method of any one of embodiments 47-192, wherein the anticoagulant comprises betrixaban at an initial dosage of about 150 mg to about 170 mg, or a dosage of about 70 mg to about 90 mg once daily.

Embodiment 194 is the method of any one of embodiments 47-193, wherein the anticoagulant comprises lepirudin at dosage of about 0.3 to about 0.5 mg/kg body weight of the subject as an initial bolus dose, or about 0.1 to about 0.2 mg/kg body weight of the subject once a day intravenously following the initial bolus dose.

Embodiment 195 is the method of any one of embodiments 47-194, wherein the anticoagulant comprises bivalirudin at a dosage of about 0.7 to about 0.8 mg/kg body weight of the subject as an initial bolus dose, or about 1.7 to about 1.8 mg/kg/hr following the initial bolus dose.

Embodiment 196 is the method of any one of embodiments 47-195, wherein the anticoagulant comprises phenprocoumon at an initial dosage of about 10 to about 20 mg, a second dosage of about 5 mg to about 10 mg, or a following dosage of about 1 to about 7 mg per day.

Embodiment 197 is the method of any one of embodiments 47-196, wherein the anticoagulant comprises acenocoumarol at an initial dosage of about 6 to about 14 mg or a following dosage of about 3 to about 10 mg once a day.

Embodiment 198 is the method of any one of embodiments 1-197, wherein the subject does not have cancer.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1

The results that follow demonstrate the impact of the thrombosomes product in an in vitro model for patients taking warfarin, a common anticoagulant drug. Warfarin inhibits the synthesis of numerous hemostatic plasma proteins in the liver that are dependent on vitamin K.

Thrombosomes and other lyophilized platelet products are designed for infusion into a patient's bloodstream following diagnosis of trauma or hemostatic failure. In the following Examples modeling patients using warfarin, thrombosomes were introduced first into a plasma-based system, followed by a whole-blood system in Example 2 to more closely mimic conditions in vivo.

In the plasma model, thrombosomes demonstrated a noticeable improvement in thrombin generation (TGA) and thromboelastography (TEG) assays.

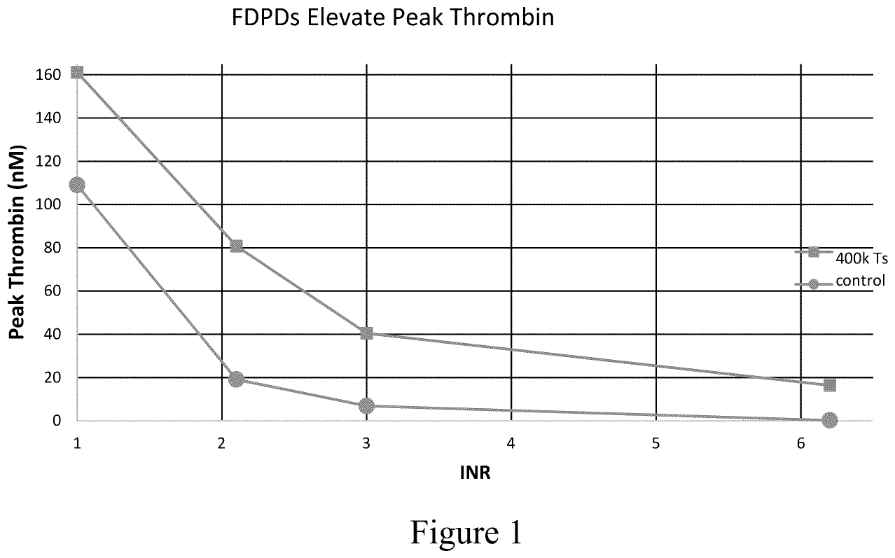

The samples used in the plasma model were prepared by combining 1:1 volumes of warfarin plasma (source: George King Biomedical, at various INR values) or platelet-rich plasma (PRP) and Control Buffer detailed below in Table 6, with or without rehydrated thrombosomes at the concentrations indicated in FIGS. 1-3. Warfarin plasma was obtained from the blood drawn from patients using the drug. Because warfarin inhibits the biological synthesis of hemostatic proteins, it cannot be added ex vivo. Thrombosomes were prepared consistent with the procedures described in U.S. Pat. No. 8,486,617 (such as, e.g., Examples 1-5) and U.S. Pat. No. 8,097,403 (such as, e.g., Examples 1-3), incorporated herein by reference in their entirety and rehydrated by addition of sterile water.

TABLE 6
Composition of Control Buffer

| | Concentration | |
|---|---|---|
| | (mg/mL, | |
| | except where | |
| | otherwise | |
| Component | indicated) | |
| NaCl | 6.08 | |
| KCl | 0.28 | |
| HEPES | 2.47 | |
| NaHCO₃ | 0.77 | |
| Dextrose | 0.41 | |
| Trehalose | 28.83 | |
| Ethanol | 0.76% | (v/v) |
| Polysucrose | 6% | (m/v) |

As INR increases, thrombin generation decreases. Across all doses, thrombosomes demonstrate notable improvement in peak thrombin. As thrombosomes show an uptick at each dose level, it is clear that their efficacy is not related to warfarin.

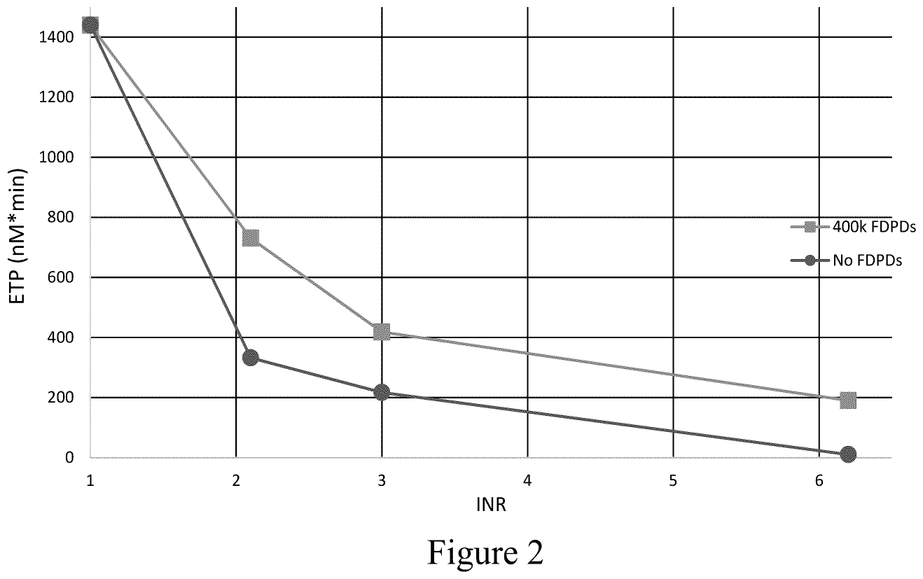

As demonstrated in FIGS. 1 and 2, thrombosomes have a positive impact on thrombin generation (a measure of clotting capability) in a model of warfarin in plasma, assessed in a thrombin generation assay (TGA) as described in Example 3.

Platelet rich plasma sample Preparation
(1) Obtain type O donor whole blood in NaCitrate (blue-top) vacutainer tubes.
(2) Centrifuge the blood at 180×g for 20 minutes.
(3) Carefully pipette off the platelet rich plasma, leaving the buffy coat intact
(4) Take a platelet count in the plasma sample
(5) Supplement appropriate number of platelets per sample to the plasma of choice

In particular, as shown in FIG. 1, peak thrombin generation is improved by adding 400×10³/μL thrombosomes to warfarin plasma. A normal range for peak thrombin was determined to be 66-166 nM, indicating that the uptick in peak thrombin at a normal blood state (INR=1) is not outside reasonable thrombin level. Similarly, FIG. 2 shows that the endogenous thrombin potential (ETP; determined as the area under the curve in the thrombin generation assay) is improved by adding 100×10³/μL thrombosomes to warfarin plasma.

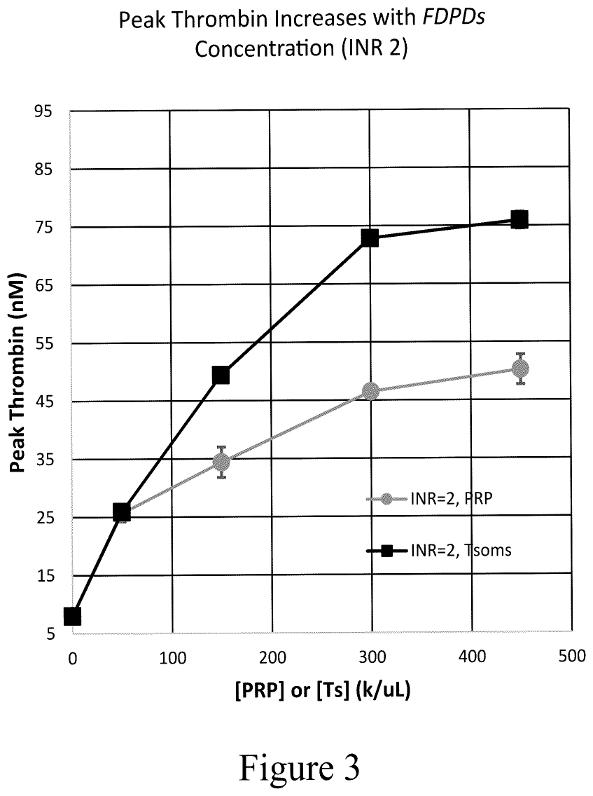

FIG. 3 shows peak thrombin generation by thrombosomes and by platelet-rich plasma (PRP) in INR 2 warfarin plasma. Thrombosomes even generate more thrombin than the platelets, and without being bound by any particular theory or mechanism, this could possibly be due to elevated activation of the thrombosomes. This forecasts a reduction in bleeding in vivo because additional thrombin generation stimulates endogenous clotting mechanisms.

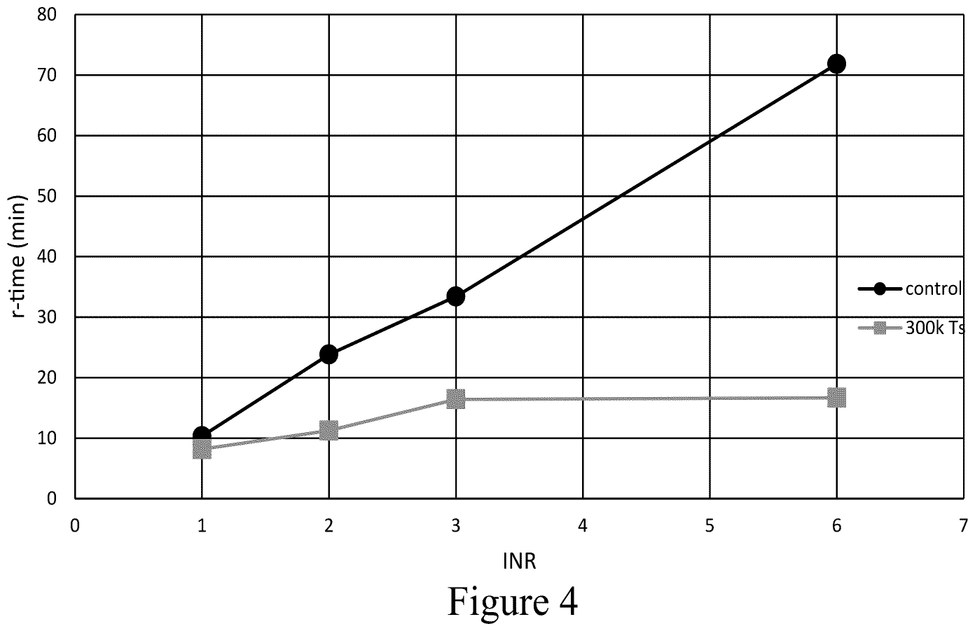

FIG. 4 features data from a thromboelastography (TEG) assay as described in Example 3, a system that measures the viscoelastic properties of blood and plasma. The R-time plotted in FIG. 4 correlates to the speed of clot generation in the plasma model. A reduction in R-time across all warfarin doses was observed with the addition of thrombosomes. In particular, the addition of 300×10³/μL thrombosomes substantially reduced R-time of the warfarin plasma samples (TEG assay). Compared to normal R-time (about 5-10 minutes), the addition of thrombosomes almost completely corrected R-time across all INR levels.

### Example 2: Whole Blood Assays

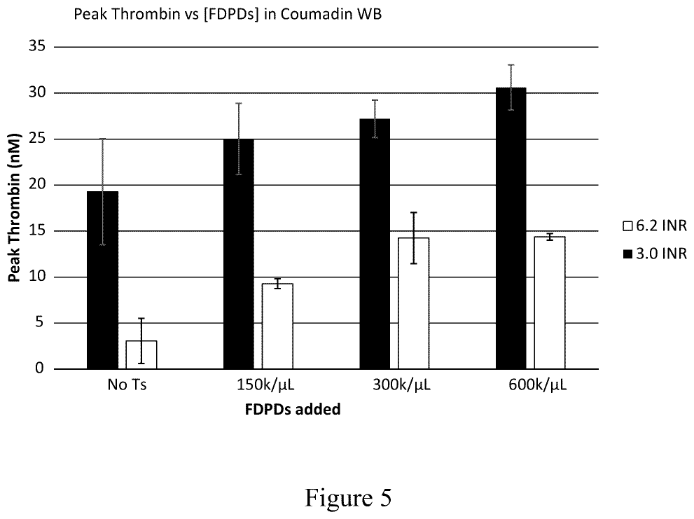

Once the impact in plasma was established, thrombosomes were introduced into a similar warfarin model using donor whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. To generate comparable anticoagulant conditions, the native plasma of type O donor blood was removed and replaced with warfarin plasma as described in Example 3. TGA assays were performed as described in Example 3. FIG. 5 shows that thrombosomes provide a dose-dependent effect on peak thrombin generation. In FIG. 5, data were collected in the background of whole blood with an endogenous platelet count of 150×10³/μL. An increase in peak thrombin was observed in particular at INR 3.0 and 6.2 in FIG. 5. The roughly 50% increase in peak thrombin (at INR 3) in vitro may translate to significantly lower bleeding in vivo as thrombin generation ultimately determines clot stability.

### Example 3: Procedures

### Whole Blood Sample Preparation

(1) Obtain type O donor whole blood in NaCitrate (blue-top) vacutainer tubes.
(2) Centrifuge the blood at 2000×g for 10 minutes.
(3) Carefully pipette off the plasma, leaving the buffy coat intact
(4) Add a volume of HEPES-buffered saline (HBS) equivalent to the removed plasma and gently resuspend the whole blood.
(5) Spin the blood again for 10 minutes at 2000×g.
(6) Carefully remove the supernatant, leaving the buffy coat intact.
(7) Incrementally resuspend the blood in warfarin plasma, normal plasma (function control), or autologous plasma (process control) until the measured hematocrit is equivalent to the hematocrit of the donor's fresh whole blood.
   a. Store at room temperature for up to 4 hours.
(8) Combine 1:1 volume with Control Buffer with or without thrombosomes immediately before running any samples.

Thromboelastography Assay (TEG® 5000 THROMBOELASTOGRAPH® Hemostasis Analyzer System)
(1) Open TEG 5000 assay software and set up instrument according to manufacturer guidelines.
(2) Thaw warfarin plasma in 37° C. water bath for 5 minutes.
(3) Rehydrate thrombosomes with cell culture grade water for 10 minutes then dilute with Control Buffer to 600×10³/μL.
(4) Add 20 μL 0.2M CaCl₂ to empty sample cups.
(5) For each sample, combine 1:1 volumes of Control Buffer with or without thrombosomes and plasma.
(6) Add 340 μL of sample to a cup then quickly load the cup into the device and start the run.
(7) The run is complete when R-time is determined or the run times out.

### Thrombin Generation Assay (on Fluoroskan ASCENT®)

(1) Open CAT software; set up instrument; and prepare PRP reagent (including Tissue Factor and some phospholipids), calibrator, and fluoro-buffer according to manufacturer guidelines.
(2) Thaw warfarin or control plasma in 37° C. water bath for 5 minutes.
(3) Rehydrate thrombosomes with cell culture grade water for 10 minutes then dilute with Control Buffer to double target concentration.
(4) For each sample, combine 1:1 volumes of Control Buffer with or without thrombosomes and plasma.
(5) Using a multichannel pipette, add 20 μL of PRP reagent to each well.
(6) Add 80 μL of sample per well. Include one calibrator well for each sample.
(7) Insert plate into tray and inject fluoro-buffer (including a fluorescent-labeled peptide, that when cleaved by thrombin, generates a fluorescent signal) into active wells.
(8) Read plate for 180 minutes at 20 s intervals to capture full thrombin generation profile.

### T-TAS®

The T-TAS® instrument was prepared for use according to the manufacturer's instructions. AR Chips (Diapharma Cat. #TC0101) and AR Chip Calcium Corn Trypsin Inhibitor (CaCTI; Diapharma Cat. #TR0101) were warmed to room temperature. 300 μL of rehydrated thrombosomes were transferred to a 1.7 mL microcentrifuge tube and centrifuged at 3900 g×10 minutes to pellet. The thrombosomes pellet was resuspended in George King (GK) pooled normal human plasma or autologous plasma with or without autologous platelets to a concentration of approximately 100,000-450,000/μL, as determined by AcT counts (Beckman Coulter AcT Diff 2 Cell Counter). 20 μL of CaCTI with 480 μL of thrombosomes sample in GK plasma were mixed with gentle pipetting. The sample was loaded and run on the T-TAS® according to the manufacturer's instructions.

Partial Thromboplastin Time (aPTT)

A protocol for measuring aPTT follows.

Turn on instrument; and prepare Reagent 1, Reagent 2, Coag control N and Coag control P according to manufacturer guidelines.

Thaw George King Pooled normal Plasma in 37° C. water bath for 5 minutes.

Place cuvette-strips in the incubation area for prewarming at 37° C. for at least 3 minutes. Dispense a ball to each cuvette.

For each sample, incubate GKP with or without a series of concentrations of Heparin and/or Protamine sulfate for 5 minutes in room temperature.

Dispense 50 μL samples and 50 μL Reagent 1 to each cuvette. Start the timer corresponding to the incubation column for an incubation of 180 seconds.

When the instrument starts to beep, transfer the cuvettes to the test-column area.

Prime the Finnpipette once with 0.025 M CaCl₂.

Activate the Finnpipette by pressing the pipette key. Dispense 50 μL 0.025 M CaCl₂ to each cuvette using Finnpipette.

### Example 4: Comparison to Fresh Platelets

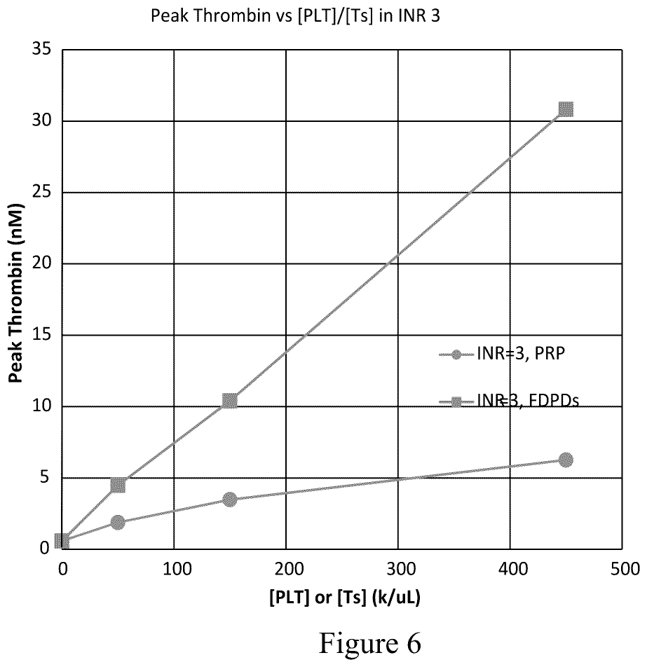

Thrombosomes elicit a specific dose-dependent recovery of thrombin generation in coumadin plasma in a manner superior to fresh platelets. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3. At a dose of INR 3, thrombosomes demonstrate a dose-dependent recovery of peak thrombin (FIG. 6). Additionally, adding Thrombosomes is more effective than an equivalent dose of fresh platelets.

### Example 5: Combination with Fresh Platelets

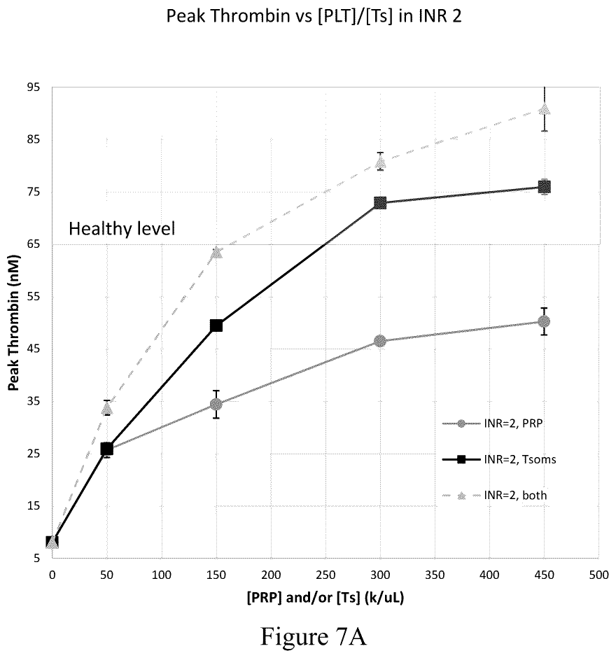

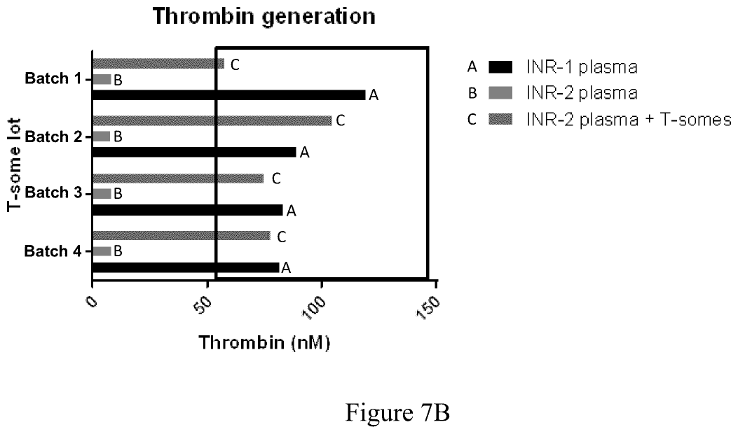

Thrombosomes cooperate with platelets increasing thrombin generation in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3) and rehydrated by addition of sterile water. TGA assays were performed as described in Example 3. Thrombosomes not only show greater efficacy, but also an additive effect with endogenous platelets (FIG. 7A). Note that thrombosomes can push the model patient back into a healthy peak thrombin range (e.g., between about 66 and 166 nM). Note that the ‘both’ line includes the two components in equal amounts in the amounts shown (e.g., at the ‘50’ value on the x-axis, the y-value represents the peak thrombin of a mixture of 50 k platelets from PRP/μL and 50 k thrombosomes/μL.

In addition, different batches of thrombosomes can also push a model patient (INR=2, treated with warfarin) back into a healthy peak thrombin range (FIG. 7B).

### Example 6: Collagen Adhesion

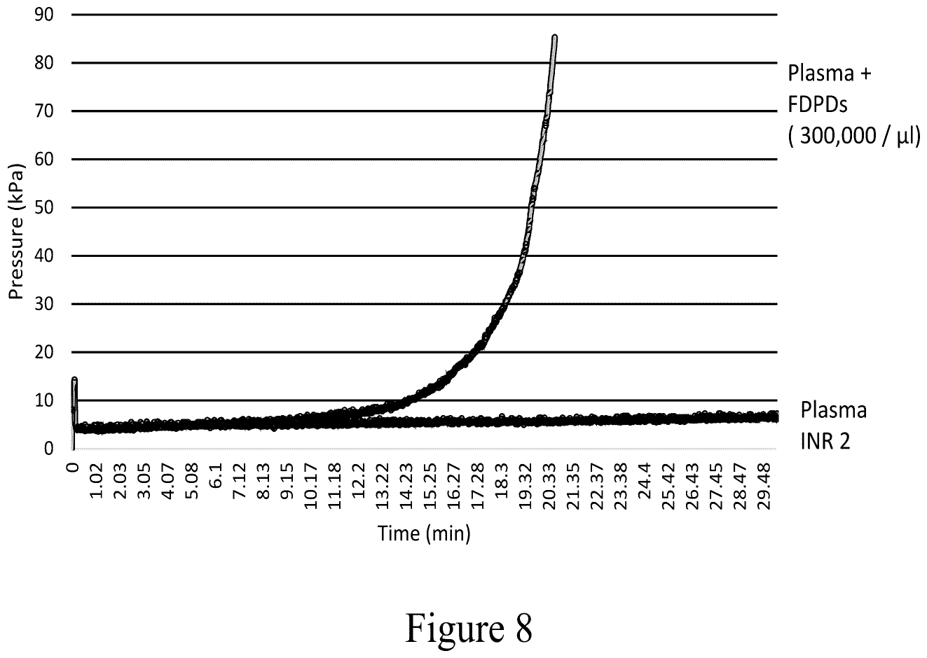

Thrombosomes adhere to and generate fibrin in warfarin plasma using shear-dependent collagen adhesion assay under flow (T-TAS®) (FIG. 8). Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were performed according to Example 3.

### Example 7. Rivaroxaban Results

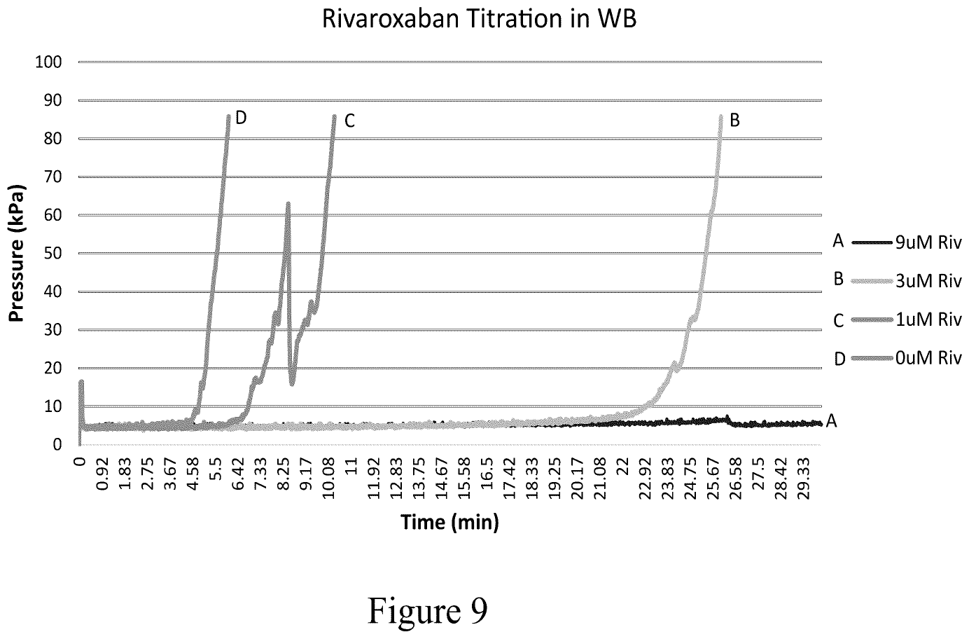

Rivaroxaban (sometimes herein called Riv) dose-response in whole blood was measured using T-TASR. An AR chip (Collagen+TF) was used. T-TAS® assays were performed according to Example 3. The donor platelets were used at 307 k/μL. A 9 UM dose (a pharmacological dose) inhibits occlusion but not all thrombus formation (FIG. 9, Table 7).

TABLE 7

| [Riv] | | | Occ Spd | AUC |
|---|---|---|---|---|
| (uM) | Occ Time | Occ Start | (kPa/min) | (kPa*min) |
| 0 | 6:04 | 4:56 | 61.8 | 1970 |
| 1 | 10:21 | 7:00 | 20.9 | 1686 |
| 3 | 26:01 | 23:32 | 28.2 | 423 |
| 9 | n/a | n/a | n/a | 13.6 |

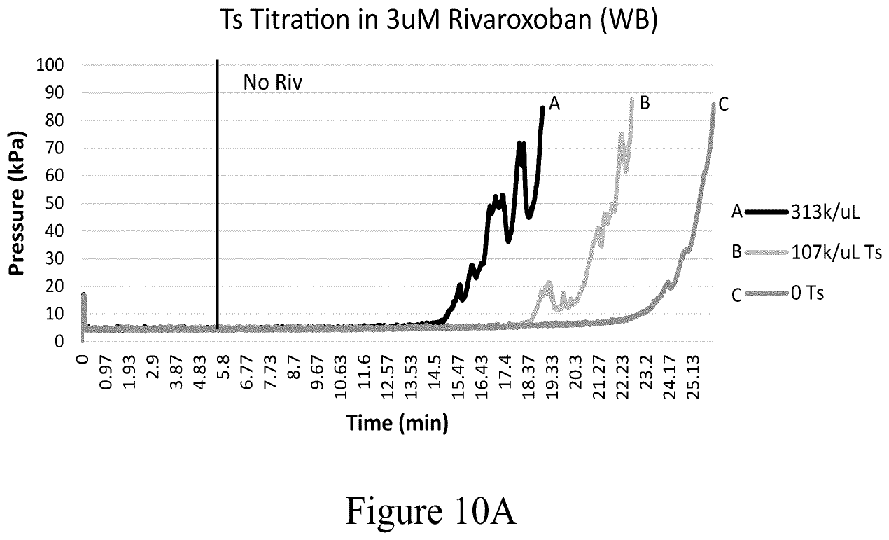

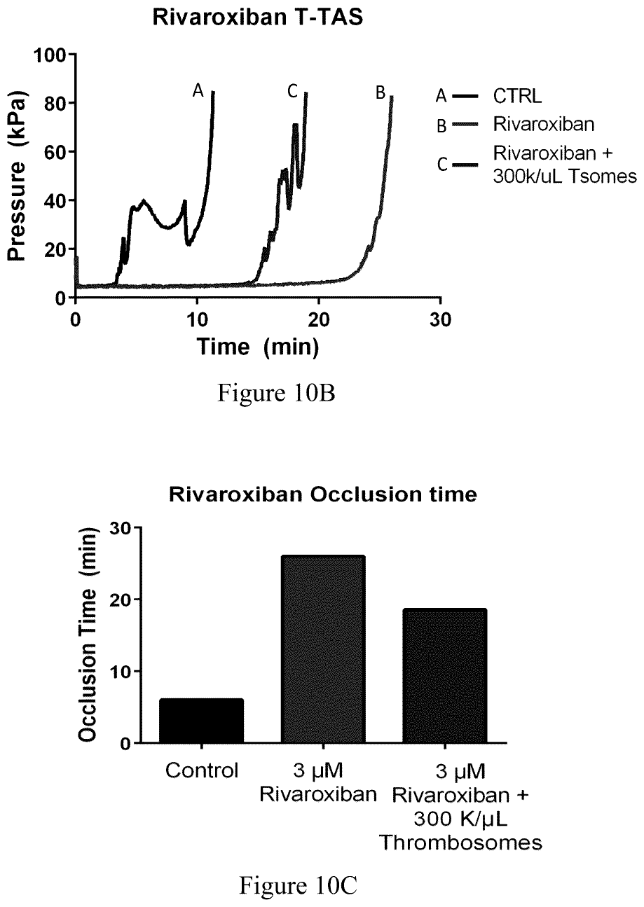

Thrombosomes partially restore thrombus formation in rivaroxaban-anticoagulated whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were run according to Example 3 using 3 μM rivaroxaban and different concentrations of thrombosomes (FIG. 10A, Table 8). The ‘No Riv’ vertical line indicates the approximate occlusion time of a sample with no added rivaroxaban.

TABLE 8

| [Ts] | | | Occ Spd | AUC |
|---|---|---|---|---|
| (k/uL) | Occ Time | Occ Start | (kPa/min) | (kPa*min) |
| 0 | 26:01 | 23:32 | 28.2 | 423 |
| 108 | 22:39 | 18:49 | 18.3 | 709 |
| 313 | 18:58 | 15:19 | 19.2 | 1033 |

In a similar experiment, T-TAS® assays were run according to Example 3 with no rivaroxaban, 3 μM rivoroxaban, and 3 μM rivoroxaban and 300×10³/μL thrombosomes. The pressure over time is shown in FIG. 10B, and the occlusion time is shown in FIG. 10C. Platelet rich plasma that was treated with 3 μM rivaroxaban extended occlusion times from 6.04 to 26.01 minutes on the T-TAS® flow system (collagen and tissue factor coated channel). The addition of 300 k/μL decreased the time back to 18.01 minutes.

### Example 8. Thromboelastography of Warfarin Plasma

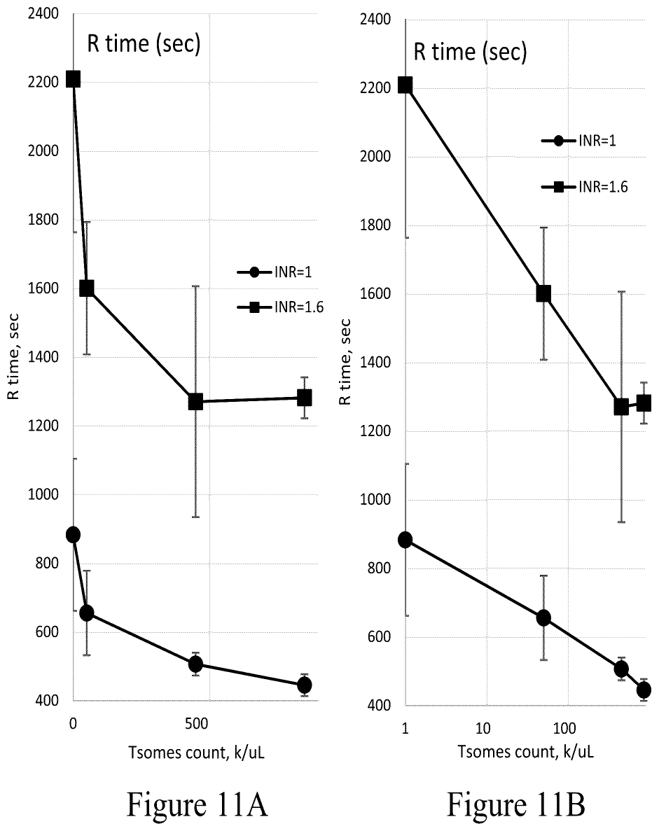

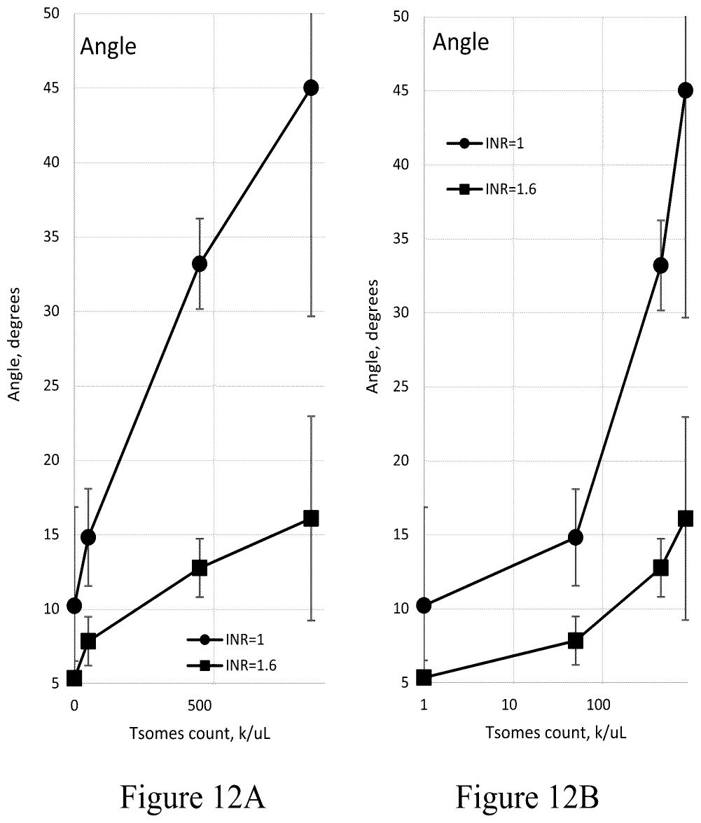

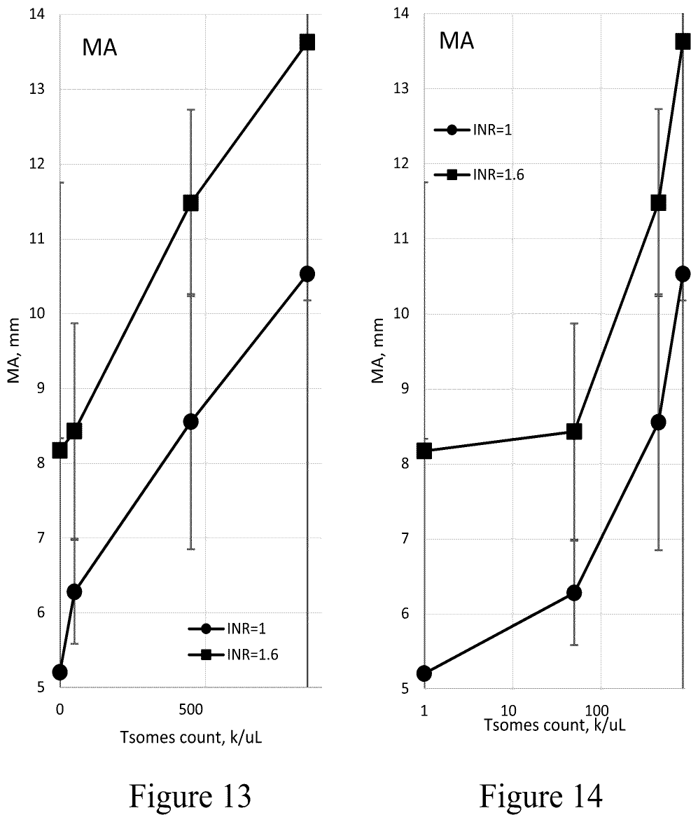

As shown in FIGS. 11-13, the effect of thrombosomes in warfarin plasma (INR=1.6) was tested and compared to standard plasma (INR=1.0), at thrombosome concentrations of 850, 450, 50 and 0 k/uL. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water.

In this experiment, +170 μL of plasma was placed in each cup; +170 μL of thrombosomes or control in each cup; and +20 μL of CaCl₂ (TEG Reagent) in each well.

Each run was performed using single replicate for each condition. Four runs were made in total. Thrombosome dilutions were prepared shortly before each run, and counts were checked immediately after each run was started. The results are shown in FIGS. 11A, 11B, 12A, 12B, 13, and 14 (thrombosomes batch 4).

### Example 9. Lag Time

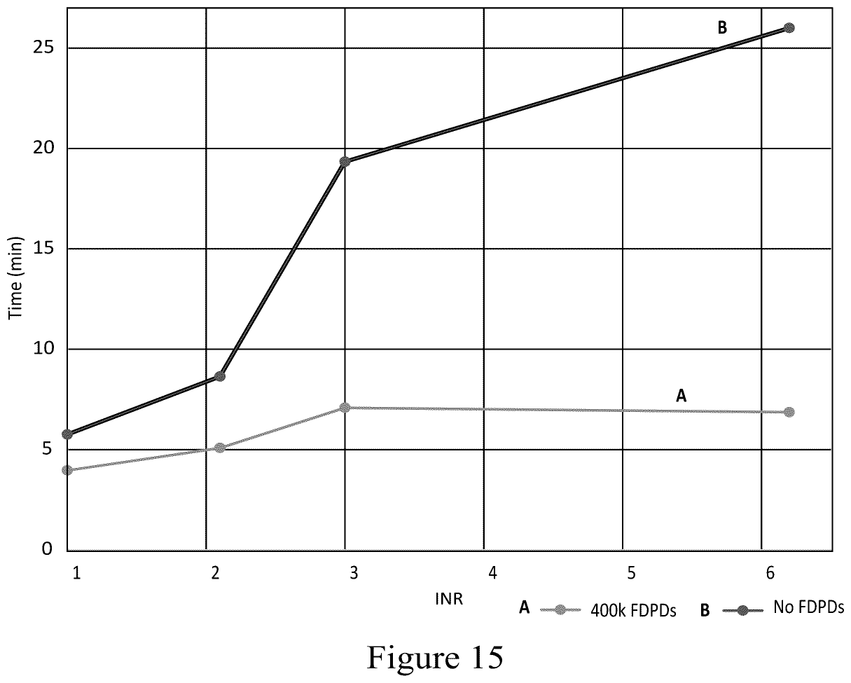

Thrombosomes decrease lag time at all tested thrombosome concentrations, and the plateau effect demonstrates no hypercoagulability (FIG. 15). Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3.

### Example 10. TEG Results

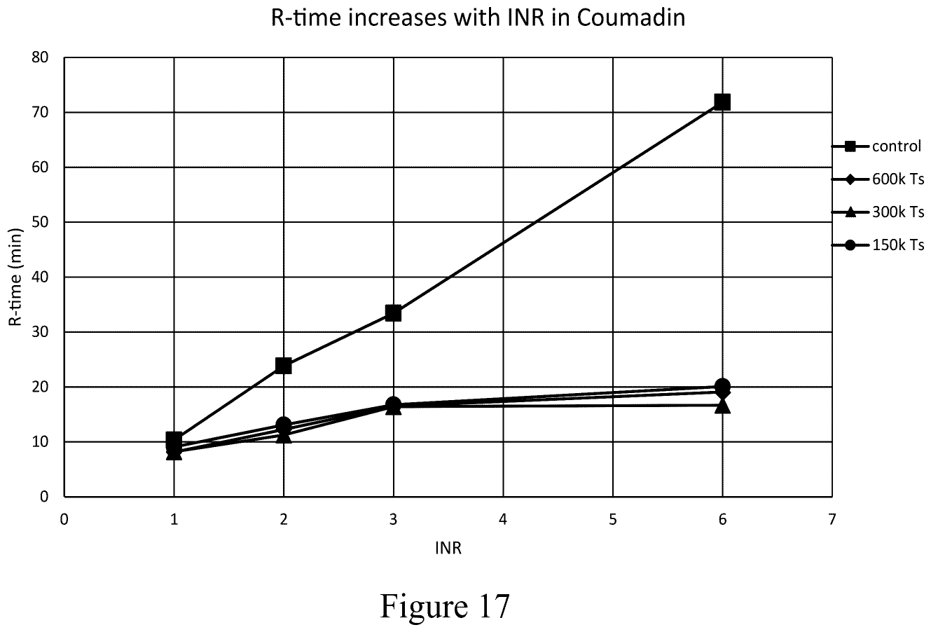

Adding various concentrations of thrombosomes decreases R-time in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were performed according to Example 3. FIG. 17 shows that thrombosomes lower R-time for various INR values. A plateau is seen before R-times of 20 min, suggesting that thrombosomes could produce therapeutically significant results.

### Example 11. Activated Clotting Time

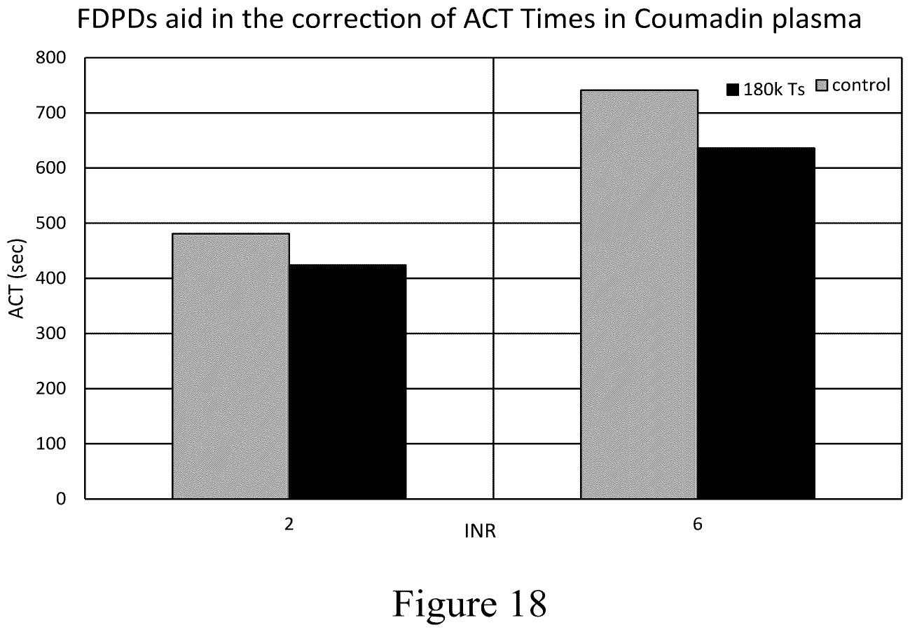

Thrombosomes exhibit an effect on activated clotting time in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. To empty MaxAct tubes, 25 μl of thrombosomes or control buffer and 25 μl of 0.2M CaCl₂ were added, followed by the addition of Whole Citrated Blood or Plasma (400 μl). The tubes were shaken once by hand then inserted into the MaxAct ACT instrument and the clotting times automatically recorded. Adding thrombosomes to physiological range improves the tACT. No change in the normal condition (INR=1) was observed. (FIG. 18).

### Example 12. Whole Blood Assays

Coumadin whole blood was prepared. Plasma from donor whole blood was removed and replaced with warfarin or control plasma as described in Example 3.

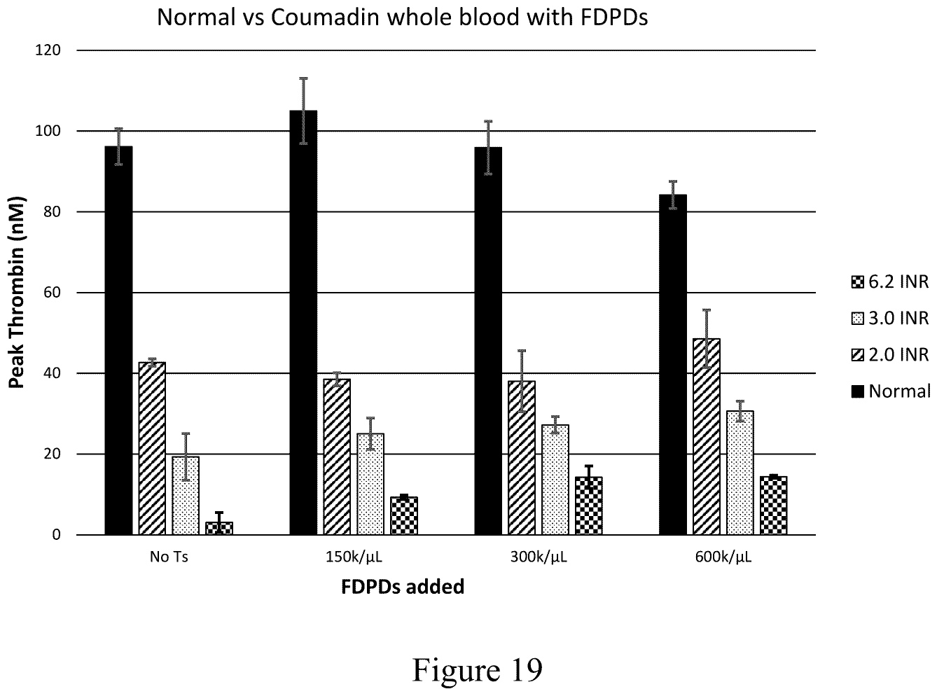

Thrombosomes increase thrombin generation in 3.0 and 6.2 INR whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3. The thrombosomes increase peak thrombin; however, the magnitude of the effect is small. The thrombosomes exhibit minimal effect on a normal blood state (FIG. 19). In these experiments, the platelet count was 150×10³/μL (as measured by CBC of the whole blood).

### Example 13. Thrombin Generation Assays

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3.

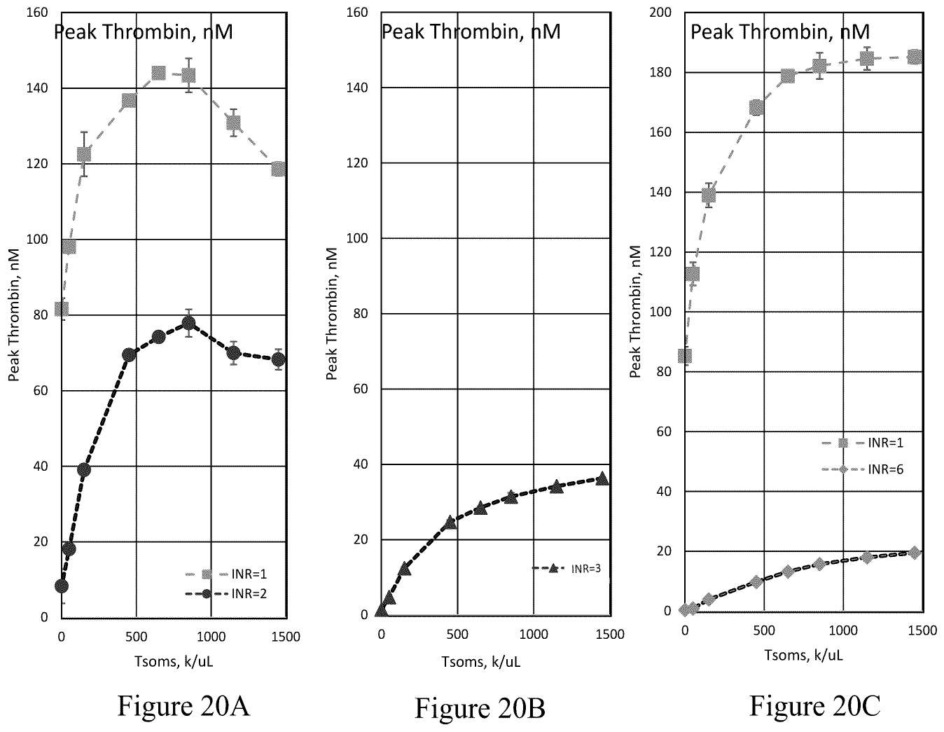

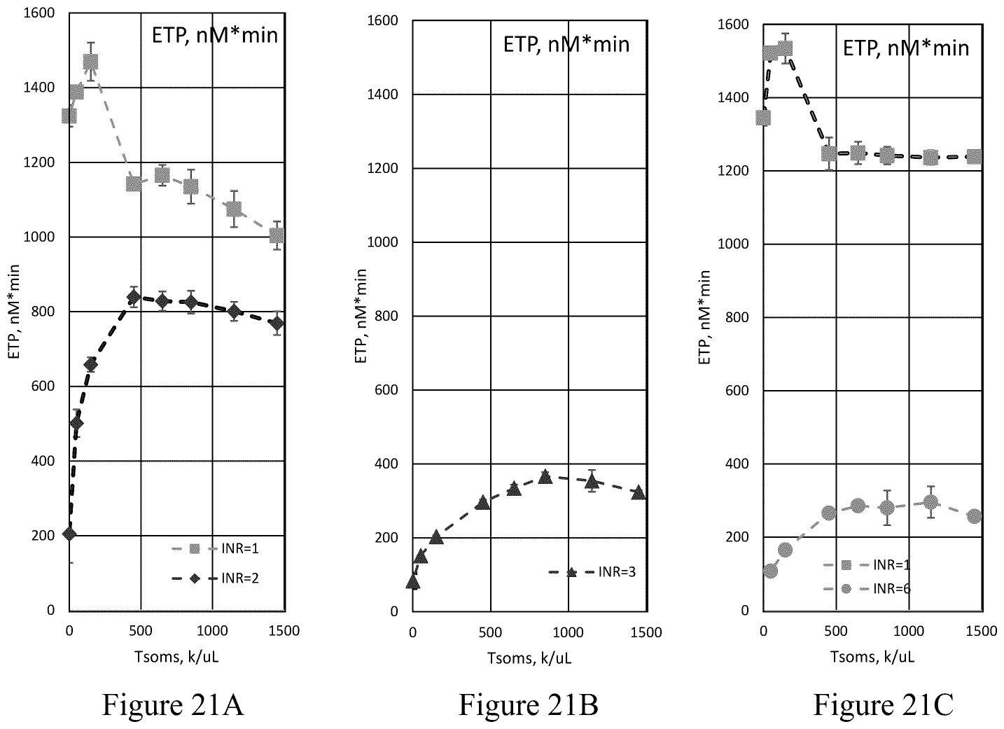

The effect of thrombosomes (batch 4) was tested and compared to standard plasma (INR=1.0) and elevated INR controls (INR=2, 3, and 6), at thrombosome concentrations of 1450, 1150, 850, 650, 450, 150, 50 and 0 k/uL. The resulting peak thrombin (FIG. 20A-C) and thrombin generation (ETP; FIG. 21A-C) values for various INR values are shown in FIGS. 20 and 21.

### Peak Thrombin Results

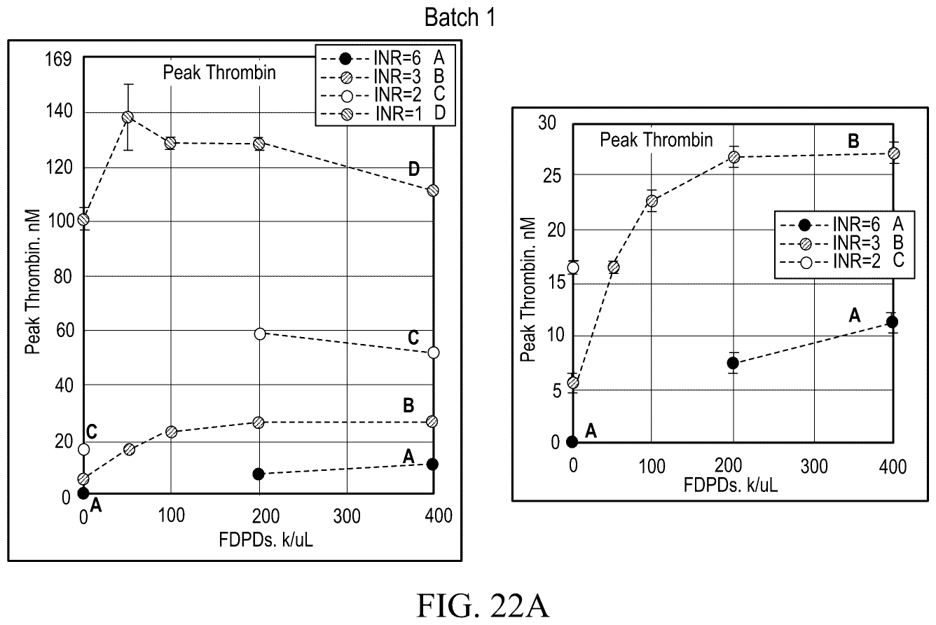

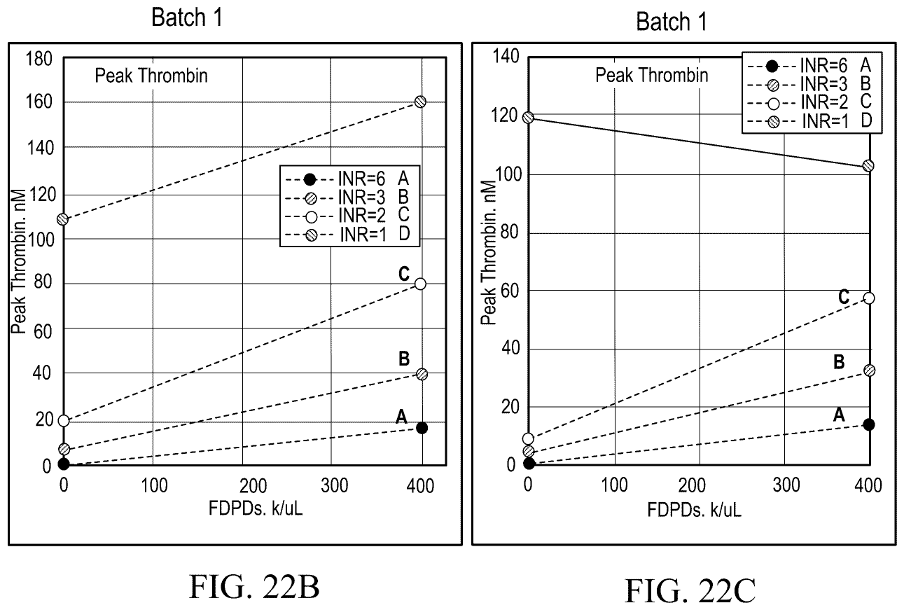

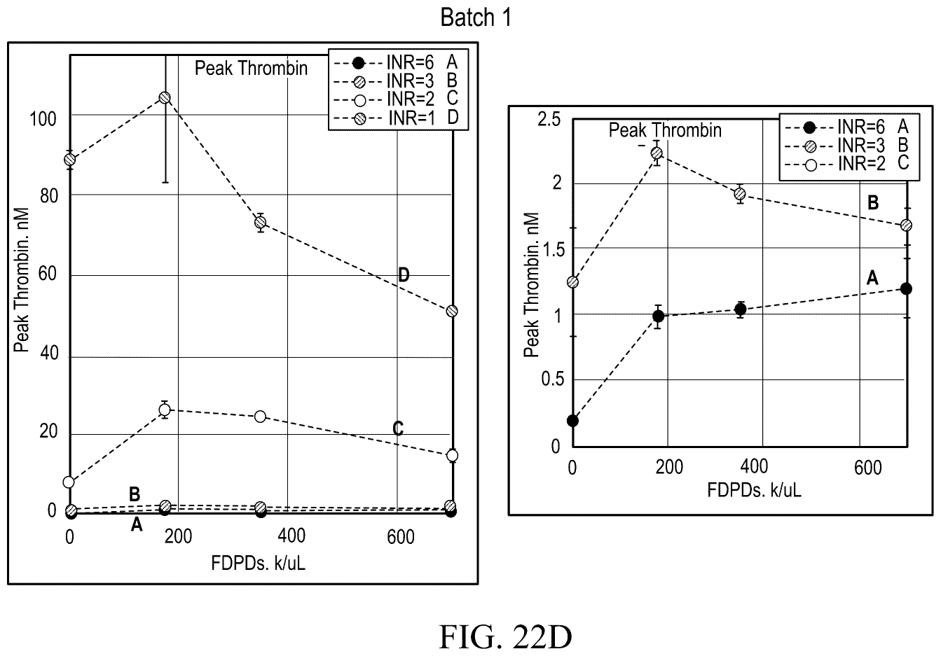

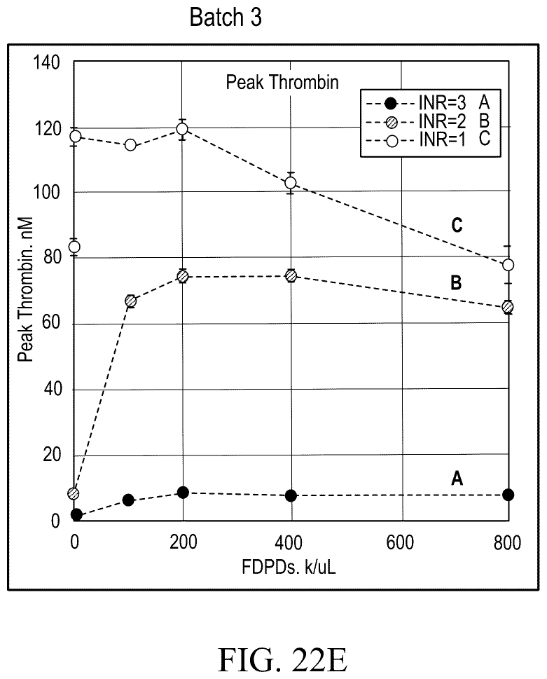

INR=1: The increase of the Peak Thrombin was saturated at about 800 k thrombosomes and was almost doubled from the normal level of about 100 nM at maximal thrombosomes concentration (FIG. 20C). Repeating the test on the same lot showed a large increase to about 145 nM at 700 k thrombosomes followed by a decrease to 120 nM at highest thrombosomes concentrations (FIG. 20A). Previous tests showed either no increase or slight increase in Peak Thrombin with following decrease at higher thrombosomes concentrations (See, e.g., FIG. 22A-E).

INR 2: Freshly prepared thrombosomes resulted in an increase of the Peak Thrombin from approximately 10 nM to about 80 nM at maximal thrombosomes concentration (FIG. 20A). Previous tests showed similar tendencies with ranges 0-20 nM to 30-80 nM (See, e.g., FIG. 22A-E).

INR 3: Freshly prepared thrombosomes resulted in an increase of the Peak Thrombin from zero to about 40 nM at maximal thrombosomes concentration (FIG. 20B). Previous tests showed similar tendencies to a maximum of about 40 nM) (batch 1; FIG. 22A-C); 1-2 nM (batch 2; FIG. 22D); 0-10 nM (batch 3; FIG. 22E).

INR 6: Freshly prepared thrombosomes resulted in an increase of the Peak Thrombin from zero to about 20 nM at maximal thrombosomes concentration (FIG. 20C). Previous tests showed similar tendencies. (See, e.g., FIG. 22A-E).

### Thrombin Generation (ETP) Results

INR 1: The ETP slightly increased at 50-150 k thrombosomes and then slightly decreased to a stable level at higher thrombosomes concentrations (FIG. 17A, FIG. 17C). Previous tests showed similar tendencies (FIGS. 21A-C). ETP range was 1000-1600 nM*min.

INR 2: The ETP increased from about 200 nM*min to about 850 nM*min at highest thrombosomes concentrations (FIG. 21A). Previous tests showed similar tendencies with ranges 200-400 nM*min to 500-900 nM*min.

INR 3: The ETP value increased from about 100 nM*min to 400 nM*min at highest thrombosomes concentrations (FIG. 21B). Previous tests showed similar tendencies with range 100-350 (batch 1); 100-200 nM*min.

INR 6: The ETP value increased from about 100 nM*min to 300 nM*min at highest thrombosomes concentrations (FIG. 21C). Previous tests showed similar tendencies with the range of 100 nM*min to 200 nM*min.

### Example 14. Thrombosomes but not Fresh Platelets Restore Thrombin Generation in Heparinized Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. aPTT and thrombin generation assays were performed as described in Example 3.

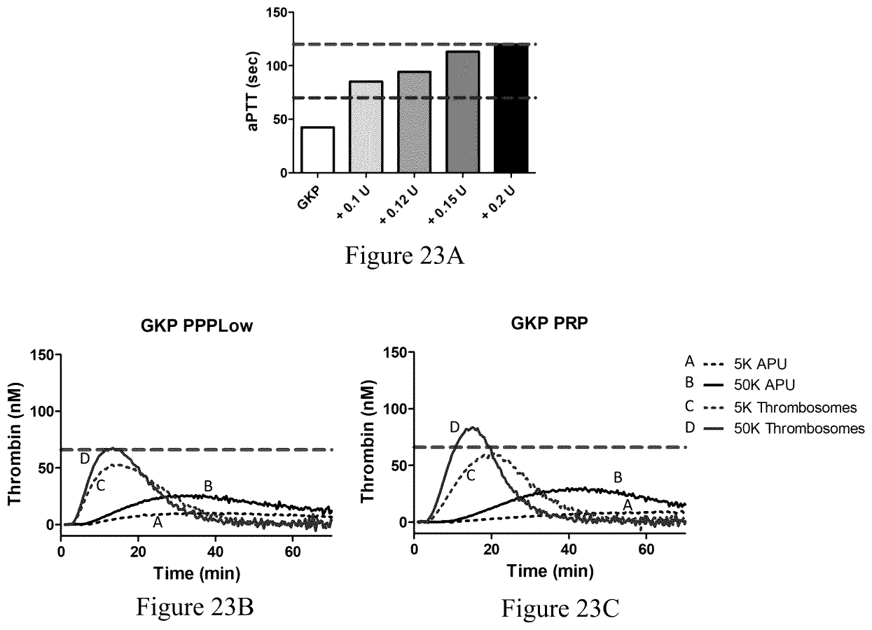

FIG. 23A shows the aPTT of George King Plasma (GKP) in the absence and presence of various concentrations of heparin as noted on the x-axis. The dashed line at approximately 70 seconds denotes the limit of abnormal aPTT and the second dashed line is the maximum time measured by the instrument (120 sec). Thrombin generation in heparin treated samples was also measured. FIG. 23B shows the effect of 0.1 U heparin in GKP on thrombin generation, in GKP, comparing apheresis units (APU) with thrombosomes at 5K (dotted lines), and 50K (solid lines) platelets or thrombosomes per μL when thrombin generation is initiated with the PPP Low reagent containing mostly phospholipids. FIG. 23C also shows thrombin generation similar to FIG. 23B, except thrombin generation is initiated by PRP reagent containing a mixture of phospholipids and tissue factor. The dashed line in FIGS. 23B and 23C denotes a typical thrombin peak value seen in this assay for control plasma. These data show that thrombosomes, but not fresh platelets, restore thrombin generation in heparinized plasma.

### Example 15. Protamine Sulfate Neutralization Restores Thrombosome-Mediated Thrombin Generation in Therapeutic Heparinized Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. aPTT and thrombin generation assays were performed as described in Example 3.

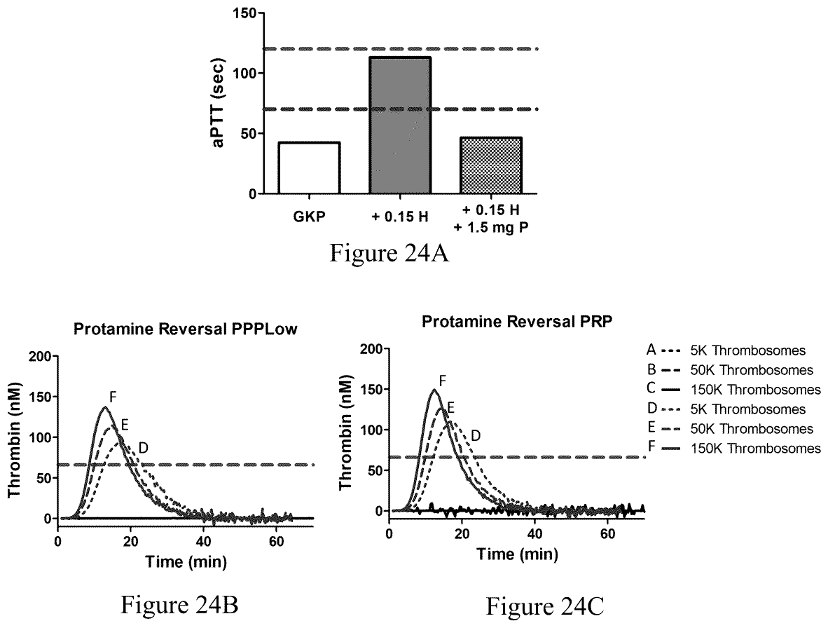

FIG. 24A shows the aPTT of George King Plasma (GKP) in the absence and presence of Heparin (H) (U/mL) and Protamine Sulfate (P) as noted on the x-axis. The dashed line at approximately 70 seconds denotes the limit of abnormal aPTT and the second dashed line is the maximum time measured by the instrument (120 sec). Thrombin generation in heparin treated samples was also measured, with and without protamine sulfate. FIG. 24B shows the effect of 2 U/mL heparin before (relatively flat lines) and after (curves) reversal by 20 μg/mL protamine sulfate on thrombin generation, in GKP, with thrombosomes at 5K (dotted line), 50K (dashed line), and 150K (solid line) thrombosomes per μL when thrombin generation is initiated with the PPP Low reagent containing mostly phospholipids. FIG. 24C also shows thrombin generation similar to FIG. 24B, except thrombin generation is initiated by PRP reagent containing a mixture of phospholipids and tissue factor. The dashed line in FIGS. 24B and 24C denotes a typical thrombin peak value seen in this assay for control plasma.

### Example 16. Thrombosomes Restore Thrombin Generation in Dabigatran-Treated Platelet Rich Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. Thrombin generation assays were performed as described in Example 3.

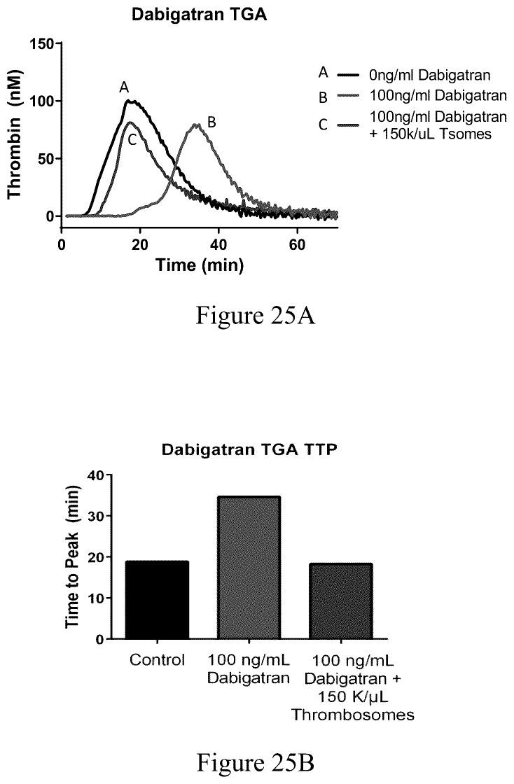

FIGS. 25A and 25B show that thrombin generation returns to normal in dabigatran treated PRP when treated with thrombosomes. Thrombin generation of PRP treated in the presence or absence of dabigatran (100 ng/mL) stimulated with PRP reagent was reversed with 150 k/μL of thrombosomes. Time to peak was increased with dabigatran to 34.67 minutes from 18.89 untreated but returned to 18.33 minutes with 150 k/μL of thrombosomes.

### Example 17. Reproducibly of FDPD Reversal of Ticagrelor and Cangrelor Inhibition of Occlusion

Human freeze-dried platelet derivatives (FDPDs), also called lyophilized human platelets (LHP), were prepared according to the procedure in Example 18. T-TAS® experiments, to measure occlusion time, using AR chips were carried out according to Example 3.

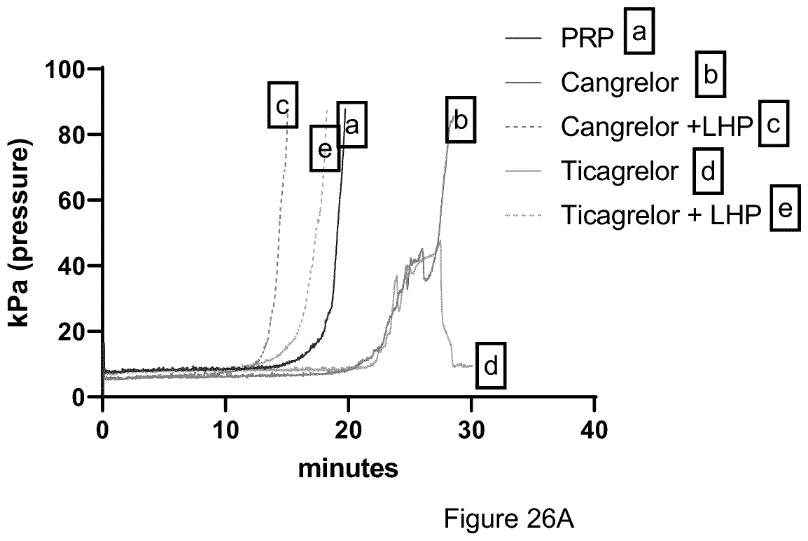

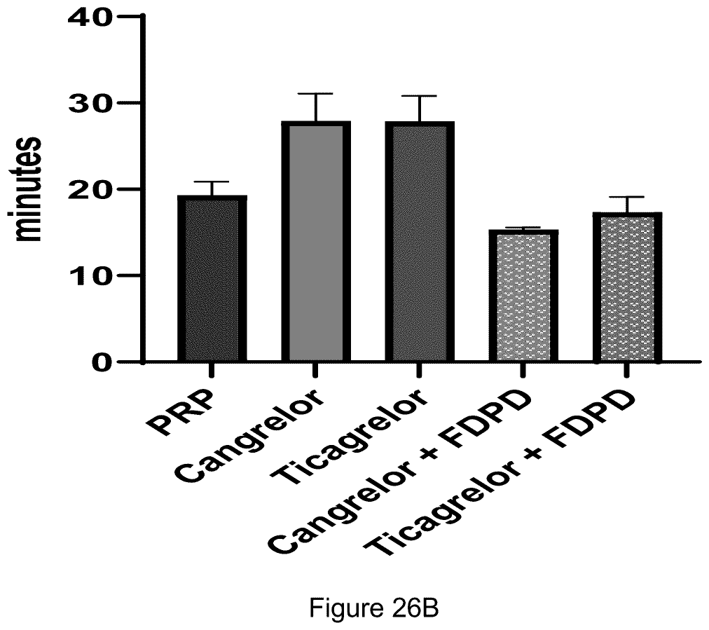

The effect of cangrelor and ticagrelor on occlusion time of Platelet Rich Plasma (PRP) with and without FDPDs was assessed using the T-TAS® assay. The concentrations of the agents were as follows: cangrelor at 1 μM, ticagrelor at 500 ng/mL, FDPDs at 150 k/μL, and ADP at 2 μM. The results are shown in FIGS. 26A and 26B. Adenosine diphosphate (ADP) stimulated PRP samples showed occlusion at 19.5±1.5 minutes (n=4) which increased to 28.0±3.0 minutes with cangrelor (n=4) or 28.0±3.0 minutes with ticagrelor (n=5) treatment (FIGS. 26A-26B). The addition of 150 k/μL lyophilized human platelets to P2Y12-inhibited PRP reduced time to thrombus formation to lower than PRP alone; 15.5±0.5 minutes in the presence of cangrelor (n=3) versus 17.5±1.5 minutes in the presence of ticagrelor (n=5) (FIG. 26). The number of trials per each assay is listed as the “(n=_)” values.

These results demonstrate that occlusion of platelets on the T-TAS® AR Chip in the presence of human FDPDs is not affected by the antiplatelet effect of cangrelor and ticagrelor. These results suggest that human FDPDS will maintain expected function when infused into patients receiving cangrelor, ticagrelor, and/or similar agents.

### Example 18. Tangential Flow Filtration (TFF) Method of Platelet Derivative Preparation

Apheresis platelets underwent tangential flow filtration in accordance with a standard operating procedure, including the following process steps: platelet dilution, platelet concentration and platelet washing.

The platelet donor units were initially pooled into a common vessel. The platelets may or may not be initially diluted with an acidified washing buffer (e.g., a control buffer) to reduce platelet activation during processing. The platelets can undergo two processing pathways; 1) either washed with control buffer until a desired residual component is reached (e.g., donor plasma) before being concentrated to a final product concentration or 2) the platelets are concentrated to a final product concentration before being washed with control buffer until a desired residual component is reached (e.g., donor plasma). TFF processed platelets are then filled into vials, lyophilized and thermally treated.

One particular protocol follows.

For all steps of the TFF process in this Example, Buffer F was used. The process was carried out at a temperature of 18-24° C.

### Buffer F

| Component | Value (±1%) | |
|---|---|---|
| HEPES | 7.6 | mM |
| NaCl | 60 | mM |
| KCl | 3.84 | mM |
| Dextrose | 2.4 | mM |
| NaHCO₃ | 9.6 | mM |
| Trehlaose | 80 | mM |
| Ethanol | 0.8% | |
| Polysucrose | 6% | (w/v) |

| | |
|---|---|
| pH | 6.6-6.8 |

Platelets were loaded onto the TFF (PendoTECH controller system (PendoTECH® Princeton, NJ; https://www.pendotech.com), which was prepared with a Repligen TFF Cassette (XPM45L01E). The TFF process was performed using a membrane with a pore size of 0.45 μm. The platelets were diluted with an equal weight (±10%) of Buffer F. The platelets were concentrated to about 2250×10³ cells/μL (±250×10³) and then washed with approximately 2 diavolumes (DV) of Buffer F. The target plasma percentage was typically less than 15% relative plasma (as determined by plasma protein content). Removal of plasma proteins was monitored through 280 nm UV absorbance against known correlations.

In some cases, samples were drawn at UV absorbance readings correlating to about 51% relative plasma volume, about 8.1% relative plasma volume, about 6.0% relative plasma volume, and about 1.3% relative plasma volume. Low volume aliquots were sampled throughout each processing step with the about 6.0% and under samples.

Following washing, if the concentration of the cells was not 2000×10³ cells/μL (±300×10³), the cells were either diluted with Buffer F or were concentrated to fall within this range. Under all circumstances whenever the cells were contacted with the Buffer F, it was done at a temperature in the range of 18-24° C. For a better clarity, the cells were loaded with the reagents of the Buffer F at a temperature in the range of 18-24° C. The cells were typically then freeze-dried (i.e. lyophilized) and subsequently heated (thermally treated) at 80° C. for 24 hours, thereby forming Freeze-dried platelet derivatives (FDPDs), which are also called THROMBOSOMES® when prepared by Cellphire, Inc. For clinical or commercial use.

The lyophilization procedure used to prepare the human FDPDs is presented in Table LA2.

TABLE LA2
LYOPHILIZER RECIPE

| 30 g Fill | | | | | | | 10 g Fill | | | | | | |
|---|---|---|---|---|---|---|---|---|---|---|---|---|---|
| Freezing | | | | | | | Freezing | | | | | | |
| Ramp to | −40° | C. | 0 | mins | o | mTorr | Ramp to | −40° | C. | 0 | mins | 0 | mTorr |
| Hold at | −40° | C. | 180 | mins | 0 | mTorr | Hold at | −40° | C. | 180 | mins | O | mTorr |
| Final Freezing | | | | | | | Final Freezing | | | | | | |
| Hold at | −40° | C. | 0 | mins | 100 | mTorr | Hold at | −40° | C. | O | mins | 100 | mTorr |
| Primary Drying | | | | | | | Primary Drying | | | | | | |
| Ramp to | .5° | C. | 420 | mins | O | mTorr | Ramp to | −10° | C. | 360 | mins | O | mTorr |
| Hold at | .5° | C. | 1200 | mins | 0 | mTorr | Hold at | −10° | C. | 360 | mins | 0 | mTorr |
| Ramp to | +5° | C. | 120 | mins | 0 | mTorr | Ramp to | +S° | C. | 180 | mins | O | mTorr |
| Hold at | +S° | C. | 1380 | mins | 0 | mTorr | Hold at | +S° | C. | 360 | mins | 0 | mTorr |
| Ramp to | +30° | C. | 300 | mins | 0 | mTorr | Ramp to | +30° | C. | 300 | mins | 0 | mTorr |
| Hold at | +30° | C. | 720 | mins | 0 | mTorr | Hold at | +30° | C. | 720 | mins | 0 | mTorr |
| Hold at | +30° | C | 720 | mins | 200 | mTorr | Hold at | +30° | C. | 720 | mins | 200 | mTorr |
| Hold at | +30° | C. | 60 | mins | 0 | mTorr | Hold at | +30° | C. | 60 | mins | 0 | mTorr |
| Secondary Drying | | | | | | | Secondary Drying | | | | | | |
| Hold at | +30° | C. | 9999 | mins | 0 | mTorr | Hold at | +30° | C. | 9999 | mins | 0 | mTorr |

| | |
|---|---|
| Total Recipe Time la −85 hours | Total Recipe Time la −54 hours |

To perform studies such as thrombin generation studies (TGPU), and aggregation studies, FDPDs were typically rehydrated with water over 10 minutes at room temperature. In general, the rehydration volume is equal to the volume used to fill each vial with cells prior to drying. The platelet derivatives which were heated (thermally treated) after lyophilization are also referred to as baked FDPDs. Whereas the FDPDs which were not heated (thermally treated) after lyophilization are referred to as unbaked FDPDs.

Human FDPDs, obtained after lyophilization in the form of a powder can be used for commercial applications, such as providing the human FDPDs (e.g. THROMBOSOMES®) in dried form in vials to, for example, a medical practitioner who can rehydrate the vials with an appropriate amount of a liquid.

### Example 19. FDPDS Restore Bleeding Time in NOD-SCID Mice Treated with Supra-Pharmacologic Clopidogrel

Human FDPDs were prepared consistent with the procedure in Example 18.

Mice were treated with clopidogrel at 5 mg/kg for 3 days. The mice were anesthetized, the tail end was snipped off at 1 mm diameter and submerged in warm saline and time to clot recorded. Animals were syringe injected, into a vein or artery, with saline or 1.6×10⁹/kg of FDPDs, at the same time the tail was snipped, and the tail snip trial commenced. The time from tail snip to tail stop bleeding was recorded by visual inspection of cessation of blood loss.

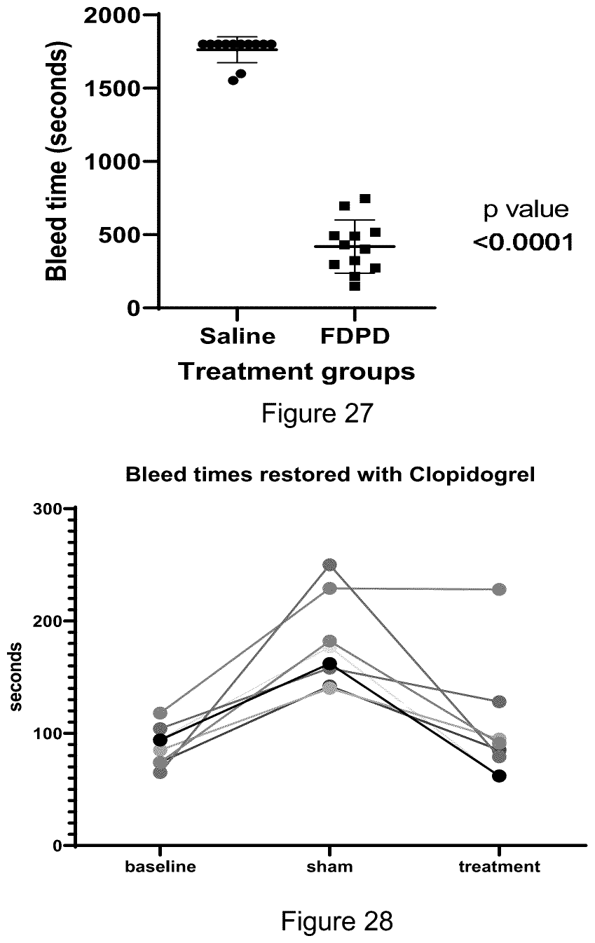

The results for this experiment are shown in FIG. 27. The bleed time (seconds) was extended to 1661+/−257.9 seconds with clopidogrel treatment verses untreated at 758.6+/−623.4 seconds. Treatment with 1.6×10⁹/kg FDPDS decreased bleeding to 417.9+/−166.6 seconds. One-way ANOVA software, was utilized for analysis of the data.

These results demonstrate that human FDPDs are resistant to clopidogrel effects and restore hemostasis in a mice tail snip model. Human FDPDs have the potential to be an effective clinical tool to stop bleeding in a patient population being treated with clopidogrel.

### Example 20. FDPDs Restore Bleeding Time in New Zealand White Rabbits Treated with Supra-Pharmacologic Clopidogrel

Additional experiments were carried out with clopidogrel treated New Zealand White Rabbits. Human FDPDs were prepared consistent with the procedure in Example 18.

Rabbits were treated with clopidogrel at 23 mg/kg for 5 days. The rabbits were anesthetized, the ear was bled and time to clot recorded. Animals were treated, injected into a vein or artery, with saline or 1.6×10⁹/kg of human FDPDs, at the same time the ear was bled, and the ear bleed trial commenced. The time from ear bleed to stop bleeding was recorded by visual inspection.

Results of this experiment (FIG. 28) showed that seven out of a eightsix clopidogrel treated rabbits had bleeding time restored when FDPDs were administered. The bleed time (seconds) was extended to 206.8+/−104.2 seconds with clopidogrel treatment verses untreated at 87.1+/−18.2 seconds. Treatment with 1.6×10⁹/kg FDPDS decreased bleeding to 417.9+/−166.6 seconds (FIG. 28). One-way ANOVA was utilized for analysis of the data.

These results demonstrate that human FDPDs are resistant to clopidogrel effects and restore hemostasis in a rabbit ear bleed model. Lyophilized human platelets have the potential to be an effective clinical tool to stop bleeding in a patient population being treated with clopidogrel.

### Example 21. FDPDs Contribution to Thrombin Generation in the Presence of 25 ng/ml Rivaroxaban Treated OCTAPLAS® PRPs Reagent

Human FDPDs were prepared according to the method as described in Example 18. The OCTAPLAS® plasma used in this example is a solvent/detergent treated, pooled human plasma available from Octapharma USA, Inc., 117 W. Century Road Paramus, NJ 07652; www.octapharmausa.com.

A Thrombin Generation Assay (TGA) was performed to detect thrombin generation and endogenous thrombin potential in (1) OCTAPLAS® with platelet rich plasma (PRP) and incrementally increasing FDPD concentration (0, 10, 20, 40, 80, 160)×10³/μL and (2) OCTAPLAS® with platelet rich plasma reagent (PRP), 25 ng/mL rivaroxaban, and incrementally increasing FDPD concentration (0, 10, 20, 40, 80, 160)×10³/μL. A 25 ng/mL dose of Rivaroxaban is within the physiological dose range and is an effective dose to inhibit thrombin generation.

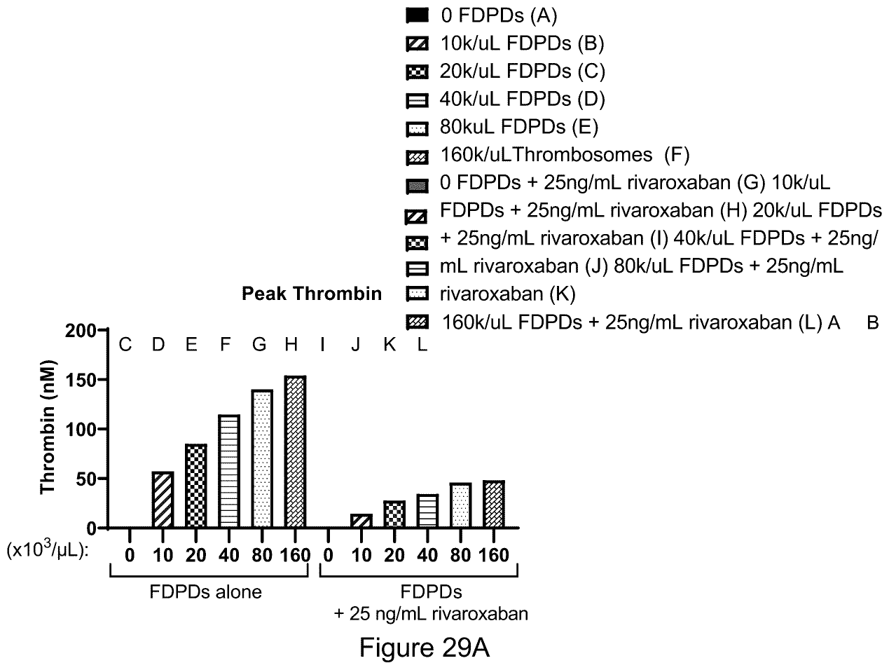

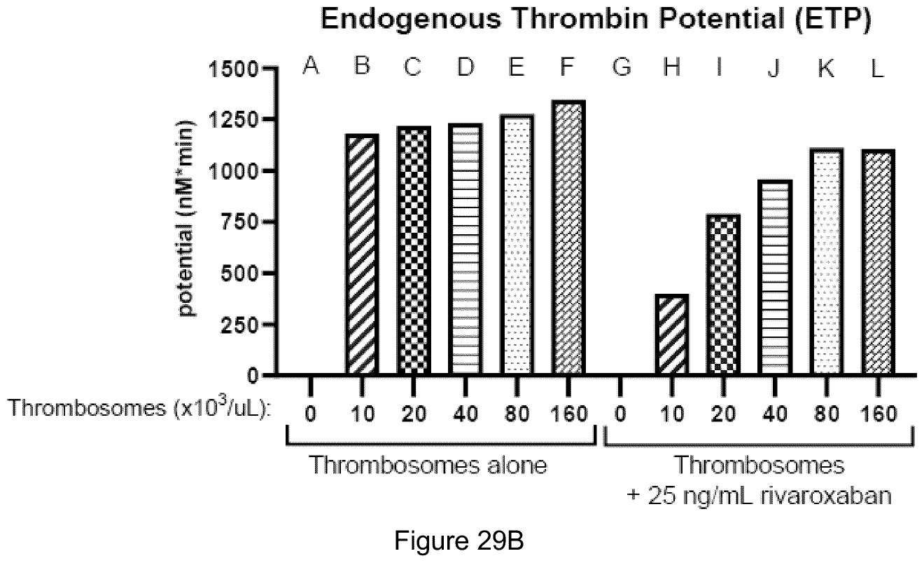

The results of this experiment are shown in FIGS. 29A and 29B. These results demonstrated that even low doses of FDPDs are capable of catalyzing some thrombin generation (FIG. 29A) and partially recovering the endogenous thrombin potential (FIG. 29B) in the presence of 25 ng/ml dose of rivaroxaban in OCTAPLAS® with PRP reagent.

### Example 22. Addition of FDPDs Shows Partial Recovery of Occlusion Time of 25 ng/ml Rivaroxaban Treated Whole Blood (WB)

Occlusion time was measured on the Total Thrombin formation Analysis System (T-TAS®) 01 using AR chips (Collagen and Tissue FactorF stimulant). The T-TAS® 01 (Diapharma®, https://diapharma.com) instrument was prepared for use according to the manufacturer's instructions. AR Chips (Diapharma #19001) and Calcium Corn Trypsin Inhibitor (CaCTI; Diapharma Cat. #TR0101) were warmed to 37° C. or room temperature, respectively. Whole blood was collected in sodium citrate tubes 30 minutes prior to the start of the assay. FDPDS were rehydrated, counted (Beckman Coulter AcT Diff 2 Cell Counter), and added to whole blood at the indicated final concentration. Rivaroxaban (Cayman Chemical cat #16043) was dissolved in 100% DMSO to make a 10 μg/mL stock solution and added to the sample at the indicated final concentration, yielding a final DMSO content of 0.25%, 480 μL of sample was mixed with 20 μL of calcium CTI reagent and run on the AR chip on T-TAS® 01.

FDPDs were prepared according to the procedures of Example 18. The TAS® 01 assays using AR chips general method and OCTAPLAS® plasma are described in Example 21 above. The TAS® 01 assays were run with no rivaroxaban, 25 ng/mL rivaroxaban, and 25 ng/mL rivaroxaban and 20 k/μL FDPDs.

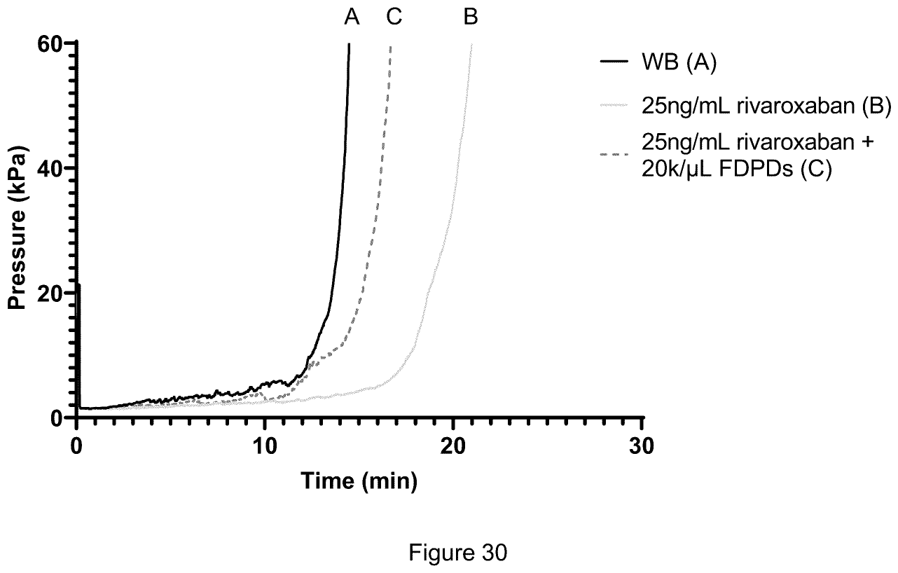

The pressure over time is shown in FIG. 30 with pressure increase being indicative of occlusion. The results show that Occlusion time was partially restored with the addition of FDPDs into rivaroxaban treated whole blood.

### Example 23. The Addition of FDPDs to Rivaroxaban Treated OCTAPLAS® or Rivaroxaban Treated Fresh PRP Show the Recovery of Fibrin and Thrombin Generation Using Thrombodynamics Analyser System (T2T)

The Thrombodynamics R Analyser System (T2T) (Diapharma®, https://diapharma.com) was used to detect thrombin generation and fibrin formation. Human FDPDs were prepared according to the procedure in Example 18.

Thrombodynamics set-up and sample prep were performed according to manufacturer's recommendation EXCEPT a phospholipid reagent was not added. The experiment was performed with either OCTAPLAS® or fresh PRP. OCTAPLAS® and PRP were each incubated with 300 ng/ml rivaroxaban for 2 minutes, FDPDSs were added to the sample, and then the sample was added to Reagent 1 (contact pathway inhibitor and thrombin fluorescent substrate of the T2T assay); 20 k/μL FDPDSs were added for OCTAPLAS® run and 2 k/μL for PRP run. The sample was incubated at 37° C. for 3 min (PRP) or 15 min (OCTAPLAS®) according to instrument protocol. Following the incubation, the sample was added into a cuvette containing TF-coated plastic insert and the run was started. The concentrations of the components in the fresh PRP run are decreased in order to see the dynamic response when using fresh platelets versus OCTAPLAS®, a detergent-treated plasma.

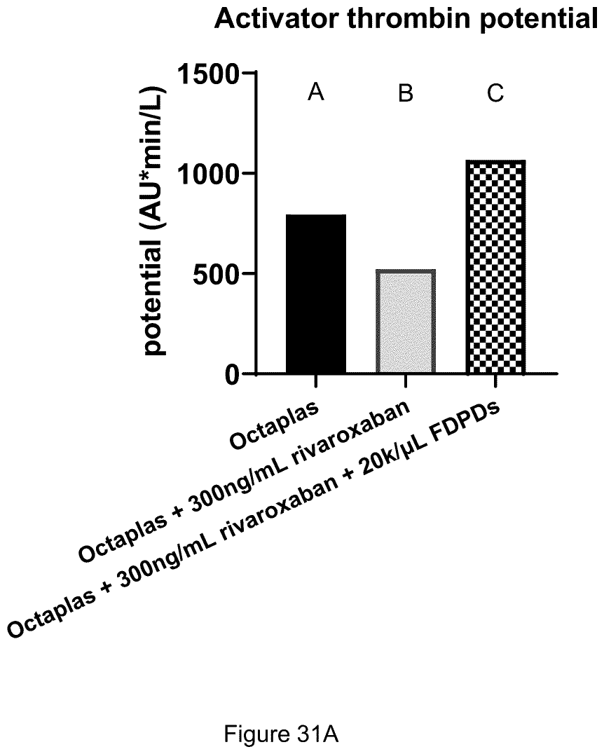

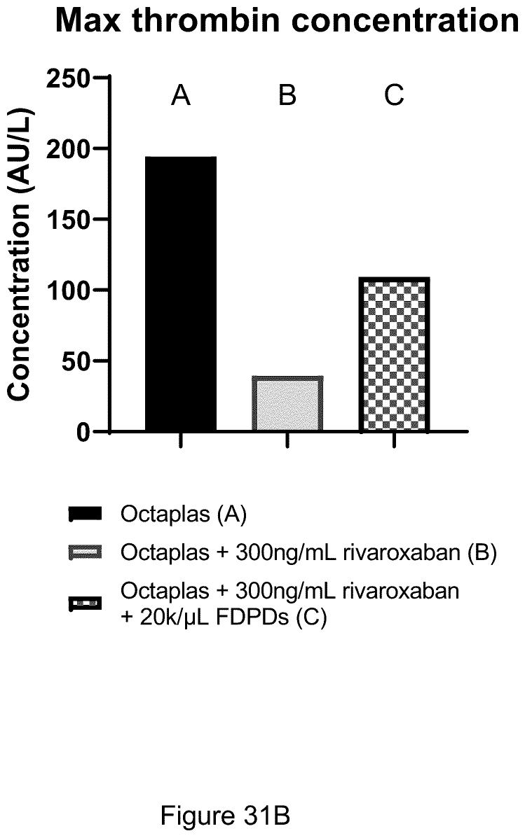

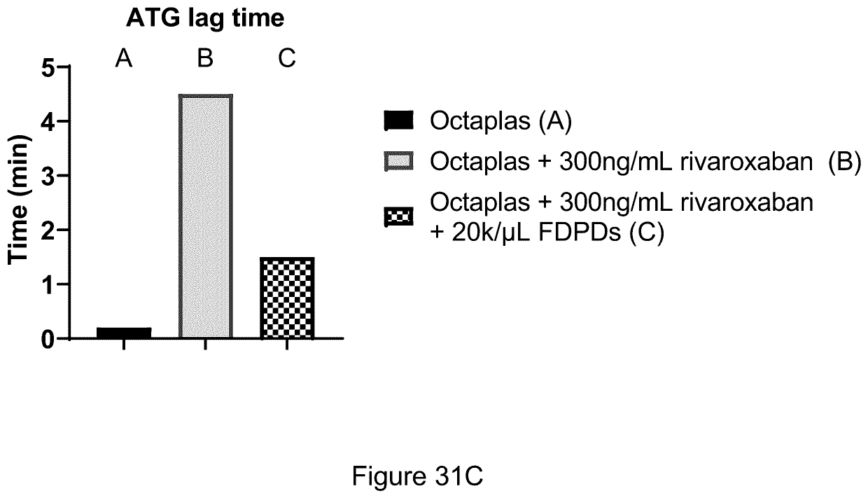

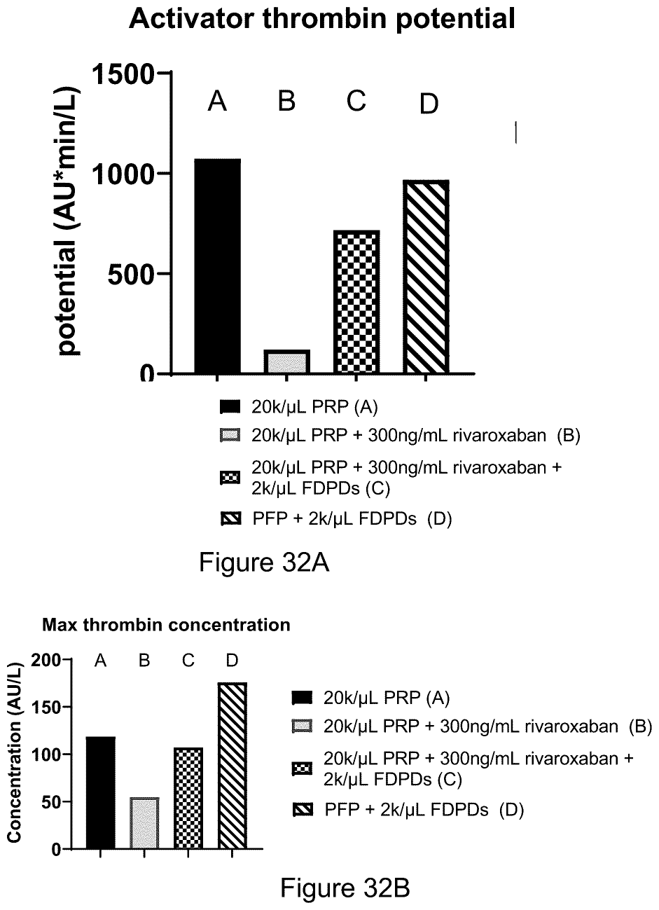

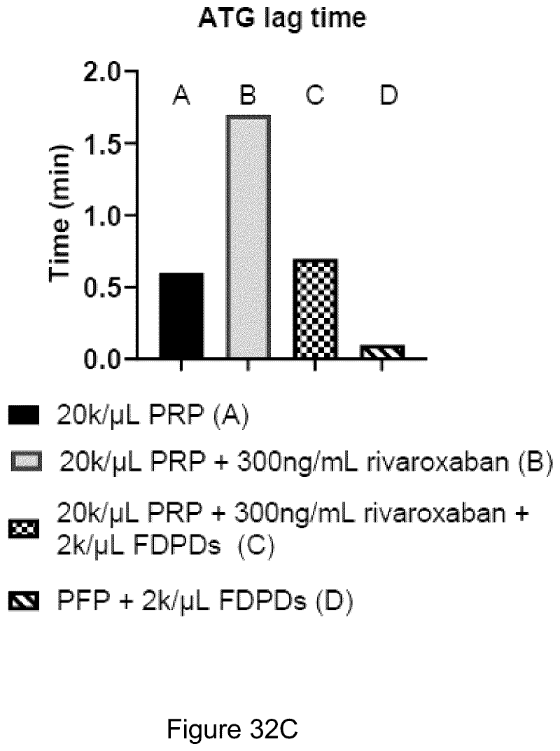

This results of this experiment showed recovery of fibrin and thrombin generation by FDPDs in rivaroxaban treated Octaplas (FIGS. 31A-31C). The results also showed the partial recovery of fibrin and thrombin generation in a rivaroxaban-treated fresh PRP sample (FIGS. 32A-32C) at the physiological ratios of endogenous platelets: FDPDs that would be expected if using clinically.

### Example 24 Addition of FDPDSs Rescues Clot Forming Capacity of a Heparin Treated Sample with Less than Clinically Suggested Dose of Protamine

This experiment assessed how clinical proportions of Heparin and Protamine affect FDPD function. FDPDs were prepared according to the procedure in Example 18. An Activating Clotting Time (ACT) assay was performed to measure time to clot and TGA was performed to measure thrombin generation. An initial Heparin titration run was performed using ACT to find the minimum Heparin dose needed to reach abnormal clot time. Results showed a minimum of 0.8 U/mL Heparin is needed to reach abnormal clot formation.

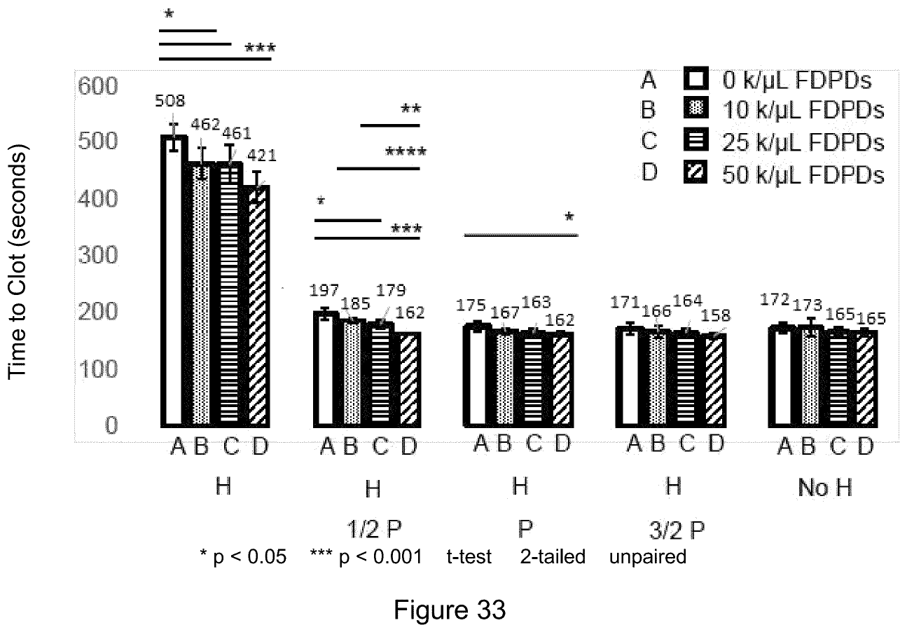

FIG. 33 shows the effect of FDPDs on time to clot in the presence of anticoagulants using the ACT test. Activated clotting time was measured using pooled normal plasma with heparin (H) and protamine (P) as labeled on the x-axis; ½ P, P, and 3/2 P represent protamine doses of 4, 8, and 12 μg/ml, respectively. FDPDs, (referred to as “Tsomes” in FIG. 33) were added at 10, 25 or 50 K/μl. Samples labeled “0” Tsomes had an equal volume of control buffer added to pooled normal plasma. “K” in the legend stands for 10³ FDPDS. All samples were run in duplicate. N=2. The experiment was repeated on separate days to improve statistical relevance.

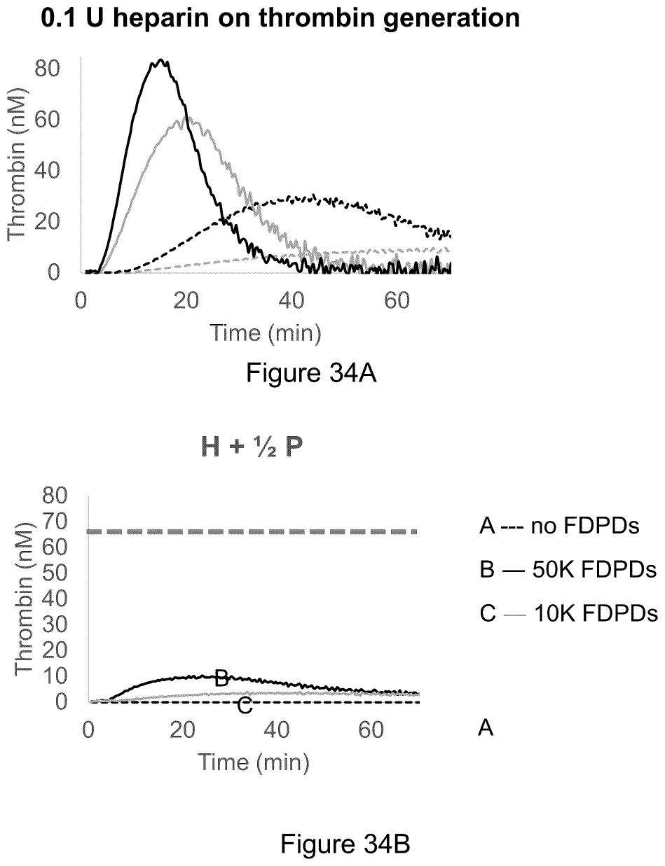

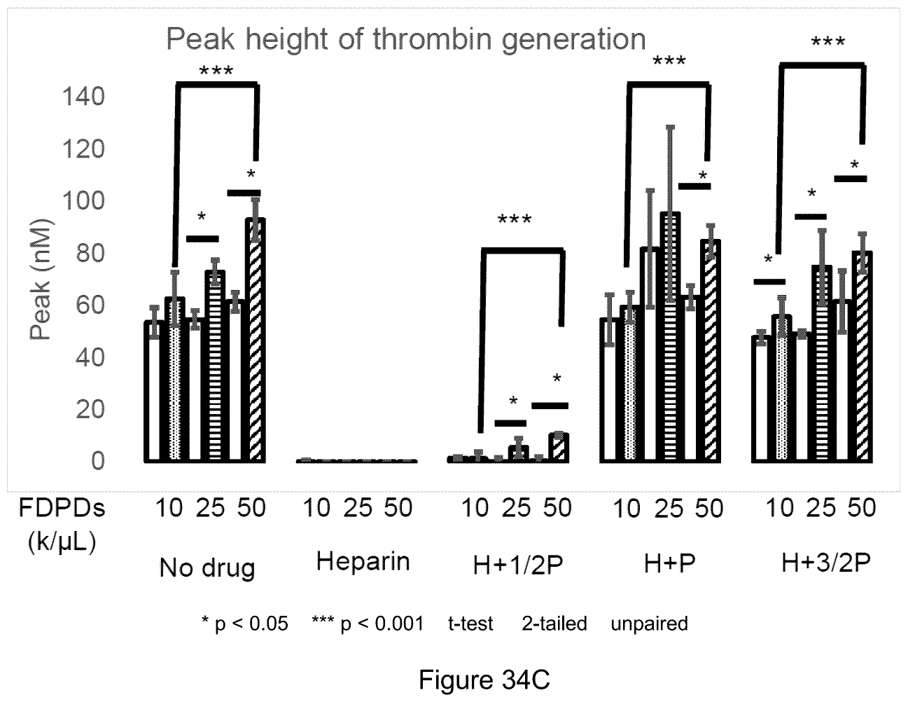

FIG. 34A-C show that FDPDs retain thrombin generation peak in the presence of heparin and protamine using TGA. FIG. 34A shows the effect of 0.1 U heparin on thrombin generation, in pooled normal plasma, comparing apheresis units (APU) with FDPDs at 5K and 50K platelets per μL. FIG. 34B shows the impact of 0.8 U/mL heparin reversed by 4 μg/ml protamine (½ of the recommended reversal doses) with FDPDs at 10K and 50K platelets per μL. TGA of FIG. 34A and FIG. 34B is initiated by PRP reagent containing a mixture of phospholipids and tissue factor. The dashed line in FIGS. 34A and 34B denotes the typical thrombin peak seen in this assay. FIG. 34C shows peak height of thrombin generation of samples which were treated with heparin and protamine as described on the x-axis. Empty (white fill) bars represent samples that were diluted with control buffer, and filled bars represent samples that were treated with FDPDs. All samples were run in triplicate. The experiment was repeated on separate days to improve statistical relevance.

The results of this experiment demonstrate that if there is a less than clinical dose of protamine present in a heparin treated sample, then FDPDs impose a dose dependent decrease of time to clot (ACT) and increase of thrombin peak height (TGA)

### Example 25. Reproducibly of FDPD Reversal of Aspirin (ASA) Inhibition of Occlusion

Human freeze-dried platelet derivatives (FDPDs) were prepared according to the procedure in Example 18. T-TAS® experiments using AR chips were carried out according to Example 3.

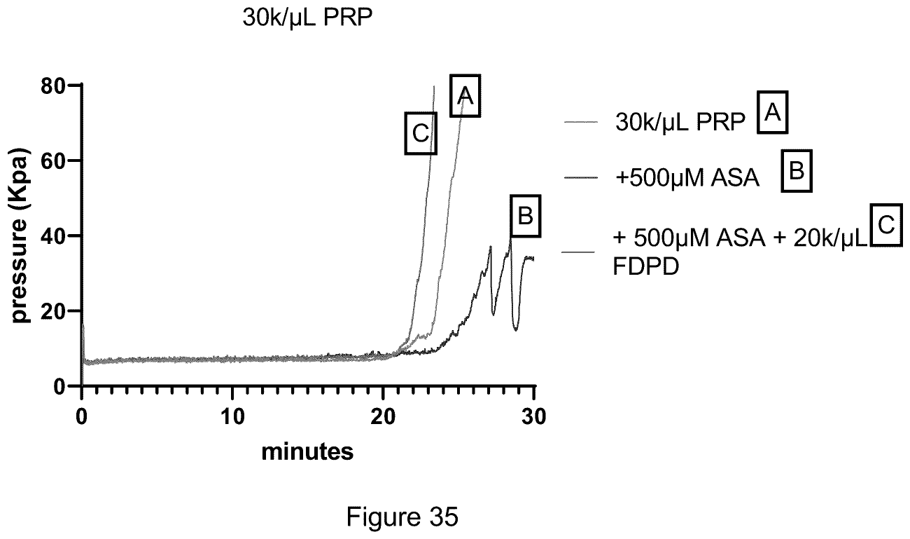

The effect of aspirin on the occlusion time of PRP with and without FDPDs was assessed using a T-TAS assay. The concentrations of the agents are as follows: PRP at 30K/μL, aspirin at 500 μM, and FDPDs at 20 k/μL. The results are shown in FIG. 35, PRP samples showed occlusion of >30 minutes with aspirin treatment versus a PRP alone time of 25:56 minutes; the addition of 20 k/μL FDPDs to P2Y12-inhibited PRP reduced time to thrombus formation to lower than PRP alone; 23:29 minutes in the presence of aspirin (FIG. 35).

These results demonstrate that the occlusion of platelets on the T-TAS® AR Chip in the presence of human FDPDs is unaffected by the antiplatelet effect of aspirin. This suggests that human FDPDS will maintain expected function when infused into patients receiving aspirin and/or similar agents.

### Example 26. Reproducibly of FDPD Reversal of Ticagrelor and Aspirin (ASA) Combined Inhibition of Occlusion

Human freeze-dried platelet derivatives (FDPDs) were prepared according to the procedure in Example 18. T-TAS® experiments using AR chips were carried out according to Example 3.

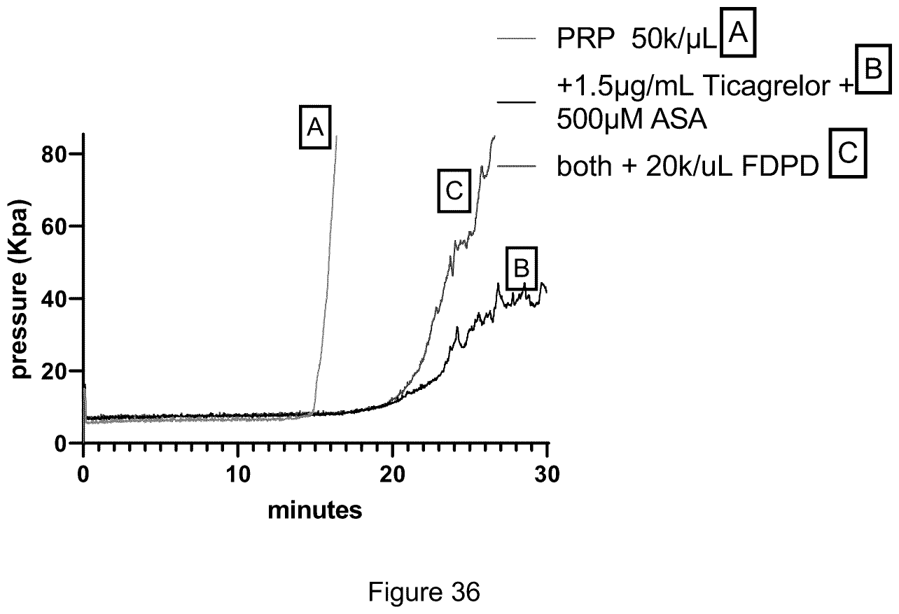

The effect of ticagrelor and aspirin together on the occlusion time of PRP with and without FDPDs was assessed using a T-TAS assay. The concentrations of the agents are as follows: PRP at 50K/μL, ticagrelor at 1.5 μg/mL, aspirin at 500 μM, and FDPDs at 50 k/μL. The results are shown in FIG. 36. The addition of 20 k/μL FDPDs to the combined ticagrelor and aspirin treated PRP reduced time to thrombus formation to 19:36 minutes versus a >30 minutes occlusion time in the presence of combined ticagrelor and aspirin alone.

These results demonstrate that the occlusion effect of platelets on the T-TAS® AR Chip in the presence of human FDPDs unaffected by the combined antiplatelet effect of ticagrelor and aspirin. This suggests that human FDPDS will maintain expected function when infused into patients receiving combined ticagrelor and aspirin treatment.

### Example 27. Inability of FDPDs to Aggregate in the Presence of Agonists and Absence of Fresh Platelets

Light transmission aggregometry (LTA) was used to observe FDPD aggregation in the presence of known platelet aggregation agonists. The FDPD aggregation data was compared to aggregation data of fresh platelets.

FDPDs, also referred as “TFF-FDPDs”, were produced by the TFF method described in Example 18. Fresh platelets in Platelet Rich Plasma (PRP) were prepared from whole blood collected in acid-citrate-dextrose (ACD) collection tubes (BD Vacutainer ACD Solution A Blood Collection Tubes ref #364606). Platelet rich plasma (PRP) was prepared by centrifugation of ACD-whole-blood at 180 g for 15 minutes at 22° C. using a Beckman Coulter Avanti J-15R centrifuge. Platelet poor plasma (PPP) was prepared by centrifugation of ACD-whole-blood at 2000 g for 20 minutes at 22° C.

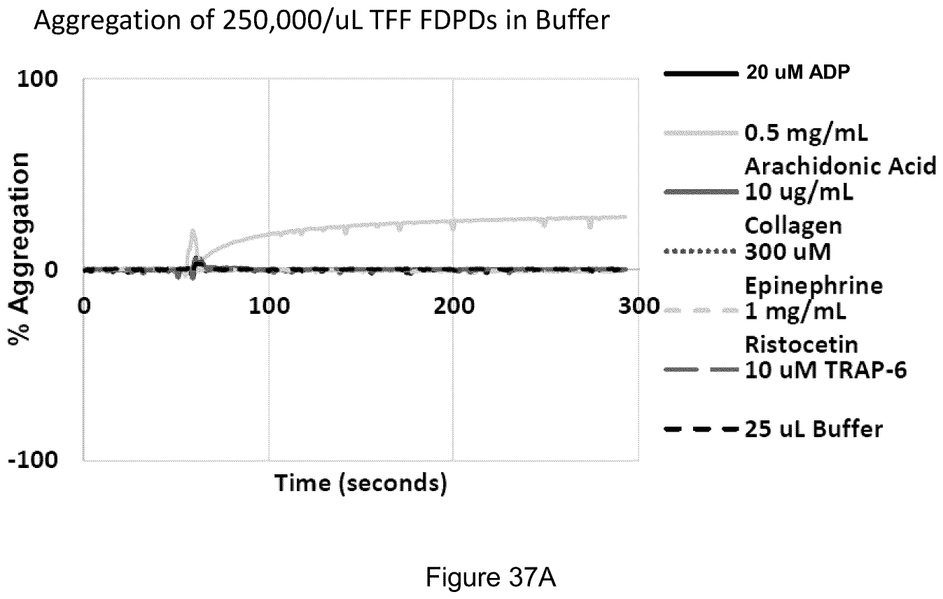

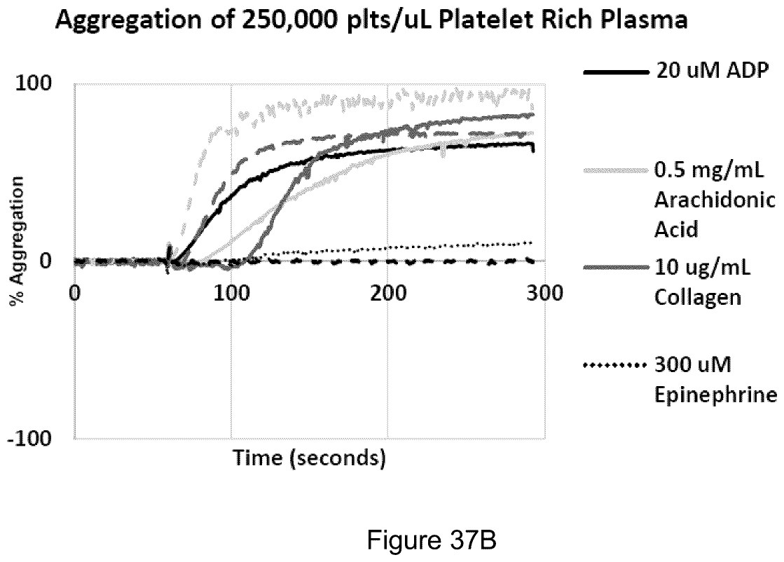

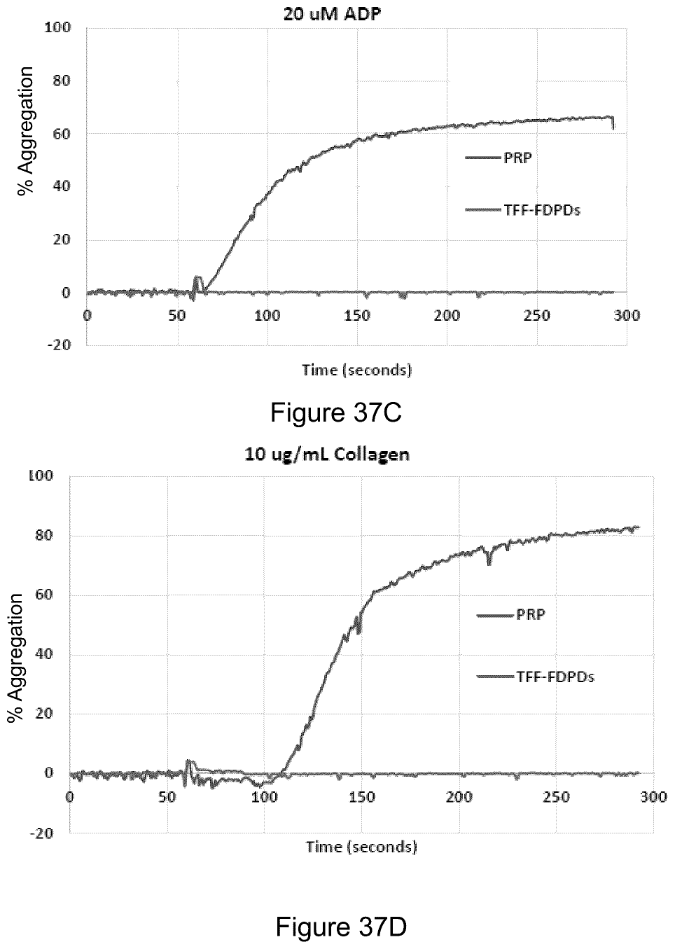

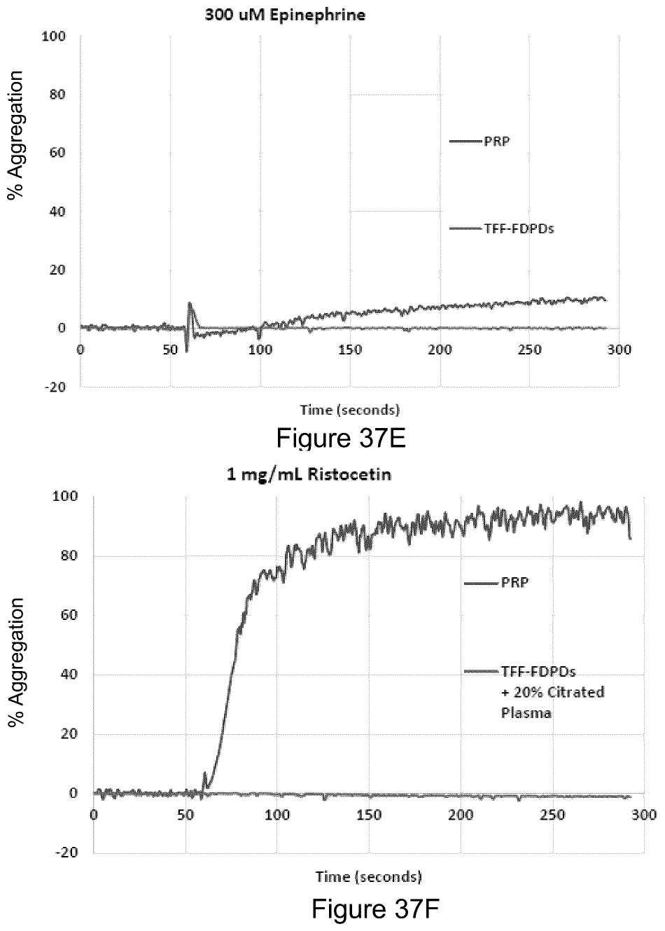

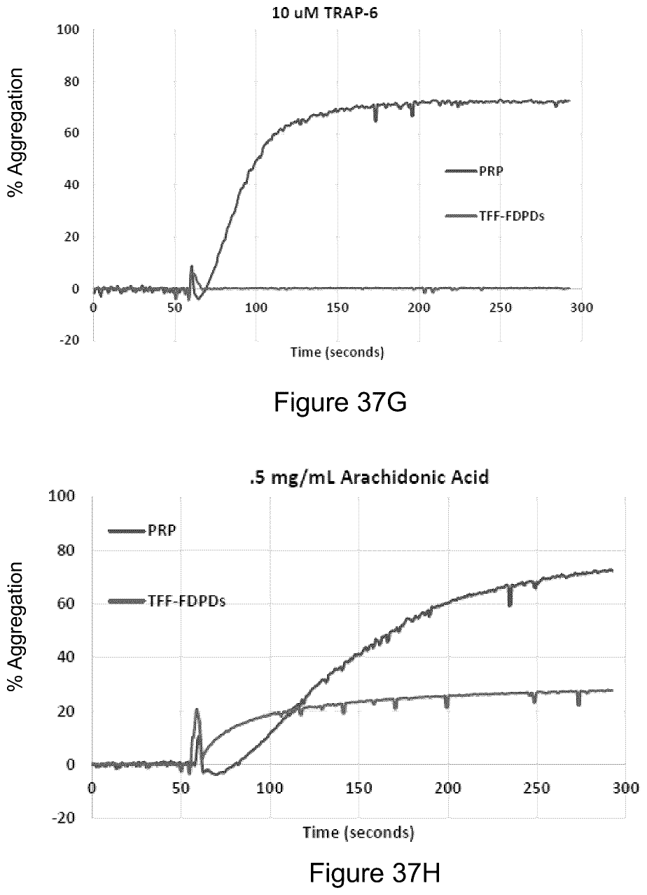

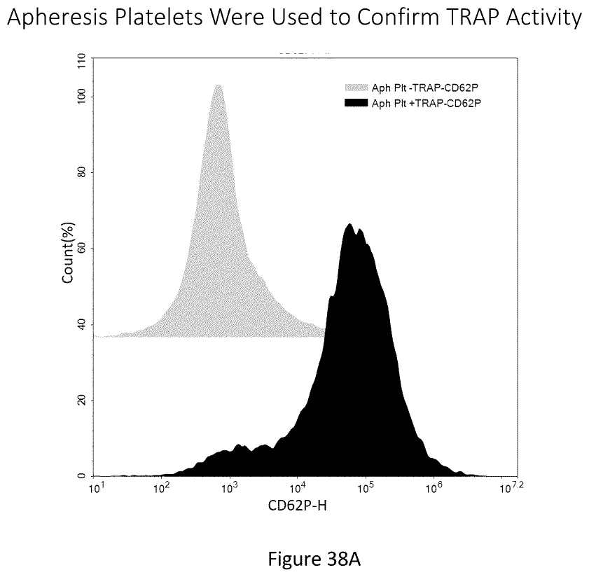

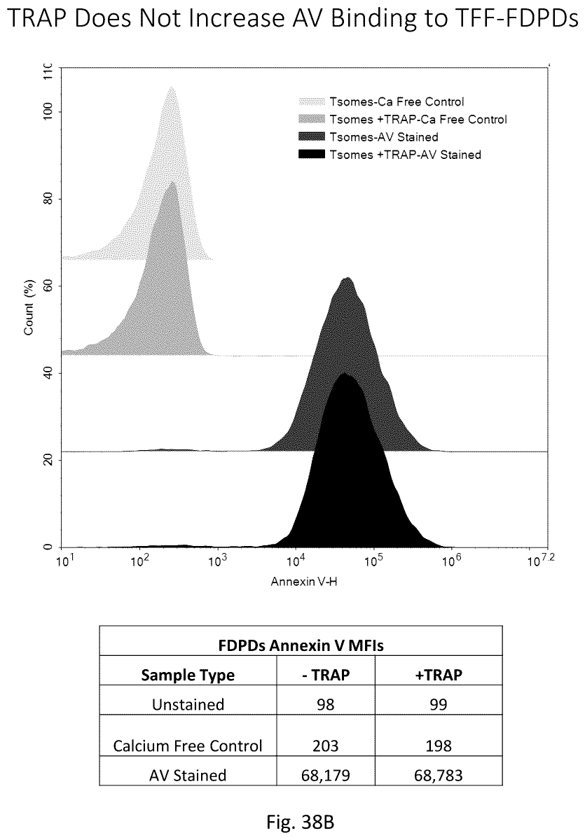

For sample preparation for aggregometry studies, PRP was diluted with PPP to a platelet concentration (plt count) of 250,000 plts/μL. Platelet count was determined using a Coulter Ac·T diff2 Hematology Analyzer. TFF FDPDs, lyophilized and thermally treated, were prepared using tangential flow filtration as described in Example 18. A 30 mL vial of FDPDs was rehydrated using 30 mL of cell culture grade water (Corning Cat #25-055-CI). The vial was incubated at room temperature for a total of 10 minutes. During the 10-minute rehydration period, the vial was gently swirled at 0, 5, and 10 minutes to promote dissolution of the lyophilizate. The aggregometry studies as per the present Example was carried out in the absence of fresh platelets. Therefore, the aggregometry studies supported only aggregation ability of the FDPDs, but not the co-aggregation ability. For sample preparation for aggregometry studies, rehydrated FDPDs were diluted in a buffer to a platelet count of 250,000/μL. FDPDs sample preparations used for ristocetin aggregation studies were composed of 20% citrated plasma (George King Bio-Medical, Inc. Pooled Normal Plasma product #0010-1) and buffer. Light transmission aggregometry (LTA) (Bio/Data PAP-8E Platelet Aggregometer catalog #106075) at 37° C. was used to observe the aggregation response of FDPDs (FIG. 37A) and PRP samples (FIG. 37B) from a final concentration of 20 μM ADP, 10 μg/mL collagen, 200 μM epinephrine (ADP, collagen, and epinephrine reagents from Helena Laboratories Platelet Aggregation Kit cat. #5369), 0.5 mg/mL arachidonic acid (Helena Arachidonic Acid Reagent cat.), 1 mg/mL ristocetin (Helena Ristocetin for Aggregation Assays cat.), and 10 μM thrombin receptor activator peptide 6 (TRAP-6) (Sigma Aldrich Cat #T1573-5 MG). PPP, buffer, or buffer with 20% citrated plasma were used as blanks for the PRP, FDPDs, and FDPDs with 20% citrated plasma samples, respectively. Prior to agonist treatment, 225 μL of FDPDs or PRP sample was reverse pipetted in a test tube containing a stir bar. The test tube was then placed into the aggregometer's non-stirred incubation well for 1 minute. The sample was then placed into a stirred incubation well for 1 minute. The sample was then placed into the stirred test well and the aggregation test was initiated. After 1-minute of baseline observation the sample was treated with agonist and the aggregation response was recorded. Using the same procedure as the test runs, a negative control of 25 μL buffer was included simultaneously with all runs to determine spontaneous baseline-aggregation responses of all sample groups.

FDPD sample preparations in 1.7 mL microcentrifuge tubes, at room temperature, were treated with an agonist at a final agonist concentration of 20 μM ADP, 0.5 mg/mL arachidonic acid, 10 μg/mL collagen, 200 μM epinephrine, 1 mg/mL ristocetin, and 10 μM TRAP-6 or 25 μL buffer. FDPD counts were determined prior to and 5-minutes after agonist treatment. ADP (FIG. 37C), collagen (FIG. 37D), epinephrine (FIG. 37E), ristocetin (FIG. 37F), and TRAP-6 (FIG. 37G) did not cause an aggregation response in TFF FDPDs when measured by LTA. TFF FDPDs' response from the aforementioned agonists was equivalent to baseline aggregation values that would be obtained from no agonist or a negative control of buffer. When TFF FDPDs were treated with arachidonic acid (AA) and observed by LTA (FIG. 37H) there was an apparent aggregation response, however after visual inspection of the aggregometry cuvette it was observed that the solution had become visibly clear and aggregates were not observed, indicating that the apparent aggregation response was from lysis of FDPDs and not AA induced aggregation. Determining aggregation by cell count for TFF FDPDs produced similar results to the LTA results for all agonists. Agonists' functionality was confirmed by performing LTA on fresh PRP (FIG. 37B). ADP, arachidonic acid, collagen, epinephrine, ristocetin and TRAP-6 caused normal aggregation profiles and magnitudes that are representative of a strong aggregation response in PRP. The aggregation response from epinephrine in PRP was reduced, however epinephrine was still able to elicit an aggregation response that was above baseline aggregation. The negative control of buffer in PRP indicated that the PRP was not activated prior to agonists additions. Visual inspection of the PRP samples after the aggregation tests indicated that no cell lysis had occurred and platelet aggregates were visually observed in the aggregation cuvettes for all agonists, indicating that all aggregations responses were from platelet aggregation. The aggregation percentage of FDPDs and fresh PRP observed in the presence of the afore-mentioned agonists have been captured in Table 9.

TABLE 9

| | | TFF FDPDs % | PRP % |
|---|---|---|---|
| | | Aggregation | Aggregation |
| Agonist | | (n = 3) | (n = 2) |
| 20 | μM ADP | 1% | 66% |
| 0.5 | mg/mL | 28%* | 73% |
| Arachidonic Acid | | | |
| 10 | μg/mL | 2% | 83% |
| Collagen | | | |
| 300 | μM | 1% | 11% |
| Epinephrine | | | |
| 1 | mg/mL | 0% | 98% |
| Ristocetin | | | |
| 10 | μM | 1% | 73% |
| TRAP-6 | | | |
| 25 | μL | 1% | 2% |

Buffer

*Due to lysis of FDPDs and not aggregation

### Example 28. FDPDs are Maximally Activated-Binding of Annexin V to FDPDs in the Presence of TRAP

FDPDs, prepared using the TFF process and treated with TRAP-6, were tested for the presence of phosphatidylserine (PS), indicative of an activated platelet, on the surface of the FDPDs. The presence of PS was assessed by analysis of Annexin V (AV) binding to the FDPDs.

One 30 mL vial of FDPDs prepared using the TFF process as described in the Example 18 was rehydrated using 30 mL of cell culture grade water (Corning Cat #25-055-CI). After water was added to the vial, the vial was incubated for 10 minutes at room temperature. Gentle swirling of the vial was performed every 2 minutes during the 10-minute period to promote full dissolution of the cake. Once the FDPDs were fully rehydrated, two 475 μL aliquots were transferred to two separate 1.7 mL microcentrifuge tubes. Twenty-five microliters of HEPES Modified Tryode's Albumin buffer (HMTA) (Cellphire RGT-004) was added to the sample in the first tube to generate FDPDs without TRAP-6. Twenty-five microliters of 400 μM Thrombin Receptor Activating Peptide 6 (TRAP-6) (Sigma Aldrich Cat #T1573-5 MG) was added to the second tube to generate FDPDs with TRAP-6. The final concentration of TRAP-6 during incubation was 20 μM. Both tubes were inverted 5 times to mix and incubated at room temperature for 10 minutes.

After incubation with HMTA buffer or TRAP-6, the samples were further diluted 1:20 by adding 10 μL of the FDPD sample to 190 μL HMTA. These diluted samples of FDPDs incubated with HMTA and FDPDs incubated with TRAP-6 were both stained in 1.7 mL microcentrifuge tubes as follows: unstained control samples were generated by combining 10 μL of FDPDs and 20 μL HMTA; calcium free control samples were generated by combining 10 μL of FDPDs, 5 μL of Annexin V-ACP (BD Pharmingen Cat #550475), and 15 μL HMTA; Annexin V (AV) stained test samples were generated by combining 10 μL of FDPDs, 5 μL of AV-ACP, and 15 μL HMTA supplemented with 9 mM CaCl₂ (Cellphire RGT-012 Lot #LAB-0047-21). The final concentration of CaCl₂ in the AV-stained test samples was 3 mM. All stained samples for both FDPDs incubated with HMTA and FDPDs incubated with TRAP-6 were generated in triplicate. The samples were incubated at room temperature, protected from light, for 20 minutes.

After incubation, 500 μL of HEPES buffered saline (HBS) (Cellphire RGT-017) was added to all unstained control and calcium free control samples. Five hundred microliters of HBS supplemented with 3 mM CaCl₂ was added to the AV-stained test samples. One hundred microliters from each sample was transferred to an individual well in a 96 well plate, and the samples were analyzed using an Agilent Quanteon flow cytometer.

TRAP-6 activity was confirmed by measuring CD62P expression in human apheresis platelets with and without exposure to TRAP-6. Two 475 μL aliquots of apheresis platelets were transferred to two separate 1.7 mL microcentrifuge tubes. Twenty-five microliters of HMTA buffer was added to the sample in the first tube to generate apheresis platelets without TRAP-6. Twenty-five microliters of 400 μM TRAP-6 was added to the second tube to generate FDPDs with TRAP-6. The final concentration of TRAP-6 during incubation was 20 μM. Both tubes were inverted 5 times to mix and incubated at room temperature for 10 minutes.

After incubation with HMTA buffer or TRAP-6, the samples were further diluted 1:20 by adding 10 μL of apheresis platelets to 190 μL HMTA. These diluted samples of apheresis platelets incubated with HMTA and apheresis platelets incubated with TRAP-6 were both stained in 1.7 mL microcentrifuge tubes as follows: unstained control samples were generated by combining 10 μL of apheresis platelets and 20 μL HMTA; Anti-CD62P stained test samples were generated by combining 10 μL of apheresis platelets, 5 μL of anti-CD62P-PE antibody (BD Pharmingen Cat #550561 Lot #6322976), and 15 μL HMTA. All stained samples for both apheresis platelets incubated with HMTA and apheresis platelets incubated with TRAP-6 were generated in triplicate. The samples were incubated at room temperature, protected from light, for 20 minutes.

After incubation, 500 μL of phosphate buffered saline (PBS) (Corning Cat #21-040-CV1) was added to all samples. One hundred microliters from each sample was transferred to an individual well in a 96 well plate, and the samples were analyzed using an Agilent Quanteon flow cytometer.

FDPDs manufactured using the TFF process were incubated with either TRAP-6 or buffer and stained with Annexin V (AV) to determine the relative presence of phosphatidylserine (PS). Apheresis platelets were used to confirm TRAP-6 activity (FIG. 38A, and Table 10), and increased expression of CD62P after the exposure to TRAP-6 confirms that TRAP-6 is capable of promoting platelet expression. PS expression on the exterior membrane leaflet is a hallmark of platelet activation and increases in membrane expression of PS result in greater amounts of AV binding. Unstained samples and samples stained with AV but without the addition of calcium were analyzed on the flow cytometer as negative controls. Unstained samples generated little to no fluorescent signal, indicating that FDPDs were not auto fluorescent at the wavelength selected to measure AV (FIG. 38B). The calcium free control samples also generated little to no fluorescent signal. Since AV binding to PS is dependent on the presence of calcium ions, a lack of signal from the calcium free control samples demonstrates that the AV-ACP conjugate was not associating with the FDPD membrane in a nonspecific manner. All samples stained with AV in the presence of calcium provided a strong fluorescent signal that was, on average, approximately 695 times brighter than the unstained controls. This result indicates that all FDPDs samples were expressing, or comprised, PS. Incubating the FDPDs with TRAP-6 did not cause a notable increase in AV binding as measured by mean fluorescent intensity (MFI) (FIG. 38B). The average MFI values for FDPDs incubated with buffer and FDPDs incubated with TRAP-6 were 68,179 and 68,783, respectively (Table 11).

TABLE 10
Apheresis Platelet CD62P MFI

| Sample Type | −TRAP | +TRAP |
|---|---|---|
| Unstained | 100 | 107 |
| CD62P Stained | 2,351 | 126,598 |

TABLE 11
FDPD Annexin V MFIs

| Sample Type | −TRAP | +TRAP |
|---|---|---|
| Unstained | 98 | 99 |
| Calcium Free Control | 203 | 198 |
| AV Stained | 68,179 | 68,783 |

FDPDs, manufactured using the TFF process, were shown to contain phosphatidylserine (PS) on the membrane as evident by the binding of Annexin V (AV) to the FDPDs. The binding of AV to activated platelets is a calcium dependent binding and therefore the calcium ion dependency of AV binding to the rehydrated FDPDs provides further support that the AV conjugate was not associating with the membrane of the FDPD in a nonspecific manner.

While TRAP-6 was shown to activate apheresis platelets, as evident by increased CD62P expression, and increased the binding of AV to the activated platelet, it was not the case for the FDPDs. The FDPDs with or without a TRAP-6 incubation exhibited same high level of AV binding, and indicate that TRAP-6 does not promote further surface expression of PS for FDPDs, likely because the FDPDs are maximally activated during the lyophilization and/or rehydration process, and further stimulation/activation is not possible.

### Example 29. Presence of Thrombospondin (TSP1) on the Surface of the FDPDs

Thrombospondin (TSP1), a glycoprotein typically found to coat external membranes of activated platelets, was found to coat FDPDs without activation. The presence of TSP1 was detected by fluorescence of anti-Thrombospondin-1 (TSP-1) antibody.

Fresh platelet rich plasma (PRP) was isolated by centrifuging whole blood collected in acid citrate dextrose (ACD) at 180 g for 10 minutes. Isolated PRP was centrifuged again at 823 g for an additional 10 minutes. The plasma was then removed and discarded, and the platelet pellet was resuspended in HEPES Modified Tyrode's Albumin (HMTA) buffer. An aliquot of the resulting washed platelet sample was activated by incubated the platelets at room temperature for 10 minutes in the presence of 2 mM GPRP peptide (BaChem Cat #H-1998.0025), 2 mM CaCl₂, 0.5 U/mL thrombin (EDM Millipore Cat #605190-1000U), and 0.5 μg/mL collagen (ChronoPar Cat #385). A separate aliquot of washed platelets was set aside to be used as a resting negative control.

All samples of FDPDs were manufactured using the TFF process as described in Example 15. The FDPDs studied in this example were baked FDPDs which were heated after lyophilization at 80° C. for 24 hours. All vials were rehydrated using the appropriate amount of cell culture grade water. After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both the resting and activated fresh washed platelet aliquots, were diluted 1:500 in triplicate using phosphate buffered saline (PBS) (Corning Cat #21-040-CV). The diluted samples were analyzed on the Quanteon flow cytometer and the concentrations of the platelets and FDPDs were determined. Based on these concentrations, an aliquot of each FDPDs or fresh platelet sample was diluted to a concentration of 100,000 FDPDs per microliter.

Stained samples from each vial of FDPDs and the resting and activated fresh platelets were generated by adding 10 μL of diluted FDPDs or platelets to 20 μL of HMTA containing 4 μg/mL of anti-Thrombospondin-1 (TSP-1) antibody (Santa Cruz Biotech Cat #sc-59887 AF594). Unstained control samples were generated by adding 10 μL of diluted FDPDs or platelets to 20 μL of HMTA. All The samples were incubated at room temperature, protected from light, for 20 minutes. After incubation, 500 μL of PBS was added to all samples. One hundred microliters from each sample were transferred to an individual well in a 96 well plate, and the samples were analyzed using an Agilent Quanteon flow cytometer.

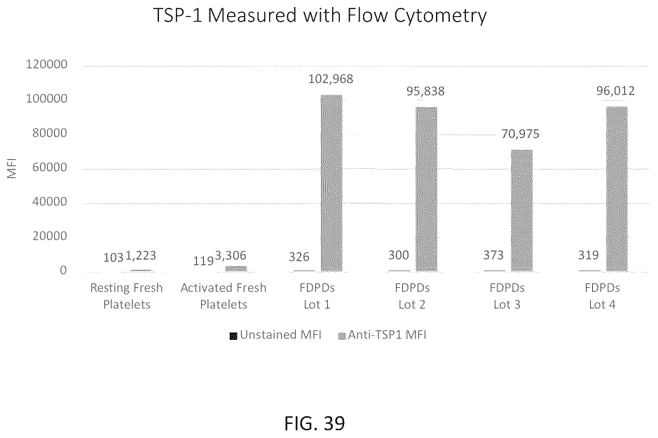

Unstained samples of fresh platelets and FDPDs generated little to no fluorescent signal, indicating that the samples were not auto fluorescent at the wavelength selected to measure TSP-1 expression or presence. Binding of the anti-TSP-1 antibody to fresh platelets increased slightly after activation with collagen and thrombin as shown by an increase in mean fluorescent intensity (MFI) when analyzed using flow cytometry (1,223 vs 3,306). Expression or presence of TSP-1 on FDPD samples varied from lot to lot with an average MFI value of 91,448 (FIG. 39). For all FDPD samples tested, the fluorescent signal was significantly higher than the signal generated by either resting or fresh platelets, indicating high amounts of TSP-1 may be bound to the surface of rehydrated FDPDs. This data suggests that the FDPDs without the requirement of an activation step exhibit properties which in certain embodiments and applications are superior to activated platelet properties.

### Example 30. Presence of Von Willebrand Factor (vWF) on the Surface of the FDPDs

Fresh platelet rich plasma (PRP) was isolated by centrifuging whole blood collected in acid citrate dextrose (ACD) at 180 g for 10 minutes. Isolated PRP was centrifuged again at 823 g for an additional 10 minutes. The plasma was then removed and discarded, and the platelet pellet was resuspended in HEPES Modified Tyrode's Albumin (HMTA) buffer. An aliquot of the resulting washed platelet sample was activated by incubating the platelets at room temperature for 10 minutes in the presence of 2 mM GPRP peptide (BaChem Cat #H-1998.0025), 2 mM CaCl₂, 0.5 U/mL thrombin (EDM Millipore Cat #605190-1000U), and 0.5 μg/mL collagen (ChronoPar Cat #385). A separate aliquot of washed platelets was set aside to be used as a resting negative control. All samples of FDPDs were prepared using the TFF process as described in Example 18. The FDPDs studied in this example were baked FDPDs which were heated after lyophilization at 80° C. for 24 hours. All vials were rehydrated using the appropriate amount of cell culture grade water (Corning Cat #25-055-CI). After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both the resting and activated fresh washed platelet aliquots, were diluted 1:500 in triplicate using phosphate buffered saline (PBS). The diluted samples were analyzed on the Quanteon flow cytometer and the concentrations were determined. Based on these concentrations, an aliquot of each FDPDs or fresh platelet sample was diluted to a concentration of 100,000 FDPDs per microliter.

Prior to staining, the anti-Von Willebrand Factor antibody (Novus Biologicals Cat #NBP2-54379PE) was diluted by a factor of 10. Stained samples from each vial of FDPDs and the resting and activated fresh platelets were generated by adding 10 μL of diluted FDPDs or platelets to 10 μL of diluted antibody and 10 μL of HMTA. Unstained control samples were generated by adding 10 μL of diluted FDPDs or platelets to 20 μL of HMTA. All The samples were incubated at room temperature, protected from light, for 20 minutes. After incubation, 500 μL of PBS was added to all samples. One hundred microliters from each sample was transferred to an individual well in a 96 well plate, and the samples were analyzed using an Agilent Quanteon flow cytometer.

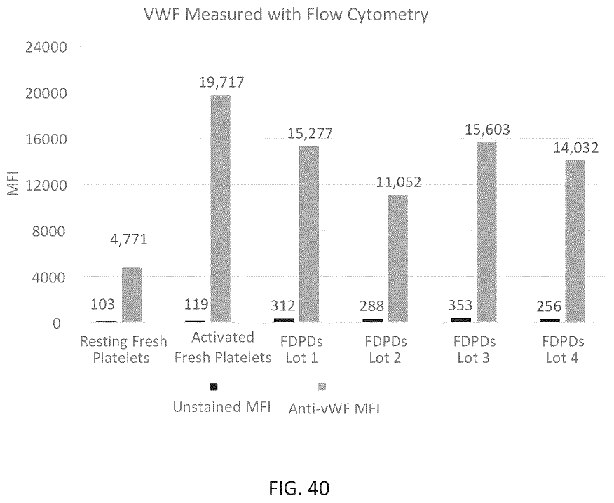

Unstained samples of fresh platelets and FDPDs generated little to no fluorescent signal, indicating that the samples were not auto fluorescent at the wavelength selected to measure vWF expression or presence. Binding of the anti-vWF antibody to fresh platelets increased after activation with collagen and thrombin as shown by an increase in mean fluorescent intensity (MFI) when analyzed using flow cytometry (4,771 vs 19,717). Expression or presence of vWF on FDPD samples varied from lot to lot with an average MFI value of 13,991 (FIG. 40). For all FDPD samples tested, the fluorescent signal fell between the signals generated by resting and activated platelets. This suggests that vWF is present on the surface of rehydrated FDPDs, and that the amount of vWF present is greater than that seen on resting platelets. The data suggests that even in the absence of any activation, the FDPDs exhibit properties that is superior to resting platelets and similar to the activated platelets.

### Example 31. FDPDs-Compromised Membrane

Membrane integrity of FDPDs, either heated at 80° C. for 24 hours (baked FDPDs) or not heated (unbaked FDPDs) after lyophilization, was tested. The baked and unbaked FDPDs of the standard formulation were analyzed by forward scatter against pre-lyophilization material and by the use of an antibody against a stable intracellular antigen, β-tubulin, to determine if FDPDs were permeable to IgGs (150 kDa). Forward scatter is a flow cytometry measurement of laser scatter along the path of the laser. Forward scatter (FSC) is commonly used as an indication of cell size as larger cells will produce more scattered light. However, forward scatter also can indicate the membrane integrity of the sample via optical density (i.e., light transmission); a cell with less cytosolic material and a porous membrane would transmit more light (have a lower FSC) than the same cell if intact, despite being the same size.

The FDPDs of Example 18 were studied to determine if FDPDs were permeable to IgGs (150 kDa) by the use of an antibody against a stable intracellular antigen, β-tubulin. Fresh platelets, unbaked FDPDs, and baked FDPDs were fixed and stained with anti-β tubulin IgG with and without cell permeabilization. Fresh platelets showed a dramatic increase in IgG binding with permeabilization, whereas both baked and unbaked FDPDs showed no change in response to permeabilization (Table 12). Results from fresh platelets and FDPDs that were fixed and then either permeabilized with 0.2% Triton-X 100 or not permeabilized and then stained with anti-β tubulin IgG conjugated to the fluorophore AF594. Unstained samples are included for background fluorescence.

TABLE 12

| | Mean | AF594 |
|---|---|---|
| Sample | FSC-H | MFI |
| Platelets Unstained | 120,301 | 115 |
| Platelets | 118,782 | 636 |
| Permeabilized Platelets | 49,062 | 9,009 |
| Unbaked FDPDs Unstained | 23,140 | 75 |
| Unbaked FDPDs | 23,280 | 546 |
| Permeabilized Unbaked FDPDs | 7,069 | 562 |
| Baked FDPDs Unstained | 49,740 | 362 |
| Baked FDPDs | 49,587 | 2,720 |
| Permeabilized Baked FDPDs | 27,527 | 2,523 |

The IgG binding studies suggest that the membrane integrity of FDPDs is severely impaired such that large molecules can pass through the cell membrane. Of additional note, permeabilization induced decreases in forward scatter value, corroborating the proposed relationship between membrane integrity and optical density for particles of the same size.

Additionally, the mean intensity of forward light scattering of FDPDs prepared by TFF method as described in Example 18 was compared to in-date human platelet apheresis units. The method is as described below.

All samples of FDPDs were manufactured using the TFF process. All vials were rehydrated using the appropriate amount of cell culture grade water (Corning Cat #25-055-CI). After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both in-date human platelet apheresis units, were diluted 1:500 in triplicate using phosphate buffered saline (PBS) (Corning Cat #21-040-CV). The diluted samples were acquired on the Quanteon flow cytometer and the concentrations were determined. Based on these concentrations, an aliquot of each FDPDs or apheresis platelet sample was diluted to a concentration of 100,000 FDPDs per microliter in HEPES Modified Tyrode's Albumin (HMTA) buffer (Cellphire RGT-004).

Unstained samples of FDPDs and human apheresis platelets containing 10⁶ total cells in HMTA were diluted with 500 μL of PB. One hundred microliters from each sample were transferred to an individual well in a 96 well plate, and the samples were analyzed using an Agilent Quanteon flow cytometer.

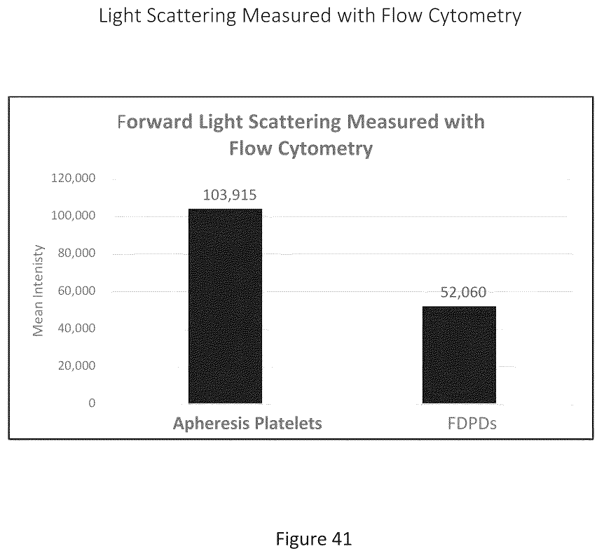

The mean intensity is depicted in FIG. 41. It can be observed that the mean intensity of forward light scattering measured with flow cytometry is distinctly lower (about 50%) for FDPDs as compared to the apheresis plasma. Therefore, corroborating with the previous result of Table 12 that a cell with less cytosolic material and a porous membrane would transmit more light (have a lower FSC) than the same cell if intact, despite being the same size.

The overall results suggest that membrane integrity is substantially degraded in FDPDs; the platelet intracellular contents have been released (e.g. LDH) and large molecules can enter the cellular cytosol (e.g. anti β-tubulin IgG). The plasma membrane of FDPDs is likely damaged by the drying (sublimation) or rehydration processes as freezing in cryopreserved platelets appears to be insufficient to induce severe membrane dysfunction. These results also imply that signal transduction from the outside of the cell is not possible in FDPDs, which is corroborated by lack of aggregation response (as observed in Example 27). Baking, although it produced an increase in optical density, did not appear to improve membrane integrity significantly (e.g., IgG β-tubulin binding). The results discussed in the present example thus show that the platelet derivatives as disclosed herein have a compromised plasma membrane.

### Example 32. Surface Markers and Thrombin Generation

FDPDs batch were produced by the TFF method described in Example 18 and assayed for cell surface marker expression or presence or absence using flow cytometry.

Flow cytometry was used to assess FDPDs for expression or presence or presence of CD41, CD62, and phosphatidylserine (PS). Samples included approximately 270,000/μL FDPDs during staining and were diluted approximately 1:34 before the sample was analyzed in the cytometer. FDPD samples were rehydrated and diluted 1:2 in deionized water. A stock of anti-CD41 was diluted by adding 47.6 μL of antibody to 52.4 μL of HMTA. Samples stained with anti-CD41 were made by adding 10 μL of diluted FDPDs to 10 μL HMTA and 10 μL of diluted CD41 antibody. An anti-CD62 master mix was prepared by combining 12 μL anti-CD62 with 23.8 μL anti-CD41 and 64.2 μL of HMTA. An isotype control mix was made in the same manner. Samples stained with anti-CD62 were made by adding 10 μL of diluted FDPDs to 20 μL of the anti-CD62 master. The isotype master mix was used to make isotype control samples in the same manner. An annexin V (AV) master mix was prepared by combining 11.7 μL of AV with 83.3 μL of anti-CD41 and 80 μL of HMTA. Sample stained with AV were made by adding 20 μL of diluted FDPDs containing 50 mM GPRP to 20 μL of HMTA containing 15 mM CaCl₂ and 20 μL of the AV master mix. Negative gating control samples were made in the same manner using HMTA without calcium to prevent AV binding to PS. All samples were incubated at room temperature for 20 minutes. After incubation 1 mL HBS was added to all samples. HBS used to dilute AV test samples contained 5 mM CaCl₂. Anti-CD41 binding was used to identify the population of interest. CD62 and PS expression or presence was assessed by anti-CD62 and AV binding within the CD41 positive population.

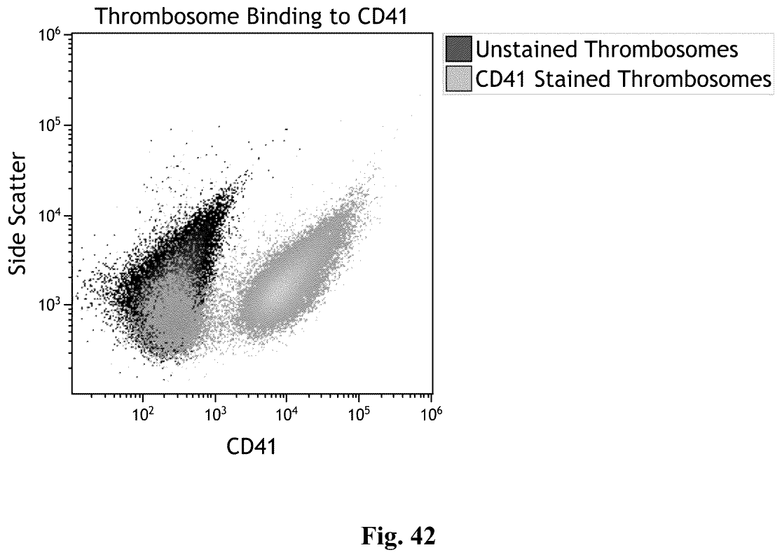

Glycoprotein IIb (GPIIb, also known as antigen CD41) expression or presence was assayed using an anti-CD41 antibody (4.8 μL, Beckman Coulter part #IM1416U). The assayed FDPDs demonstrated CD41 positivity (Table 13; FIG. 42)

TABLE 13

| | CD41 |
|---|---|
| | Positivity |
| Batch | (%) |
| 1 | 81.5 |
| 2 | 79.4 |
| 3 | 85.7 |
| 4 | 78.2 |
| 5 | 81.5 |
| 6 | 84.0 |
| 7 | 78.5 |
| Mean | 81.3 |

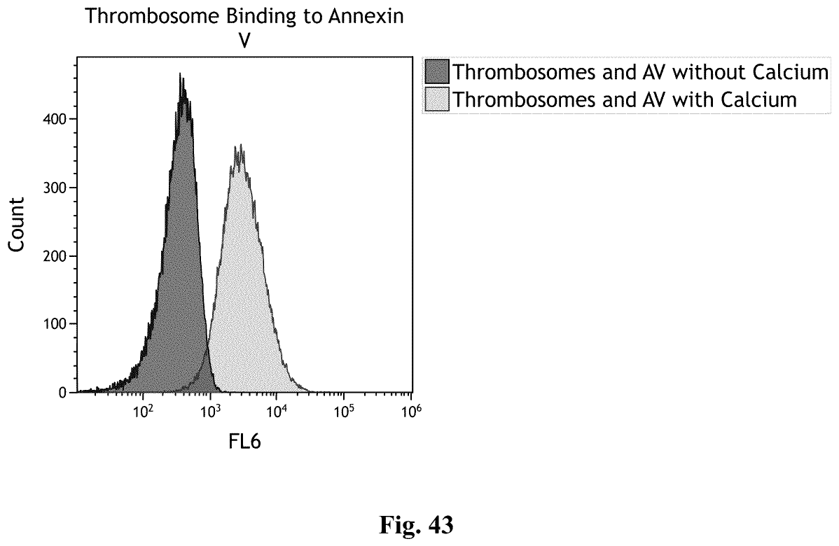

Phosphatidylserine (PS) expression or presence was assayed using annexin V (AV) (1.3 μL, BD Biosciences Cat. No. 550475). AV is a calcium-dependent phospholipid binding protein. The assayed FDPDs demonstrated AV positivity (Table 14; FIG. 43).

TABLE 14

| | AV |
|---|---|
| | Positivity |
| Batch | (%) |
| 1 | 96.7 |
| 2 | 89.9 |
| 3 | 95.3 |
| 4 | 95.4 |
| 5 | 95.9 |
| 6 | 96.2 |
| 7 | 93.5 |
| Mean | 94.7 |

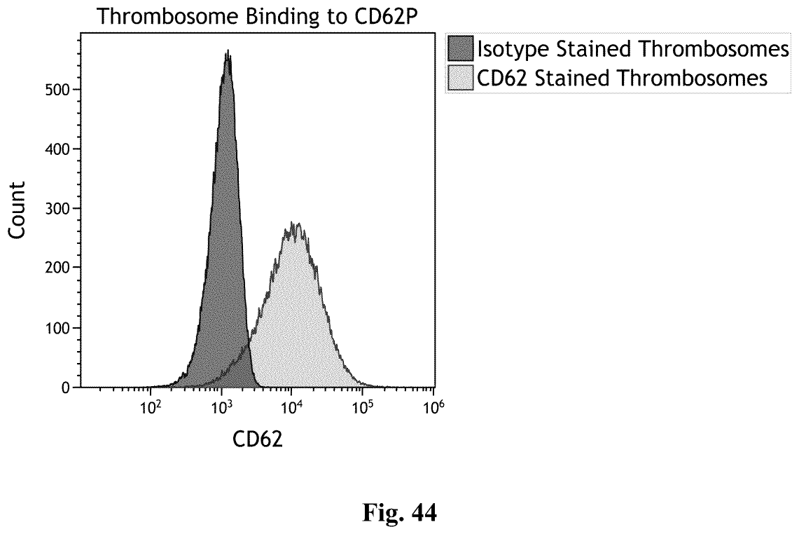

P-selectin (also called CD62P) expression or presence was assayed using an anti-CD62P antibody (2.4 μL, BD Biosciences Cat. No. 550888). The assayed FDPDs demonstrated CD62 positivity (Table 15, FIG. 44)

TABLE 15

| | CD62 |
|---|---|
| | Positivity |
| Batch | (%) |
| 1 | 94.2 |
| 2 | 93.1 |
| 3 | 89.8 |
| 4 | 92.4 |
| 5 | 92.5 |
| 6 | 87.3 |
| 7 | 90.7 |
| Mean | 91.4 |

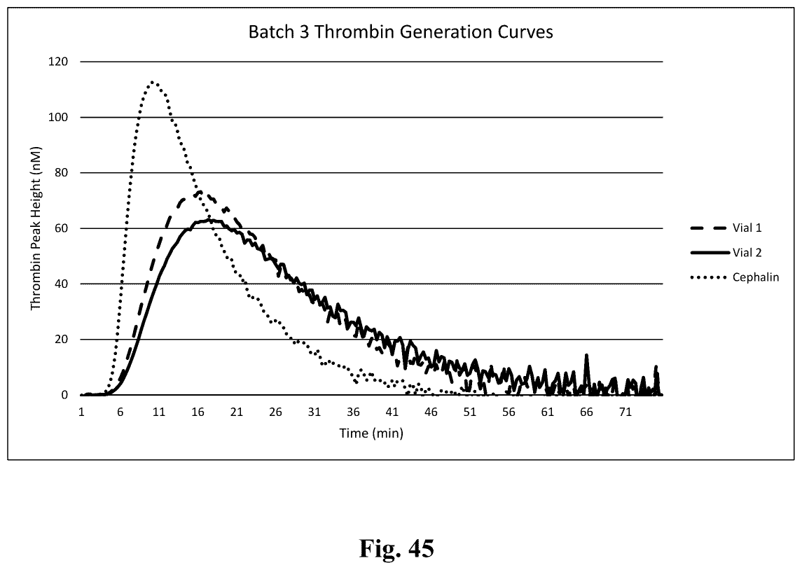

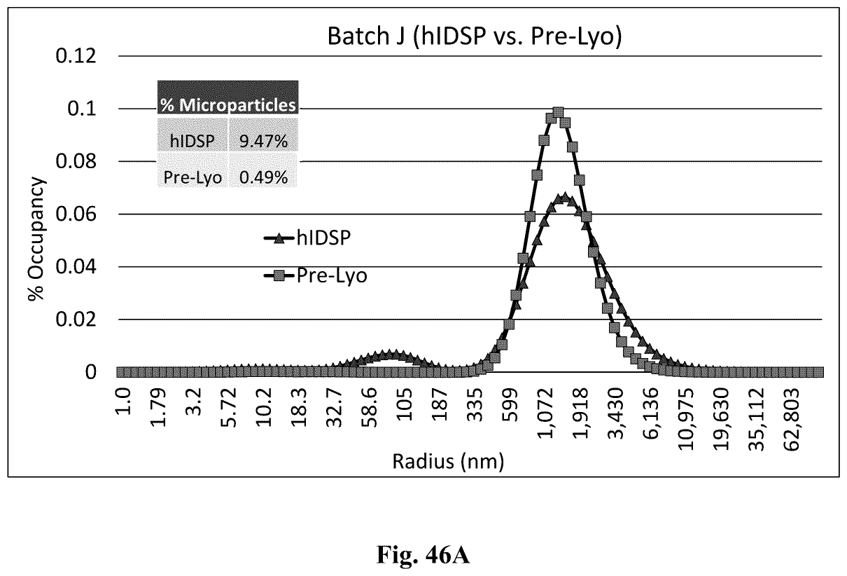

Thrombin generation was measured at 4.8×10³ FDPDs/μl in the presence of PRP Reagent containing tissue factor and phospholipids using the below protocol. On average, the Thrombin Peak Height (TPH) for a FDPDs sample was 60.3 nM. Cephalin was used as a positive control. (Table 16; FIG. 45)

For each vial tested, a rehydrated sample of FDPDs was diluted to 7,200 particles per μL based on the flow cytometry particle count using 30% solution of Octaplas in control buffer. In a 96 well plate, sample wells were generated by adding 20 μL of PRP reagent (Diagnostica Stago Catalog No. 86196) and 80 μL of diluted FDPDs. Calibrator wells were generated by adding 20 μL of Thrombin Calibrator reagent (Diagnostica Stago Catalog No. 86197) to 80 μL of diluted FDPDs. The plate was loaded into the plate reader and incubated in the dark at 40° C. for 10 minutes. During sample incubation, FluCa solution was prepared by adding 40 μL of FluCa substrate (Diagnostica Stago Catalog No. 86197) to 1.6 mL of Fluo-Buffer (Diagnostica Stago Catalog No. 86197) warmed to 37° C. and vortexed to mix. The FluCa solution was aspirated in to the dispensing syringe and 20 μL was mechanically dispensed in to each reaction well, bringing the final FDPDs concentration in each well to 4,800 particles per μL and starting the thrombin generation reaction. Thrombin generation was measured via fluorescence in each well over the course of 75 minutes.

An exemplary step-by-step protocol follows:

Open CAT software; set up instrument; and prepare PRP reagent (including Tissue Factor and some phospholipids), calibrator, and fluo-buffer and fluo-substrate according to manufacturer guidelines.

Thaw Octaplas and TGA dilution buffer in 37° C. water bath for 10 minutes.

Add thawed Octaplas to TGA dilution buffer to create a buffer containing 30% Octaplas.

Use the 30% Octaplas mix to dilute reconstituted cephalin 1:50 to be used as a positive control.

Rehydrate FDPDs with cell culture grade water for 10 minutes then dilute with 30% Octaplas to 7,200 FDPDs/μL.

Using a multichannel pipette, add 20 μL of PRP reagent to each test well. Add 20 μL of Calibrator to each calibration well.

Add 80 μL of sample to each test and calibration well. Add 80 μL of 30% Octaplas to negative control wells and 1:50 cephalin to positive control wells.

Insert plate into tray and incubate for 10 minutes at 40° C. After incubation, dispense fluo-buffer and fluo-substrate mixture (including a fluorescent-labeled peptide, that when cleaved by thrombin, generates a fluorescent signal) into active wells.

Read plate for 75 minutes at 20 s intervals to capture full thrombin generation profile.

TABLE 16

| Batch | TPH (nM) |
|---|---|
| 1 | 61.5 |
| 2 | 71.4 |
| 3 | 67.8 |
| 4 | 52.0 |
| 5 | 60.2 |
| 6 | 54.7 |
| 7 | 54.4 |
| Mean | 60.3 |

Data from these assays is summarized in Table 17.

TABLE 17
TFF Batches

| | | | Average AV | Average CD62 |
|---|---|---|---|---|
| | Average | Average | Positivity | Positivity |
| | TPH | CD41 | (0.5 μm- | (0.5 μm- |
| Batch | (nM) | Positivity | 2.5 μm)¹ | 2.5 μm)¹ |
| Batch B | 61.5 | 81.5 | 96.7 | 94.2 |
| Batch C | 71.4 | 79.4 | 89.9 | 93.1 |
| Batch D | 67.8 | 85.7 | 95.3 | 89.8 |
| Batch E | 52.0 | 78.2 | 95.4 | 92.4 |
| Batch F | 60.2 | 81.5 | 95.9 | 92.5 |
| Batch G | 54.7 | 84.0 | 96.2 | 87.3 |
| Batch H | 54.4 | 78.5 | 93.5 | 90.7 |
| Mean | 60.3 | 81.3 | 94.7 | 91.4 |

¹Particle diameter as assessed using sizing beats on the flow cytometry forward scatter.

### Example 33. Microparticle Content Reduction

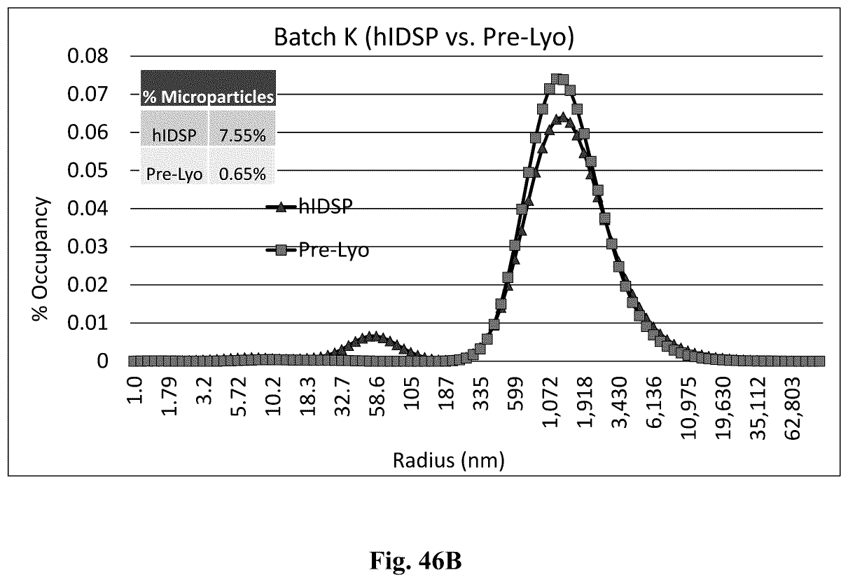

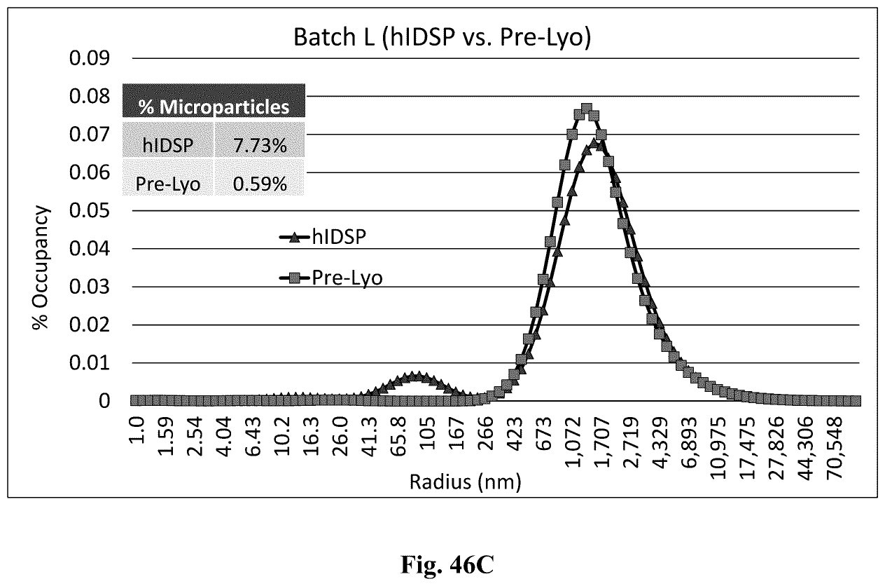

The microparticle content of human in-date stored platelets (hIDSP) compared to FDPDs prepared according to Example 18 (but not lyophilized) were compared using dynamic light scattering. The results are shown in FIGS. 46A-C and Table 18. FIGS. 46A-C are histograms that are normalized to a relative intensity so that the sum of the intensity of each data point equals 1.0. For example, if a particular data point has a y-axis value of 0.1 then it can be typically interpreted that the data point makes up 10% of the scattering intensity of the sample.

A pool of the apheresis units used to manufacture a batch of FDPDs was made for analysis. This sample type is denoted as “hIDSP.” A 1 mL aliquot of this hIDSP (human In-Date Stored Platelets) pool was taken for dynamic light scattering (DLS; Thrombolux—Light Integra) analysis. A sample from this aliquot was then drawn into a capillary and inserted into the DLS instrument. The capillary sat in the instrument for 1 minute to allow the temperature and movement to equilibrate. The internal temperature of the machine is 37° C. After 1 minute of equilibration, the viscosity setting for the sample was chosen. The DLS instrument has a built-in viscosity setting for samples that are in plasma, such as apheresis units. This viscosity setting was used for hIDSP samples. The viscosity of this setting is 1.060 cP (centipoise). After the plasma viscosity setting was selected, the sample was analyzed. From the same hIDSP aliquot, a 2^{nd} and 3^{rd} sample were drawn into a capillary and analyzed with this hIDSP protocol, for triplicate analysis. Microparticle percentage was then determined from the data. “Pre-Lyo” samples are an in-process sample from the FDPDs manufacturing process. This sample type is the material taken right before lyophilization. A viscosity measurement of the sample was taken in order to analysis these samples with DLS. The viscometer (Rheosense μVISC) has a built-in oven that is used to bring the sample to the temperature of the DLS instrument (37° C.). Prior to viscosity analysis of the sample the oven must be heated to 37° C. To determine the viscosity of the pre-lyo sample a 400-350 μL sample was drawn into a syringe and inserted into the viscometer. After inserting the sample into the viscometer, the instrument temperature needs to reach 37° C. again. After the oven reaches 37° C. the sample was analyzed with all settings on AUTO except for “Measurement Volume” which was set to 400 μL. This viscosity was used for the DLS measurement of the same sample. A 1 mL aliquot of this pre-lyo sample was taken for dynamic light scattering (DLS; Thrombolux—LightIntegra) analysis. A sample from this aliquot was then drawn into a capillary and inserted into the DLS instrument. The capillary sat in the instrument for 1 minute to allow the temperature and movement to equilibrate. The internal temperature of the machine is 37° C. After 1 minute of equilibration, the previously measured viscosity was put into the viscosity setting of the DLS instrument. After the viscosity was entered, the sample was analyzed. From the same pre-lyo aliquot, a 2^{nd} and 3^{rd} sample were drawn into a capillary and analyzed with this Pre-Lyo Protocol, for triplicate analysis. Microparticle percentage was then determined from the data.

FDPDs were rehydrated according to standard protocol and diluted 1:5 in a mixture of SeraSub (CST Technologies, Inc.) and ACD. The SeraSub/ACD diluent consists of a 1:9 dilution of ACD in SeraSub. 1 mL of the 1:5 dilution of FDPDs was prepared for analysis by DLS. A sample of the FDPDs dilution was drawn into the capillary and inserted into the DLS instrument. The capillary sat in the instrument for 1 minute to allow the temperature and movement to equilibrate. The internal temperature of the machine is 37° C. After 1 minute of equilibration, the viscosity setting for the sample was chosen. The viscosity used for the sample was 1.200 cP. After the viscosity was entered, the sample was analyzed. A 2^{nd}, 3^{rd}, and 4^{th} sample were drawn into a capillary and analyzed with this FDPDs protocol, for quadruplicate analysis. Microparticle percentage was then determined from the data (and platelet radius where applicable).

TABLE 18

| Batch | hIDSP | Pre-Lyo |
|---|---|---|
| Number | % MP | % MP |
| Batch J | 9.47% | 0.49% |
| Batch K | 7.55% | 0.65% |
| Batch L | 7.73% | 0.59% |
| Average | 8.25% | 0.58% |

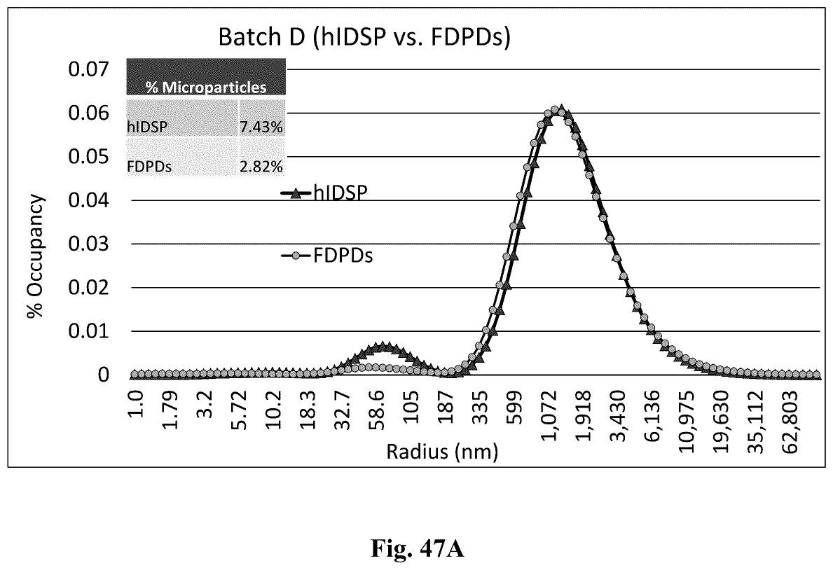

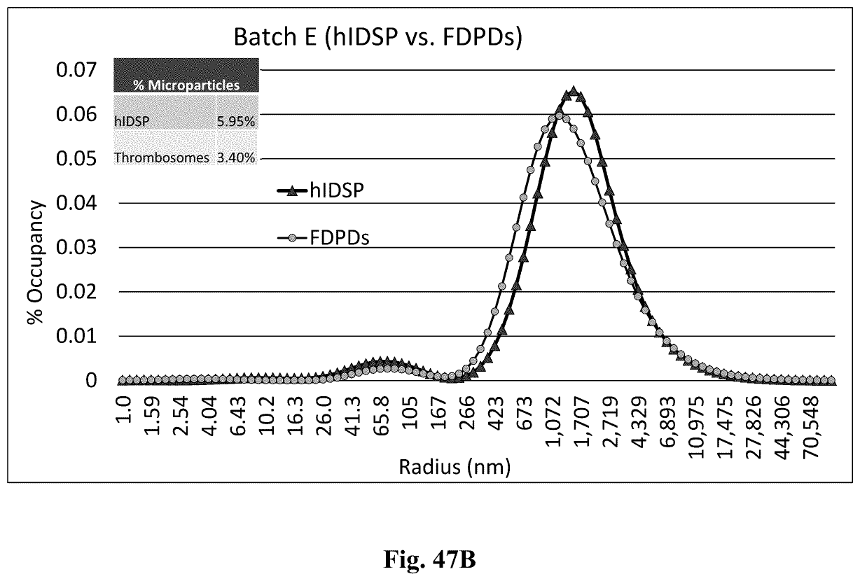

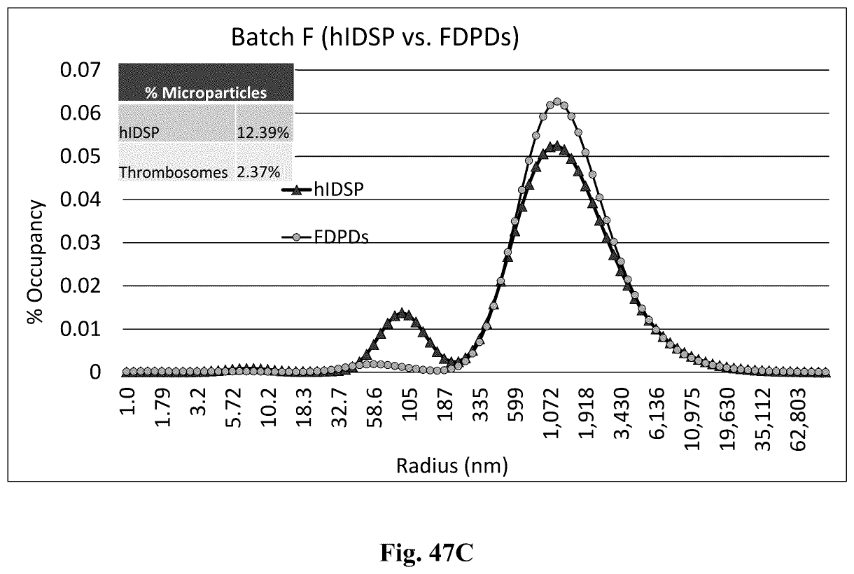

In additional experiments, the microparticle content of human in-date stored platelets (hIDSP) compared to rehydrated FDPDs prepared according to Example 18 were compared using dynamic light scattering (DLS). The results are shown in FIGS. 47A-C and Table 19.

TABLE 19

| Batch | hIDSP | FDPDs |
|---|---|---|
| Number | % MP | % MP |
| Batch D | 7.43% | 2.82% |
| Batch E | 5.95% | 3.40% |
| Batch F | 12.39% | 2.37% |
| Average | 8.59% | 2.86% |

### Example 34. 9F9 and PAC-1 Binding

Aggregation of activated platelets is mediated by the formation of the GPIIb/IIIa complex, which can bind to fibrinogen (also called Factor 1) and form a clot. GPIIb/IIIa is a platelet fibrinogen receptor also known as CD41/CD61 complex. In this process, ADP promotes the active form of the GPIIb/IIIa complex. Antibody 9F9 binds to fibrinogen associated with the cell membrane. The presence of fibrinogen on the cell membrane is thus indicative of FDPDs capable of forming clots.

A vial of FDPDs prepared according to Example 18 was rehydrated using 10 mL of deionized water. An aliquot of FDPDs was diluted to a final concentration of 1×10⁵ particles/μL using HMTA (HEPES Modified Tyrode's Albumin). Samples were prepared as shown in Table 20. Unstained samples were prepared by adding 10 μL of diluted FDPDs to 20 μL of HMTA. FITC isotype control samples were prepared by adding 10 μL of diluted FDPDs to 10 μL of the isotype control antibody (BD Biosciences Cat. No. 555748) and 10 μL of HMTA. Samples stained with 9F9 were prepared by adding 10 μL of diluted FDPDs to 10 μL of the 9F9 antibody (BD Biosciences Cat. No. 340507 and 10 μL of HMTA. Samples stained with PAC-1 were prepared by adding 10 μL of diluted FDPDs to 5 μL of the isotype control antibody and 15 μL of HMTA. All samples were prepared in duplicated using a total of 1×10⁶ particles per reaction mixture. Samples were incubated at room temperature for 20 minutes away from open light. After incubation, all samples were diluted with 1 mL of HBS and analyzed using the ACEA NovoCyte flow cytometer. The fluorescent signal generated by PAC-1 was used to determine the expression or presence of activated GPIIb/IIIa receptors without bound fibrinogen. The fluorescent signal from 9F9 was used to determine binding of fibrinogen to the surface receptors on FDPDs.

### HTMA (HEPES Modified Tyrode's Albumin).

| | Concentration |
|---|---|
| | (mM, |
| | except |
| | where |
| | otherwise |
| Component | indicated) |
| HEPES | 9.5 |
| NaCl | 145.0 |
| KCl | 4.8 |
| NaHCO₃ | 12.0 |
| Dextrose | 5.0 |
| Bovine Serum Albumin | 0.35% w/v |

TABLE 20

| | Unstained | FITC Iso | 9F9 | PAC-1 |
|---|---|---|---|---|
| Cells (μL) | 10 | 10 | 10 | 10 |
| HMTA (μL) | 20 | 10 | 10 | 15 |
| Antibody (μL) | 0 | 10 | 10 | 5 |

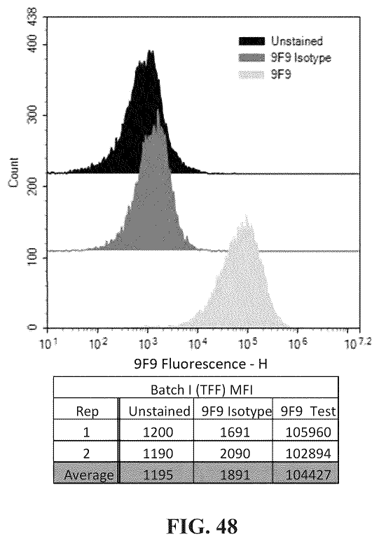

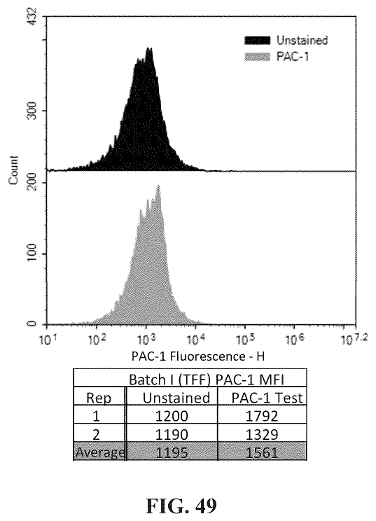

The samples were assayed by flow cytometry, and it was demonstrated that there is surface-bound fibrinogen post rehydration (FIG. 48), while the anti-PAC-1 antibody shows no significant binding (FIG. 49). This is further evidence that the FDPDs prepared by TFF include fibrinogen bound to the active form of GPIIb/GPIIIa, as PAC-1 binds to the same complex.

### Example 35. Pathogen Reduction

The reduction of pathogens is generally desirable in blood products. One method of pathogen reduction involves the use of a photosensitive nucleic acid-intercalating compound to alter the nucleic acids of pathogens upon illumination with an appropriate wavelength.

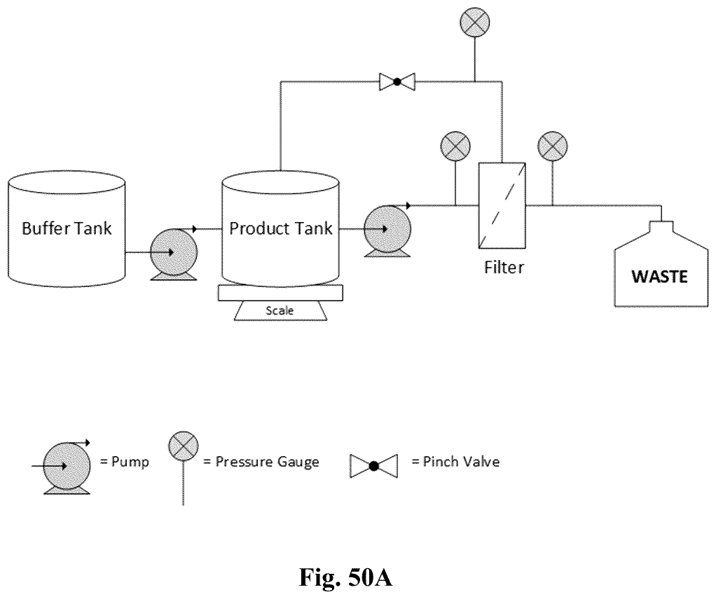

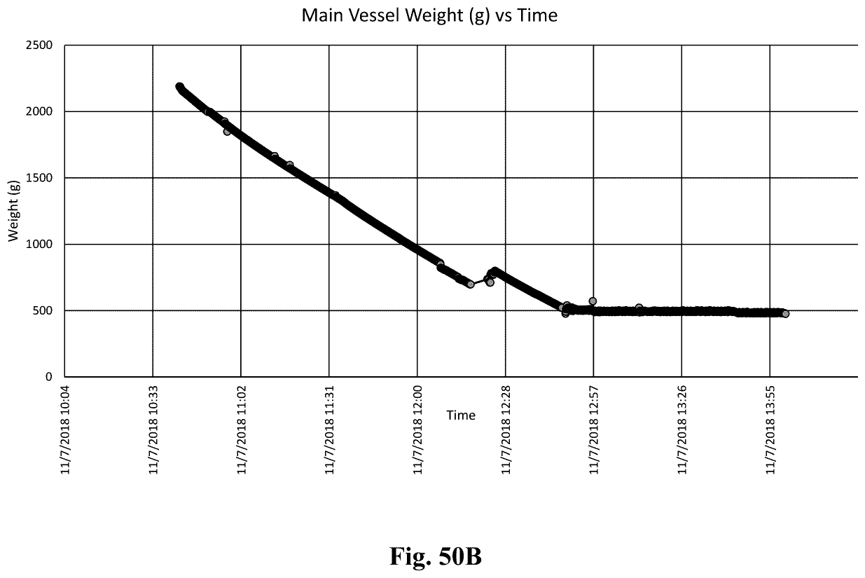

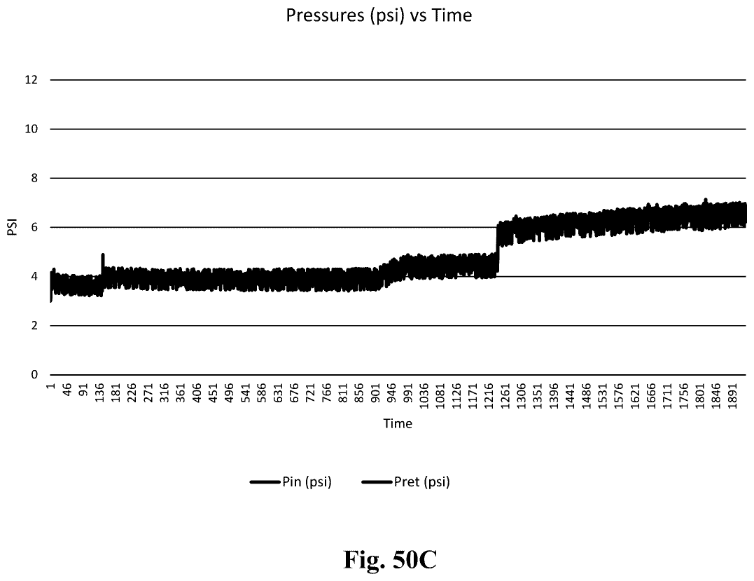

The INTERCEPT® system (made by Cerus) uses amotosalen, a nucleic acid intercalating compound that forms cross-links in nucleic acid upon illumination with UVA. Exemplary parameters for use of this system are shown in Table 21 and a schematic of the system is shown in FIG. 50A, while exemplary process data are shown in FIGS. 50B-C for 2.6 L of processed material in 198 minutes (approx. 14/min average).

DLS was performed as described in Example 30.

TABLE 21

| Process Parameter | Specification |
|---|---|
| Feed Pump | 600 ml/min |
| | (3/8” Tube) |
| Retentate Pressure | Target = 4 to 6 PSI |
| | Criteria = 2 to 8 PSI |
| Buffer Pump | 100 ml/min (noncritical) |
| DiaVolumes | X2 DVs |
| Concentration Factor | ~4 (from initial dilution) |

Exemplary comparative data of pH and metabolites of thrombosomes prepared as in Example 15, with or without treatment with the INTERCEPT® system is shown in Table 22.

TABLE 22

| İSTAT | Raw Material | | Initial Dilution | | End of Con | | End of DV (Pre-Lyo) | |
|---|---|---|---|---|---|---|---|---|
| CG4+ | Control | Treated | Control | Treated | Control | Treated | Control | Treated |
| pH | 7.2 | 7.2 | 7.3 | 7.1 | 7.3 | 7.3 | 7.3 | 7.2 |
| pCO2 | 32.9 | 25.7 | 16.5 | 29.1 | 16.1 | 14.4 | 11.3 | 12.4 |
| (mmHg) | | | | | | | | |
| pO2 | 67 | 149 | 167 | 150 | 142 | 155 | 145 | 153 |
| (mmHg) | | | | | | | | |

Exemplary comparative data of functional characterization (AcT count and aggregation parameters) and cell-surface markers are shown in Tables 23 (hIDSPs), 24 (prior to lyophilization) and Table 25 (following lyophilization and rehydration in 10 mL sterile water for injection to a concentration of approximately 1.8×10⁶/μL (individual sample counts are shown in Table 25).

TABLE 23

| | | Raw Material (hIDSP) | |
|---|---|---|---|
| | | Batch M | Batch N |
| | | “Control” | “Treated” |
| AcT Counts | AVG (x 10\hat{ }3) | 1212 | 1120 |
| Aggregation (%) | Collagen AVG | 22 | 21 |
| or | (aggregation) | | |
| thrombin-induced | Plasma-A.Acid AVG | 75 | 84 |
| trapping (%) | (aggregation) | | |
| | 250 k Thrombin AVG | 97 | 97 |
| | (thrombin-induced | | |
| | trapping) | | |
| | Buffer-A.Acid AVG | 94 | 92 |
| | (aggregation) | | |
| Flow (percent | CD41 | 93.5 | 97.6 |
| positivity) | CD42 | 91.4 | 95.8 |
| | Double Positive % | 92.0 | 95.6 |
| | CD62 | 23.9 | 42.5 |
| | Annexin V (AV5) | 3.8 | 8.4 |

TABLE 24

| | | Pre-Lyophilization | |
|---|---|---|---|
| | | Batch M | Batch N |
| | | “Control” | “Treated” |
| AcT Counts | AVG (x 10\hat{ }3) | 1787 | 2057 |
| Aggregation (%) | Collagen AVG | 81 | 82 |
| or | (aggregation) | | |
| thrombin-induced | Plasma-A.Acid AVG | 93 | 84 |
| trapping (%) | (aggregation) | | |
| | 250 k Thrombin AVG | 97 | 90 |
| | (thrombin-induced | | |
| | trapping) | | |
| | Buffer-A.Acid AVG | 89 | 95 |
| | (aggregation) | | |
| Flow (percent | CD41 | 98.4 | 97.0 |
| positivity) | CD42 | 98.2 | 95.4 |
| | Double Positive % | 97.5 | 94.3 |
| | CD62 | 26.7 | 41.6 |
| | AV5 | 10.6 | 13.7 |

TABLE 25

| | | Final Product QC | | |
|---|---|---|---|---|
| | | Batch M | | |
| | | “Control” | | Batch N |
| | | V1 | V2 | V1 |
| AcT Counts | AVG (x 10\hat{ }3) | 1765 | 1767 | 1720 |
| thrombin-induced | 375K Thrombin | 84 | 66 | 74 |
| trapping (%) | | | | |
| Flow (percent | CD41 | 85.5 | 79.5 | 91.2 |
| positivity) | CD42 | 85.1 | 79.2 | 90.6 |
| | Double Positive % | 84.6 | 78.8 | 90.1 |
| | CD62 | 87.0 | 93.2 | 87.1 |
| | AV5 | 95.4 | 95.0 | 92.6 |
| TGA | 4.8K TPH | 72.3 | 71.5 | 74.8 |

| | | |
|---|---|---|
| Residual Plasma % | 7.0% | 8.4% |

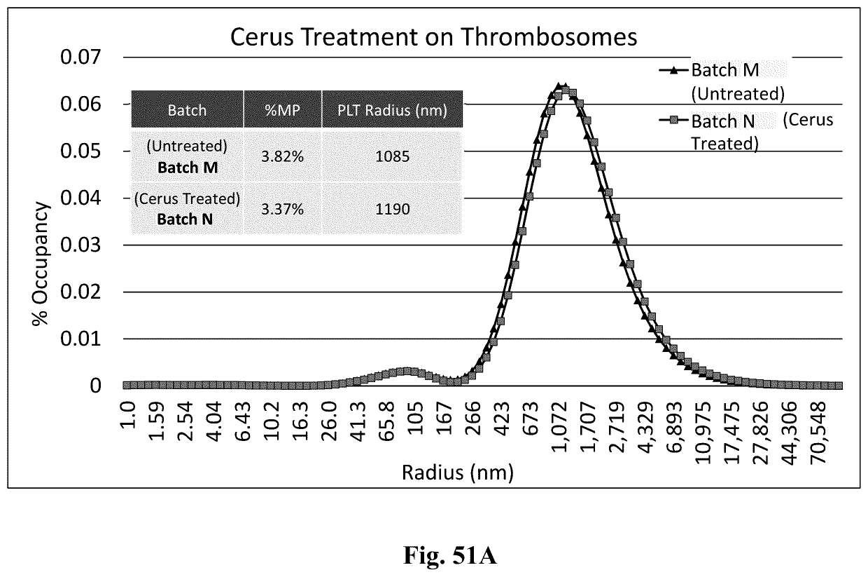

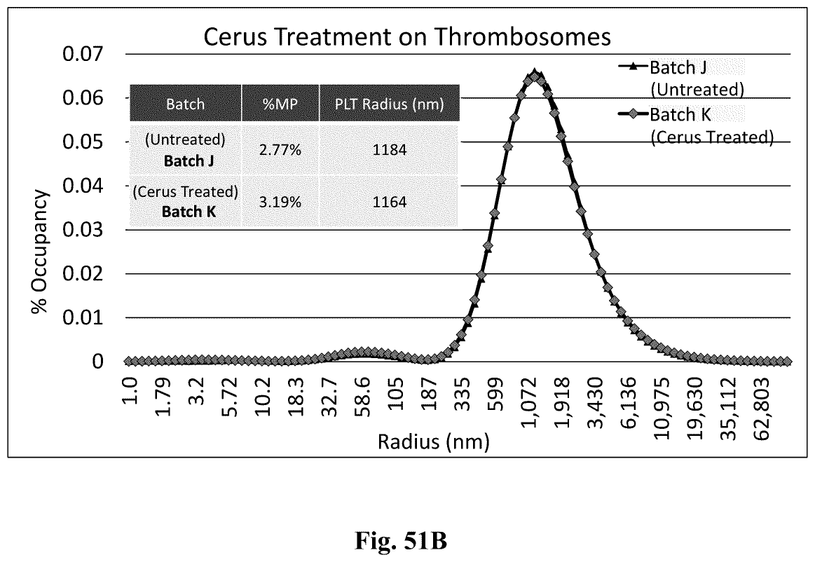

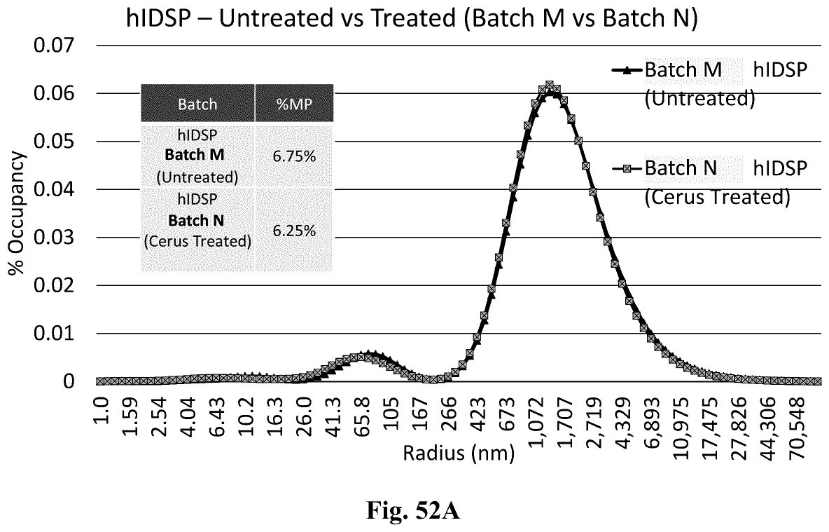

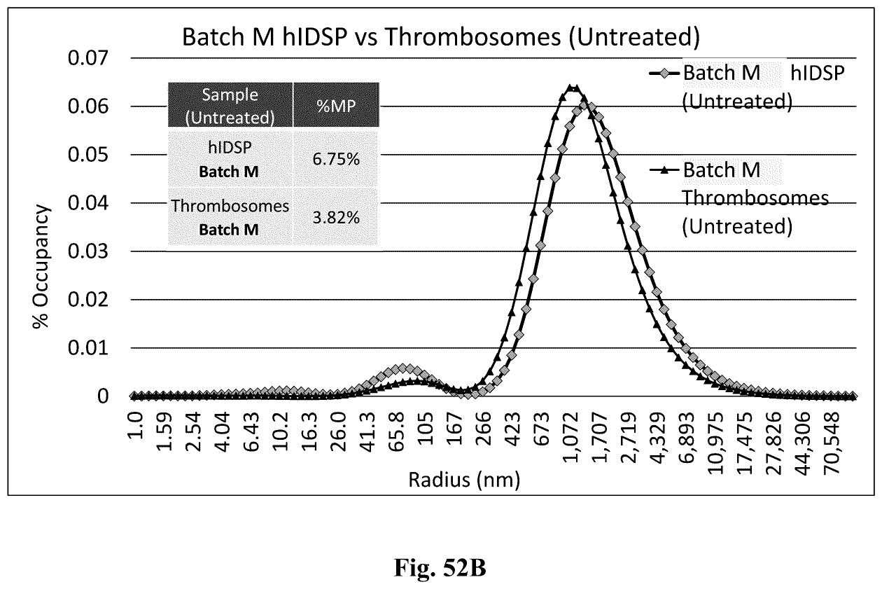

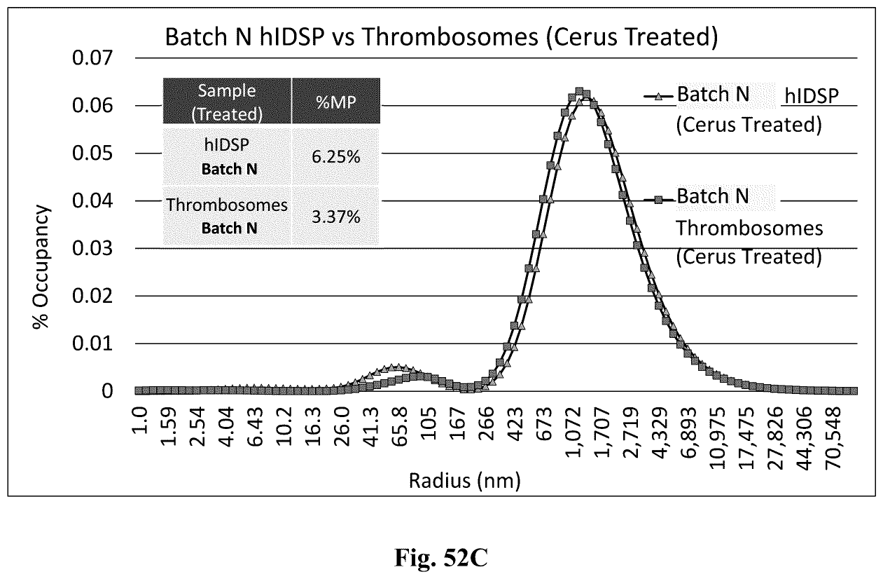

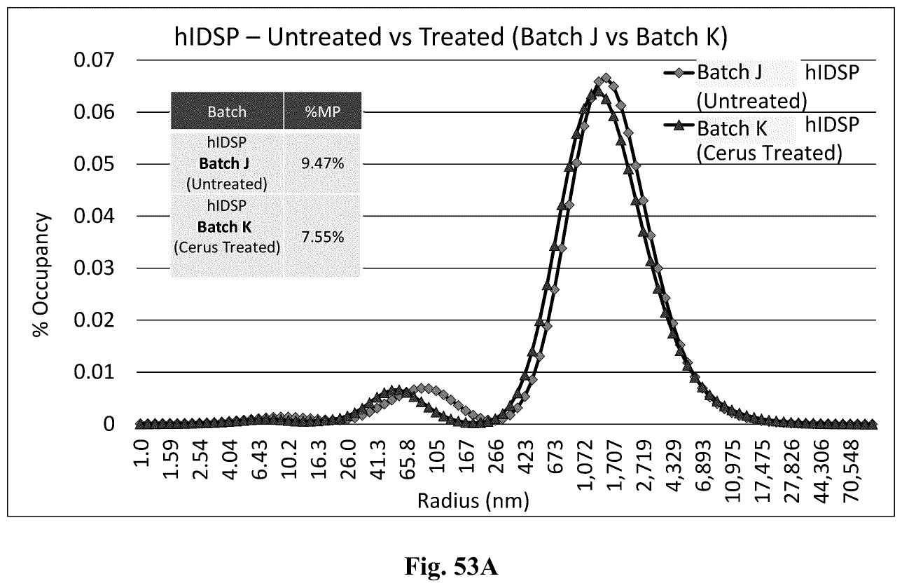

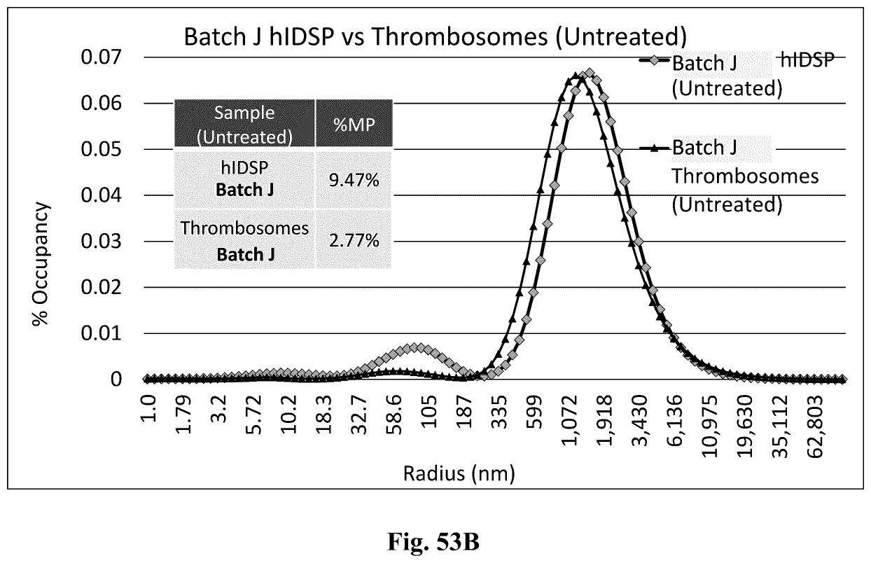

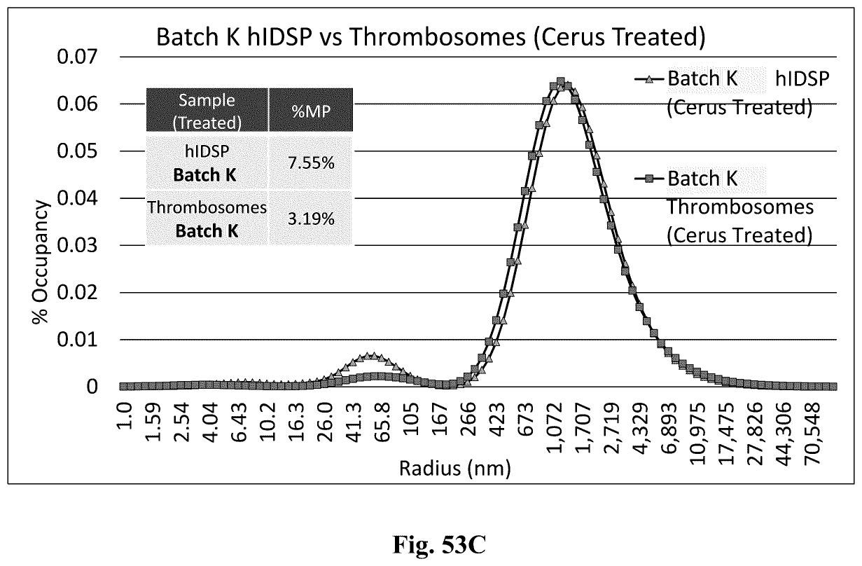

The microparticle content at various stages of the preparation of thrombosomes was also determined as described in Example 33. FIGS. 51A-B show the similarity of rehydrated thrombosomes prepared with and without pathogen reduction treatment. A summary of these data is shown in Table 26 FIG. 52A shows the microparticle content of hiDSPs with or without pathogen reduction treatment. FIGS. 52B-C compare the microparticle content of the hiDSPs shown in FIG. 52A and rehydrated thrombosomes prepared therefrom. A summary of these data is shown in Table 27. FIG. 53A shows the microparticle content of hiDSPs with or without pathogen reduction treatment. FIGS. 53B-C compare the microparticle content of the hiDSPs shown in FIG. 53A and rehydrated thrombosomes prepared therefrom. A summary of these data is shown in Table 28

TABLE 26

| | | Particle |
|---|---|---|
| | | Radius |
| Batch | % MP | (nm) |
| (Untreated) | 3.82% | 1085 |
| Batch M | | |
| (Cerus Treated) | 3.37% | 1190 |
| Batch N | | |
| (Untreated) | 2.77% | 1184 |
| Batch J | | |
| (Cerus Treated) | 3.19% | 1164 |
| Batch K | | |

TABLE 27

| Batch | % MP |
|---|---|
| hIDSP | 6.75% |
| Batch M | |
| (Untreated) | |
| Thrombosomes | 3.82% |
| Batch M | |
| (Untreated) | |
| hIDSP | 6.25% |
| Batch N | |
| (Treated) | |
| Thrombosomes | 3.37% |
| Batch N | |
| (Treated) | |

TABLE 28

| Batch | % MP |
|---|---|
| hIDSP | 9.47% |
| Batch J | |
| (Untreated) | |
| Thrombosomes | 2.77% |
| Batch J | |
| (Untreated) | |
| hIDSP | 7.55% |
| Batch K | |
| (Treated) | |
| Thrombosomes | 3.19% |
| Batch K | |
| (Treated) | |

### Example 36. T-TAS® Thrombosome Data

In the Total Thrombus-formation Analysis System (T-TAS®, FUJIMORI KOGYO CO., LTD), the sample is forced through collagen-coated microchannels using mineral oil. Changes in pressure are used to assess thrombus formation. The Occlusion Start Time is time it takes to reach Δ10 kPa, and the Occlusion Time=time it takes to each Δ80 kPa using an AR chip (Zacros Item No, TC0101).

According to FUJIMORI KOGYO CO., LTD, an AR chip can be used for analyzing the formation of a mixed white thrombus consisting chiefly of fibrin and activated platelets. It has a flow path (300 μm wide by 50 μm high) coated with collagen and tissue factors and can be used to analyze the clotting function and platelet function. In comparison, a PL chip can be used for analyzing the formation of a platelet thrombus consisting chiefly of activated platelets. A PL chip has a flow path coated with collagen only and can be used to analyze the platelet function.

T-TAS® reagents (CaCTI, AR Chip) were warmed to 37° C. and thrombosomes were rehydrated according to standard protocol. An aliquot of the rehydrated thrombosomes was washed by centrifugation at 3900 g×10 minutes and resuspended to approximately 300,000 cells/μL in sodium citrate anticoagulated platelet-poor plasma (PPP). CaCTI (20 μL) was mixed with thrombosomes in PPP (480 μL) and run through the T-TAS AR Chip under high shear. Pressure in the system was monitored over 30 minutes or until the maximum backpressure in the channel was achieved.

The T-TAS® instrument was prepared for use according to the manufacturer's instructions. AR Chips (Diapharma Cat. #TC0101) and AR Chip Calcium Corn Trypsin Inhibitor (CaCTI; Diapharma Cat. #TR0101) were warmed to room temperature. 300 μL of rehydrated thrombosomes were transferred to a 1.7 mL microcentrifuge tube and centrifuged at 3900 g×10 minutes to pellet. The thrombosomes pellet was resuspended in George King (GK) pooled normal human plasma or autologous plasma with or without autologous platelets to a concentration of approximately 100,000-450,000/μL, as determined by AcT counts (Beckman Coulter AcT Diff 2 Cell Counter). 20 μL of CaCTI with 480 μL of thrombosomes sample in GK plasma were mixed with gentle pipetting. The sample was loaded and run on the T-TAS® according to the manufacturer's instructions.

Table 29 shows T-TAS® results from citrated whole blood, platelet-reduced citrated whole blood supplemented with varying concentrations of thrombosomes as prepared in Example 18, and George King Platelet Poor Plasma (GK PPP) supplemented with varying concentrations of thrombosomes as prepared in Example 18 in experiments run according to the manufacturer's instructions using the AR chip and High Shear instrument settings.

TABLE 29
T-TAS AR Chip Results

| | Actual Tsome | Base | Occlusion | Occlusion | Occlusion | Area |
|---|---|---|---|---|---|---|
| | Concentration | Pressure | Start Time | Time | Speed | Under |
| Sample Type | (×10\hat{ }3/μL) | (kPa) | (hh:mm:ss) | (hh:mm:ss) | (kPa/min) | Curve |
| Citrated | 0 | 3.2 | 0:11:19 | 0:14:03 | 25.6 | 1393.9 |
| Whole Blood | | | | | | |
| Platelet | 0 | 3.3 | 0:12:41 | 0:16:57 | 16.4 | 1180.6 |
| Reduced | 73 | 3.2 | 0:11:11 | 0:13:47 | 26.9 | 1380.9 |
| Citrated | 173 | 3.4 | 0:09:37 | 0:13:22 | 18.7 | 1498.5 |
| Whole Blood | 255 | 3.4 | 0:08:36 | 0:10:40 | 33.9 | 1653.1 |
| GK PPP | 0 | 2.7 | 0:25:34 | 0:00:00^{†} | 0 | 138.8 |
| | 45 | 2.8 | 0:27:22 | 0:28:48 | 48.8 | 190.6 |
| | 193* | 2.9 | 0:12:41 | 0:00:00^{†} | 0 | 775.3 |
| | 384 | 2.8 | 0:10:54 | 0:12:20 | 48.8 | 1479.8 |

*Test peaked at~75 kPa before rapidly dropping off. Possible erroneous result.
^{†}Test timed out.

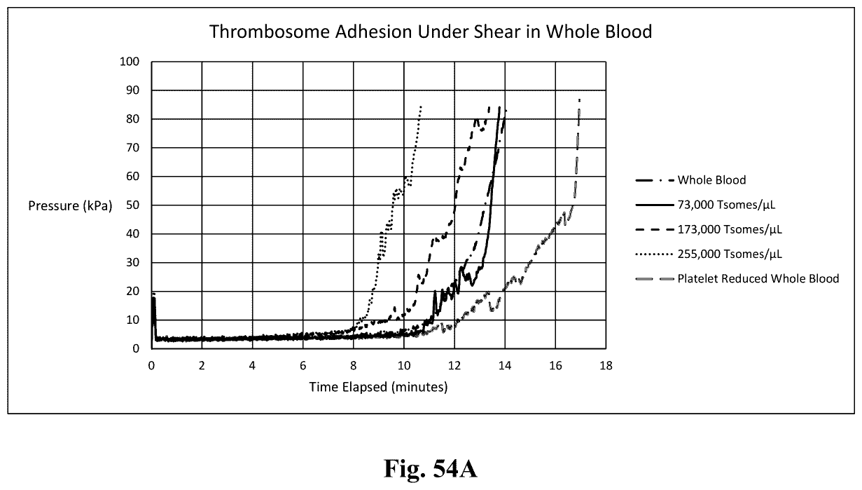

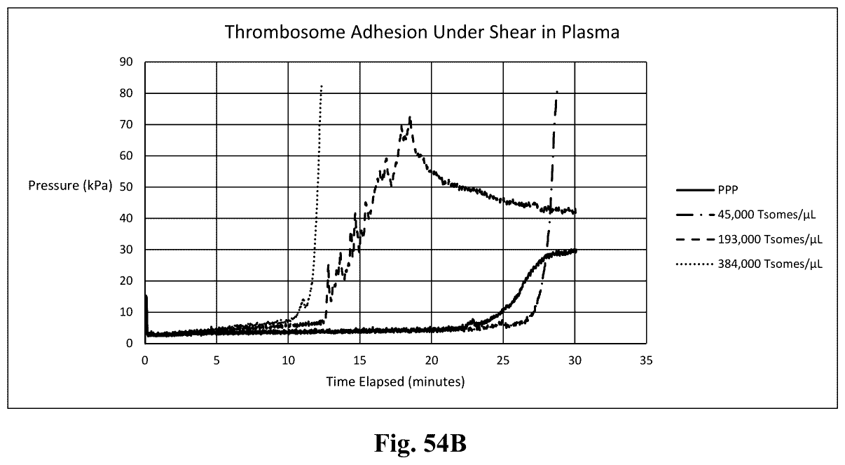

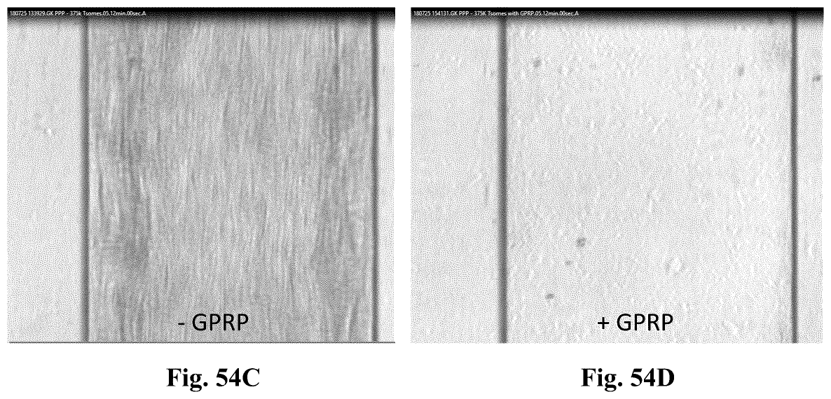

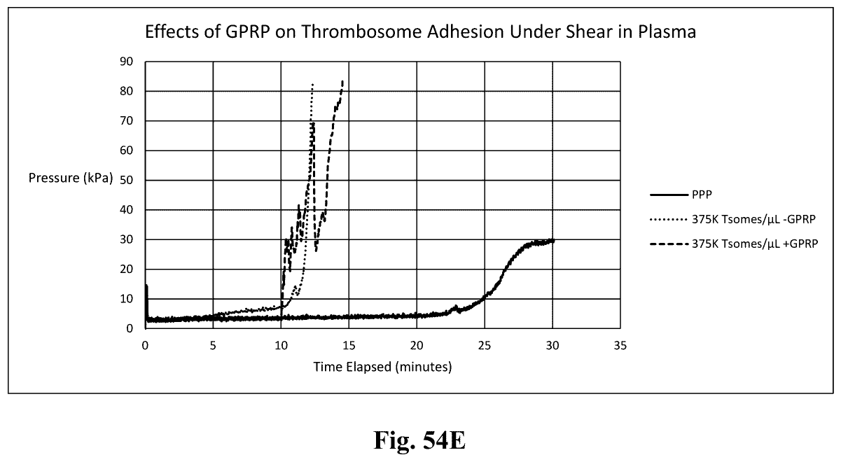

Time-elapsed results are shown in FIGS. 54A-B. Increasing the concentration of thrombosomes in platelet-reduced whole blood promoted more robust thrombus formation as measured by shortened occlusion times (FIG. 54A). Increasing the concentration of thrombosomes in platelet poor plasma (PPP) promoted more robust thrombus formation as measured by shortened occlusion times (FIG. 54B).

The effect of GPRP (1 mM) on occlusion activity was also assayed. Table 30 shows T-TAS® results for platelet-poor plasma, with and without thrombosomes in the presence and absence of GPRP. Adding GPRP to prevent fibrinogen formation did not prevent the thrombosome-containing sample from reaching occlusion pressure. While the addition of GPRP to thrombosome samples in plasma prevents the formation of fibrin in the microcapillary channel (FIGS. 54C (no GPRP) and 54D (GPRP), both in GK PPP), the addition of GPRP to thrombosomes (PPP) did not prevent thrombus formation (FIG. 54E).

TABLE 30
AR Chip: GPRP Comparison

| | Actual Tsome | Base | Occlusion | Time | Occlusion | Area |
|---|---|---|---|---|---|---|
| | Concentration | Pressure | Start Time | Occlusion | Speed | Under |
| Sample Type | (×10\hat{ }3/μL) | (kPa) | (hh:mm:ss) | (hh:mm:ss) | (kPa/min) | Curve |
| GK PPP | 0 | 2.7 | 0:25:34 | 0:00:00^{†} | 0 | 138.8 |
| (No Tsomes) | | | | | | |
| GK PPP + 1 mM | 0 | 3.5 | 0:00:00 | 0:00:00^{†} | 0 | 52.43 |
| GPRP (No Tsomes) | | | | | | |
| GK PPP + 375 k | 384 | 2.8 | 0:10:54 | 0:12:20 | 48.8 | 1479.8 |
| Tsomes | | | | | | |
| GK PPP + 37 5k | 380 | 3.2 | 0:10:09 | 0:14:32 | 16 | 1426.9 |
| Tsomes with 1 mM | | | | | | |
| GPRP | | | | | | |

^{†}Test timed out

### Example 37: Anti-Human Factor XI Antibody Confirms Blocking of the Function of Factor XI

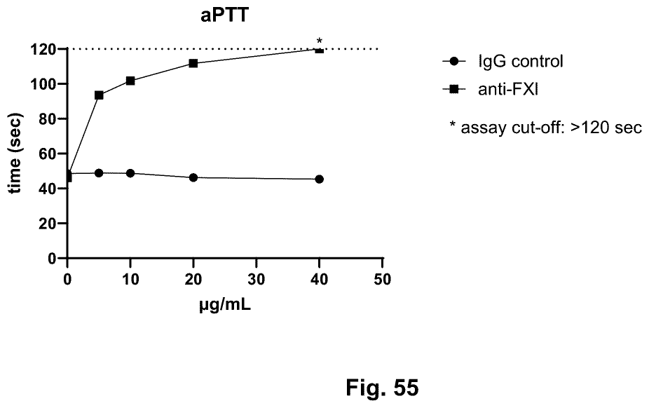

The activated partial thromboplastin time (aPTT) was performed to confirm blocking of the function of anti-human FXI antibody. A 9.4 mg/mL working stock of anti-human FXI antibody (Prolytix, cat #AHXI-5061, www.goprolytix.com) or an isotype control mouse IgG (Thermo Fisher, cat #500-M00-1MG, www.thermofisher.com) was created. The anti-human FXI or IgG was tittered into Octaplas® plasma (Octapharma, www.octapharmausa.com) and samples were incubated on the rocker at 37° C. for 30 minutes. The aPTT was calculated using the STart 4 instrument (Stago, www.stago-us.com) and C.K. Prest reagent (Stago, www.stago-us.com) according to manufacturer's directions. FIG. 55 shows the aPTT of anti-FXI antibody and IgG control. Isotype control has no effect on the aPTT while anti-FXI antibody confirms blocking the function of FXI with increased aPTT time. In fact, at 5 μg/mL of anti-FXI antibody aPTT doubled (48.9 sec vs. 93.6 sec). Therefore, the amount of 5 μg/mL of anti-FXI antibody was selected for further experiments. In addition, higher or lower amounts of anti-FXI antibody can be used to titer the severity of FXI inhibition, if desired.

### Example 38: Activity of Freeze-Dried Platelet Derived Hemostat (FPH) in the Presence of FXI Inhibition Alone Measured Using Thrombin Generation Assay (TGA)

A vial of FDPDs (also referred to herein as FPH) prepared according to Example 18 was rehydrated using 10 mL of deionized water. FPH TGA activity was tested in the presence of FXI inhibition alone. Octaplas® (Octapharma, www.octapharmausa.com) was treated with 5 μg/mL anti-FXI (Prolytix cat #AHXI-5061, www.goprolytix.com) or 5 μg/mL IgG control (Thermo Fisher cat #500-M00-1MG, www.thermofisher.com) and samples were incubated on a rocker at 37° C. for 30 minutes. Following incubation, aPTT was performed using the STart 4 instrument (Stago, www.stago-us.com) and C.K. Prest reagent (Stago, www.stago-us.com) according to manufacturer's directions to confirm inhibition.

Samples were diluted in TGA dilution buffer to achieve a final concentration of 30% Octaplas®. FPH was added to samples to achieve a final concentration of 7,200 particles/μL. Cephalin (UPTT reagent, Bio/data Corporation, www.biodatacorp.com) was diluted 1:50 in Octaplas® and TGA buffer for a final concentration of 30% Octaplas®. 80 μL of each sample was added to 96-well U-bottom plate in six wells, with 20 μL PRP reagent (Stago, www.stago-us.com) added to three wells and thrombin calibrator (Stago, www.stago-us.com) added to three wells. To initiate the reaction, 20 μL FluCa buffer (Stago, www.stago-us.com) was injected into each well and the run was initiated per the Calibrated Automated Thrombinoscope method (Stago, www.stago-us.com). Data was collected every 60 seconds for 120 minutes total. The final concentration of FPH in each well after all reagents were added was 4,800 particles/μL. Two lots of FPH were tested.

Table 31 below shows the aPTT results to confirm anti-FXI inhibition. The inhibition by anti-FXI can be observed by the increase in aPTT as compared to the untreated, and anti-IgG samples. The other controls—normal and abnormal were provided by the manufacturer.

TABLE 31

| | aPTT |
|---|---|
| Sample | (sec) |
| Un-treated | 41.8 |
| (UT) (only | |
| Octaplas) | |
| Anti-FXI | 83.2 |
| Anti-IgG | 40.9 |
| Normal control | 31.3 |
| (provided by the | |
| manufacturer) | |
| Abnormal control | 65.0 |
| (provided by the | |
| manufacturer) | |

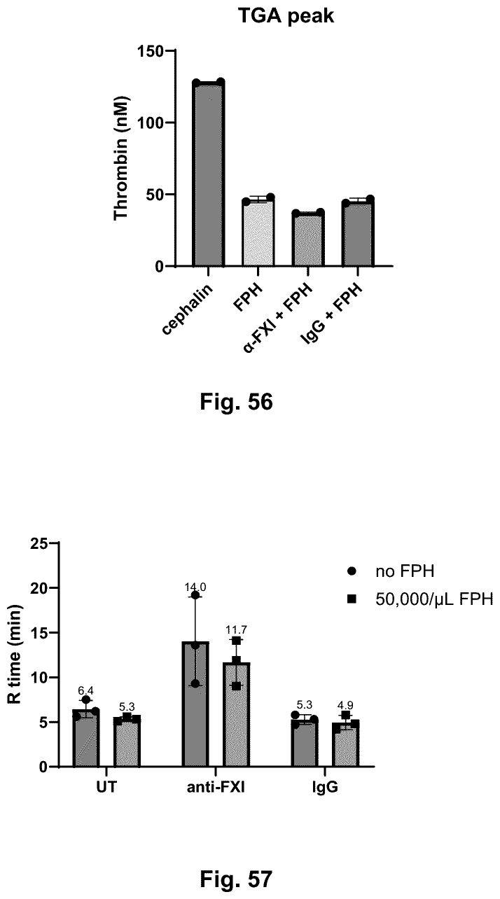

FIG. 56 shows FPH peak thrombin activity in the presence of FXI inhibition. FPH thrombin generation is slightly decreased (˜20%) in the presence of FXI inhibition. It can be observed that FPH generated 50 nM peak thrombin at 5,000 particles/ml, approximately 80% of which was retained in the presence of anti-FXI. Therefore, it can be observed that the FXI inhibition does not significantly alter FPH thrombin generation as shown in FIG. 56. Hence, FPH is able to generate thrombin in the presence of inhibitory FXI antibody.

### Example 39: Activity of FPH in the Presence of FXI Inhibition Measured Using Thromboelastography (TEG) Assay

TEG assay was performed to test the activity of FPH in the presence of Factor XI inhibition. A vial of FDPDs (also referred to herein as FPH) prepared according to Example 18 was rehydrated using 10 mL of deionized water. Fresh drawn whole blood was collected into 3.2% sodium citrate tubes. Whole blood was treated with 5 μg/mL anti-FXI (Prolytix cat #AHXI-5061, www.goprolytix.com) or 5 μg/mL IgG control (Thermo Fisher cat #500-M00-1 MG, www.thermofisher.com) and placed on a rocker for 30 minutes. Following incubation, samples were tested on the TEG5000 (Haemonetics, hospital.haemonetics.com) using kaolin and calcium activation according to manufacturer's recommendations. Three blood donors and three lots of FPH were tested. FIG. 57 shows the FPH TEG activity (R time) in the presence of FXI inhibition (anti-FXI). Anti-FXI prolonged WB kaolin R-time from 6.4 (UT with no FPH) to 14.0 min (anti-FXI with no FPH), which was partially corrected to 11.7 min (˜80%) (anti-FXI with FPH) in the presence of 50,000 particles/μl FPH. IgG was inert. Anti-FXI antibody treatment increases the R-time, and FPH decreases the R time in all treatment groups (n=3). R-time was partially corrected by FPH in the presence of inhibitory anti-FXI.

### Example 40: Activity of FPH in the Presence of a Combination of Dual Antiplatelet Therapy (DAPT) and Anti-Factor XI Measured Using ADP-Modified Thrombin Generation Assay (TGA)

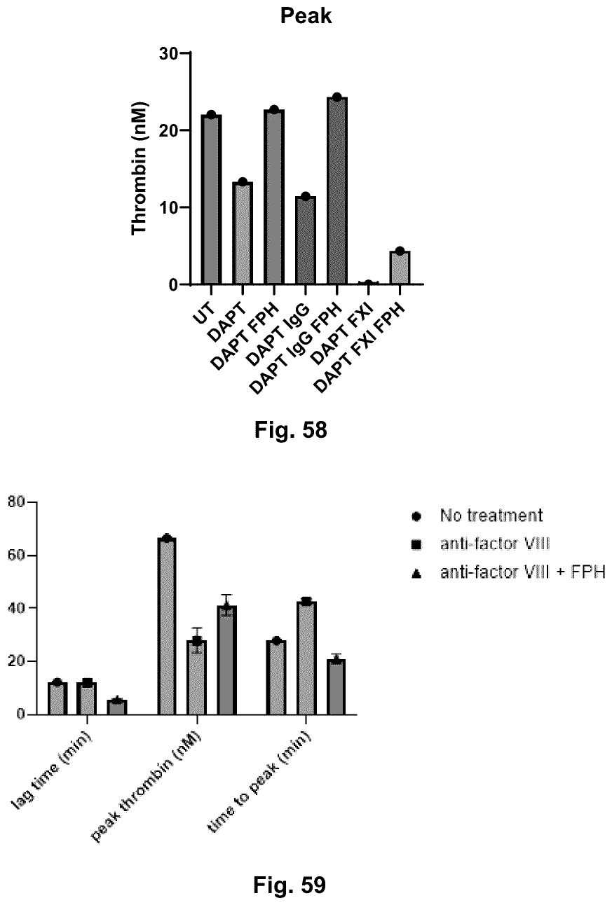

ADP-modified TGA assay was performed to test the activity of FPH in the presence of DAPT and Factor XI inhibition. Blood was collected in 3.2% sodium citrate tubes and centrifuged to obtain PRP (180×g, 10 minutes) and PPP (1800×g, 10 minutes). PRP was adjusted to contain 200,000 platelets/μL with autologous PPP. PRP was treated with DAPT (1 μg/mL ticagrelor, 100 μM ASA (aspirin)), 5 μg/mL anti-FXI (Prolytix cat #AHXI-5061, www.goprolytix.com) or 5 μg/mL IgG isotype (Thermo Fisher cat #500-M00-1 MG, www.thermofisher.com) and/or FPH at 20,000 particles/μL. Efficacy of DAPT treatment was tested via traditional light-transmission aggregometry to 10 μM ADP and 0.5 mM arachidonic acid (A.A), and efficacy of anti-FXI was tested by aPTT (Stago, www.stago-us.com). Treated PRP was diluted 1:10 in detergent-treated Octoplas® plasma (Octapharma, www.octapharmausa.com) and 80 μL was added to each well. Wells received either 20 μL phosphate-buffed saline (PBS) or thrombin calibrator (Stago, www.stago-us.com), performed in triplicate for every sample. The reaction was initiated with FluCa buffer (Stago, www.stago-us.com) containing ADP and PGE1 for a final concentration of 10 UM and 1 μM in the assay wells, respectively. The Calibrated Automated Thrombinoscope method (Stago, www.stago-us.com) was performed. Data was collected every 60 seconds for 180 minutes. One lot of FPH was tested. FIG. 58 shows FPH peak thrombin in the presence of samples treated with DAPT and anti-Factor XI. The untreated (UT), DAPT, and DAPT plus FPH are similar to historical data, confirming that the assay is working. FXI inhibition on top of DAPT completely flat-lined the sample. Using ADP agonist, DAPT reduced peak thrombin by 50% while anti-FXI and DAPT abolished TGA. FPH restored DAPT/FXI TGA by ˜25%. FPH recovered slightly in comparison to DAPT alone inhibition. IgG control had no effect on DAPT or DAPT plus FPH on thrombin, as expected. FPH was able to partially recover TGA (˜25%) in the presence of anti-FXI and DAPT. Therefore, this Example demonstrates that the FPH can restore the thrombin generation in the presence of both antiplatelet agent (two antiplatelet agents) and anticoagulant that inhibits Factor XI.

It can be understood that the anti-human FXI antibody used herein in Examples 38-40 is to show the activity of FDPDs, or FPH herein in the presence of dysfunctional or inhibited FXI. The Examples herein demonstrate that the FDPD, or FPH herein can be used in a similar manner to reverse, or counter the effects of an anticoagulant that inhibits the activity of FXI, at least in a manner to generate thrombin in the presence of the agent (anticoagulant, or antibody) that inhibits the activity of FXI, and provides a proof-of-concept supporting the effectiveness of FDPDs, or FPH herein at restoring hemostasis in the presence of FXI Inhibitors, alone or combined with dual anti-platelet therapy (DAPT).

### Example 41: FPH Partially Recovers Thrombin Generation in an In Vitro Surrogate Model of Acquired Hemophilia for Clotting Factors VIII and IX

Hemophilia A is caused by a deficiency in clotting factor VIII, while hemophilia B is caused by a deficiency in clotting factor IX. Further, acquired hemophilia occurs when the body produces antibodies (inhibitors) that block the clotting factors essential for blood clotting. This example presents in vitro surrogate models of acquired hemophilia A (deficiency in clotting factor VIII), and hemophilia B (deficiency in clotting factor IX).

A vial of FDPDs (also referred to herein as FPH) prepared according to Example 18 herein was rehydrated using 10 mL of deionized water. Thrombin generation assay (TGA) was performed with sheep polyclonal anti-human factors VIII and IX antibodies (Prolytix, https://goprolytix.com) to mimic acquired hemophilia disease. Samples were run with and without 50,000 particles/μL of FPH. Octaplas® (Octapharma, https://octapharmausa.com) plasma samples were spiked with anti-factor VIII or anti-factor IX antibodies at 10 μg/mL. Samples were pipetted into the wells of a 96-well plate at 80 μL and 20 μL of platelet low plasma (PPP) low reagent (Stago, https://stago-us.com) was added to each well. Calibrator wells were also created for each sample by adding 20 μL of thrombin calibrator (Stago, https://stago-us.com) to each sample instead of the PPP low reagent. The plate was then loaded into the plate reader and the run was initiated. The reaction was initiated by the instrument by injection of 20 μL of FluCa reagent (Stago, https://stago-us.com). Data was collected every 60 seconds for 60 minutes total. Thrombinoscope software computed the lag time (time to start clot formation), peak thrombin and time to peak thrombin.

FIG. 59 shows the lag time, peak thrombin and time to peak formation of un-treated plasma sample, a 10 μg/mL anti-factor VIII antibody-treated plasma sample, and a 10 μg/mL anti-factor VIII antibody-treated plasma sample with the addition of 50,000 particles/μL of FPH. The lag time was unaffected by the blocking antibody to factor VIII (anti-factor VIII antibody) but was shortened with the addition of FPH from 12.11±1.34 minutes to 5.67±0.27 minutes. Peak thrombin decreased significantly in the presence of antibody to factor VIII from 66.47±0.58 nM to 27.91±4.74 nM; but increased with the addition of FPH to 41.26±3.92 nM. The time to peak was delayed in the presence of anti-factor VIII antibody from 27.89±0.57 minutes to 42.67±0.82 minutes, but improved with the addition of FPH to 21±1.78 minutes.

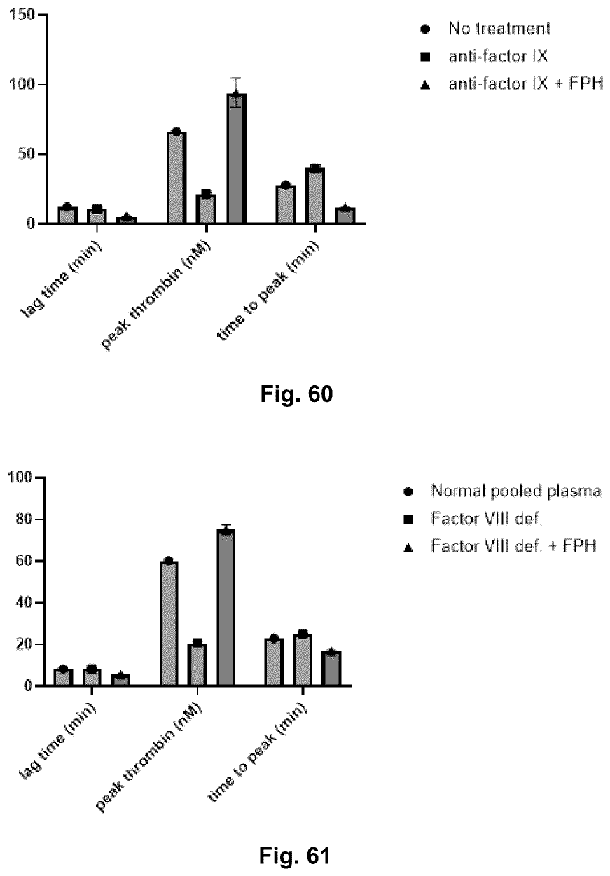

FIG. 60 shows the lag time, peak thrombin and time to peak formation of un-treated plasma sample, a 100 μg/mL anti-factor IX antibody-treated plasma sample, and a 100 μg/mL anti-factor IX antibody treated plasma sample with the addition of 50,000 particles/μL of FPH. The lag time was unaffected by the blocking antibody to factor IX (anti-factor IX antibody) but was shortened with the addition of FPH from 10.78±1.13 minutes to 5.44±0.31 minutes. Peak thrombin decreased significantly in the presence of antibody to Factor IX from 66.47±0.58 nM to 21.57±1.23; but increased with the addition of FPH to 94.2±10.39 nM. The time to peak was delayed in the presence of anti-factor IX antibody from 27.89±0.57 minutes to 40.11±2.31 minutes, but improved with the addition of FPH to 12.00±0.47 minutes.

Therefore, the presence of FPH speeds up the production of thrombin, therefore, restoring, or improving the thrombin generation even in the presence of anti-factor VIII antibody, or anti-factor IX antibody as demonstrated in the TGA assay.

### Example 42: FPH Partially Recovers Thrombin Generation in an In Vitro Surrogate Model of Congenital Hemophilia for Clotting Factors VIII and IX

Congenital hemophilia is a genetic, inherited bleeding disorder caused by a deficiency in a clotting factor, usually present at birth. This example presents in vitro surrogate models of congenital hemophilia A and hemophilia B.

A vial of FDPDs (also referred to herein as FPH) prepared according to Example 18 herein was rehydrated using 10 mL of deionized water. Thrombin generation assay (TGA) was performed using depleted factor VIII plasma (i.e. plasma with depleted factor VIII), or depleted factor IX plasma (i.e., plasma with depleted factor IX) that was compared to pooled normal plasma from George King (Bio-Medical Inc, https://kingbiomed.com). Samples were pipetted into the wells of a 96-well plate at 80 μL and 20 μL of platelet poor plasma (PPP) low reagent (Stago, https://stago-us.com) was added to each well. Calibrator wells were also created for each sample by adding 20 μL of thrombin calibrator (Stago, https://stago-us.com) to each sample instead of the PPP low reagent. The plate was then loaded into the plate reader and the run was initiated. The reaction was initiated by the instrument by injection of 20 μL of FluCa reagent (Stago, https://stago-us.com). Data was collected every 60 seconds for 60 minutes total. Thrombinoscope software computed the lag time (time to start clot formation), peak thrombin and time to peak thrombin. FIG. 61 shows the lag time, peak thrombin and time to peak formation of normal human plasma (n=3), 95% depleted factor VIII human plasma, and 95% depleted factor VIII human plasma with the addition of 40,000 particles/μL of FPH. There was no change in lag time with factor VIII depleted plasma and normal plasma, but the addition of FPH partially reduced the lag time. The peak thrombin for normal plasma was 60.12±0.99 nM, and was significantly reduced in the case of factor VIII depleted plasma to 20.81±0.87 nM. However the addition of FPH to the factor VIII depleted plasma partially restored the peak thrombin to 75.06±2.37 nM. The time to peak was not changed for factor VIII depleted plasma compared to normal plasma at 23.78±0.68 minutes, However the addition of FPH to the factor VIII depleted plasma decreased the time to peak to 16.76±0.72 minutes.

FIG. 62 shows the lag time, peak thrombin and time to peak formation of normal human plasma (n=3), factor IX depleted human plasma, and factor IX depleted human plasma with the addition of 40,000 particles/μL of FPH. There was no change in lag time with factor IX depleted plasma and normal plasma, but the addition of FPH partially reduced the lag time. The peak thrombin for normal plasma was 60.12±0.99 nM, and was significantly reduced in the case of factor IX depleted plasma to 7.00±0.80 nM. However, the addition of FPH to the factor IX depleted plasma partially restored the peak thrombin to 33.45±0.61 nM. The time to peak for Factor IX depleted plasma compared to normal plasma was at 35.01±2.53 minutes and 23.78±0.68 minutes, respectively. However, the addition of FPH to the factor IX depleted plasma decreased the time to peak to 26.33±0.57 minutes, therefore, partially restoring the peak thrombin to its original value for normal pooled plasma.

Therefore, the addition of FPH decreased the time to thrombin production and increased the amount of thrombin produced in the presence of low functioning factor VIII or IX.

### Example 43: FPH Shortens the Time to Clot Formation (R-Time) in the Presence of Antibodies to Either Factor VIII or Factor IX in Platelet Rich Plasma Samples in Thromboelastography (TEG) Assay

A vial of FDPDs (also referred to herein as FPH) prepared according to Example 18 herein was rehydrated using 10 mL of deionized water. Platelet rich plasma (PRP) (n=2) was treated with increasing amounts of anti-factor VIII antibody or anti-factor IX antibody with and without FPH. Thromboelastography was run using the TEG5000 (Haemonetics, https://hospital.haemonetics.com). The PRP was added to the reaction cup and assay initiated by kaolin and calcium. The data reported by the software for R-time (reaction time) was recorded and compared to normal PRP.

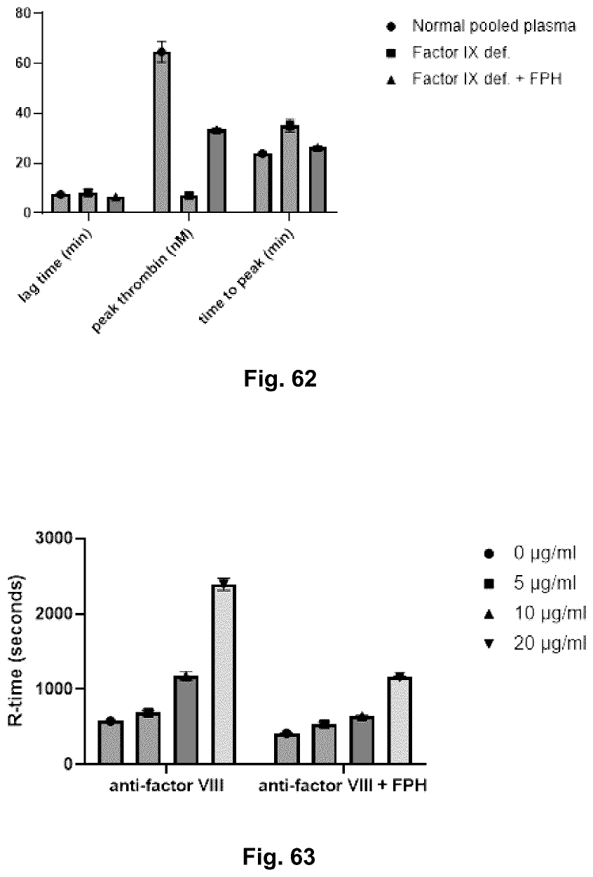

FIG. 63 shows the time to clot formation (R-time) for the increasing amounts of anti-factor VIII antibody with and without 50,000 particles/μL of FPH. The antibody to factor VIII showed an increased the reaction time (R-time) from 577.5±3.53 seconds to 687.5±31.8, 1175±56.56, 2392.5±81.3 second with increasing antibody concentrations from 5, 10 and 20 μg/mL. The addition of FPH decreased the R-time at each antibody concentration to 537.5±31.82 (5 μg/mL), 640±21.2 (10 μg/mL) and 1160.0±14.14 (20 μg/mL) seconds. It can be observed that even at the highest concentration (20 μg/ml) of anti-factor VIII antibody, 50K/μl of FPH were able to decrease the time to clot formation, thereby, speeding the clot formation.

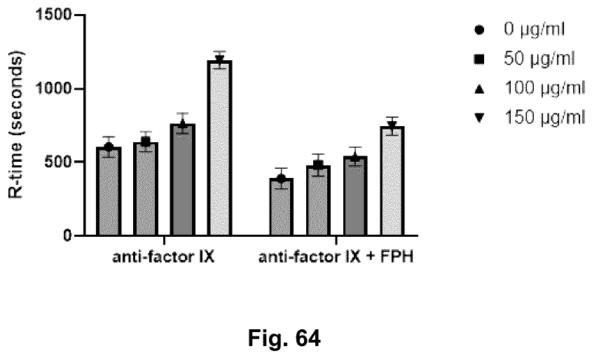

FIG. 64 shows the time to clot formation (R-time) for the increasing amounts of anti-factor IX antibody with and without 50,000 particles/μL of FPH. The R-time at 0, 50, 100 and 150 μg/mL was 605±70.55, 640±69.1, 765±69.6 and 1192.5±58.7 seconds, respectively. The addition of FPH to each sample decreased the R-time to 390±69.55, 482.5±76.1, 540±64.75, 745±61.95 seconds, respectively. It can be observed that even at the highest concentration (150 μg/ml) of anti-factor IX antibody, 50K/μl of FPH were able to decrease the time to clot formation, thereby, speeding the clot formation.

Therefore, the decrease in the time to clot formation (R-time) in the presence of FPH demonstrates that FPH in the presence of various concentrations of anti-factor IX antibody speeds up clot formation as demonstrated here by TEG assay.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above. Furthermore, one having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. Embodiments of the invention so claimed are inherently or expressly described and enabled herein. In order to determine the metes and bounds of the invention, therefore, reference should be made to the appended claims.

What is claimed is:

1. A method for administering platelet derivatives to a subject, wherein the method comprises:
rehydrating a composition in the form of a powder, said composition comprising the platelet derivatives and an incubating agent comprising one or more saccharides, one or more salts, and a buffer to form a rehydrated platelet derivative composition, and
administering intravenously a dose of the rehydrated platelet derivative composition to the subject, wherein the dose comprises a first dose of the rehydrated platelet derivatives, wherein the first dose comprises an amount of rehydrated platelet derivatives between 1.0×10⁷ particles/kg and 1.0×10¹¹ particles/kg of the subject,
wherein the subject has been treated or is being treated with aspirin, at least one anticoagulant agent, and at least one other antiplatelet agent, and
wherein the at least one anticoagulant agent comprises a Factor XI inhibitor.

2. A method for administering platelet derivatives to a subject, wherein the method comprises:
rehydrating a composition in the form of a powder, said composition comprising the platelet derivatives and an incubating agent comprising one or more saccharides, one or more salts, and a buffer to form a rehydrated platelet derivative composition, and
administering intravenously a dose of the rehydrated platelet derivative composition to the subject, wherein the dose comprises a first dose of the rehydrated platelet derivatives, wherein the first dose comprises an amount of rehydrated platelet derivatives between 1.0×10⁷ particles/kg and 1.0×10¹¹ particles/kg of the subject,
wherein the subject has been treated or is being treated with at least one anticoagulant agent, and at least one antiplatelet agent, and
wherein the at least one anticoagulant agent comprises a Factor XI inhibitor.

3. The method of claim 1, wherein the administering the dose comprises administering between 2 and 10 doses of the rehydrated platelet derivative composition, wherein each dose that is administered between 2 and 10 times is an amount of rehydrated platelet derivatives between 1.0×10⁷ particles/kg and 1.0×10¹¹ particles/kg of the subject.

4. The method of claim 1, wherein the at least one other antiplatelet agent is selected from the group consisting of cangrelor, ticagrelor, clopidogrel, prasugrel, eptifibatide, tirofiban, abciximab, terutroban, picotamide, ibuprofen, vorapaxar, atopaxar, cilostazol, prostaglandin E1, epoprostenol, dipyridamole, treprostinil sodium, and sarpogrelate.

5. The method of claim 1, wherein the at least one other antiplatelet agent is selected from the group consisting of cangrelor, ticagrelor, clopidogrel, and prasugrel.

6. The method of claim 2, wherein the at least one antiplatelet agent is one or two antiplatelet agents selected from the group consisting of aspirin, cangrelor, ticagrelor, clopidogrel, prasugrel, eptifibatide, tirofiban, abciximab, terutroban, picotamide, ibuprofen, vorapaxar, atopaxar, cilostazol, prostaglandin E1, epoprostenol, dipyridamole, treprostinil sodium, and sarpogrelate.

7. The method of claim 2, wherein the administering the dose comprises administering between 2 and 10 doses of the rehydrated platelet derivative composition, wherein each dose that is administered between 2 and 10 times is an amount of rehydrated platelet derivatives between 1.0×10⁷ particles/kg and 1.0×10¹¹ particles/kg of the subject

8. The method of claim 2, wherein the at least one antiplatelet agent comprises aspirin.

9. The method of claim 1, wherein the subject has a bleeding of grade 2, 3, or 4 based on the WHO bleeding scale before the administering of the rehydrated platelet derivative composition.

10. The method of claim 1, wherein after the administering of the rehydrated platelet derivative composition, the subject has a reduced bleeding such that the subject has one grade, two grades, or three grades less bleeding based on the WHO bleeding scale as compared to the bleeding before the administering of the rehydrated platelet derivative composition.

11. The method of claim 1, wherein the administering the rehydrated platelet derivative composition restores hemostasis in the subject.

12. The method of claim 2, wherein the subject is bleeding before the administering of the rehydrated platelet derivative composition, and the administering the rehydrated platelet derivative composition restores hemostasis in the subject.

13. The method of claim 1, wherein the subject is being treated with one anticoagulant agent that is a Factor XI inhibitor, aspirin, and one other antiplatelet agent.

14. The method of claim 2, wherein the subject is being treated with one anticoagulant agent that is a Factor XI inhibitor, and one antiplatelet agent.

15. The method of claim 1, wherein the dose comprises an amount of the rehydrated platelet derivatives between 1.0×10⁹ particles/kg and 1.0×10¹⁰ particles/kg of the subject.

16. The method of claim 1, wherein the anticoagulant agent, aspirin, or the other antiplatelet agent is present in the blood of the subject in an amount sufficient to cause a delay in the time which it takes to stop the bleeding in the subject.

17. The method of claim 1, wherein the anticoagulant agent, aspirin, or the antiplatelet agent is present at a highest concentration (Cmax) in the blood of the subject within 8, 7, 6, 5, 4, 3, 2 hours, or 1 hour of administering the dose of the rehydrated platelet derivatives.

18. The method of claim 2, wherein the subject is being treated with the anticoagulant agent, and the antiplatelet agent such that a recent dose of the anticoagulant agent, or the antiplatelet agent is administered to the subject within 1 week of administering the dose of the rehydrated platelet derivatives.

19. The method of claim 1, wherein the one or more saccharides comprise trehalose in an amount in the range of 10 mM to 500 mM, and polysucrose in an amount in the range of 3% to 10% w/v.

20. The method of claim 1, wherein the platelet derivative composition in the form of the powder is prepared by a process, comprising:
performing tangential flow filtration (TFF) of a platelet composition comprising platelets in a preparation agent comprising a buffering agent, trehalose in an amount in the range of 10 mM to 500 mM, and polysucrose in an amount in the range of 3% to 10%, thereby preparing a TFF-treated composition comprising at least 1000×10³ platelets/μl in an aqueous medium having less than or equal to 7.5% plasma protein and having less than 5.0% microparticles by scattering intensity;
freeze drying the TFF-treated composition comprising platelets in the aqueous medium to form a freeze-dried platelet derivative composition comprising platelet derivatives; and
heating the freeze-dried platelet derivative composition at a temperature in the range of 60° C. to 90° C. for at least 1 hour to not more than 36 hours to thermally treat the platelet derivatives in the freeze-dried platelet derivative composition to form the platelet derivative composition in the form of the powder.
